(19) United States
(12) Patent Application Publication
(10) Pub. No.: US 2010/0292255 A1
(43) Pub. Date: Nov. 18, 2010

(54) Therapeutic Compounds and Their Use in Treating Diseases and Disorders

(75) Inventors: Ashok C. Bajji, Salt Lake City, UT (US); Se-Ho Kim, Salt Lake City, UT (US); Rajendra Tangallapally, Salt Lake City, UT (US); Benjamin J. Markovitz, Columbus, OH (US); Richard Trovato, Salt Lake City, UT (US); Mark B. Anderson, Oakland, CA (US); Daniel A. Wettstein, Salt Lake City, UT (US); Mark D. Shenderovich, Salt Lake City, UT (US)

Correspondence Address: Myrexis, Inc.;c/o CPA Global, P.O. Box 52050, Minneapolis, MN, 55402, US

(73) Assignee: Myriad Pharmaceuticals, Inc., Salt Lake City, UT (US)

(21) Appl. No.: 12/780,828

(22) Filed: May 14, 2010

Related U.S. Application Data
(63) Continuation of application No. PCT/US2008/083636, filed on Nov. 14, 2008.
(60) Provisional application No. 60/988,069, filed on Nov. 14, 2007.

(51) Int. Cl. A61K 31/52; C07D 473/36; A61P 35/00

(52) U.S. Cl. 514/263.22; 544/276

(57) ABSTRACT

The invention provides novel therapeutic compounds, pharmaceutical compositions comprising these compounds, and methods for using these compounds and compositions to treat diseases and disorders, such as cancer.

### RELATED PRIORITY APPLICATION

This application is a continuation of International Application PCT/US2008/083636, filed Nov. 14, 2008, and published as WO 2009/065035 on May 22, 2009, which claims the benefit of U.S. Provisional Application Ser. No. 60/988,069 filed Nov. 14, 2007; both of which are hereby incorporated by reference herein in their entirety.

### FIELD OF THE INVENTION

The invention relates to novel therapeutic compounds that inhibit Heat Shock Protein 90 (Hsp90). The invention also relates to pharmaceutical compositions comprising these compounds, and methods of treating diseases and disorders, such as cancers, that respond favorably to the inhibition of Hsp90.

### BACKGROUND OF THE INVENTION

Cancer is prevalent: Among United States citizens that live to be 70 years older and older, the probability of developing invasive cancer is 38% for females and 46% for males. According to the American Cancer Society, there will be about 1.4 million new cases of cancer in the United States alone in 2006. Although the five year survival rate for all cancers is now 65%, up from about 50% in the mid-nineteen seventies, cancer remains a leading killer today. Indeed, it is estimated that 565,000 people in the United States will die from cancer in 2006. (American Cancer Society, Surveillance Research, 2006). Although numerous treatments are available for various cancers, the fact remains that many cancers remain incurable, untreatable, and/or become resistant to standard therapeutic regimens. Thus, there is a clear need for new cancer treatments employing novel chemotherapeutic compounds.

Inhibitors of the molecular chaperone protein Hsp90 are being developed as one class of pharmacological weaponry in the anticancer chemotherapeutic arsenal. Consequently, there is a clear need for additional, novel, Hsp90 inhibitors for the treatment of diseases and disorders, such as cancer, that respond favorably to the inhibition of Hsp90.

### BRIEF SUMMARY OF THE INVENTION

The invention relates to compounds of Formulae Ia, Ib, IIa, IIb, IIIa, IIIc, IVa, IVb, Va, Vb, VIIa, VIIb, VIIa, VIIb, VIIIa, VIIIb, IXa, IXb, Xa, Xb, XIa, XIb, XIIa, XIIb, XIIIa, XIIIb, XIVa, XIVb, XVa, XVb, XVIa, XVIb, XVIII, and XIX as described below, and to pharmaceutically-acceptable salts thereof. The invention also relates to pharmaceutical compositions comprising one or more compounds of these Formulae, and one or more pharmaceutically-acceptable carriers or excipients. The compounds of Formulae Ia, Ib, IIa, IIb, IIIa, IIIc, IVa, IVb, Va, Vb, VIIa, VIIb, VIIa, VIIb, VIIIa, VIIIb, IXa, IXb, Xa, Xb, XIa, XIb, XIIa, XIIb, XIIIa, XIIIb, XIVa, XIVb, XVa, XVb, XVIa, XVIb, XVIII, and XIX were discovered by the inventors to inhibit Hsp90. Consequently, the compounds of Formulae Ia, Ib, IIa, IIb, IIIa, IIIc, IVa, IVb, Va, Vb, VIIa, VIIb, VIIa, VIIb, VIIIa, VIIIb, IXa, IXb, Xa, Xb, XIa, XIb, XIIa, XIIb, XIIIa, XIIIb, XIVa, XIVb, XVa, XVb, XVIa, XVIb, XVIII, and XIX exhibit pharmacological activity that indicates that they are useful for the treatment of cancer, and other diseases and disorders that respond favorably to the inhibition of Hsp90.

Specifically, in one aspect the present invention relates to compounds according to Formulae Ia and Ib:

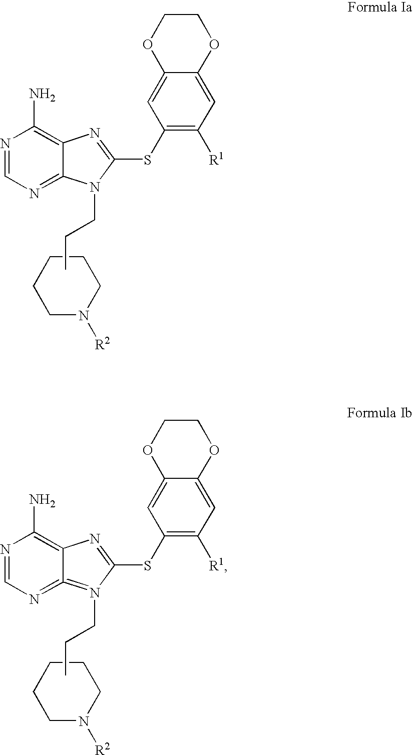

or pharmaceutically acceptable salts thereof; wherein

R¹ is halo, nitro, cyano, —C(═O)R¹¹ wherein R¹¹ is hydro or optionally substituted C₁-C₆ alkoxy; for example, R¹ can be —C(═O)H, —C(═O)OCH₃, or —C(═O)OC₂H₅; and

R² is selected from

(a) hydro;
(b) C₁-C₆ alkyl optionally substituted with 1, 2, 3, 4, or 5 substituents each independently chosen from the group of halo, hydroxyl, amino, cyano, and —C(═O)R²¹ wherein R²¹ is amino;
(c) —C(═O)R³, wherein R³ is selected from the group consisting of:

(1) hydro,

(2) C₁-C₁₀ (e.g., C₁-C₆) alkyl optionally substituted with 1, 2, 3, 4, or 5 substituents each independently chosen from the group of (i) halo, (ii) hydroxyl, (iii) thiol, (iv) cyano, (v) C₁-C₆ haloalkyl (e.g., trifluoromethyl), (vi) C₁-C₆ alkoxy (e.g., methoxy) optionally substituted with C₁-C₆ alkoxy (e.g., methoxy), (vii) C-amido, (viii) N-amido, (ix) sulfonyl, and (x) —N(R²²)(R²³) wherein R²² and R²³ are independently hydro, C₁-C₆ alkyl, sulfonyl, and C-carboxy,

(3) C₁-C₆ cycloalkyl optionally substituted with 1, 2, 3, 4, or 5 substituents each independently chosen from the group of halo, hydroxyl, amino, cyano, and C₁-C₆ haloalkyl (e.g., trifluoromethyl), and

(4) C₁-C₆ alkoxy optionally substituted with 1, 2, 3, 4, or 5 substituents each independently chosen from halo, hydroxyl, amino, cyano, and C₁-C₆ haloalkyl (e.g., trifluoromethyl),

(d) heterocycle or heterocyclylalkyl, optionally substituted with 1, 2, 3, 4, or 5 substituents independently chosen from halo, hydroxyl, amino, cyano, trihalomethyl, and C₁-C₄ alkyl optionally substituted with 1, 2, 3, or 4 substituents independently chosen from halo, hydroxyl, amino, cyano, C₁-C₆ haloalkyl (e.g., trifluoromethyl) (e.g., tetrazole-5-yl optionally substituted with 1, 2, 3, or 4 C₁-C₄ alkyl);
(e) sulfonyl; and
(f) optionally substituted heteroaryl;

with the proviso that the compound according to Formulae Ia, is not

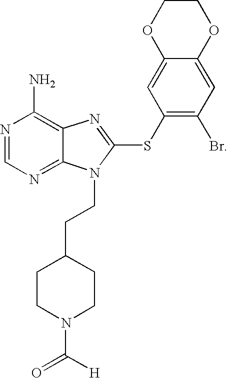

In another aspect, the present invention relates to compounds according to Formulae IIa and IIb:

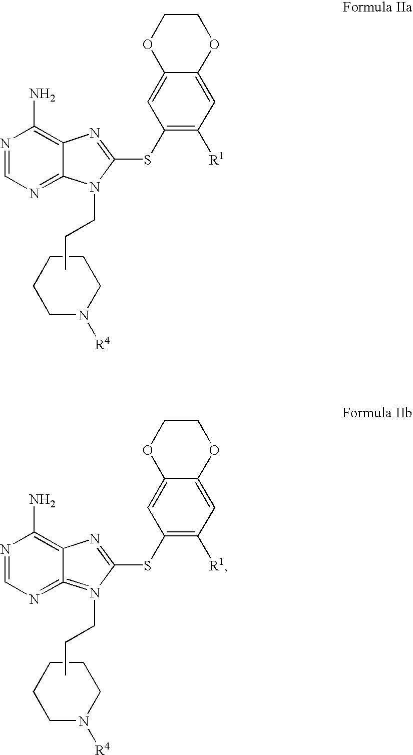

or pharmaceutically acceptable salts thereof; wherein

R¹ is halo, nitro, cyano, —C(═O)H, —C(═O)OCH₃, or —C(═O)OC₂H₅; and

R⁴ is

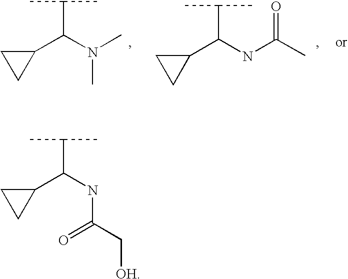

In another aspect, the present invention relates to compounds according to

Formulae IIIa and IIIb:

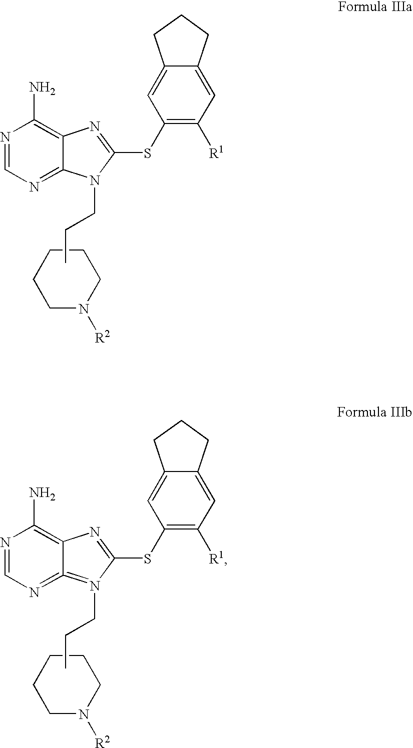

or pharmaceutically acceptable salts thereof; wherein

R¹ and R² are as defined above for the compounds of Formulae Ia and Ib.

In another aspect, the present invention relates to compounds according to Formulae IVa and IVb:

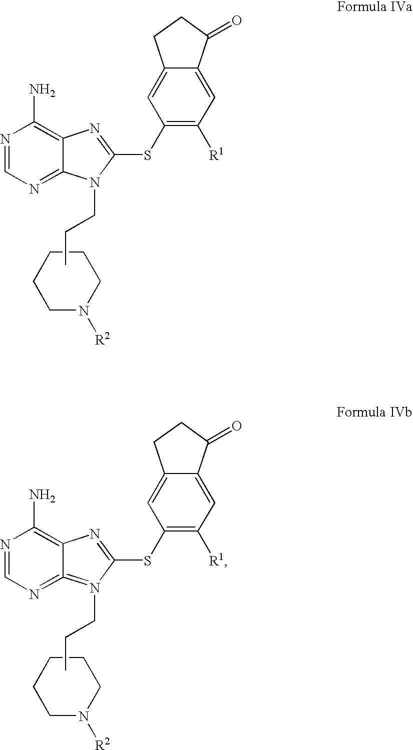

or pharmaceutically acceptable salts thereof; wherein

R¹ and R² are as defined above for the compounds of Formulae Ia and Ib.

In another aspect, the present invention relates to compounds according to Formulae Va and Vb:

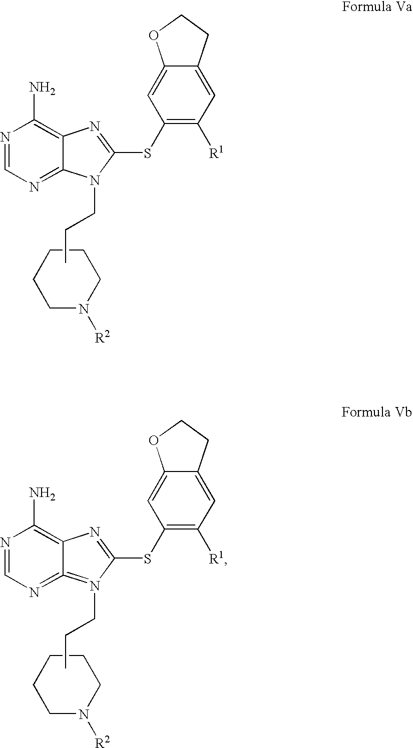

or pharmaceutically acceptable salts thereof; wherein

R¹ and R² are as defined above for the compounds of Formulae Ia and Ib.

In another aspect, the present invention relates to compounds according to Formulae VIa and VIb:

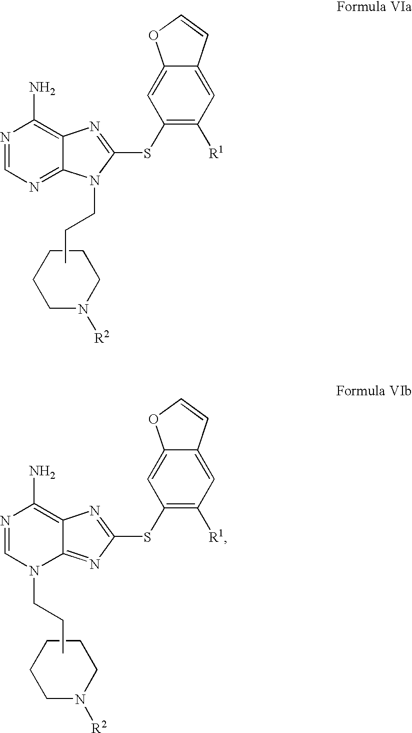

or pharmaceutically acceptable salts thereof; wherein

R¹ and R² are as defined above for the compounds of Formulae Ia and Ib.

In another aspect, the present invention relates to compounds according to Formulae VIIa and VIIb:

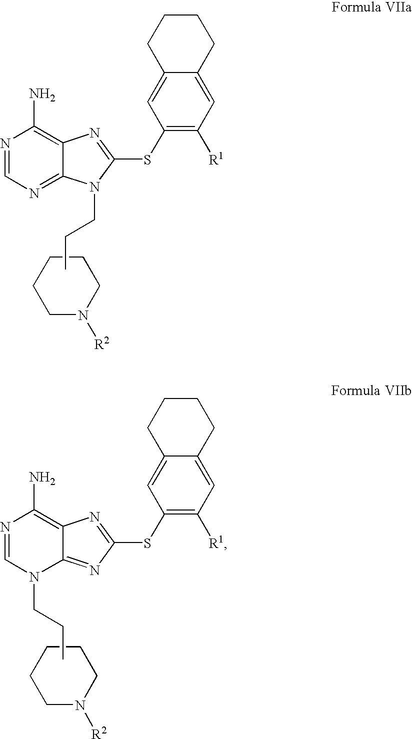

or pharmaceutically acceptable salts thereof; wherein

R¹ and R² are as defined above for the compounds of Formulae Ia and Ib.

In another aspect, the present invention relates to compounds according to Formulae VIIIa and VIIIb:

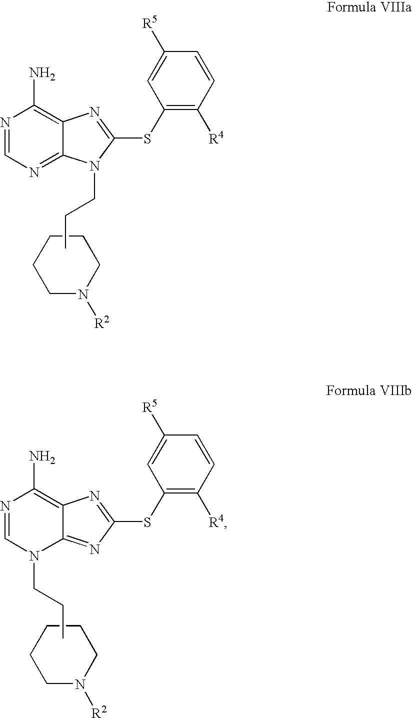

or pharmaceutically acceptable salts thereof; wherein

R² is as defined above for the compounds of Formulae Ia and Ib;

R⁴ is halo, trihalomethoxy, or cyano; and

R⁵ is ethyl, methoxy or nitro.

In another aspect, the present invention relates to compounds according to Formulae IXa and IXb:

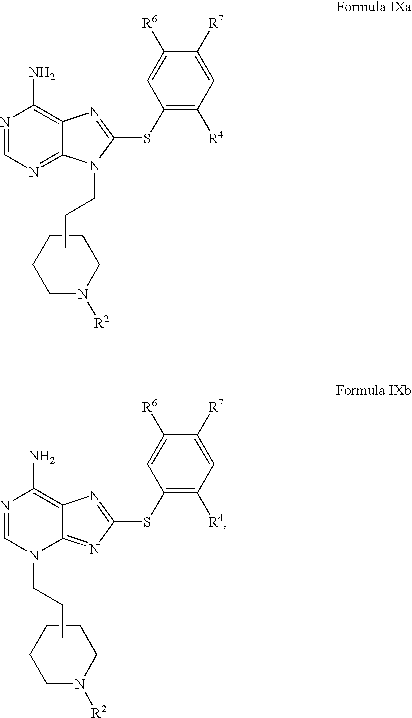

or pharmaceutically acceptable salts thereof; wherein

R² is as defined above for the compounds of Formulae Ia and Ib;

R⁴ is halo, methyl, trihalomethyl, or cyano; and

R⁶ and R⁷ are, independently, hydroxyl or methyl.

In another aspect, the present invention relates to compounds according to Formulae Xa and Xb:

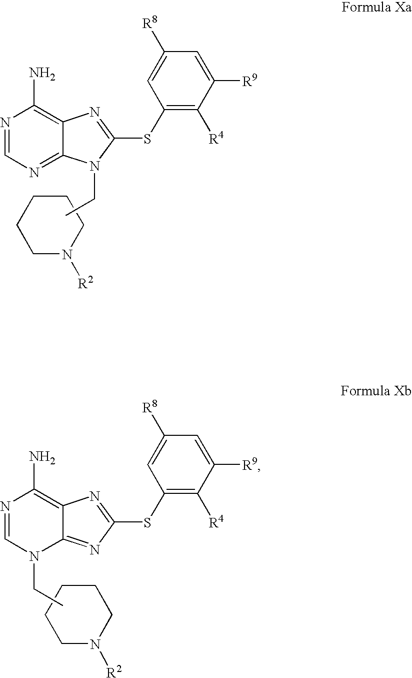

or pharmaceutically acceptable salts thereof; wherein

R² is as defined above for the compounds of Formulae Ia and Ib;

R⁴ is halo, trihalomethyl, or cyano;

R⁸ is hydroxyl, methoxy, or nitro; and

R⁹ is methoxy.

In another aspect, the present invention relates to compounds according to Formulae XIa and XIb:

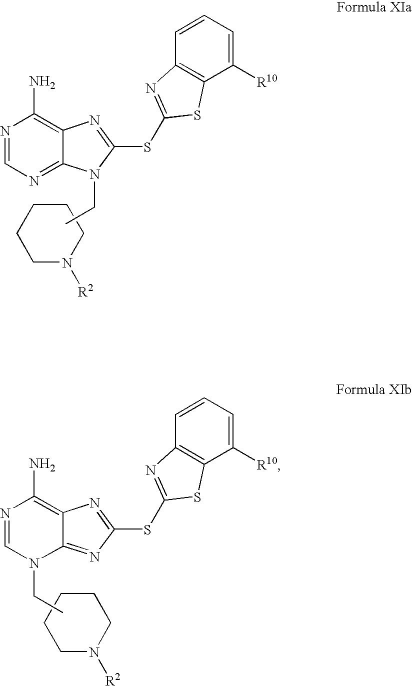

or pharmaceutically acceptable salts thereof; wherein

R² is as defined above for the compounds of Formulae Ia and Ib; and

R¹⁰ is halo.

In another aspect, the present invention relates to compounds according to Formulae XIIa and XIIb:

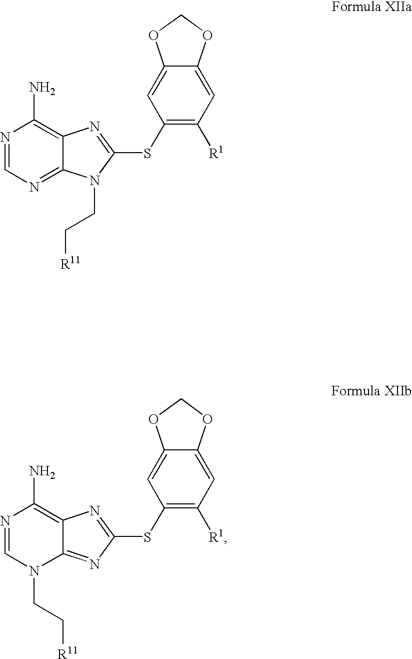

or pharmaceutically acceptable salts thereof; wherein

R¹ is halo, nitro, cyano, —C(═O)H, —C(═O)OCH₃, or —C(═O)OC₂H₅; and

R¹¹ is

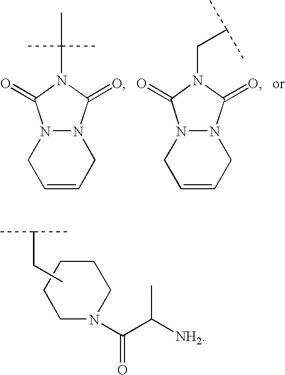

In another aspect, the present invention relates to compounds according to Formulae XIIIa and XIIIb:

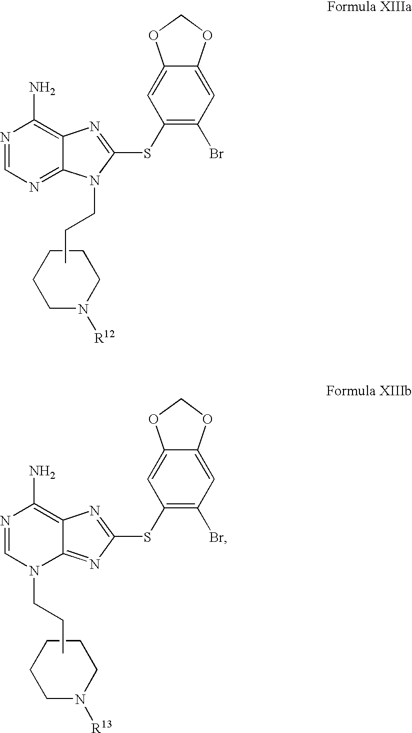

or pharmaceutically acceptable salts thereof; wherein

R¹² is hydro,

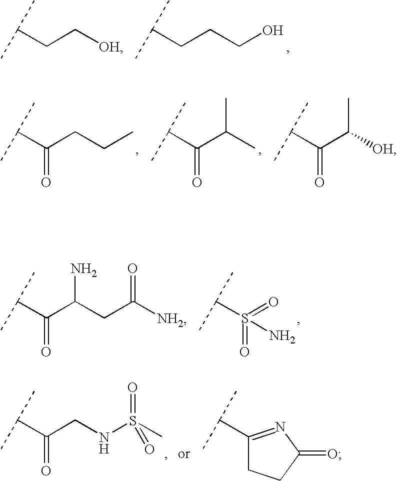

and

R¹³ is

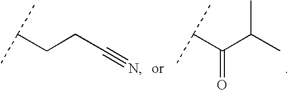

In another aspect, the present invention relates to compounds according to Formulae XIVa and XIVb:

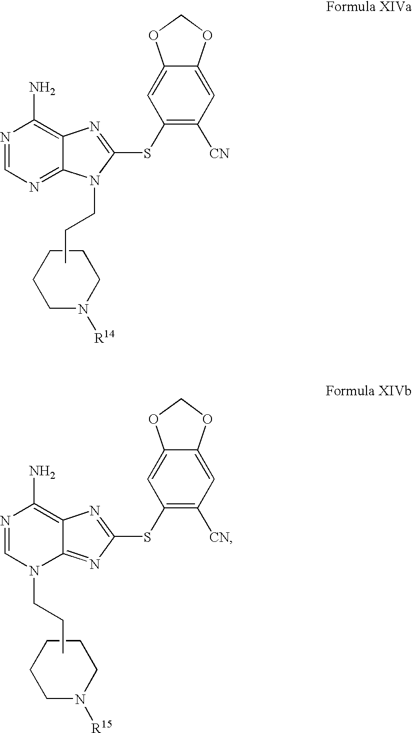

or pharmaceutically acceptable salts thereof; wherein

R¹⁴ is

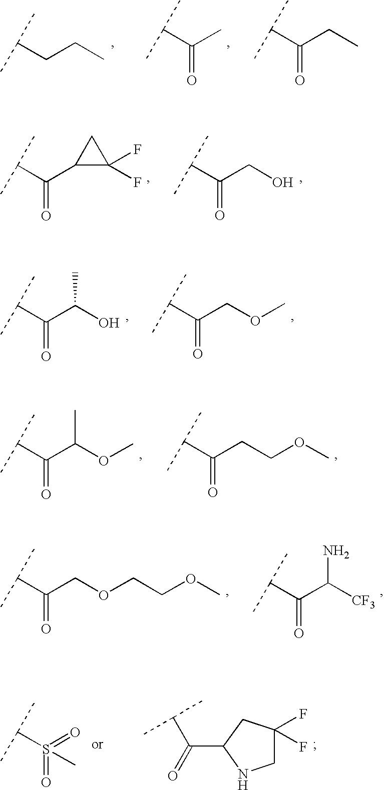

and

R¹⁵ is

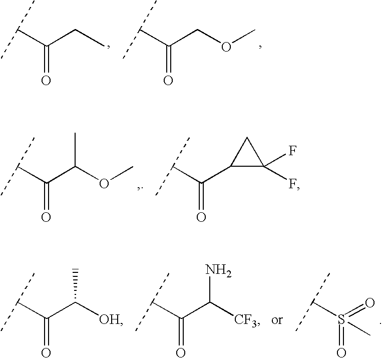

In another aspect, the present invention relates to compounds according to Formulae XVa, and XVb:

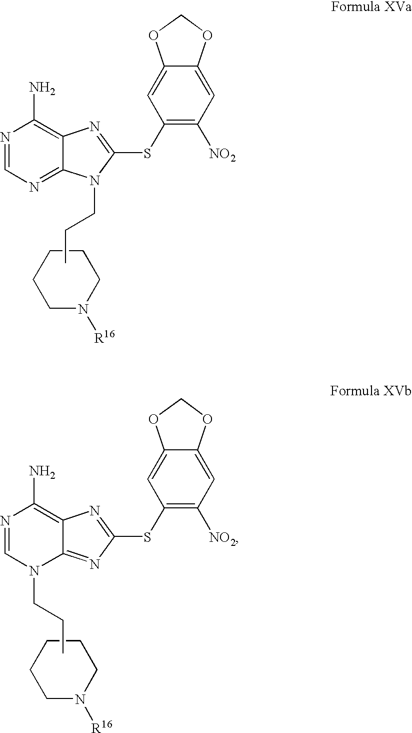

or pharmaceutically acceptable salts thereof; wherein

R¹⁶ is

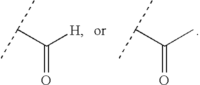

In another aspect, the present invention relates to compounds according to Formulae XVIa and XVIb:

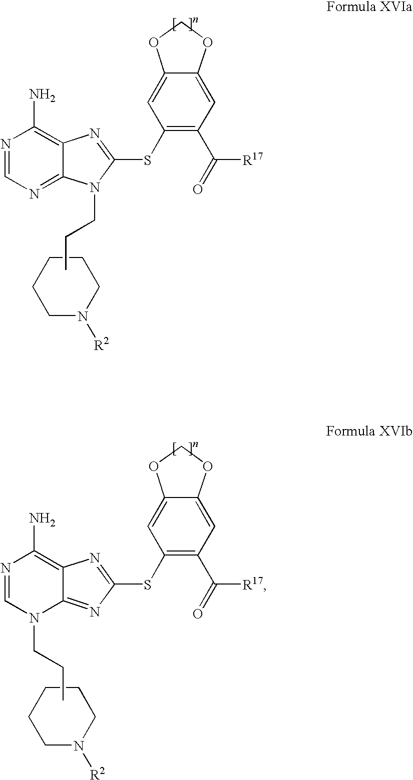

or pharmaceutically acceptable salts thereof; wherein

n is the integer 1 or 2;

R² is as defined above for the compounds of Formulae Ia and Ib; and

R¹⁷ is hydro, —N(CH₃)₂, or —OR¹⁸, wherein

R¹⁸ is C₁-C₆ alkyl (i.e., methyl or ethyl) optionally substituted with 1, 2, 3, 4, or 5 substituents chosen from halo, hydroxyl, amino, cyano, or trihalomethyl.

The invention also relates to compounds of Formulae XVIIa and XVIIb,

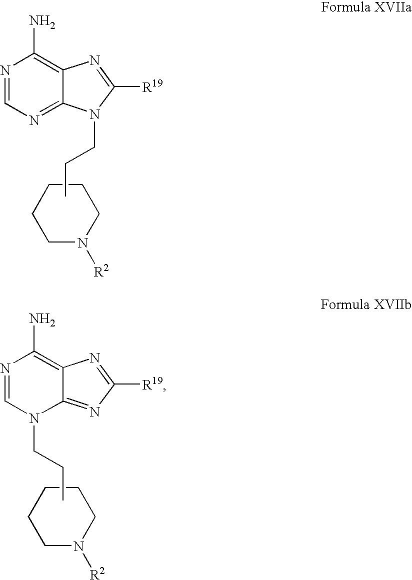

wherein

R² is as defined above for the compounds of Formulae Ia and Ib; and

R¹⁹ is hydro, bromo, or

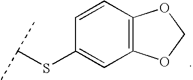

The compounds of Formulae XVIIa and XVIIb, as described above, can serve as intermediates in the synthesis of various specific compounds of Formulae Ia, Ib, IIa, IIb, IIIa, IIIc, IVa, IVb, Va, Vb, VIIa, VIIb, VIIa, VIIb, VIIIa, VIIIb, IXa, IXb, XIIIa, XIIIb, XIVa, XIVb, XVa, XVb, XVIa, XVIb, and XIX.

The invention also relates to compounds of Formula XVIII,

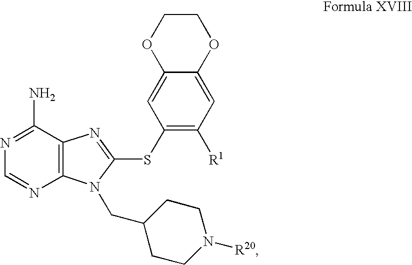

wherein

R¹ is as defined above for the compounds of Formulae Ia and Ib; and

R²⁰ is d- or l-alanine linked to the piperidinyl residue via a peptide bond, and

optionally substituted with:

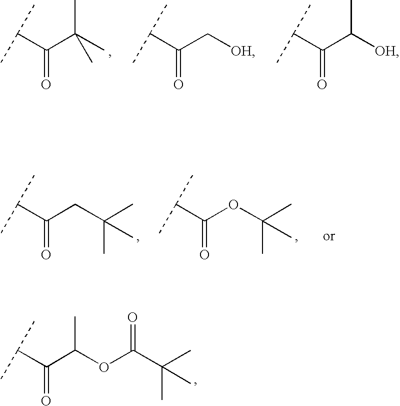

via a second peptide bond.

The invention also relates to compounds of Formula XIX,

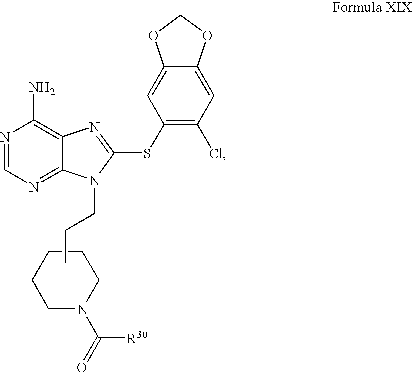

wherein R³⁰ is selected from

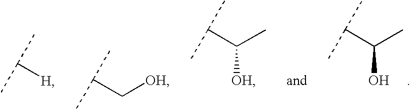

As noted above, the invention also includes pharmaceutical compositions having one or more compounds of Formulae Ia, Ib, IIa, IIb, IIIa, IIIc, IVa, IVb, Va, Vb, VIIa, VIIb, VIIa, VIIb, VIIIa, VIIIb, IXa, IXb, Xa, Xb, XIa, XIb, XIIa, XIIb, XIIIa, XIIIb, XIVa, XIVb, XVa, XVb, XVIa, XVIb, XVIII, and XIX, or a pharmaceutically-acceptable salt thereof, and one or more pharmaceutically acceptable carriers or excipients.

In one set of aspects, the present invention is directed to pharmaceutical compositions comprising the compounds of the invention, in particular, one or more compounds of Formulae Ia, Ib, IIa, IIb, IIIa, IIIc, IVa, IVb, Va, Vb, VIIa, VIIb, VIIa, VIIb, VIIIa, VIIIb, IXa, IXb, Xa, Xb, XIa, XIb, XIIa, XIIb, XIIIa, XIIIb, XIVa, XIVb, XVa, XVb, XVIa, XVIb, XVIII, and XIX, or a pharmaceutically-acceptable salt thereof, and one or more pharmaceutically acceptable carriers or excipients, for use in treatment or prevention of diseases or disorders that respond favorably to the inhibition of the 90 kDa heat shock protein, Hsp90, or orthologs and paralogs thereof. Such diseases and disorders are referred to herein as “Hsp90 inhibitor-sensitive diseases and disorders.”

In another set of aspects, the invention features a method of treating an individual having an Hsp90 inhibitor-sensitive disease or disorder by administering to the individual a pharmaceutical composition that comprises a pharmaceutically effective amount of one or more compounds of Formulae Ia, Ib, IIa, IIb, IIIa, IIIc, IVa, IVb, Va, Vb, VIIa, VIIb, VIIa, VIIb, VIIIa, VIIIb, IXa, IXb, Xa, Xb, XIa, XIb, XIIa, XIIb, XIIIa, XIIIb, XIVa, XIVb, XVa, XVb, XVIa, XVIb, XVIII, and XIX, or a pharmaceutically-acceptable salt thereof, and one or more pharmaceutically acceptable carriers or excipients.

In certain aspects, the invention provides a method for treating an individual having a Hsp90 inhibitor-sensitive disease or disorder chosen from inflammatory diseases, infections, autoimmune disorders, stroke, ischemia, cardiac disorders, neurological disorders, fibrogenetic disorders, proliferative disorders, tumors, leukemias, neoplasms, cancers, carcinomas, metabolic diseases, and malignant disease.

In yet set of aspects, the invention provides a method for treating an individual having a Hsp90 inhibitor-sensitive fibrogenetic disorder, such as, for example, scleroderma, polymyositis, systemic lupus, rheumatoid arthritis, liver cirrhosis, keloid formation, interstitial nephritis and pulmonary fibrosis.

In another set of aspects, the invention provides combination therapy comprising the administration of a pharmaceutically effective amount of a compound of Formulae Ia, Ib, IIa, IIb, IIIa, IIIc, IVa, IVb, Va, Vb, VIIa, VIIb, VIIa, VIIb, VIIIa, VIIIb, IXa, IXb, Xa, Xb, XIa, XIb, XIIa, XIIb, XIIIa, XIIIb, XIVa, XIVb, XVa, XVb, XVIa, XVIb, XVIII, and XIX, or a pharmaceutically-acceptable salt thereof, or a polymorph, solvate, ester, tautomer, diastereomer, enantiomer, or a pharmaceutically-acceptable salt or prodrug thereof, according to any of the preceding aspects or embodiments, and at least one therapeutic agent selected from the group of cytotoxic agents, anti-angiogenesis agents and anti-neoplastic agents. The anti-neoplastic agent may be selected from the group of alkylating agents, anti-metabolites, epidophyllotoxins antineoplastic enzymes, topoisomerase inhibitors, procarbazines, mitoxantrones, platinum coordination complexes, biological response modifiers and growth inhibitors, hormonal/anti-hormonal therapeutic agents, and haematopoietic growth factors.

The present invention also includes a therapeutic method comprising administering to an animal (e.g., a patient, in need of such treatment) a therapeutically effective amount of one or more compounds of Formulae Ia, Ib, IIa, IIb, IIIa, IIIc, IVa, IVb, Va, Vb, VIIa, VIIb, VIIa, VIIb, VIIIa, VIIIb, IXa, IXb, Xa, Xb, XIa, XIb, XIIa, XIIb, XIIIa, XIIIb, XIVa, XIVb, XVa, XVb, XVIa, XVIb, XVIII, and XIX, and/or a pharmaceutically acceptable salt thereof.

In certain aspects of the present invention, the therapeutic methods are useful in the treatment of Hsp90 inhibitor-sensitive cancers, which comprise a group of diseases characterized by the uncontrolled growth and spread of abnormal cells that respond to treatment with Hsp90 inhibitors. Such Hsp90 inhibitor-sensitive cancers can include, but are not limited to, Hodgkin's disease, non-Hodgkin's lymphoma, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, and prostatic carcinoma.

The materials, methods, and examples recited herein are illustrative only and not intended to be limiting. Other features and advantages of the invention will be apparent from the following detailed description, and from the claims.

### DETAILED DESCRIPTION OF THE INVENTION

### 1. Definitions

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control.

The term “bioisostere,” as used herein, generally refers to compounds or moieties that have chemical and physical properties producing broadly similar biological properties. For example, —COOH bioisosteres include, but are not limited to, a carboxylic acid ester, amide, tetrazole, oxadiazole, isoxazole, hydroxythiadiazole, thiazolidinedione, oxazolidinedione, sulfonamide, sulfonylcarboxamide, phosphonic acid, phosphonamide, phosphinic acid, sulfonic acid, acyl sulfonamide, mercaptoazole, and cyanamide.

As used herein, the term “alkyl” as employed herein by itself or as part of another group refers to a saturated aliphatic hydrocarbon straight chain or branched chain group having, unless otherwise specified, 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms). An alkyl group may be in unsubstituted form or substituted form with one or more substituents (generally one to three substitutents except in the case of halogen substituents, e.g., perchloro). For example, a C₁₋₆ alkyl group (“lower alkyl”) refers to a straight or branched aliphatic group containing 1 to 6 carbon atoms (e.g., methyl, ethyl, propyl, isopropyl, sec-butyl, tert-butyl, isobutyl, n-butyl, 3-pentyl, hexyl, etc.), which may be optionally substituted.

The term “alkenyl” as employed herein by itself or as part of another group means a straight or branched chain radical of 2-10 carbon atoms, unless the chain length is limited thereto, including at least one double bond between two of the carbon atoms in the chain. An alkenyl group may be in unsubstituted form or substituted form with one or more substituents (generally one to three substitutents except in the case of halogen substituents, e.g., perchloro or perfluoroalkyls). For example, a C₁₋₆ alkenyl group refers to a straight or branched chain radical containing 1 to 6 carbon atoms and having at least one double bond between two of the carbon atoms in the chain (e.g., ethenyl, 1-propenyl, 2-propenyl, 2-methyl-1-propenyl, 1-butenyl and 2-butenyl, which may be optionally substituted).

The term “alkynyl” as used herein by itself or as part of another group means a straight or branched chain radical of 2-10 carbon atoms, unless the chain length is limited thereto, wherein there is at least one triple bond between two of the carbon atoms in the chain. An alkynyl group may be in unsubstituted form or substituted form with one or more substituents (generally one to three substitutents except in the case of halogen substituents, e.g., perchloro or perfluoroalkyls). For example, a C₁₋₆ alkynyl group refers to a straight or branched chain radical containing 1 to 6 carbon atoms and having at least one triple bond between two of the carbon atoms in the chain (e.g., ethynyl, 1-propynyl, 1-methyl-2-propynyl, 2-propynyl, 1-butynyl and 2-butynyl, which may be optionally substituted).

The term “carbocycle” as used herein by itself or as part of another group means cycloalkyl and non-aromatic partially saturated carbocyclic groups such as cycloalkenyl and cycloalkynyl. A carbocycle may be in unsubstituted form or substituted form with one or more substituents so long as the resulting compound is sufficiently stable and suitable for the treatment method of the present invention.

The term “cycloalkyl” as used herein by itself or as part of another group refers to a fully saturated 3- to 8-membered cyclic hydrocarbon ring (i.e., a cyclic form of an unsubstituted alkyl) alone (“monocyclic cycloalkyl”), or fused to another cycloalkyl, cycloalkynyl, cycloalkenyl, heterocycle, aryl or heteroaryl ring (i.e., sharing an adjacent pair of carbon atoms with such other rings) (“polycyclic cycloalkyl”). Thus, a cycloalkyl may exist as a monocyclic ring, bicyclic ring, polycyclic or a spiral ring. When a cycloalkyl is recited as a substituent on a chemical entity, it is intended that the cycloalkyl moiety is attached to the entity through a carbon atom within the fully saturated cyclic hydrocarbon ring of the cycloalkyl. In contrast, a substituent on a cycloalkyl can be attached to any carbon atom of the cycloalkyl. A cycloalkyl may be in unsubstituted form or substituted form with one or more substituents so long as the resulting compound is sufficiently stable and suitable for the treatment method of the present invention. Examples of cycloalkyl groups include cyclopropyl, cyclobutyl, cyclopentyl, cyclohexyl and cycloheptyl.

The term “cycloalkenyl” as used herein by itself or as part of another group refers to a non-aromatic partially saturated 3- to 8-membered cyclic hydrocarbon ring (i.e., a cyclic form of an unsubstituted alkenyl) alone (“monocyclic cycloalkenyl”), or fused to another cycloalkyl, cycloalkynyl, cycloalkenyl, heterocycle, aryl or heteroaryl ring (i.e., sharing an adjacent pair of carbon atoms with such other rings) (“polycyclic cycloalkenyl”). Thus, a cycloalkenyl may exist as a monocyclic ring, bicyclic ring, polycyclic or a spiral ring. When a cycloalkenyl is recited as a substituent on a chemical entity, it is intended that the cycloalkenyl moiety is attached to the entity through a carbon atom within the fully saturated cyclic hydrocarbon ring of the cycloalkenyl. In contrast, a substituent on a cycloalkenyl can be attached to any carbon atom of the cycloalkyl. A cycloalkenyl group may be unsubstituted or substituted with one or more substitutents. Examples of cycloalkenyl groups include cyclopentenyl, cycloheptenyl and cyclooctenyl.

The term “heterocycle” (or “heterocyclyl” or “heterocyclic”) as used herein by itself or as part of another group means a saturated or partially saturated 3- to 7-membered non-aromatic cyclic ring formed with carbon atoms and from one to four heteroatoms independently selected from the group consisting of O, N, and S, wherein the nitrogen and sulfur heteroatoms can be optionally oxidized, and the nitrogen can be optionally quaternized (“monocyclic heterocycle”). The term “heterocycle” also encompasses a group having the non-aromatic heteroatom-containing cyclic ring above fused to another monocyclic cycloalkyl, cycloalkynyl, cycloalkenyl, heterocycle, aryl or heteroaryl ring (i.e., sharing an adjacent pair of carbon atoms with such other rings) (“polycyclic heterocycle”). Thus, a heterocycle may exist as a monocyclic ring, bicyclic ring, polycyclic or a spiral ring. When a heterocycle is recited as a substituent on a chemical entity, it is intended that the heterocycle moiety is attached to the entity through an atom within the saturated or partially saturated ring of the heterocycle. In contrast, a substituent on a heterocycle can be attached to any suitable atom of the heterocycle. In a “saturated heterocycle” the non-aromatic heteroatom-containing cyclic ring described above is fully saturated, whereas a “partially saturated heterocyle” contains one or more double or triple bonds within the non-aromatic heteroatom-containing cyclic ring regardless of the other ring it is fused to. A heterocycle may be in unsubstituted form or substituted form with one or more substituents so long as the resulting compound is sufficiently stable and suitable for the treatment method of the present invention.

Some examples of saturated or partially saturated heterocyclic groups include tetrahydrofuranyl, pyranyl, piperidinyl, piperazinyl, pyrrolidinyl, imidazolidinyl, imidazolinyl, indolinyl, isoindolinyl, quinuclidinyl, morpholinyl, isochromanyl, chromanyl, pyrazolidinyl, pyrazolinyl, tetronoyl and tetramoyl groups.

As used herein, “aryl” by itself or as part of another group means an all-carbon aromatic ring with up to 7 carbon atoms in the ring (“monocylic aryl”). In addition to monocyclic aromatic rings, the term “aryl” also encompasses a group having the all-carbon aromatic ring above fused to another cycloalkyl, cycloalkynyl, cycloalkenyl, heterocycle, aryl or heteroaryl ring (i.e., sharing an adjacent pair of carbon atoms with such other rings) (“polycyclic aryl”). When an aryl is recited as a substituent on a chemical entity, it is intended that the aryl moiety is attached to the entity through an atom within the all-carbon aromatic ring of the aryl. In contrast, a substituent on an aryl can be attached to any suitable atom of the aryl. Examples, without limitation, of aryl groups are phenyl, naphthalenyl and anthracenyl. An aryl may be in unsubstituted form or substituted form with one or more substituents so long as the resulting compound is sufficiently stable and suitable for the treatment method of the present invention.

The term “heteroaryl” as employed herein refers to a stable aromatic ring having up to 7 atoms with 1, 2, 3 or 4 heteroactoms which are oxygen, nitrogen or sulfur or a combination thereof (“monocylic heteroaryl”). In addition to monocyclic hetero aromatic rings, the term “heteroaryl” also encompasses a group having the monocyclic hetero aromatic ring above fused to another cycloalkyl, cycloalkynyl, cycloalkenyl, heterocycle, aryl or heteroaryl ring (i.e., sharing an adjacent pair of carbon atoms with such other rings) (“polycyclic heteroaryl”). When a heteroaryl is recited as a substituent on a chemical entity, it is intended that the heteroaryl moiety is attached to the entity through an atom within the hetero aromatic ring of the heteroaryl. In contrast, a substituent on a heteroaryl can be attached to any suitable atom of the heteroaryl. A heteroaryl may be in unsubstituted form or substituted form with one or more substituents so long as the resulting compound is sufficiently stable and suitable for the treatment method of the present invention.

Useful heteroaryl groups include thienyl (thiophenyl), benzo[b]thienyl, naphtho[2,3-b]thienyl, thianthrenyl, furyl (furanyl), isobenzofuranyl, chromenyl, xanthenyl, phenoxanthiinyl, pyrrolyl, including without limitation 2H-pyrrolyl, imidazolyl, pyrazolyl, pyridyl (pyridinyl), including without limitation 2-pyridyl, 3-pyridyl, and 4-pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, β-carbolinyl, phenanthridinyl, acrindinyl, perimidinyl, phenanthrolinyl, phenazinyl, isothiazolyl, phenothiazinyl, isoxazolyl, furazanyl, phenoxazinyl, 1,4-dihydroquinoxaline-2,3-dione, 7-amino-isocoumarin, pyrido[1,2-c]pyrimidin-4-one, pyrazolo[1,5-c]pyrimidinyl, including without limitation pyrazolo[1,5-c]pyrimidin-3-yl, 1,2-benzoisoxazol-3-yl, benzimidazolyl, 2-oxindolyl and 2-oxobenzimidazolyl. Where the heteroaryl group contains a nitrogen atom in a ring, such nitrogen atom may be in the form of an N-oxide, e.g., a pyridyl N-oxide, pyrazinyl N-oxide and pyrimidinyl N-oxide.

As used herein, the term “halo” refers to chloro, fluoro, bromo, and iodo.

As used herein, the term “hydro” refers to a hydrogen atom (—H group).

As used herein, the term “hydroxyl” refers to an —OH group.

As used herein, unless otherwise specified, the term “alkoxy” refers to a —O—C₁₋₁₂ alkyl.

As used herein, the term “cycloalkyloxy” refers to an —O-cycloalkyl group.

As used herein, the term “aryloxy” refers to an —O-aryl group.

As used herein, the term “heteroaryloxy” refers to both an —O-heteroaryl group.

Useful acyloxy groups are any C₁₋₆ acyl (alkanoyl) attached to an oxy (—O—) group, e.g., formyloxy, acetoxy, propionoyloxy, butanoyloxy, pentanoyloxy and hexanoyloxy. An acyloxy group may be unsubstituted or substituted form with one or more substituents so long as the resulting compound is sufficiently stable and suitable for the treatment method of the present invention.

As used herein, the term “mercapto” group refers to an —SH group.

As used herein, the term “alkylthio” group refers to an —S-alkyl group.

As used herein, the term “arylthio” group refers to both an —S-aryl group.

The term “arylalkyl” is used herein to mean an above-defined alkyl group substituted by an aryl group defined above. Examples of arylalkyl groups include benzyl, phenethyl and naphthylmethyl, etc. An arylalkyl group may be unsubstituted or substituted with one or more substituents so long as the resulting compound is sufficiently stable and suitable for the treatment method of the present invention.

The term “heteroarylalkyl” is used herein to mean an alkyl group defined above substituted by any heteroaryl groups. A heteroarylalkyl may be unsubstituted or substituted with one or more substituents so long as the resulting compound is sufficiently stable and suitable for the treatment method of the present invention.

The term “arylalkenyl” is used herein to mean an alkenyl group defined above substituted by any aryl groups defined above.

The term “heteroarylalkenyl” is used herein to mean any of the above-defined alkenyl groups substituted by any of the above-defined heteroaryl groups.

The term “arylalkynyl” is used herein to mean any of the above-defined alkynyl groups substituted by any of the above-defined aryl groups.

The term “heteroarylalkynyl” is used herein to mean any of the above-defined alkynyl groups substituted by any of the above-defined heteroaryl groups.

The term “aryloxy” is used herein to mean aryl-O— wherein aryl is as defined above. Useful aryloxy groups include phenoxy and 4-methylphenoxy.

The term “heteroaryloxy” is used herein to mean heteroaryl-O— wherein heteroaryl is as defined above.

The term “arylalkoxy” is used herein to mean an alkoxy group substituted by an aryl group as defined above. Useful arylalkoxy groups include benzyloxy and phenethyloxy.

“Heteroarylalkoxy” is used herein to mean any of the above-defined alkoxy groups substituted by any of the above-defined heteroaryl groups.

“Haloalkyl” means an alkyl group substituted by one or more (1, 2, 3, 4, 5 or 6) fluorine, chlorine, bromine or iodine atoms, e.g., fluoromethyl, difluoromethyl, trifluoromethyl, pentafluoroethyl, 1,1-difluoroethyl, chloromethyl, chlorofluoromethyl and trichloromethyl groups.

Useful acylamino (acylamido) groups are any C₁₋₆ acyl (alkanoyl) attached to an amino nitrogen which is in turn attached to the main structure, e.g., acetamido, chloroacetamido, propionamido, butanoylamido, pentanoylamido and hexanoylamido, as well as aryl-substituted C₁₋₆ acylamino groups, e.g., benzoylamido, and pentafluorobenzoylamido.

As used herein, the term “carbonyl” group refers to a —C(═O)R″ group, where R″ is selected from the group consisting of hydro, alkyl, cycloalkyl, aryl, heteroaryl (bonded through a ring carbon) and heterocyclic (bonded through a ring carbon), as defined herein.

As used herein, the term “aldehyde” group refers to a carbonyl group where R″ is hydro.

As used herein, the term “cycloketone” refer to a cycloalkyl group in which one of the carbon atoms which form the ring has a “═O” bonded to it; i.e. one of the ring carbon atoms is a —C(═O)— group.

As used herein, the term “thiocarbonyl” group refers to a —C(═S)R″ group, with R″ as defined herein.

As used herein, the term “O-carboxy” group refers to a R″C(═O)O— group, with R″ as defined herein.

As used herein, the term “C-carboxy” group refers to a —C(═O)OR″ groups with R″ as defined herein.

As used herein, the term “ester” is a C-carboxy group, as defined herein, wherein R″ defined above except that it is not hydro (e.g., methyl, ethyl, lower alkyl).

As used herein, the term “C-carboxy salt” refers to a —C(═O)O⁻M⁺ group wherein M⁺ is selected from the group consisting of lithium, sodium, magnesium, calcium, potassium, barium, iron, zinc and quaternary ammonium.

As used herein, the term “acetyl” group refers to a —C(═O)CH₃ group.

As used herein, the term “carboxyalkyl” refers to —(CH₂)ᵣC(═O)OR″ wherein r is 1-6 and R″ is as defined above.

As used herein, the term “carboxyalkyl salt” refers to a —(CH₂)ᵣC(═O)O⁻M⁺ wherein M⁺ is selected from the group consisting of lithium, sodium, potassium, calcium, magnesium, barium, iron, zinc and quaternary ammonium.

As used herein, the term “carboxylic acid” refers to a C-carboxy group in which R″ is hydro.

As used herein, the term “trihalomethanesulfonyl” refers to a X₃CS(═O)₂— group with X is a halo as defined above.

As used herein, the term “cyano” refers to a —C≡N group.

As used herein, the term “cyanato” refers to a —CNO group.

As used herein, the term “isocyanato” refers to a —NCO group.

As used herein, the term “thiocyanato” refers to a —CNS group.

As used herein, the term “isothiocyanato” refers to a —NCS group.

As used herein, the term “sulfinyl” refers to a —S(═O)R″ group, with R″ as defined herein.

As used herein, the term “sulfonyl” refers to a —S(═O)₂R″ group, with R″ as defined herein.

As used herein, the term “sulfonamide” refers to a —S(═O)₂N(R¹⁷)(R¹⁸), with R¹⁷ and R¹⁸ as defined herein.

As used herein, the term “trihalomethanesulfonamido” refers to a X₃CS(═O)₂NR¹⁷— group with X is halo as defined above and R¹⁷ as defined herein.

As used herein, the term “O-carbamyl” refers to a —OC(═O)N(R¹⁷)(R¹⁸) group with R¹⁷ and R¹⁸ as defined herein.

As used herein, the term “N-carbamyl” refers to a R¹⁸OC(═O)NR¹⁷— group, with R¹⁷ and R¹⁸ as defined herein.

As used herein, the term “O-thiocarbamyl” refers to a —OC(═S)N(R¹⁷)(R¹⁸) group with R¹⁷ and R¹⁸ as defined herein.

As used herein, the term “N-thiocarbamyl” refers to a R¹⁷OC(═S)NR¹⁸— group, with R¹⁷ and R¹⁸ as defined herein.

As used herein, the term “amino” refers to an —N(R¹⁷)(R¹⁸) group, with R¹⁷ and R¹⁸ as defined herein.

As used herein, the term “aminoalkyl” refers to a moiety wherein an amino group as defined herein attached through the nitrogen atom to an alkyl group as defined above.

As used herein, the term “C-amido” refers to a —C(═O)N(R¹⁷)(R¹⁸) group with R¹⁷ and R¹⁸ as defined herein. An “N-amido” refers to a R¹⁷C(═O)NR¹⁸— group with R¹⁷ and R¹⁸ as defined herein.

As used herein, the term “C-amidoalkyl” refers to a —C₁₋₆ alkyl-CO₂N(R¹⁷)(R¹⁸) group with R¹⁷ and R¹⁸ as defined herein.

As used herein, the term “nitro” refers to a —NO₂ group.

As used herein, the term “quaternary ammonium” refers to a —N(R¹⁷)(R¹⁸)(R¹⁹) group wherein R¹⁷, R¹⁸, and R¹⁹ are as defined herein.

R¹⁷, R¹⁸, and R¹⁹ are independently selected from the group consisting of hydro and unsubstituted lower alkyl (i.e., C₁₋₆ alkyl).

As used herein, the term “methylenedioxy” refers to a —OCH₂O— group wherein the oxygen atoms are bonded to adjacent ring carbon atoms.

As used herein, the term “ethylenedioxy” refers to a —OCH₂CH₂O— group wherein the oxygen atoms are bonded to adjacent ring carbon atoms.

### 2. Therapeutic Compounds

In one aspect, the present invention relates to compounds according to Formulae Ia and Ib:

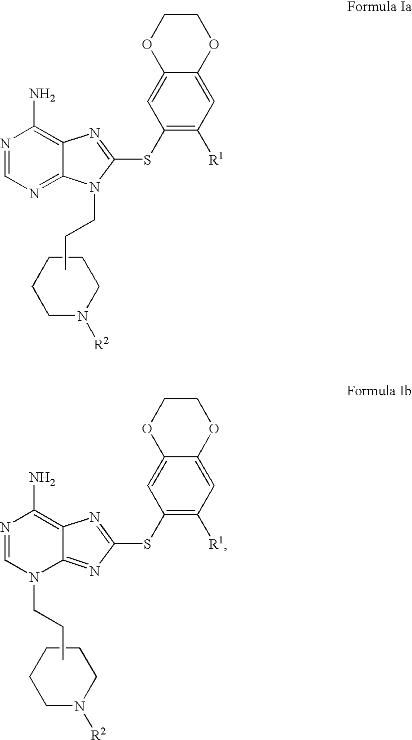

or pharmaceutically acceptable salts thereof; wherein

R¹ is halo, nitro, cyano, —C(═O)R¹¹ wherein R¹¹ is hydro or optionally substituted C₁-C₆ alkoxy; for example, R¹ can be —C(═O)H, —C(═O)OCH₃, or —C(═O)OC₂H₅; and

R² is selected from

(a) hydro;
(b) C₁-C₆ alkyl optionally substituted with 1, 2, 3, 4, or 5 substituents each independently chosen from the group of halo, hydroxyl, amino, cyano, and —C(═O)R²¹ wherein R²¹ is amino;
(c) —C(═O)R³, wherein R³ is selected from the group consisting of:

(1) hydro,

(2) C₁-C₁₀ (e.g., C₁-C₆) alkyl optionally substituted with 1, 2, 3, 4, or 5 substituents each independently chosen from the group of (i) halo, (ii) hydroxyl, (iii) thiol, (iv) cyano, (v) C₁-C₆ haloalkyl (e.g., trifluoromethyl), (vi) C₁-C₆ alkoxy (e.g., methoxy) optionally substituted with C₁-C₆ alkoxy (e.g., methoxy), (vii) C-amido, (viii) N-amido, (ix) sulfonyl, and (x) —N(R²²)(R²³) wherein R²² and R²³ are independently hydro, C₁-C₆ alkyl, sulfonyl, and C-carboxy,

(3) C₁-C₆ cycloalkyl optionally substituted with 1, 2, 3, 4, or 5 substituents each independently chosen from the group of halo, hydroxyl, amino, cyano, and C₁-C₆ haloalkyl (e.g., trifluoromethyl), and

(4) C₁-C₆ alkoxy optionally substituted with 1, 2, 3, 4, or 5 substituents each independently chosen from halo, hydroxyl, amino, cyano, and C₁-C₆ haloalkyl (e.g., trifluoromethyl),

(d) heterocycle or heterocyclylalkyl, optionally substituted with 1, 2, 3, 4, or 5 substituents independently chosen from halo, hydroxyl, amino, cyano, trihalomethyl, and C₁-C₄ alkyl optionally substituted with 1, 2, 3, or 4 substituents independently chosen from halo, hydroxyl, amino, cyano, C₁-C₆ haloalkyl (e.g., trifluoromethyl) (e.g., tetrazole-5-yl optionally substituted with 1, 2, 3, or 4 C₁-C₄ alkyl);
(e) sulfonyl; and
(f) optionally substituted heteroaryl;

with the proviso that the compound according to Formulae Ia, is not

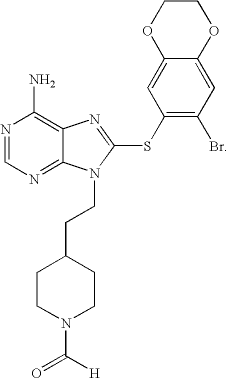

In a subset of this aspect of the present invention, R² is

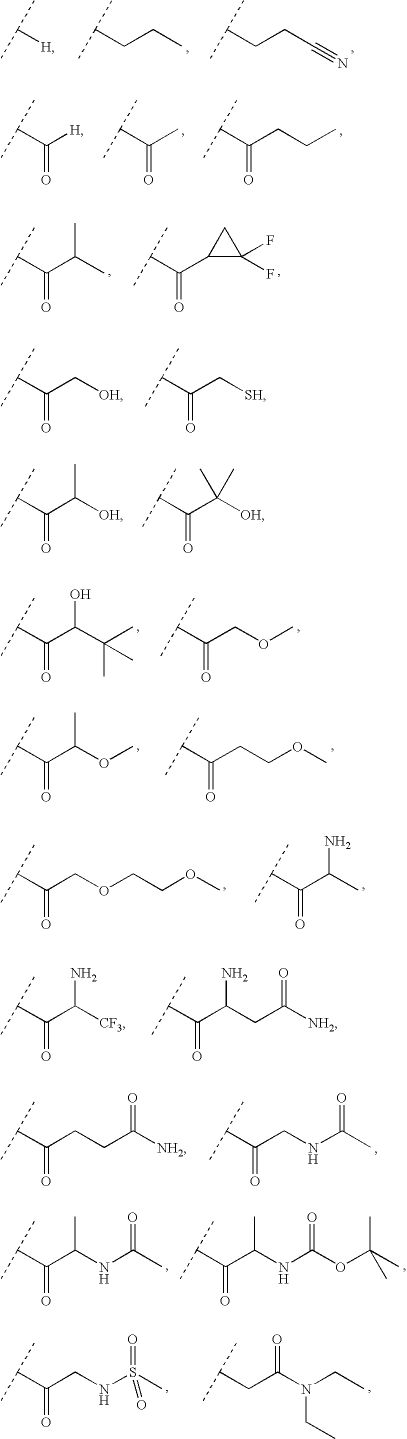

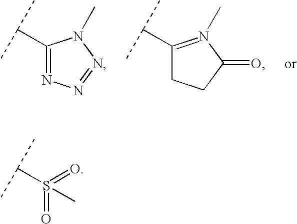

In specific embodiments of this aspect of the present invention, compounds according to Formulae Ia and Ib include, for example: 8-[(7-Bromo-2,3-dihydro-1,4-benzodioxin-6-yl)thio]-9-(2-{1-[(2-methoxyethoxy)acetyl]piperidin-4-yl}ethyl)-9H-purin-6-amine; 8-[(7-Bromo-2,3-dihydro-1,4-benzodioxin-6-yl)thio]-9-(2-{1-[(2S)-2-methoxypropanoyl]piperidin-4-yl}ethyl)-9H-purin-6-amine; 8-[(7-Bromo-2,3-dihydro-1,4-benzodioxin-6-yl)thio]-9-{2-[1-(3-methoxypropanoyl)piperidin-4-yl]ethyl}-9H-purin-6-amine; 8-[(7-Bromo-2,3-dihydro-1,4-benzodioxin-6-yl)thio]-9-{2-[1-(methoxyacetyl)piperidin-4-yl]ethyl}-9H-purin-6-amine; 8-[(7-Bromo-2,3-dihydro-1,4-benzodioxin-6-yl)thio]-9-(2-{1-[(2R)-2-methoxypropanoyl]piperidin-4-yl}ethyl)-9H-purin-6-amine; 8-[(7-Bromo-2,3-dihydro-1,4-benzodioxin-6-yl)thio]-9-(2-{1-[(2,2-difluorocyclopropyl)carbonyl]piperidin-4-yl}ethyl)-9H-purin-6-amine; 8-[(7-Bromo-2,3-dihydro-1,4-benzodioxin-6-yl)thio]-3-(2-{1-[(2,2-difluorocyclopropyl)carbonyl]piperidin-4-yl}ethyl)-3H-purin-6-amine; 8-[(7-Bromo-2,3-dihydro-1,4-benzodioxin-6-yl)thio]-9-{2-[1-(methylsulfonyl)piperidin-2-yl]ethyl}-9H-purin-6-amine; 8-[(7-Bromo-2,3-dihydro-1,4-benzodioxin-6-yl)thio]-3-{2-[1-(methylsulfonyl)piperidin-2-yl]ethyl}-3H-purin-6-amine; 8-[(7-Bromo-2,3-dihydro-1,4-benzodioxin-6-yl)thio]-9-{2-[1-(methylsulfonyl)piperidin-3-yl]ethyl}-9H-purin-6-amine; 8-[(7-Bromo-2,3-dihydro-1,4-benzodioxin-6-yl)thio]-3-{2-[1-(methylsulfonyl)piperidin-3-yl]ethyl}-3H-purin-6-amine; 8-[(7-Bromo-2,3-dihydro-1,4-benzodioxin-6-yl)thio]-9-[2-(1-propylpiperidin-2-yl)ethyl]-9H-purin-6-amine; 8-[(7-Bromo-2,3-dihydro-1,4-benzodioxin-6-yl)thio]-9-{2-[1-(methylsulfonyl)piperidin-4-yl]ethyl}-9H-purin-6-amine; 8-[(7-Bromo-2,3-dihydro-1,4-benzodioxin-6-yl)thio]-3-[2-(1-propylpiperidin-2-yl)ethyl]-3H-purin-6-amine; 8-[(7-Bromo-2,3-dihydro-1,4-benzodioxin-6-yl)thio]-3-{2-[1-(methylsulfonyl)piperidin-4-yl]ethyl}-3H-purin-6-amine; 8-[(7-Bromo-2,3-dihydro-1,4-benzodioxin-6-yl)thio]-9-{2-[1-(1-methyl-1H-tetrazol-5-yl)piperidin-4-yl]ethyl}-9H-purin-6-amine; 8-[(7-Bromo-2,3-dihydro-1,4-benzodioxin-6-yl)thio]-3-{2-[1-(1-methyl-1H-tetrazol-5-yl)piperidin-4-yl]ethyl}-3H-purin-6-amine; 8-[(7-Bromo-2,3-dihydro-1,4-benzodioxin-6-yl)thio]-9-[2-(1-isobutyrylpiperidin-4-yl)ethyl]-9H-purin-6-amine; 8-[(7-Bromo-2,3-dihydro-1,4-benzodioxin-6-yl)thio]-3-[2-(1-isobutyrylpiperidin-4-yl)ethyl]-3H-purin-6-amine; 2-[4-(2-{6-Amino-8-[(7-bromo-2,3-dihydro-1,4-benzodioxin-6-yl)thio]-9H-purin-9-yl}ethyl)piperidin-1-yl]-N,N-diethylacetamide; 2-[4-(2-{6-Amino-8-[(7-bromo-2,3-dihydro-1,4-benzodioxin-6-yl)thio]-3H-purin-3-yl}ethyl)piperidin-1-yl]-N,N-diethylacetamide; 7-{[6-Amino-9-(2-{1-[(2S)-2-methoxypropanoyl]piperidin-4-yl}ethyl)-9H-purin-8-yl]thio}-2,3-dihydro-1,4-benzodioxine-6-carbonitrile; 7-{[6-Amino-9-(2-{1-[(2R)-2-methoxypropanoyl]piperidin-4-yl}ethyl)-9H-purin-8-yl]thio}-2,3-dihydro-1,4-benzodioxine-6-carbonitrile; 7-[(6-Amino-9-{2-[1-(methoxyacetyl)piperidin-4-yl]ethyl}-9H-purin-8-yl)thio]-2,3-dihydro-1,4-benzodioxine-6-carbonitrile; 7-[(6-Amino-9-{2-[1-(methylsulfonyl)piperidin-2-yl]ethyl}-9H-purin-8-yl)thio]-2,3-dihydro-1,4-benzodioxine-6-carbonitrile; 7-[(6-Amino-9-{2-[1-(methylsulfonyl)piperidin-3-yl]ethyl}-9H-purin-8-yl)thio]-2,3-dihydro-1,4-benzodioxine-6-carbonitrile; 7-[(6-Amino-3-{2-[1-(methylsulfonyl)piperidin-3-yl]ethyl}-3H-purin-8-yl)thio]-2,3-dihydro-1,4-benzodioxine-6-carbonitrile; 7-[(6-Amino-9-{2-[1-(methylsulfonyl)piperidin-4-yl]ethyl}-9H-purin-8-yl)thio]-2,3-dihydro-1,4-benzodioxine-6-carbonitrile; 7-[(6-Amino-3-{2-[1-(methylsulfonyl)piperidin-4-yl]ethyl}-3H-purin-8-yl)thio]-2,3-dihydro-1,4-benzodioxine-6-carbonitrile; 7-({6-Amino-3-[2-(1-formylpiperidin-4-yl)ethyl]-3H-purin-8-yl}thio)-2,3-dihydro-1,4-benzodioxine-6-carbonitrile; 7-({6-Amino-9-[2-(1-formylpiperidin-4-yl)ethyl]-9H-purin-8-yl}thio)-2,3-dihydro-1,4-benzodioxine-6-carbonitrile; 7-[(6-Amino-9-{2-[1-(1-methyl-1H-tetrazol-5-yl)piperidin-4-yl]ethyl}-9H-purin-8-yl)thio]-2,3-dihydro-1,4-benzodioxine-6-carbonitrile; 7-[(6-Amino-3-{2-[1-(1-methyl-1H-tetrazol-5-yl)piperidin-4-yl]ethyl}-3H-purin-8-yl)thio]-2,3-dihydro-1,4-benzodioxine-6-carbonitrile; 7-({9-[2-(1-Acetylpiperidin-4-yl)ethyl]-6-amino-9H-purin-8-yl}thio)-2,3-dihydro-1,4-benzodioxine-6-carbonitrile; 4-(2-{6-Amino-8-[(7-nitro-2,3-dihydro-1,4-benzodioxin-6-yl)thio]-9H-purin-9-yl}ethyl)piperidine-1-carbaldehyde; 4-(2-{6-Amino-8-[(7-nitro-2,3-dihydro-1,4-benzodioxin-6-yl)thio]-3H-purin-3-yl}ethyl)piperidine-1-carbaldehyde; 9-[2-(1-Acetylpiperidin-4-yl)ethyl]-8-[(7-nitro-2,3-dihydro-1,4-benzodioxin-6-yl)thio]-9H-purin-6-amine; 3-[2-(1-Acetylpiperidin-4-yl)ethyl]-8-[(7-nitro-2,3-dihydro-1,4-benzodioxin-6-yl)thio]-3H-purin-6-amine; 3-[2-(1-Acetylpiperidin-4-yl)ethyl]-8-[(7-chloro-2,3-dihydro-1,4-benzodioxin-6-yl)thio]-3H-purin-6-amine; 9-[2-(1-Acetylpiperidin-4-yl)ethyl]-8-[(7-chloro-2,3-dihydro-1,4-benzodioxin-6-yl)thio]-9H-purin-6-amine; 8-[(7-Bromo-2,3-dihydro-1,4-benzodioxin-6-yl)thio]-9-[2-(1-butyrylpiperidin-4-yl)ethyl]-9H-purin-6-amine; 8-[(7-Bromo-2,3-dihydro-1,4-benzodioxin-6-yl)thio]-3-[2-(1-butyrylpiperidin-4-yl)ethyl]-3H-purin-6-amine; (2S)-1-[4-(2-{6-Amino-8-[(7-bromo-2,3-dihydro-1,4-benzodioxin-6-yl)thio]-9H-purin-9-yl}ethyl)piperidin-1-yl]-1-oxopropan-2-ol; (2S)-1-[4-(2-{6-amino-8-[(7-bromo-2,3-dihydro-1,4-benzodioxin-6-yl)thio]-3H-purin-3-yl}ethyl)piperidin-1-yl]-1-oxopropan-2-ol; 2-[4-(2-{6-Amino-8-[(7-bromo-2,3-dihydro-1,4-benzodioxin-6-yl)thio]-9H-purin-9-yl}ethyl)piperidin-1-yl]-2-oxoethanol; 2-[4-(2-{6-Amino-8-[(7-bromo-2,3-dihydro-1,4-benzodioxin-6-yl)thio]-3H-purin-3-yl}ethyl)piperidin-1-yl]-2-oxoethanol; 1-[4-(2-{6-Amino-8-[(7-bromo-2,3-dihydro-1,4-benzodioxin-6-yl)thio]-9H-purin-9-yl}ethyl)piperidin-1-yl]-2-methyl-1-oxopropan-2-ol; 1-[4-(2-{6-Amino-8-[(7-bromo-2,3-dihydro-1,4-benzodioxin-6-yl)thio]-3H-purin-3-yl}ethyl)piperidin-1-yl]-2-methyl-1-oxopropan-2-ol; 7-[(6-Amino-9-{2-[1-(2-hydroxy-2-methylpropanoyl)piperidin-4-yl]ethyl}-9H-purin-8-yl)thio]-2,3-dihydro-1,4-benzodioxine-6-carbonitrile; 7-{[6-Amino-9-(2-{1-[(2S)-2-hydroxypropanoyl]piperidin-4-yl}ethyl)-9H-purin-8-yl]thio}-2,3-dihydro-1,4-benzodioxine-6-carbonitrile; 7-{[6-Amino-3-(2-{1-[(2S)-2-hydroxypropanoyl]piperidin-4-yl}ethyl)-3H-purin-8-yl]thio}-2,3-dihydro-1,4-benzodioxine-6-carbonitrile; 7-({6-Amino-9-[2-(1-glycoloylpiperidin-4-yl)ethyl]-9H-purin-8-yl}thio)-2,3-dihydro-1,4-benzodioxine-6-carbonitrile; (2S)-1-[4-(2-{6-Amino-8-[(7-bromo-2,3-dihydro-1,4-benzodioxin-6-yl)thio]-9H-purin-9-yl}ethyl)piperidin-1-yl]-3,3-dimethyl-1-oxobutan-2-ol; (2S)-1-[4-(2-{6-Amino-8-[(7-bromo-2,3-dihydro-1,4-benzodioxin-6-yl)thio]-3H-purin-3-yl}ethyl)piperidin-1-yl]-3,3-dimethyl-1-oxobutan-2-ol; (2R)-1-[4-(2-{6-Amino-8-[(7-bromo-2,3-dihydro-1,4-benzodioxin-6-yl)thio]-9H-purin-9-yl}ethyl)piperidin-1-yl]-1-oxopropan-2-ol; 9-[2-(1-Acetylpiperidin-4-yl)ethyl]-8-[(7-bromo-2,3-dihydro-1,4-benzodioxin-6-yl)thio]-9H-purin-6-amine; 8-[(7-bromo-2,3-dihydro-1,4-benzodioxin-6-yl)thio]-9-(2-piperidin-4-ylethyl)-9H-purin-6-amine; (3S)-3-Amino-4-[4-(2-{6-amino-8-[(7-bromo-2,3-dihydro-1,4-benzodioxin-6-yl)thio]-9H-purin-9-yl}ethyl)piperidin-1-yl]-4-oxobutanamide; 4-[4-(2-{6-Amino-8-[(7-bromo-2,3-dihydro-1,4-benzodioxin-6-yl)thio]-9H-purin-9-yl}ethyl)piperidin-1-yl]-4-oxobutanamide; 5-[4-(2-{6-amino-8-[(7-bromo-2,3-dihydro-1,4-benzodioxin-6-yl)thio]-9H-purin-9-yl}ethyl)piperidin-1-yl]-3,4-dihydro-2H-pyrrol-2-one; N-{2-[4-(2-{6-Amino-8-[(7-bromo-2,3-dihydro-1,4-benzodioxin-6-yl)thio]-9H-purin-9-yl}ethyl)piperidin-1-yl]-2-oxoethyl}methanesulfonamide; N-{2-[4-(2-{6-Amino-8-[(7-bromo-2,3-dihydro-1,4-benzodioxin-6-yl)thio]-9H-purin-9-yl}ethyl)piperidin-1-yl]-2-oxoethyl}acetamide; N-{(1S)-2-[4-(2-{6-Amino-8-[(7-bromo-2,3-dihydro-1,4-benzodioxin-6-yl)thio]-9H-purin-9-yl}ethyl)piperidin-1-yl]-1-methyl-2-oxoethyl}acetamide; N-{(1R)-2-[4-(2-{6-Amino-8-[(7-bromo-2,3-dihydro-1,4-benzodioxin-6-yl)thio]-9H-purin-9-yl}ethyl)piperidin-1-yl]-1-methyl-2-oxoethyl}acetamide; 2-[4-(2-{6-Amino-8-[(7-bromo-2,3-dihydro-1,4-benzodioxin-6-yl)thio]-9H-purin-9-yl}ethyl)piperidin-1-yl]-2-oxoethanethiol; and 4-(2-{6-Amino-8-[(7-fluoro-2,3-dihydro-1,4-benzodioxin-6-yl)thio]-9H-purin-9-yl}ethyl)piperidine-1-carbaldehyde.

In another aspect, the present invention relates to compounds according to Formulae IIa and IIb:

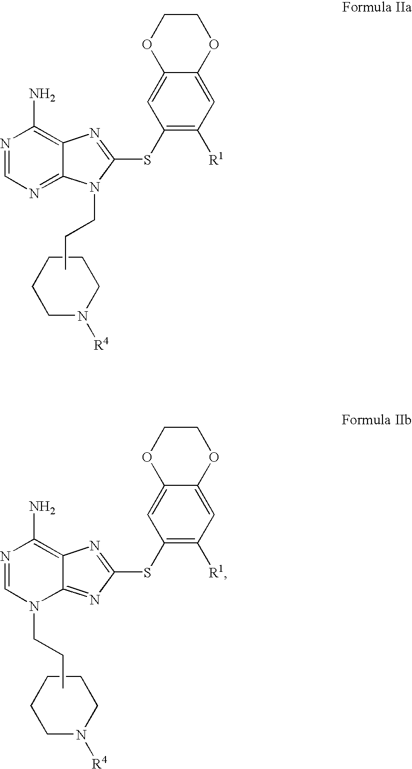

or pharmaceutically acceptable salts thereof; wherein

R¹ is halo, nitro, cyano, —C(═O)H, —C(═O)OCH₃, or —C(═O)OC₂H₅; and

R⁴ is

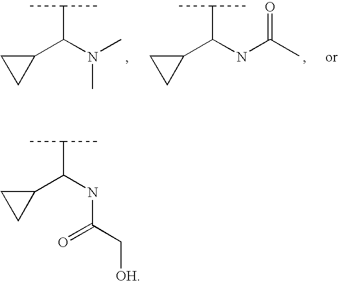

In specific embodiments of this aspect of the present invention, compounds according to Formulae IIa and IIb include, for example: N-{9-[2-(1-acetylpiperidin-4-yl)ethyl]-8-[(7-bromo-2,3-dihydro-1,4-benzodioxin-6-yl)thio]-9H-purin-6-yl}acetamide; 8-[(7-Bromo-2,3-dihydro-1,4-benzodioxin-6-yl)thio]-9-[3-cyclopropyl-3-(dimethylamino)propyl]-9H-purin-6-amine; or 8-[(7-bromo-2,3-dihydro-1,4-benzodioxin-6-yl)thio]-3-[3-cyclopropyl-3-(dimethylamino)propyl]-3H-purin-6-amine.

In another aspect, the present invention relates to compounds according to Formulae IIIa and IIIb:

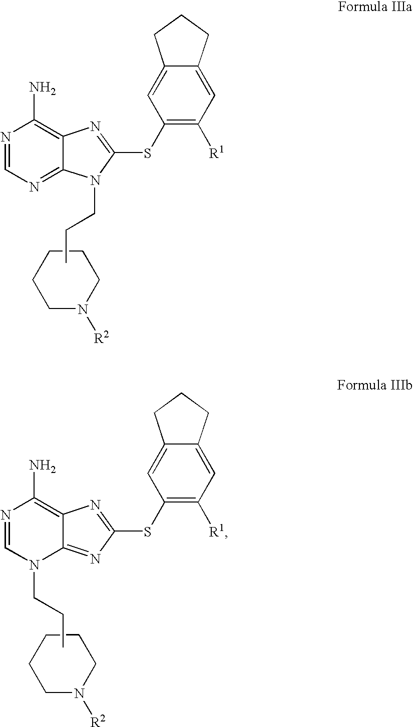

or pharmaceutically acceptable salts thereof; wherein

R¹ and R² are as defined above for the compounds of Formulae Ia and Ib.

In specific embodiments of this aspect of the present invention, compounds according to Formulae IIIa and Mb include, for example: 4-(2-{6-Amino-8-[(6-iodo-2,3-dihydro-1H-inden-5-yl)thio]-9H-purin-9-yl}ethyl)piperidine-1-carbaldehyde; 4-{2-[6-Amino-8-(6-bromo-indan-5-ylsulfanyl)-purin-9-yl]-ethyl}-piperidine-1-carbaldehyde; 4-{2-[6-Amino-8-(6-bromo-indan-5-ylsulfanyl)-purin-3-yl]-ethyl}-piperidine-1-carbaldehyde; (S)-1-(4-{2-[6-Amino-8-(6-bromo-indan-5-ylsulfanyl)-purin-9-yl]-ethyl}-piperidin-1-yl)-2-hydroxy-propan-1-one; and (S)-1-(4-{2-[6-Amino-8-(6-bromo-indan-5-ylsulfanyl)-purin-3-yl]-ethyl}-piperidin-1-yl)-2-hydroxy-propan-1-one.

In another aspect, the present invention relates to compounds according to Formulae IVa and IVb:

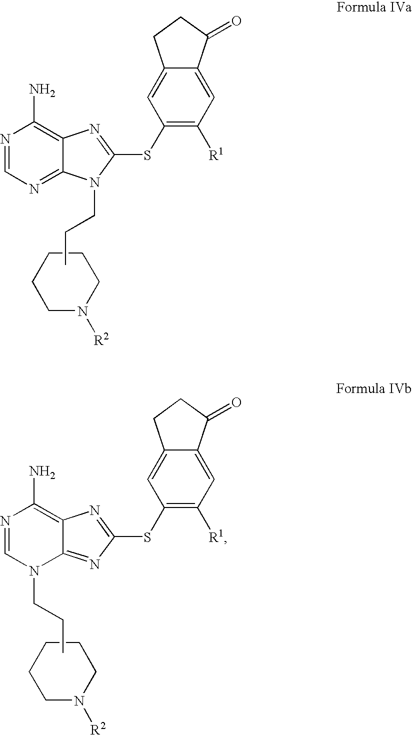

or pharmaceutically acceptable salts thereof; wherein

R¹ and R² are as defined above for the compounds of Formulae Ia and Ib.

In specific embodiments of this aspect of the present invention, compounds according to Formulae IVa and IVb include, for example: 6-({6-Amino-9-[2-(1-formylpiperidin-4-yl)ethyl]-9H-purin-8-yl}thio)-3-oxoindane-5-carbonitrile; 6-({6-amino-3-[2-(1-formylpiperidin-4-yl)ethyl]-3H-purin-8-yl}thio)-3-oxoindane-5-carbonitrile; 6-({6-Amino-3-[2-(1-propionylpiperidin-4-yl)ethyl]-3H-purin-8-yl}thio)-3-oxoindane-5-carbonitrile; 4-(2{6-Amino-8-[(6-bromo-1-oxo-2,3-dihydro-1H-inden-5-yl)thio]-9H-purin-9-yl}ethyl)piperidine-1-carbaldehyde; and 4-(2-{6-Amino-8-[(6-bromo-1-oxo-2,3-dihydro-1H-inden-5-yl)thio]-3H-purin-3-yl}ethyl)piperidine-1-carbaldehyde.

In another aspect, the present invention relates to compounds according to Formulae Va and Vb:

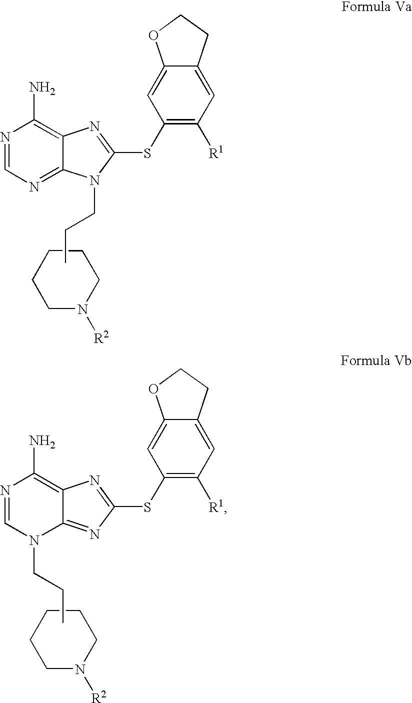

or pharmaceutically acceptable salts thereof; wherein

R¹ and R² are as defined above for the compounds of Formulae Ia and Ib.

In specific embodiments of this aspect of the present invention, compounds according to Formulae Va and Vb include, for example: 4-(2-{6-Amino-8-[(5-bromo-2,3-dihydro-1-benzofuran-6-yl)thio]-9H-purin-9-yl}ethyl)piperidine-1-carbaldehyde; tert-Butyl 4-(2-{6-amino-8-[(5-bromo-2,3-dihydro-1-benzofuran-6-yl)thio]-9H-purin-9-yl}ethyl)piperidine-1-carboxylate; 4-(2-{6-Amino-8-[(5-chloro-2,3-dihydro-1-benzofuran-6-yl)thio]-9H-purin-9-yl}ethyl)piperidine-1-carbaldehyde; and (2S)-1-[4-(2-{6-Amino-8-[(5-bromo-2,3-dihydro-1-benzofuran-6-yl)thio]-9H-purin-9-yl}ethyl)piperidin-1-yl]-1-oxopropan-2-ol.

In another aspect, the present invention relates to compounds according to Formulae VIa and VIb:

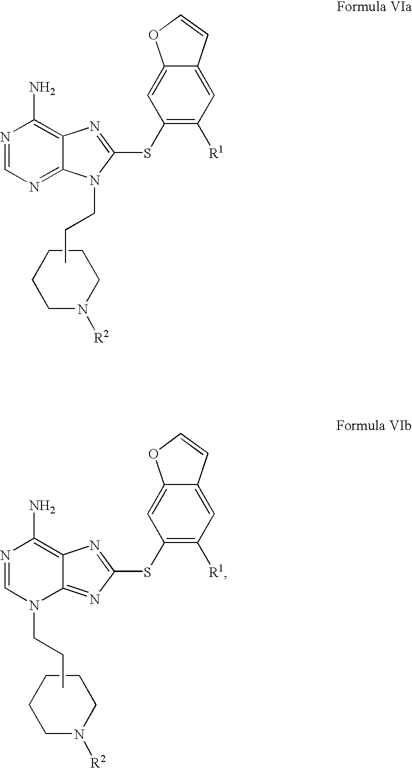

or pharmaceutically acceptable salts thereof; wherein

R¹ and R² are as defined above for the compounds of Formulae Ia and Ib.

In specific embodiments of this aspect of the present invention, compounds according to Formulae VIa and VIb include, for example: 4-(2-{6-Amino-8-[(5-bromo-1-benzofuran-6-yl)thio]-9H-purin-9-yl}ethyl)piperidine-1-carbaldehyde; 4-(2-{6-Amino-8-[(5-chloro-1-benzofuran-6-yl)thio]-9H-purin-9-yl}ethyl)piperidine-1-carbaldehyde; tert-Butyl 4-(2-{6-amino-8-[(5-bromo-1-benzofuran-6-yl)thio]-9H-purin-9-yl}ethyl)piperidine-1-carboxylate; 2-[4-(2-{6-Amino-8-[(5-bromo-1-benzofuran-6-yl)thio]-9H-purin-9-yl}ethyl)piperidin-1-yl]-2-oxoethyl acetate; 2-[4-(2-{6-Amino-8-[(5-bromo-1-benzofuran-6-yl)thio]-9H-purin-9-yl}ethyl)piperidin-1-yl]-2-oxoethanol; 9-[2-(1-Acetylpiperidin-4-yl)ethyl]-8-[(5-bromo-1-benzofuran-6-yl)thio]-9H-purin-6-amine; 8-[(5-Bromo-1-benzofuran-6-yl)thio]-9-(2-piperidin-4-ylethyl)-9H-purin-6-amine; (1S)-2-[4-(2-{6-Amino-8-[(5-bromo-1-benzofuran-6-yl)thio]-9H-purin-9-yl}ethyl)piperidin-1-yl]-1-methyl-2-oxoethyl acetate; (2S)-1-[4-(2-{6-Amino-8-[(5-bromo-1-benzofuran-6-yl)thio]-9H-purin-9-yl}ethyl)piperidin-1-yl]-1-oxopropan-2-ol; (1R)-2-[4-(2-{6-Amino-8-[(5-bromo-1-benzofuran-6-yl)thio]-9H-purin-9-yl}ethyl)piperidin-1-yl]-1-methyl-2-oxoethyl pivalate; and (2R)-1-[4-(2-{6-Amino-8-[(5-bromo-1-benzofuran-6-yl)thio]-9H-purin-9-yl}ethyl)piperidin-1-yl]-1-oxopropan-2-ol.

In another aspect, the present invention relates to compounds according to Formulae VIIa and VIIb:

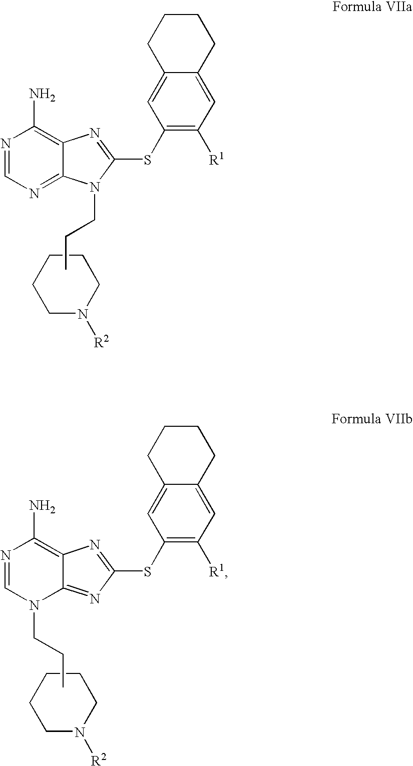

or pharmaceutically acceptable salts thereof; wherein

R¹ and R² are as defined above for the compounds of Formulae Ia and Ib.

In specific embodiments of this aspect of the present invention, compounds according to Formulae VIIa and VIIb include, for example: (2S)-1-[4-(2-{6-amino-8-[(3-bromo-5,6,7,8-tetrahydronaphthalen-2-yl)thio]-9H-purin-9-yl}ethyl)piperidin-1-yl]-1-oxopropan-2-ol; and (2S)-1-[4-(2-{6-amino-8-[(3-bromo-5,6,7,8-tetrahydronaphthalen-2-yl)thio]-3H-purin-3-yl}ethyl)piperidin-1-yl]-1-oxopropan-2-ol.

In another aspect, the present invention relates to compounds according to Formulae VIIIa and VIIIb:

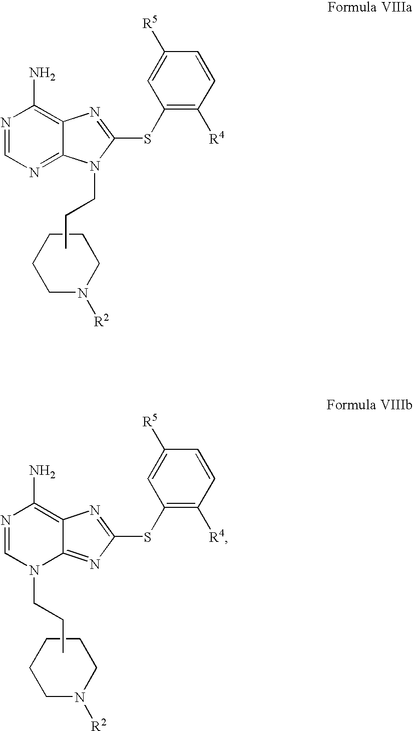

or pharmaceutically acceptable salts thereof; wherein

R² is as defined above for the compounds of Formulae Ia and Ib;

R⁴ is halo, trihalomethoxy, or cyano; and

R⁵ is ethyl, methoxy or nitro.

In specific embodiments of this aspect of the present invention, compounds according to Formulae VIIIa and VIIIb include, for example: 9-[2-(1-Acetylpiperidin-4-yl)ethyl]-8-[(2-bromo-5-methoxyphenyl)thio]-9H-purin-6-amine; 3-[2-(1-Acetylpiperidin-4-yl)ethyl]-8-[(2-bromo-5-methoxyphenyl)thio]-3H-purin-6-amine; 4-(2-{6-Amino-8-[(2-chloro-5-nitrophenyl)thio]-9H-purin-9-yl}ethyl)piperidine-1-carbaldehyde; 4-[2-(6-Amino-8-{[5-methoxy-2-(trifluoromethoxy)phenyl]thio}-9H-purin-9-yl)ethyl]piperidine-1-carbaldehyde; 2-({6-Amino-9-[2-(1-formylpiperidin-4-yl)ethyl]-9H-purin-8-yl}thio)-4-methoxybenzonitrile; 2-({6-Amino-3-[2-(1-formylpiperidin-4-yl)ethyl]-3H-purin-8-yl}thio)-4-methoxybenzonitrile; 2-[4-(2-{6-Amino-8-[(2-bromo-5-methoxyphenyl)thio]-3H-purin-3-yl}ethyl)piperidin-1-yl]-2-oxoethanol; 2-{4-[2-(6-Amino-8-{[5-methoxy-2-(trifluoromethoxy)phenyl]thio}-9H-purin-9-yl)ethyl]piperidin-1-yl}-2-oxoethanol; 2-{4-[2-(6-Amino-8-{[5-methoxy-2-(trifluoromethoxy)phenyl]thio}-3H-purin-3-yl)ethyl]piperidin-1-yl}-2-oxoethanol; 2-({6-Amino-9-[2-(1-glycoloylpiperidin-4-yl)ethyl]-9H-purin-8-yl}thio)-4-methoxybenzonitrile; (2S)-1-[4-(2-{6-Amino-8-[(2-bromo-5-ethylphenyl)thio]-3H-purin-3-yl}ethyl)piperidin-1-yl]-1-oxopropan-2-ol; and 2-({6-Amino-3-[2-(1-glycoloylpiperidin-4-yl)ethyl]-3H-purin-8-yl}thio)-4-methoxybenzonitrile.

In another aspect, the present invention relates to compounds according to Formulae IXa and IXb:

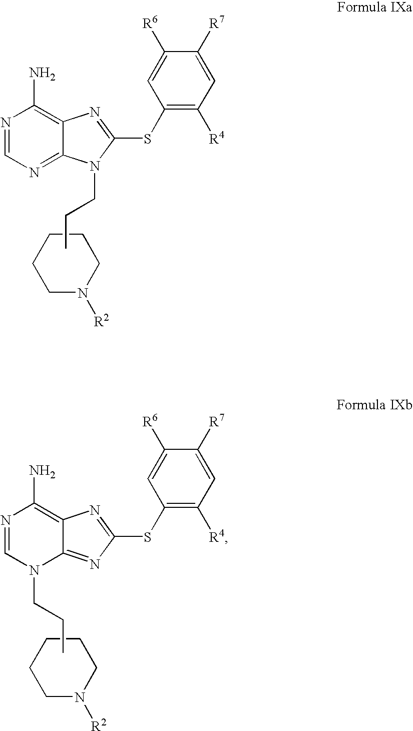

or pharmaceutically acceptable salts thereof; wherein

R² is as defined above for the compounds of Formulae Ia and Ib;

R⁴ is halo, methyl, trihalomethyl, or cyano; and

R⁶ and R⁷ are, independently, hydroxyl or methyl.

In specific embodiments of this aspect of the present invention, compounds according to Formulae IXa and IXb include, for example: 4-(2-{6-Amino-8-[(2-bromo-4,5-dihydroxyphenyl)thio]-9H-purin-9-yl}ethyl)piperidine-1-carbaldehyde; 4-(2-{6-amino-8-[(2-bromo-4,5-dihydroxyphenyl)thio]-3H-purin-3-yl}ethyl)piperidine-1-carbaldehyde; (2S)-1-[4-(2-{6-amino-8-[(2-bromo-4,5-dimethylphenyl)thio]-9H-purin-9-yl}ethyl)piperidin-1-yl]-1-oxopropan-2-ol; (2S)-1-[4-(2-{6-Amino-8-[(2,4,5-trimethylphenyl)thio]-9H-purin-9-yl}ethyl)piperidin-1-yl]-1-oxopropan-2-ol; (2S)-1-[4-(2-{6-Amino-8-[(2,4,5-trimethylphenyl)thio]-3H-purin-3-yl}ethyl)piperidin-1-yl]-1-oxopropan-2-ol; (2S)-1-[4-(2-{6-amino-8-[(2-bromo-4,5-dimethylphenyl)thio]-3H-purin-9-yl}ethyl)piperidin-1-yl]-1-oxopropan-2-ol; and 4-{[6-Amino-9-(2-{1-[(2S)-2-hydroxypropanoyl]piperidin-4-yl}ethyl)-9H-purin-8-yl]thio}-5-bromobenzene-1,2-diol.

In another aspect, the present invention relates to compounds according to Formulae Xa and Xb:

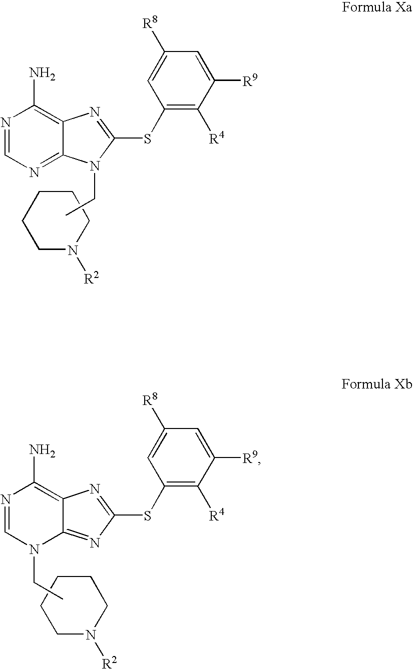

or pharmaceutically acceptable salts thereof; wherein

R² is as defined above for the compounds of Formulae Ia and Ib;

R⁴ is halo, trihalomethyl, or cyano;

R⁸ is hydroxyl, methoxy, or nitro; and

R⁹ is methoxy.

In specific embodiments of this aspect of the present invention, compounds according to Formulae Xa and Xb include, for example: tert-Butyl {(1S)-2-[3-({6-amino-8-[(2-chloro-3,5-dimethoxyphenyl)thio]-9H-purin-9-yl}methyl)piperidin-1-yl]-1-methyl-2-oxoethyl}carbamate; tert-Butyl {(1S)-2-[3-({6-amino-8-[(2-chloro-3,5-dimethoxyphenyl)thio]-3H-purin-3-yl}methyl)piperidin-1-yl]-1-methyl-2-oxoethyl}carbamate; tert-Butyl {(1R)-2-[4-({6-amino-8-[(2-chloro-3,5-dimethoxyphenyl)thio]-3H-purin-3-yl}methyl)piperidin-1-yl]-1-methyl-2-oxoethyl}carbamate; and 9-({1-[(2S)-2-Aminopropanoyl]piperidin-3-yl}methyl)-8-[(2-chloro-3,5-dimethoxyphenyl)thio]-9H-purin-6-amine.

In another aspect, the present invention relates to compounds according to Formulae XIa and XIb:

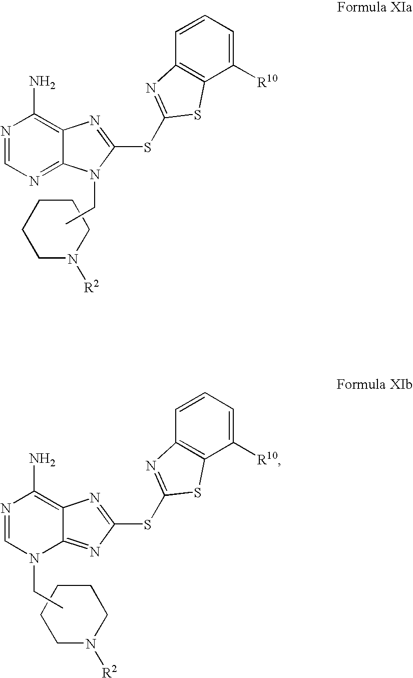

or pharmaceutically acceptable salts thereof; wherein

R² is as defined above for the compounds of Formulae Ia and Ib; and

R¹⁰ is halo.

In specific embodiments of this aspect of the present invention, compounds according to Formulae XIa and XIb include, for example: tert-Butyl {(1R)-2-[4-({6-amino-8-[(7-chloro-1,3-benzothiazol-2-yl)thio]-9H-purin-9-yl}methyl)piperidin-1-yl]-1-methyl-2-oxoethyl}carbamate; tert-butyl {(1R)-2-[4-({6-amino-8-[(7-chloro-1,3-benzothiazol-2-yl)thio]-3H-purin-3-yl}methyl)piperidin-1-yl]-1-methyl-2-oxoethyl}carbamate; and tert-Butyl {(1S)-2-[3-({6-amino-8-[(7-chloro-1,3-benzothiazol-2-yl)thio]-3H-purin-3-yl}methyl)piperidin-1-yl]-1-methyl-2-oxoethyl}carbamate.

In another aspect, the present invention relates to compounds according to Formulae XIIa and XIIb:

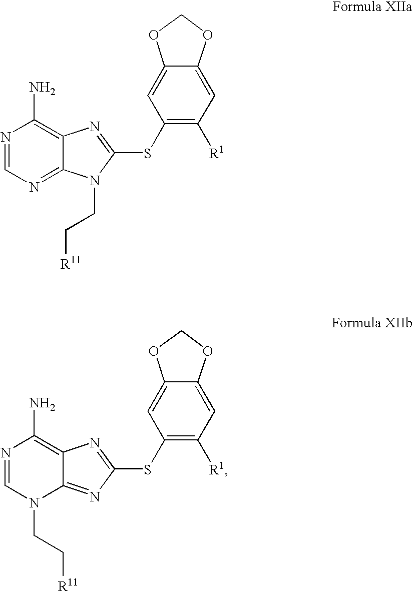

or pharmaceutically acceptable salts thereof; wherein

R¹ is halo, nitro, cyano, —C(═O)H, —C(═O)OCH₃, or —C(═O)OC₂H₅; and

R¹¹ is

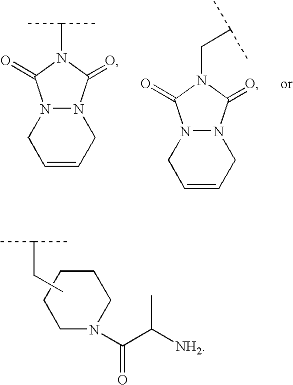

In specific embodiments of this aspect of the present invention, compounds according to Formulae XIIa and XIIb include, for example: 2-(2-{6-Amino-8-[(6-bromo-1,3-benzodioxol-5-yl)thio]-9H-purin-9-yl}ethyl)-5,8-dihydro-1H-[1,2,4]triazolo[1,2-a]pyridazine-1,3(2H)-dione; 2-(2-{6-Amino-8-[(6-bromo-1,3-benzodioxol-5-yl)thio]-3H-purin-3-yl}ethyl)-5,8-dihydro-1H-[1,2,4]triazolo[1,2-a]pyridazine-1,3(2H)-dione; 2-(3-{6-Amino-8-[(6-bromo-1,3-benzodioxol-5-yl)thio]-9H-purin-9-yl}propyl)-5,8-dihydro-1H-[1,2,4]triazolo[1,2-a]pyridazine-1,3(2H)-dione; 6-({6-Amino-3-[2-(1,3-dioxo-5,8-dihydro-1H-[1,2,4]triazolo[1,2-a]pyridazin-2(3H)-yl)ethyl]-3H-purin-8-yl}thio)-1,3-benzodioxole-5-carbonitrile; and 9-(3-{1-[(2R)-2-Aminopropanoyl]piperidin-3-yl}propyl)-8-[(6-bromo-1,3-benzodioxol-5-yl)thio]-9H-purin-6-amine.

In another aspect, the present invention relates to compounds according to Formulae XIIIa and XIIIb:

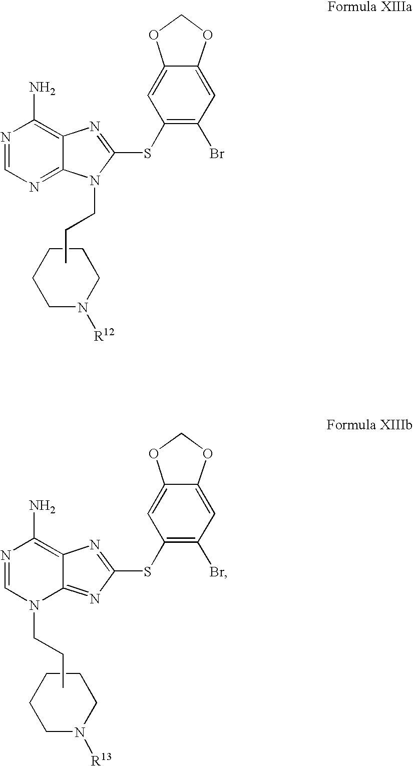

or pharmaceutically acceptable salts thereof; wherein

R¹² is hydro,

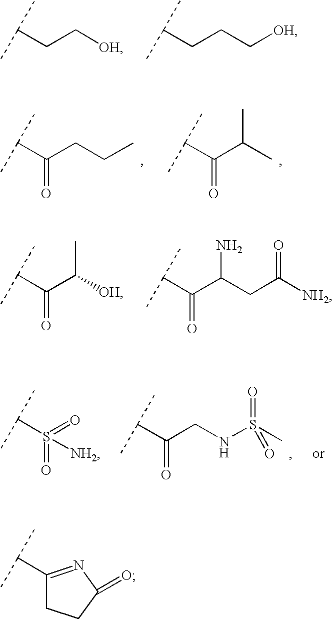

and

R¹³ is

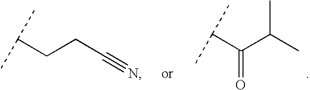

In specific embodiments of this aspect of the present invention, compounds according to Formulae XIIIa and XIIIb include, for example: 8-[(6-Bromo-1,3-benzodioxol-5-yl)thio]-9-[2-(1-isobutyrylpiperidin-4-yl)ethyl]-9H-purin-6-amine; 8-[(6-Bromo-1,3-benzodioxol-5-yl)thio]-3-[2-(1-isobutyrylpiperidin-4-yl)ethyl]-3H-purin-6-amine; 3-[4-(2-{6-Amino-8-[(6-bromo-1,3-benzodioxol-5-yl)thio]-3H-purin-3-yl}ethyl)piperidin-1-yl]propanenitrile; 8-[(6-Bromo-1,3-benzodioxol-5-yl)thio]-9-[2-(1-butyrylpiperidin-4-yl)ethyl]-9H-purin-6-amine; (3S)-3-Amino-4-[4-(2-{6-amino-8-[(6-bromo-1,3-benzodioxol-5-yl)thio]-9H-purin-9-yl}ethyl)piperidin-1-yl]-4-oxobutanamide; 4-[4-(2-{6-Amino-8-[(6-bromo-1,3-benzodioxol-5-yl)thio]-9H-purin-9-yl}ethyl)piperidin-1-yl]-4-oxobutanamide; 5-[4-(2-{6-amino-8-[(6-bromo-1,3-benzodioxol-5-yl)thio]-9H-purin-9-yl}ethyl)piperidin-1-yl]-3,4-dihydro-2H-pyrrol-2-one; 8-[(6-Bromo-1,3-benzodioxol-5-yl)thio]-9-(2-piperidin-4-ylethyl)-9H-purin-6-amine; 4-(2-{6-Amino-8-[(6-bromo-1,3-benzodioxol-5-yl)thio]-9H-purin-9-yl}ethyl)piperidine-1-sulfonamide; 2-[4-(2-{6-Amino-8-[(6-bromo-1,3-benzodioxol-5-yl)thio]-9H-purin-9-yl}ethyl)piperidin-1-yl]ethanol; 3-[4-(2-{6-Amino-8-[(6-bromo-1,3-benzodioxol-5-yl)thio]-9H-purin-9-yl}ethyl)piperidin-1-yl]propan-1-ol; N-{2-[4-(2-{6-Amino-8-[(6-bromo-1,3-benzodioxol-5-yl)thio]-9H-purin-9-yl}ethyl)piperidin-1-yl]-2-oxoethyl}methanesulfonamide; and (2S)-1-[3-(2-{6-Amino-8-[(6-bromo-1,3-benzodioxol-5-yl)thio]-9H-purin-9-yl}ethyl)piperidin-1-yl]-1-oxopropan-2-ol; (2S)-1-[(2R)-2-(2-{6-Amino-8-[(6-bromo-1,3-benzodioxol-5-yl)thio]-9H-purin-9-yl}ethyl)piperidin-1-yl]-1-oxopropan-2-ol; and (2S)-1-[(2S)-2-(2-{6-Amino-8-[(6-bromo-1,3-benzodioxol-5-yl)thio]-9H-purin-9-yl}ethyl)piperidin-1-yl]-1-oxopropan-2-ol.

In another aspect, the present invention relates to compounds according to Formulae XIVa and XIVb.

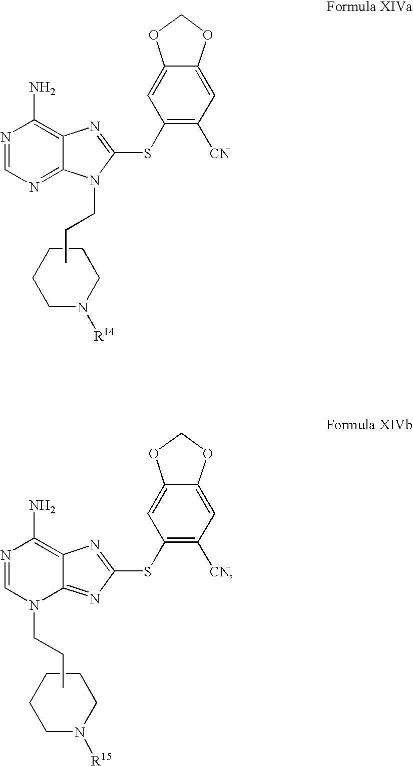

or pharmaceutically acceptable salts thereof; wherein

R¹⁴ is

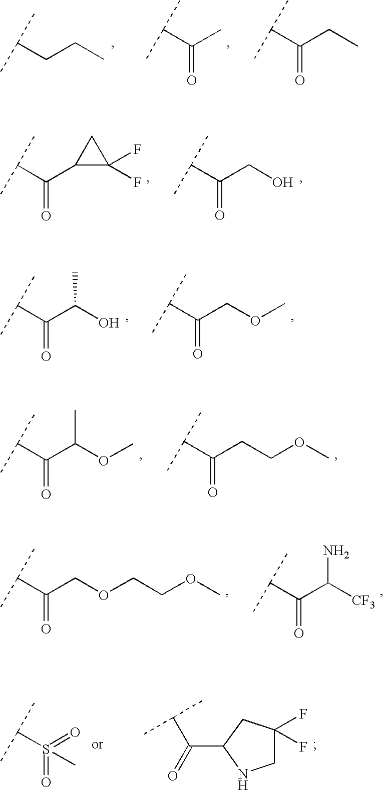

and

R¹⁵ is

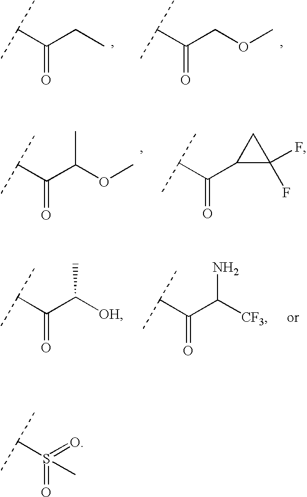

In specific embodiments of this aspect of the present invention, compounds according to Formulae XIVa and XIVb include, for example: 6-[(6-Amino-9-{2-[1-(methoxyacetyl)piperidin-4-yl]ethyl}-9H-purin-8-yl)thio]-1,3-benzodioxole-5-carbonitrile; 6-[(6-Amino-3-{2-[1-(methoxyacetyl)piperidin-4-yl]ethyl}-3H-purin-8-yl)thio]-1,3-benzodioxole-5-carbonitrile; 6-{[6-Amino-9-(2-{1-[(2R)-2-methoxypropanoyl]piperidin-4-yl}ethyl)-9H-purin-8-yl]thio}-1,3-benzodioxole-5-carbonitrile; 6-{[6-Amino-3-(2-{1-[(2R)-2-methoxypropanoyl]piperidin-4-yl}ethyl)-3H-purin-8-yl]thio}-1,3-benzodioxole-5-carbonitrile; 6-{[6-Amino-9-(2-{1-[(2S)-2-methoxypropanoyl]piperidin-4-yl}ethyl)-9H-purin-8-yl]thio}-1,3-benzodioxole-5-carbonitrile; 6-{[6-Amino-3-(2-{1-[(2S)-2-methoxypropanoyl]piperidin-4-yl}ethyl)-3H-purin-8-yl]thio}-1,3-benzodioxole-5-carbonitrile; 6-{[6-Amino-9-(2-{1-[(2,2-difluorocyclopropyl)carbonyl]piperidin-4-yl}ethyl)-9H-purin-8-yl]thio}-1,3-benzodioxole-5-carbonitrile; 6-{[6-Amino-3-(2-{1-[(2,2-difluorocyclopropyl)carbonyl]piperidin-4-yl}ethyl)-3H-purin-8-yl]thio}-1,3-benzodioxole-5-carbonitrile; 6-[(6-Amino-9-{2-[1-(3-methoxypropanoyl)piperidin-4-yl]ethyl}-9H-purin-8-yl)thio]-1,3-benzodioxole-5-carbonitrile; 6-{[6-Amino-9-(2-{1-[(2-methoxyethoxy)acetyl]piperidin-4-yl}ethyl)-9H-purin-8-yl]thio}-1,3-benzodioxole-5-carbonitrile; 6-[(6-Amino-9-{2-[1-(methylsulfonyl)piperidin-2-yl]ethyl}-9H-purin-8-yl)thio]-1,3-benzodioxole-5-carbonitrile; 6-[(6-Amino-9-{2-[1-(methylsulfonyl)piperidin-3-yl]ethyl}-9H-purin-8-yl)thio]-1,3-benzodioxole-5-carbonitrile; 6-[(6-Amino-3-{2-[1-(methylsulfonyl)piperidin-3-yl]ethyl}-3H-purin-8-yl)thio]-1,3-benzodioxole-5-carbonitrile; 6-[(6-Amino-9-{2-[1-(methylsulfonyl)piperidin-4-yl]ethyl}-9H-purin-8-yl)thio]-1,3-benzodioxole-5-carbonitrile; 6-({6-Amino-9-[2-(1-propylpiperidin-2-yl)ethyl]-9H-purin-8-yl}thio)-1,3-benzodioxole-5-carbonitrile; 6-({6-Amino-9-[2-(1-propionylpiperidin-4-yl)ethyl]-9H-purin-8-yl}thio)-1,3-benzodioxole-5-carbonitrile; 6-({6-Amino-3-[2-(1-propionylpiperidin-4-yl)ethyl]-3H-purin-8-yl}thio)-1,3-benzodioxole-5-carbonitrile; [6-({9-[2-(1-Acetylpiperidin-4-yl)ethyl]-6-amino-9H-purin-8-yl}thio)-1,3-benzodioxol-5-yl]acetonitrile; 6-{[6-Amino-9-(2-{1-[(2S)-2-hydroxypropanoyl]piperidin-4-yl}ethyl)-9H-purin-8-yl]thio}-1,3-benzodioxole-5-carbonitrile; 6-{[6-Amino-3-(2-{1-[(2S)-2-hydroxypropanoyl]piperidin-4-yl}ethyl)-3H-purin-8-yl]thio}-1,3-benzodioxole-5-carbonitrile; 6-({6-Amino-9-[2-(1-glycoloylpiperidin-4-yl)ethyl]-9H-purin-8-yl}thio)-1,3-benzodioxole-5-carbonitrile; 6-[(6-Amino-9-{2-[1-(3,3,3-trifluoroalanyl)piperidin-4-yl]ethyl}-9H-purin-8-yl)thio]-1,3-benzodioxole-5-carbonitrile; 6-[(6-amino-3-{2-[1-(3,3,3-trifluoroalanyl)piperidin-4-yl]ethyl}-3H-purin-8-yl)thio]-1,3-benzodioxole-5-carbonitrile; and 6-[(6-amino-9-{2-[1-(4,4-difluoro-L-prolyl)piperidin-4-yl]ethyl}-9H-purin-8-yl)thio]-1,3-benzodioxole-5-carbonitrile.

In another aspect, the present invention relates to compounds according to Formulae XVa and XVb:

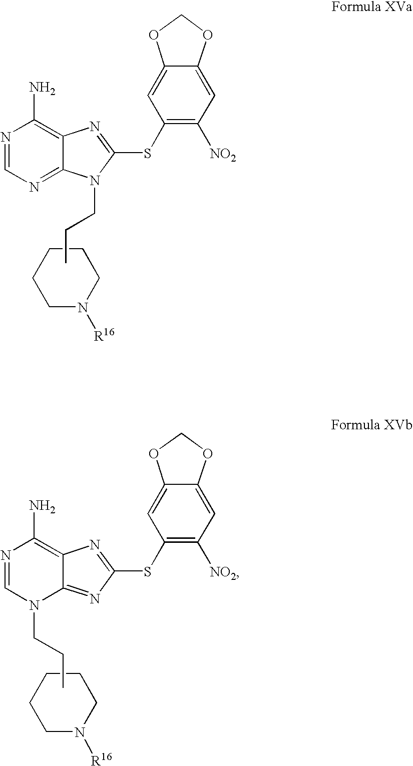

or pharmaceutically acceptable salts thereof; wherein

R¹⁶ is

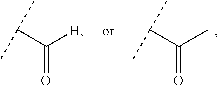

In specific embodiments of this aspect of the present invention, compounds according to Formulae XVa and XVb include, for example: 4-(2-{6-Amino-8-[(6-nitro-1,3-benzodioxol-5-yl)thio]-9H-purin-9-yl}ethyl)piperidine-1-carbaldehyde; 4-(2-{6-Amino-8-[(6-nitro-1,3-benzodioxol-5-yl)thio]-3H-purin-3-yl}ethyl)piperidine-1-carbaldehyde; 9-[2-(1-Acetylpiperidin-4-yl)ethyl]-8-[(6-nitro-1,3-benzodioxol-5-yl)thio]-9H-purin-6-amine; and 3-[2-(1-Acetylpiperidin-4-yl)ethyl]-8-[(6-nitro-1,3-benzodioxol-5-yl)thio]-3H-purin-6-amine.

In another aspect, the present invention relates to compounds according to Formulae XVIa and XVIb:

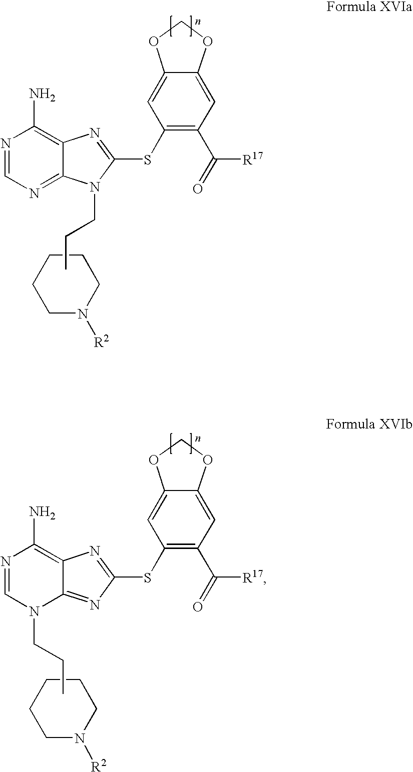

or pharmaceutically acceptable salts thereof; wherein

n is the integer 1 or 2;

R² is as defined above for the compounds of Formulae Ia and Ib; and

R¹⁷ is hydro, —N(CH₃)₂, or —OR¹⁸, wherein

R¹⁸ is C₁-C₆ alkyl (i.e., methyl or ethyl) optionally substituted with 1, 2, 3, 4, or 5 substituents chosen from halo, hydroxyl, amino, cyano, or trihalomethyl.

In specific embodiments of this aspect of the present invention, compounds according to Formulae XVIa and XVIb include, for example: tert-Butyl 4-(2-{6-amino-8-[(6-formyl-1,3-benzodioxol-5-yl)thio]-9H-purin-9-yl}ethyl)piperidine-1-carboxylate; methyl 6-({6-amino-9-[2-(1-formylpiperidin-4-yl)ethyl]-9H-purin-8-yl}thio)-1,3-benzodioxole-5-carboxylate; ethyl 6-({6-amino-9-[2-(1-formylpiperidin-4-yl)ethyl]-9H-purin-8-yl}thio)-1,3-benzodioxole-5-carboxylate; methyl 6-{[6-amino-9-(2-piperidin-4-ylethyl)-9H-purin-8-yl]thio}-1,3-benzodioxole-5-carboxylate; ethyl 6-{[6-amino-9-(2-piperidin-4-ylethyl)-9H-purin-8-yl]thio}-1,3-benzodioxole-5-carboxylate; and 6-({6-amino-9-[2-(1-formylpiperidin-4-yl)ethyl]-9H-purin-8-yl}thio)-N,N-dimethyl-1,3-benzodioxole-5-carboxamide.

In another aspect, the present invention relates to compounds according to Formula XVIII:

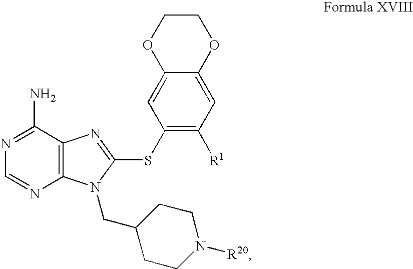

wherein

R¹ is as defined above for the compounds of Formulae Ia and Ib; and

R²⁰ is d- or l-alanine linked to the piperidinyl residue via a peptide bond, and optionally substituted with:

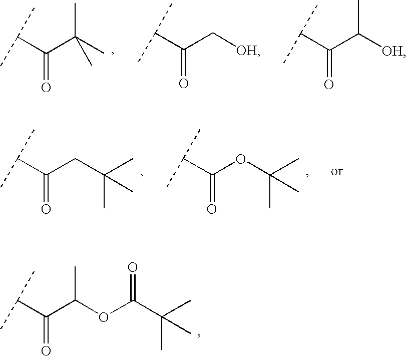

via a second peptide bond.

In specific embodiments of this aspect of the present invention, compounds according to Formula XVIII include, for example: tert-Butyl {(1R)-2-[4-({6-amino-8-[(7-bromo-2,3-dihydro-1,4-benzodioxin-6-yl)thio]-9H-purin-9-yl}methyl)piperidin-1-yl]-1-methyl-2-oxoethyl}carbamate; tert-Butyl {(1S)-2-[4-({6-amino-8-[(7-bromo-2,3-dihydro-1,4-benzodioxin-6-yl)thio]-9H-purin-9-yl} methyl)piperidin-1-yl]-1-methyl-2-oxoethyl}carbamate; 9-({1-[(2R)-2-Aminopropanoyl]piperidin-4-yl}methyl)-8-[(7-bromo-2,3-dihydro-1,4-benzodioxin-6-yl)thio]-9H-purin-6-amine; 9-({1-[(2S)-2-Aminopropanoyl]piperidin-4-yl}methyl)-8-[(7-bromo-2,3-dihydro-1,4-benzodioxin-6-yl)thio]-9H-purin-6-amine; (2S)—N-{(1R)-2-[4-({6-Amino-8-[(7-bromo-2,3-dihydro-1,4-benzodioxin-6-yl)thio]-9H-purin-9-yl}methyl)piperidin-1-yl]-1-methyl-2-oxoethyl}-2-hydroxypropanamide; (1R)-2-({(1R)-2-[4-({6-amino-8-[(7-bromo-2,3-dihydro-1,4-benzodioxin-6-yl)thio]-9H-purin-9-yl}methyl)piperidin-1-yl]-1-methyl-2-oxoethyl}amino)-1-methyl-2-oxoethyl pivalate; N-{(1R)-2-[4-({6-amino-8-[(7-bromo-2,3-dihydro-1,4-benzodioxin-6-yl)thio]-9H-purin-9-yl}methyl)piperidin-1-yl]-1-methyl-2-oxoethyl}-2,2-dimethylpropanamide; N-{(1R)-2-[4-({6-Amino-8-[(7-bromo-2,3-dihydro-1,4-benzodioxin-6-yl)thio]-9H-purin-9-yl}methyl)piperidin-1-yl]-1-methyl-2-oxoethyl}-3,3-dimethylbutanamide; (2S)—N-{(1S)-2-[4-({6-Amino-8-[(7-bromo-2,3-dihydro-1,4-benzodioxin-6-yl)thio]-9H-purin-9-yl}methyl)piperidin-1-yl]-1-methyl-2-oxoethyl}-2-hydroxypropanamide; N-{(1S)-2-[4-({6-Amino-8-[(7-bromo-2,3-dihydro-1,4-benzodioxin-6-yl)thio]-9H-purin-9-yl}methyl)piperidin-1-yl]-1-methyl-2-oxoethyl}-2-hydroxyacetamide; and (2R)—N-{(1R)-2-[4-({6-Amino-8-[(7-bromo-2,3-dihydro-1,4-benzodioxin-6-yl)thio]-9H-purin-9-yl}methyl)piperidin-1-yl]-1-methyl-2-oxoethyl}-2-hydroxypropanamide.

In another aspect, the present invention relates to compounds according to Formula XIX:

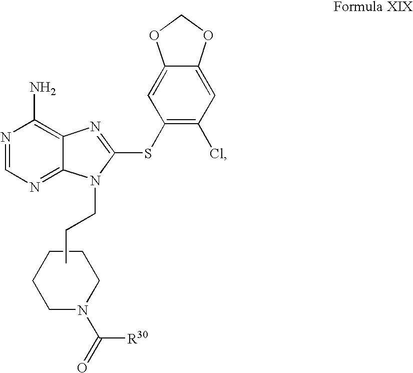

wherein

R³⁰ is selected from

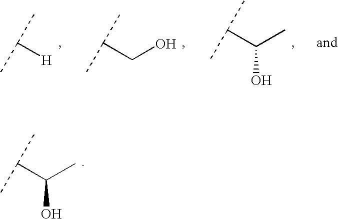

In specific embodiments of this aspect of the present invention, compounds according to Formula XIX include, for example: 4-(2-{6-Amino-8-[(6-chloro-1,3-benzodioxol-5-yl)thio]-9H-purin-9-yl}ethyl)piperidine-1-carbaldehyde; (2S)-1-[4-(2-{6-Amino-8-[(6-chloro-1,3-benzodioxol-5-yl)thio]-9H-purin-9-yl}ethyl)piperidin-1-yl]-1-oxopropan-2-ol; (2R)-1-[4-(2-{6-Amino-8-[(6-chloro-1,3-benzodioxol-5-yl)thio]-9H-purin-9-yl}ethyl)piperidin-1-yl]-1-oxopropan-2-ol; and 2-[4-(2-{6-Amino-8-[(6-chloro-1,3-benzodioxol-5-yl)thio]-9H-purin-9-yl}ethyl)piperidin-1-yl]-2-oxoethanol.

In preferred embodiments, compounds of Formulae Ia, Ib, IIa, IIb, IIIa, IIIc, IVa, IVb, Va, Vb, VIIa, VIIb, VIIa, VIIb, VIIIa, VIIIb, IXa, IXb, Xa, Xb, XIa, XIb, XIIa, XIIb, XIIIa, XIIIb, XIVa, XIVb, XVa, XVb, XVIa, XVIb, XVIII, and XIX, having an IC₅₀ of less than 2,500 nM, 500 nM, 300 nM, 200 nM, preferably less than 100 nM, and most preferably less than 50 nM, as determined by the Hsp90 binding assay, which is described in the “Biological and Pharmacological Assays and Examples” section below, are used as the therapeutic compounds of the invention. The activities of exemplary compounds, as revealed by this assay, are provided in Table 10, below.

As used herein, the phrase “treating . . . with . . . a compound” means either administering the therapeutic compound to cells or an animal, or administering to cells or an animal another agent to cause the presence of, or the formation of, the therapeutic compound inside the cells or the animal. Preferably, the methods of the present invention comprise administering to cells in vitro or to a warm-blood animal, particularly a mammal, more particularly a human, a pharmaceutical composition comprising an effective amount of a compound according to the present invention.

A pharmaceutically acceptable salt of the compound of the present invention is exemplified by a salt with an inorganic acid and/or a salt with an organic acid that are known in the art. In addition, pharmaceutically acceptable salts include acid salts of inorganic bases, such as salts containing alkaline cations, alkaline earth cations, as well as acid salts of organic bases. Their hydrates, solvates, and the like are also encompassed in the compound of the present invention. In addition, N-oxide compounds are also encompassed in the compound of the present invention.

Additionally, the compounds of the present invention can contain asymmetric carbon atoms and can therefore exist in racemic and optically active forms. Thus, optical isomers or enantiomers, racemates, and diastereomers are also encompassed. The methods of present invention include the use of all such isomers and mixtures thereof. The present invention encompasses any isolated racemic or optically active form of the compounds described above, or any mixture thereof, which possesses Hsp90 inhibitory activity, or anti-cancer activity.

In preferred embodiments, compounds of Formulae Ia, Ib, IIa, IIb, IIIa, IIIc, IVa, IVb, Va, Vb, VIIa, VIIb, VIIa, VIIb, VIIIa, VIIIb, IXa, IXb, Xa, Xb, XIa, XIb, XIIa, XIIb, XIIIa, XIIIb, XIVa, XIVb, XVa, XVb, XVIa, XVIb, XVIII, and XIX are provided having an IC₅₀ of less than 2,500 nM, 500 nM, 300 nM, 200 nM, preferably less than 100 nM, and most preferably less than 50 nM, as determined in the Hsp90 binding assay, which is described in the “Biological and Pharmacological Assays and Examples” section below. Such activities of exemplary compounds are provided in Table 10, below.

Note that some of the names listed in the Markush groups above are derived from the names of the specific “R”-groups whereas the names accompanying the structures in the tables below were generated for the entire pictured molecule. For example the Example Compound 88 below is named (2S)-1-[4-(2-{6-Amino-8-[(7-bromo-2,3-dihydro-1,4-benzodioxin-6-yl)thio]-9H-purin-9-yl}ethyl)piperidin-1-yl]-1-oxopropan-2-ol. This name, as are all names presented in this application, was generated using either ACD/Name chemical naming software (version 8.08), available from Advanced Chemistry Development, Inc. (Toronto, Ontario, Canada), or the Autonom 2000 plug-in for the Isis™/Draw 2.5 SP1 chemical drawing program, available from MDL Information Systems, a division of Symyx Technologies, Inc. (Santa Clara, Calif.).

Also note that specific example compounds of the compounds of Formulae Ia, Ib, IIa, IIb, IIIa, IIIc, IVa, IVb, Va, Vb, VIa, VIb, VIIa, VIIb, VIIIa, VIIIb, IXa, IXb, Xa, Xb, XIa, XIb, XIIa, XIIb, XIIIa, XIIIb, XIVa, XIVb, XVa, XVb, XVIa, XVIb, XVIII, and XIX are sequentially numbered herein for the sake of convenience, but that these numbers do not refer to any preferences by the inventors, or any specific ranking of their efficacy.

As the skilled artisan readily recognizes, in the different aspects and embodiments of the present invention, any undefined substituent can be chosen from any of the other suitable specific embodiments or aspects of the invention, unless otherwise specified. Furthermore, in certain drawings and representations of the compounds of the invention, the skilled artisan would readily recognize that the valances of some atoms are filled by the formation of a covalent bond to a hydrogen atom, which may or may not be depicted by an —H in the drawings.

Furthermore, as is understood by the skilled artisan, certain variables in the list of substituents are either repetitive, or redundant (i.e., different names for identical substituents), or generic to other terms in the list, or partially overlap in content with other terms. In the compounds of the present invention, the skilled artisan recognizes that substituents may be attached to the remainder of the molecule via a number of positions and the preferred positions are as illustrated in the “example compounds” presented.

Unless specifically stated otherwise or indicated by a bond symbol (dash or double dash), the connecting point to a recited group will be on the right-most stated group. Thus, for example, a hydroxyalkyl group is connected to the main structure through the alkyl and the hydroxyl is a substituent on the alkyl.

### 3. Pharmaceutical Compositions

In another aspect, the present invention provides a medicament or pharmaceutical composition having a therapeutically or prophylactically effective amount of a therapeutic compound according to the present invention.

Typically, therapeutic compounds according to the present invention can be effective at an amount of from about 0.01 μg/kg to about 100 mg/kg per day based on total body weight. The active ingredient may be administered at once, or may be divided into a number of smaller doses to be administered at predetermined intervals of time. The suitable dosage unit for each administration can be, e.g., from about 1 μg to about 2000 mg, preferably from about 5 μg to about 1000 mg. In the case of combination therapy, a therapeutically effective amount of one or more other anticancer compounds can be administered in a separate pharmaceutical composition, or alternatively included in the pharmaceutical composition according to the present invention which contains a compound according to the present invention. The pharmacology and toxicology of many of such other anticancer compounds are known in the art. See e.g., Physicians Desk Reference, Medical Economics, Montvale, N.J.; and The Merck Index, Merck & Co., Rahway, N.J. The therapeutically effective amounts and suitable unit dosage ranges of such compounds used in art can be equally applicable in the present invention.

It should be understood that the dosage ranges set forth above are exemplary only and are not intended to limit the scope of this invention. The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can be adjusted as the various factors change over time.

In the pharmaceutical compositions, the active agents can be in any pharmaceutically acceptable salt form. As used herein, the term “pharmaceutically acceptable salts” refers to the relatively non-toxic, organic or inorganic salts of the active compounds, including inorganic or organic acid addition salts of the compound.

For oral delivery, the active compounds can be incorporated into a formulation that includes pharmaceutically acceptable carriers such as binders, lubricants, disintegrating agents, and sweetening or flavoring agents, all known in the art. The formulation can be orally delivered in the form of enclosed gelatin capsules or compressed tablets. Capsules and tablets can be prepared in any conventional techniques. The capsules and tablets can also be coated with various coatings known in the art to modify the flavors, tastes, colors, and shapes of the capsules and tablets. In addition, liquid carriers such as fatty oil can also be included in capsules.

Suitable oral formulations can also be in the form of suspension, syrup, chewing gum, wafer, elixir, and the like. If desired, conventional agents for modifying flavors, tastes, colors, and shapes of the special forms can also be included.

The active compounds can also be administered parenterally in the form of solution or suspension, or in lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacteria agents, surfactants, and antioxidants can all be included. The parenteral formulations can be stored in any conventional containers such as vials and ampoules.

Routes of topical administration include nasal, bucal, mucosal, rectal, or vaginal applications. For topical administration, the active compounds can be formulated into lotions, creams, ointments, gels, powders, pastes, sprays, suspensions, drops and aerosols. Thus, one or more thickening agents, humectants, and stabilizing agents can be included in the formulations. A special form of topical administration is delivery by a transdermal patch. Methods for preparing transdermal patches are disclosed, e.g., in Brown, et al., Annual Review of Medicine, 39:221-229 (1988), which is incorporated herein by reference.

Subcutaneous implantation for sustained release of the active compounds may also be a suitable route of administration. This entails surgical procedures for implanting an active compound in any suitable formulation into a subcutaneous space, e.g., beneath the anterior abdominal wall. See, e.g., Wilson et al., J. Clin. Psych. 45:242-247 (1984). Hydrogels can be used as a carrier for the sustained release of the active compounds. Hydrogels are generally known in the art. They are typically made by crosslinking high molecular weight biocompatible polymers into a network, which swells in water to form a gel like material. Preferably, hydrogels are biodegradable or biosorbable. See, e.g., Phillips et al., J. Pharmaceut. Sci., 73:1718-1720 (1984).

The active compounds can also be conjugated, to a water soluble non-immunogenic non-peptidic high molecular weight polymer to form a polymer conjugate. For example, an active compound is covalently linked to polyethylene glycol to form a conjugate. Typically, such a conjugate exhibits improved solubility, stability, and reduced toxicity and immunogenicity. Thus, when administered to a patient, the active compound in the conjugate can have a longer half-life in the body, and exhibit better efficacy. See generally, Burnham, Am. J. Hosp. Pharm., 15:210-218 (1994). PEGylated proteins are currently being used in protein replacement therapies and for other therapeutic uses. For example, PEGylated interferon (PEG-INTRON A®) is clinically used for treating Hepatitis B. PEGylated adenosine deaminase (ADAGEN®) is being used to treat severe combined immunodeficiency disease (SCIDS). PEGylated L-asparaginase (ONCAPSPAR®) is being used to treat acute lymphoblastic leukemia (ALL). It is preferred that the covalent linkage between the polymer and the active compound and/or the polymer itself is hydrolytically degradable under physiological conditions. Such conjugates known as “prodrugs” can readily release the active compound inside the body. Controlled release of an active compound can also be achieved by incorporating the active ingredient into microcapsules, nanocapsules, or hydrogels generally known in the art.

Liposomes can also be used as carriers for the active compounds of the present invention. Liposomes are micelles made of various lipids such as cholesterol, phospholipids, fatty acids, and derivatives thereof. Various modified lipids can also be used. Liposomes can reduce the toxicity of the active compounds, and increase their stability. Methods for preparing liposomal suspensions containing active ingredients therein are generally known in the art. See, e.g., U.S. Pat. No. 4,522,811; Prescott, Ed., Methods in Cell Biology, Volume XIV, Academic Press, New York, N.Y. (1976).

The active compounds can also be administered in combination with another active agent that synergistically treats or prevents the same symptoms or is effective for another disease or symptom in the patient treated, so long as the other active agent does not interfere with, or adversely affect, the effects of the active compounds of this invention. Such other active agents include but are not limited to anti-inflammation agents, antiviral agents, antibiotics, antifungal agents, antithrombotic agents, cardiovascular drugs, cholesterol lowering agents, anti-cancer drugs, hypertension drugs, and the like.

### 4. Therapeutic Methods

The present invention provides therapeutic methods comprising administering to an animal (e.g., a patient, in need of such treatment) a therapeutically effective amount of one or more compounds of Formulae Ia, Ib, IIa, IIb, IIIa, IIIc, IVa, IVb, Va, Vb, VIIa, VIIb, VIIa, VIIb, VIIIa, VIIIb, IXa, IXb, Xa, Xb, XIa, XIb, XIIa, XIIb, XIIIa, XIIIb, XIVa, XIVb, XVa, XVb, XVIa, XVIb, XVIII, and XIX, as defined above, and/or a pharmaceutically acceptable salt thereof. The therapeutic methods are particularly useful in the treatment of Hsp90 inhibitor-sensitive cancers, which comprise a group of diseases characterized by the uncontrolled growth and spread of abnormal cells. Such diseases include, but are not limited to, Hodgkin's disease, non-Hodgkin's lymphoma, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, and prostatic carcinoma.

In another aspect, the invention provides a method for treating an individual having an Hsp90 inhibitor-sensitive disease or disorder chosen from inflammatory diseases, viral or bacterial infections, autoimmune disorders, stroke, ischemia, cardiac disorders, neurological disorders, proliferative disorders, neoplasms, malignant diseases, and metabolic diseases.

In yet another aspect, the invention provides a method for treating an individual having an Hsp90 inhibitor-sensitive fibrogenetic disorder, such as, for example, scleroderma, polymyositis, systemic lupus, rheumatoid arthritis, liver cirrhosis, keloid formation, interstitial nephritis and pulmonary fibrosis.

### 5. Chemical Intermediates

The invention also relates to compounds of Formulae XVIIa and XVIIb,

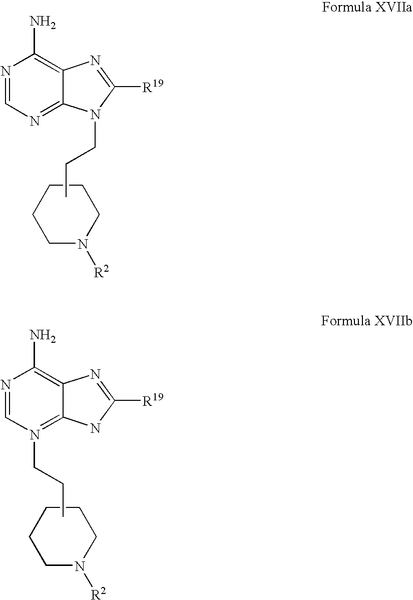

wherein

R² is as defined above for the compounds of Formulae Ia and Ib; and

R¹⁹ is hydro, bromo, or

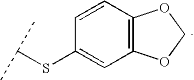

The compounds of Formulae XVIIa and XVIIb, as described above, can serve as intermediates in the synthesis of various specific therapeutic compounds of Formulae Ia, Ib, IIa, IIb, IIIa, IIIc, IVa, IVb, Va, Vb, VIIa, VIIb, VIIa, VIIb, VIIIa, VIIIb, IXa, IXb, XIIIa, XIIIb, XIVa, XIVb, XVa, XVb, XVIa, XVIb, and XIX using the methods described in detail below.

### EXAMPLES

### Chemical Synthesis and Purification of Example Compounds

All reactions were performed in flame-dried or oven-dried glassware under a positive pressure of dry nitrogen or dry argon and were stirred magnetically unless otherwise indicated. Chemicals were purchased from standard commercial vendors and used as received unless otherwise noted. Otherwise their preparation is facile and known to one of ordinary skill in the art, or it is referenced or described herein. Yields are not optimized. The names of example compounds and intermediates were generated using either the “ACD/Name” chemical naming software (version 8.08) available from Advanced Chemistry Development, Inc. (Toronto, Ontario, Canada), or the “Autonom 2000” plug-in for the Isis™/Draw 2.5 SP1 chemical drawing program, available from MDL Information Systems, a division of Symyx Technologies, Inc. (Santa Clara, Calif.).

Analytical TLC plates (Silica Gel 60 F254 or Merck EM-5715-7, EM Science, Gibbstown, N.J.) were used to follow the course of reactions, and the MPLC system used for purifications (Isco Foxy Jr fraction collector, UA-6 detector) was from Teledyne Isco, Inc. (Lincoln, Nebr.), using Isco silica gel flash columns. ¹H NMR spectra were recorded on a Varian Mercury 400 MHz instrument (Varian Inc., Palo Alto, Calif.) and chemical shifts are expressed in parts per million (ppm, δ) relative to TMS as the internal standard. Mass spectra were obtained on a Thermo Finnigan LCQ-Deca (injection volume 5 uL, XTerra MS-C₁₈ 3.5 μm 2.1×50 mm column, XTerra MS-C₁₈ 5 μm 2.1×20 mm guard column) (Thermo Finnigan, Austin, Tex.), ESI source, analytical HPLC was performed on an Agilent HP1100 (injection volume 5 μl, XTerra RP-C₁₈ 5 μm 4.6×250 mm column, with an XTerra MS-C₁₈ 5 μm 2.1×20 mm guard column) (Agilent Technologies, Santa Clara, Calif.). Preparative HPLC purifications were performed using either Agilent HP-1100 preparative LC or SFC-70 from Thar Technologies (Pittsburgh, Pa.). The sample preparations and the conditions were described below.

### Method A: Agilent HP-1100 Preparative LC:

Samples were dissolved in dimethylsulfoxide and injected on a phenyl-Hexyl column (Phenomenex, Torrance, Calif.) 10×250 mm, 5μ particle was used. The column was eluted with a mixture of acetonitril and water (both containing 0.01% v/v trifluoroacetic acid) in a flow rate of 10 ml/min and a gradient of 15% 100% acetonitril over a period of 20 min.

### Method B: SFC-70; Thar Technologies:

Samples were dissolved in dimethylsulfoxide and injected on a pyridine column (Princeton Chromatography, Cranbury, N.J.) 21.2×250 mm, 5μ particle size, temp of 40° C. and a back pressure of 200 bar. Column was eluted with a mixture of liquid CO₂ and methanol in a flow rate of 50 gm/min. Methanol was used as a modifier in a gradient of 5% to 50% over a period of 18 min.

### Abbreviations and Acronyms

When the following abbreviations are used herein, they have the following meanings:

| | |
|---|---|
| Ac₂O | acetic anhydride |
| anhy | Anhydrous |
| n-BuOH | n-butanol |
| t-BuOH | t-butanol |
| CD₃OD | methanol-d₄ |
| Celite ® | diatomaceous earth filter agent, ® Celite Corp. |
| CH₂Cl₂ | methylene chloride |
| DCM | dichloromethane |
| CI-MS | chemical ionization mass spectroscopy |
| conc | concentrated |
| dec | decomposition |
| bs | broad singlet |
| br | broad |
| DME | dimethoxyethane |
| DMF | N,N-dimethylformamide |
| DMSO | dimethylsulfoxide |
| DMSO-d₆ | dimethylsulfoxide-d₆ |
| ELSD | evaporative light scattering device |
| EtOAc | ethyl acetate |
| EtOH | ethanol (100%) |
| Et₂O | diethyl ether |
| Et₃N | triethylamine |
| HPLC ESI-MS | high performance liquid chromatography-electrospray |
| | mass spectroscopy |
| MPLC | medium pressure liquid chromatography |
| NMR | nuclear magnetic resonance spectroscopy |
| TOF-MS | time-of-flight-mass spectroscopy |
| NMM | 4-methylmorpholine |
| Ph₃P | triphenylphosphine |
| Pd(dppf)Cl₂ | [1,1′- |
| | bis(diphenylphosphino)ferrocene]dichloropalladium(II) |
| Pd(PPh₃)₄ | tetrakis(triphenylphosphine)palladium(0) |
| Pd(OAc)₂ | palladium(II) acetate |
| P(O)Cl₃ | phosphorous oxychloride |
| R_{f} | TLC retention factor |
| RT | retention time (HPLC) |
| rt | room temperature |
| THF | tetrahydrofuran |
| TFA | trifluoroacetic acid |
| TLC | thin layer chromatography |
| LC-MS (ESI) | liquid chromatography-mass spectroscopy (electrospray |
| | ionization) |
| DIEA | Diisopropylethylamine |
| TFAA | trifluoroacetic anhydride |
| MsCl | Methanesulfonylchloride |
| AcOH | acetic acid |
| HCl | hydrochloric acid |
| H₂SO₄ | sulfuric acid |
| HNO₃ | nitric acid |
| HBr | hydrobromic acid |
| CDCl₃ | chloroform-d₃ |
| CHCl₃ | Chloroform |
| H₂O | Water |
| NaOAc | sodium acetate |
| KOH | potassium hydroxide |
| NaOH | sodium hydroxide |
| NaCl | sodium chloride |
| NaHCO₃ | sodium bicarbonate |
| Na₂CO₃ | sodium carbonate |
| K₂CO₃ | potassium carbonate |
| Na₂SO₄ | sodium sulfate |
| MgSO₄ | magnesium sulfate |
| MeOH | Methanol |
| SiO₂ | silica gel |
| K₃PO₄ | potassium phosphate |
| NH₄Cl | ammonium chloride |
| AIBN | 2,2′-axo bisisobutyronitrile |
| Barton's base | 2-t-butyl-1,1,3,3-tetramethylguanidine |
| DMAP | N,N-Dimethyl aminopyridine |
| LG | leaving group |
| MsCl | methanesulfonyl chloride |
| TsCl | p-toluenesulfonyl chloride |
| PG | protecting group |
| Xantphos | 4,5-bis(diphenylphosphino)-9,9-dimethyl xanthane |
| TBAH | Tetra butyl ammonium hydroxide |

The substituted alcohols are either commercially available or prepared according to published procedures. These substituted alcohols are converted to corresponding leaving group (Cl, OTs) in accordance with synthetic methods well known to the skilled atrisan. General methods for the preparation of the compounds are given below, and the preparation of representative compounds is specifically illustrated in the following Examples.

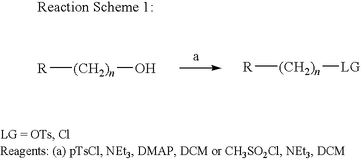

The alcohols were converted to the corresponding chlorides or tosylates by treating with either p-toluene sulfonyl chloride, triethyl amine and DMAP in DCM or methane sulfonyl chloride and triethyl amine in DCM at 0-25° C. for 1-16 h.

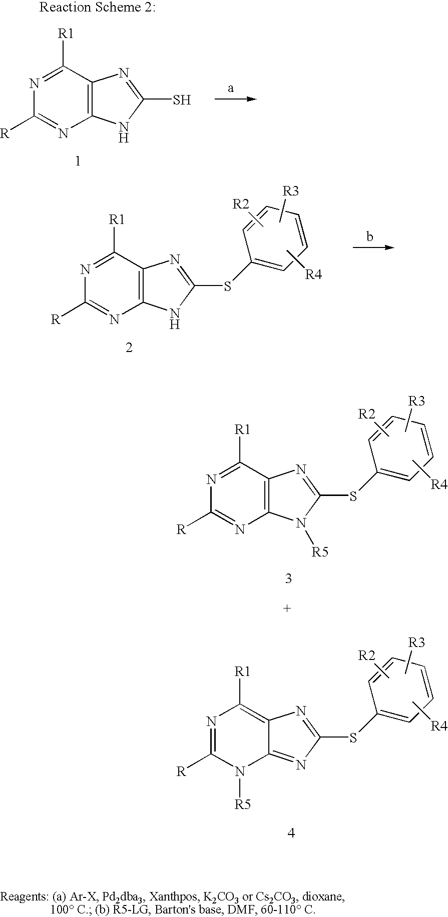

The compound 2 can be prepared by palladium catalyzed coupling of aryl halides with mercaptoadenine 1. The derivatives of 8-arylsulfanyl adenine 2 were alkylated using various alkylating agents in the presence of base at 30-110° C. in DMF for 1-18 h. Formation of the mixture of regioisomers 3 and 4 were observed by HPLC and LC-MS analysis. At the end of this period solvent was evaporated or after aqueous and organic work up, the organic layer was collected and was dried over Na₂SO₄. After removal organic solvent and preparative HPLC [X-Terra prep-RP18 10 um, 19×250 mm (Waters Corporation, Milford, Mass.), Mobile phase: solvent A: Water HPLC grade containing 0.01% TFA, and solvent B: acetonitrile containing 0.01% TFA, general eluting gradient—solvent B 15% to 80% over 15 to 25 minutes run time] purification, N-3 and N-9 alkylated products are isolated as a trifluoroacetate salt.

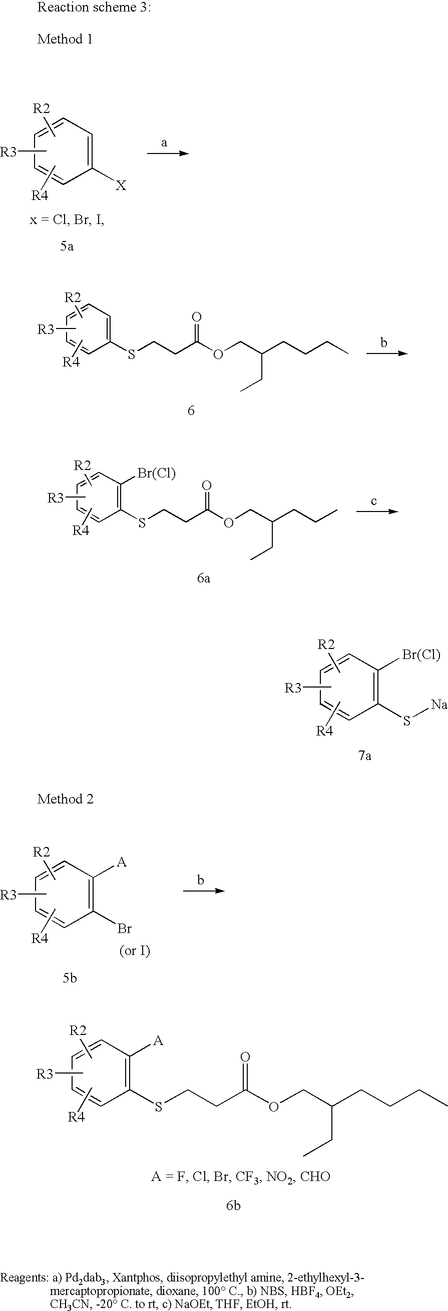

The thiophenol surrogates 6 can be synthesized by palladium catalyzed coupling reactions of aryl halides with 2-ethylhexyl-3-mercaptopropionate or its analogs by adaption of known procedure (J. Org. Chem., 71:2003 (2006)). The aryl halides are commercially available or can be synthesized by known methods in the art. The protected arylthiols 6 can be further functionalized via halogenation or other standard transformations. Alternatively, aryl bromides 5b containing halogen atoms or other groups positioned at ortho to bromine or iodine, can be directly used for palladium catalyzed coupling reactions to synthesize 6b and further transformation of group X can be possible based on the properties of X. The protected thiols 6 can be converted to their corresponding salts or free base 7 by treatment of appropriate bases.

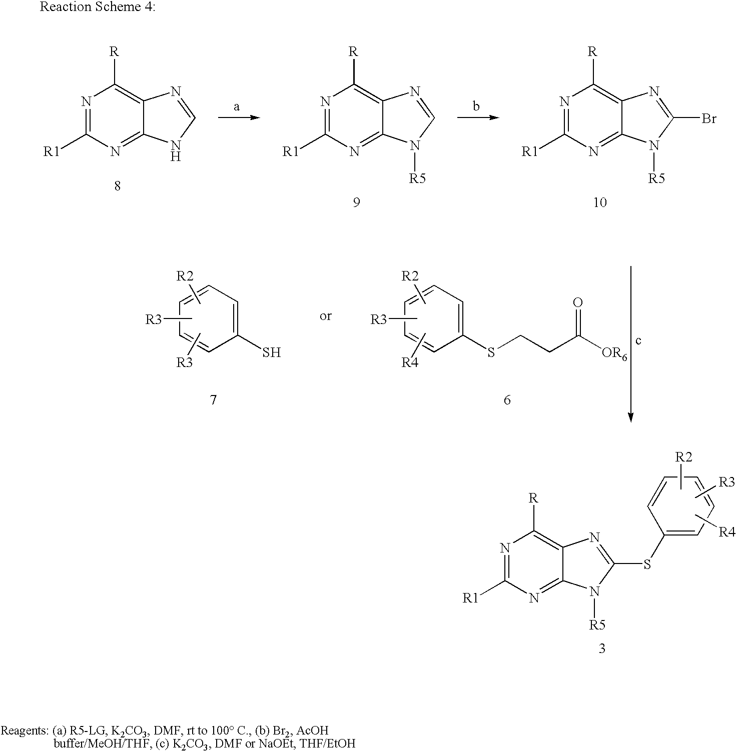

As shown in Reaction scheme 4, an alternative synthetic method can be used to obtain the target compounds 3, starting from alkylation of adenines 8 with various alkyling agents. The alkylated adenines 9 can be converted to the corresponding bromides 10 by treatment of bromine in NaOAc buffer (J. Med. Chem. 49:817 (2006)). Substitution of bromine in the compounds 10 with thiophenols 7 or their surrogates 6 under basic conditions can provide the target compounds 3, where thiophenols 7 or 6 are commercially available or can be synthesized as illustrated in reaction scheme 3.

### General Method of Preparation of Intermediates

### Intermediate 1

### 6-Bromo-3-oxoindane-5-carbonitrile

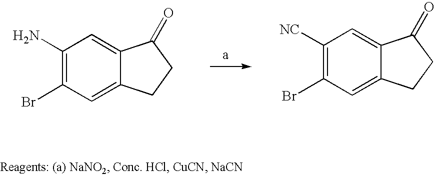

To a solution of 6-amino-5-bromo-2,3-dihydro-1H-indan-1-one (3.0 g, 12.3 mmol) in concentrated HCl (6 mL) and H₂O (15 mL) was added a solution of NaNO₂ (1.01 g, 14.60 mmol) in H₂O (15 mL) at 0° C. The resulting mixture was neutralized with solid NaHCO₃ (5.15 g) and kept the temperature below 5° C. The resulting dizaonium solution was then added drop wise to a mixture of CuCN (4.75 g, 53.08 mmol) and KCN (4.32 g, 66.35 mmol) in H₂O (36 mL)/toluene (10 mL) and slowly warmed up to 50° C. After stirring for 1 h at 50° C., the mixture was stirred at rt for ˜10 h, filtered, and washed with CH₂Cl₂. The combined filtrates were washed with brine, dried over Na₂SO₄, filtered, and concentrated in vacuo. The residue was purified by column chromatography on SiO₂ to provide the example intermediate (2.1 g, 67%).

### Intermediate 2

### (6-Bromo-1,3-benzodioxol-5-yl)acetonitrile

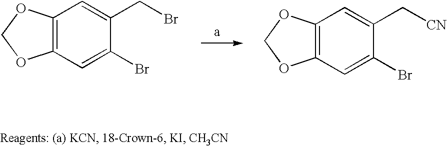

To a solution of 5-bromo-6-bromomethyl-1,3-benzodioxole (5.0 g, 17 mmol) in CH₃CN (60 mL) was added 18-crown-6 ether (0.90 g, 1.70 mmol), KCN (2.47 g, 38.0 mmol), KI (282 mg, 1.70 mmol), and water (4.8 mL). After stirring for 10 h, the mixture extracted with EtOAc, washed with brine, dried over Na₂SO₄, filtered, and concentrated in vacuo. The residue was purified by column chromatograph on SiO₂ using 10-50% gradient of EtOAc in hexanes to provide the example intermediate (3.67 g, 90%)

### Intermediate 3

### 2-Iodo-4-methoxybenzonitrile

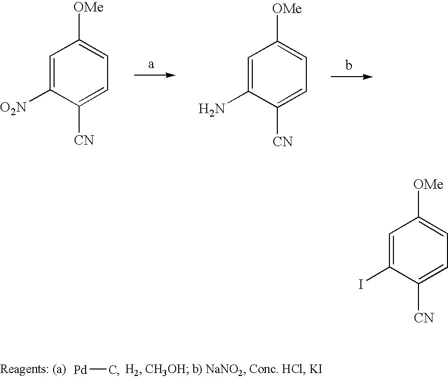

To a solution of 4-methoxy-2-nitrobenzonitrile (5.00 g, 22.46 mmol) in EtOH (93 mL) was added 5% Pd—C (0.500 g) under nitrogen atmosphere. The resulting mixture was hydrogenated at 1 atm. After stirring for 10 h, the mixture was filtered through celite, washed with EtOAc/MeOH. The combined filtrates were concentrated in vacuo and the residue was purified by column chromatograph on SiO₂ using 80 to 100% gradient of EtOAc in hexanes to afford 4-methoxy-2-aminobenzonitrile (3.23 g, 99%). 4-Methoxy-2-aminobenzonitrile (1.0 g, 6.58 mmol) was dissolved in a mixture of H₂O (7 mL), acetic acid (7 mL), and conc. HCl at rt, followed by addition of a solution of NaNO₂ (0.513 g, 7.43 mmol) in H₂O (2 mL) at 0° C. for 5 min. To the above mixture KI (2.18 g, 13.2 mmol) was added and after stirring for 10 h at rt, the resulting mixture was treated with solid NaHSO₃ and diluted with water. The product portion was extracted with CH₂Cl₂, washed with satd. NaHCO₃, brine, dried (Na₂SO₄), filtered, and concentrated in vacuo. The residue was purified by column chromatography on SiO₂ using 10 to 50% gradient of EtOAc in hexane to provide the example intermediate (1.0 g, 59%).

### Intermediate 4

### 6-Bromo-1,3-benzodioxole-5-carbonitrile

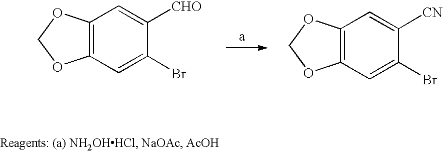

A mixture of 6-bromopiperonal (10 g, 44 mmol), hydroxyamine HCl (6.1 g, 87 mmol) and NaOAc (7.1 g, 87 mmol) in acetic acid (44 mL) was heated at 125° C. for 12 h. The excess acetic acid was removed at reduced pressure and the residue was diluted with CH₂Cl₂, washed with brine, dried over Na₂SO₄, filtered, and concentrated in vacuo and purified by column chromatography on SiO₂ using 10 to 40% EtOAc in hexanes to provide the example intermediate (8.47 g, 85%).

### Intermediate 5

### 2-Bromo-4-methoxy-1-(trifluoromethoxy)benzene

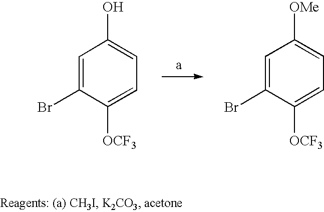

A mixture of 3-bromo-4-(trifluoromethoxy)phenol (2.0 g, 7.8 mmol), K₂CO₃ (1.62 g, 11.72 mmol), and MeI (2.43 mL, 39.06 mmol) in acetone (13 mL) was stirred for 10 h. After removal of acetone under reduced pressure, the crude was diluted with CH₂Cl₂, washed with brine, dried over Na₂SO₄, and concentrated in vacuo. The residue was purified by column chromatography on SiO₂ using gradient of 0 to 50% EtOAc in hexanes to provide the example intermediate (2.10 g, 99%).

### Intermediate 6

### 6-Bromo-7-chloro-2,3-dihydro-1,4-benzodioxine

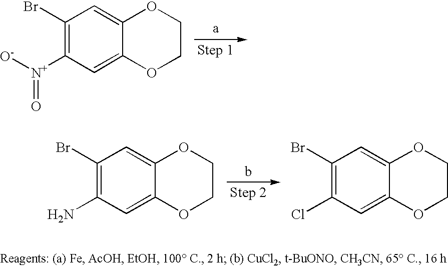

Step 1. A mixture of 6-bromo-7-nitro-2,3-dihydro-1,4-benzodioxine (1.79 g, 6.89 mmol), iron powder (1.59 g, 28.4 mmol 4.12) in a 1:1 mixture of glacial acetic acid (14.0 mL) and absolute ethanol (14.0 mL) was refluxed for 2 h. The reaction mixture was cooled to room temperature and diluted with water and neutralized with solid potassium carbonate. The mixture was filtered and the filtrate was extracted with CH₂Cl₂ (2×100 mL). The combined organic layer was washed with brine (2×100 mL). The organic layer was dried over Na₂SO₄, filtered and solvent was evaporated in vacuo to afford 7-bromo-2,3-dihydro-1,4-benzodioxin-6-amine was obtained as a yellow oil (0.76 g, 48%); GC-MS m/z 229. This product is sufficiently pure for the next step and used without further purification.

Step 2. A solution of 7-bromo-2,3-dihydro-1,4-benzodioxin-6-amine (0.76 g, 3.29 mmol) in anhydrous acetonitrile (6.5 mL) was added to a solution of cupric chloride (0.58 g, 4.13 mmol) and tert-butyl nitrite (0.63 mL, 5.33 mmol) in anhydrous acetonitrile (8.5 mL) at 65° C. and stirring continued overnight at 65° C. The mixture was cooled to room temperature and the solvent was removed in vacuo. The residue was purified by column chromatography on SiO₂ using a gradient of 0-100% EtOAc in hexanes to provide 6-bromo-7-chloro-2,3-dihydro-1,4-benzodioxine (0.53 g, 65%); GC-MS m/z 248.

### Intermediate 7

### 5,6-Diiodoindane

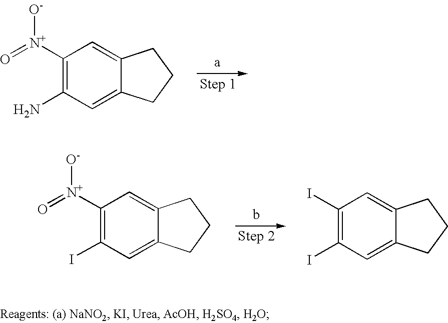

(b) (i) Fe, AcOH, EtOH, 100° C., 2 h, (ii) NaNO₂, AcOH, H₂SO₄, urea, KI, H₂O

Step 1. To a solution of 6-nitroindan-5-amine (1.5 g, 8.4 mmol) in glacial acetic acid (11.0 mL) was added a solution of sodium nitrite (0.844 g, 12.2 mmol) in conc. sulfuric acid (4.0 mL) at 0° C. To the above mixture additional quantity of conc. sulfuric acid (25.0 mL) was added and stirring continued at room temperature overnight. At the end of this period water (10 mL) followed by urea (0.887 g, 14.78 mmol) were added portion wise and stirred at room temperature for 5 minutes. To the above mixture a solution of potassium iodide (2.02 g, 12.15 mmol) in water (10 mL) was added, stirring continued for 10 min and neutralize with sodium bicarbonate. The reaction mixture was filtered to remove solids and extract with dichloromethane (2×200 mL). The organic layer was washed with brine (50 mL) and dried over Na₂SO₄, filtered, and the solvent was evaporated under reduced pressure to provide 5-iodo-6-nitroindane (0.558 g, 26%) as oil; GC-MS m/z 288.

Step 2. 5-Iodo-6-nitroindane was converted to the corresponding amine using reduction conditions used for intermediate 6 (Step 1). The corresponding amine was used for the next step without further purifications. The amine (0.558 g, 2.16 mmol) was converted to 5,6-diiodoindane (0.327 g, 41%) by similar reaction conditions described for in Step 1. GC-MS m/z 370.

### Intermediate 8

### 5-Bromo-6-iodoindane

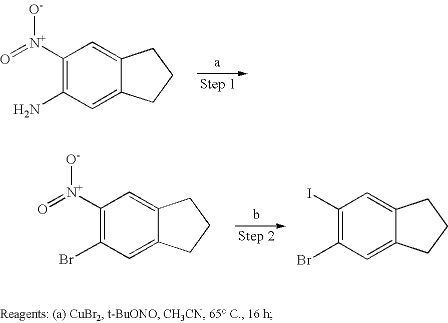

(b) (i) Fe, AcOH, EtOH, 100° C. 2 h, (ii) NaNO₂, AcOH, H₂SO₄, KI, urea, H₂O

Step 1. A mixture of cupric bromide (2.86 g, 12.8 mmol) and tert-butyl nitrite (1.90 mL, 16.0 mmol) in anhydrous acetonitrile (40 mL) was heated to 65° C. until it becomes a clear solution. To the above clear solution of 6-nitroindan-5-amine (1.91 g, 10.67 mmol) in acetonitrile (5 mL) was added and stirring continued overnight at 65° C. The reaction mixture was cooled to room temperature and aqueous hydrochloric acid (30 mL, 20%; v/v) was added and the mixture was extracted with diethyl ether (2×50 mL). The combined organic layer was washed with brine (50 mL) and dried over Na₂SO₄, filtered, and solvent was evaporated under reduced pressure. The product was chromatographed over SiO₂ using gradient of 0-100% ethyl acetate in hexanes to provide 5-bromo-6-nitroindane (2.15 g, 83%); GC-MS m/z 243.

Step 2. 5-Bromo-6-iodoindane (0.333 g, 26%) was prepared according to the procedure described for intermediate 7 using 5-bromo-6-nitroindane (2.15 g, 8.89 mmol); GC-MS m/z 322.

### Intermediate 9

### 2-Bromo-1-iodo-4-methoxybenzene

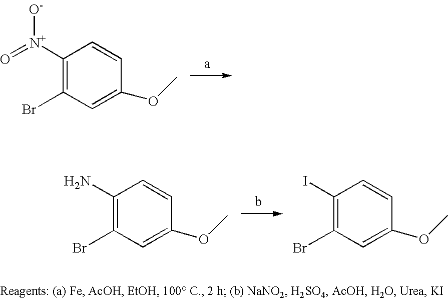

The example intermediate (1.149 g, 17%) was prepared in two-step sequential reaction starting from 2-bromo-4-methoxy-1-nitrobenzene (5.043 g, 21.73 mmol) according to the procedure described for intermediate 8; GC-MS m/z 202.

### Intermediate 10

### 7-Bromo-2,3-dihydro-benzo[1,4]dioxine-6-carbonitrile

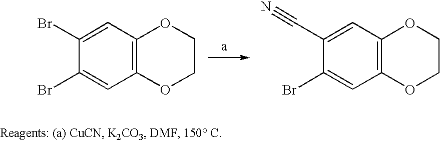

The mixture of 6,7-Dibromo-2,3-dihydro-benzo[1,4]dioxine (7.5 g, 25.5 mmol), CuCN (3.42 g, 38.27 mmol) and K₂CO₃ (5.28 g, 38.27 mmol) in dry dimethylformamide (50 mL) under N₂ atmosphere was heated to 150° C. for 2 days. Then the reaction mixture was cooled to room temperature and filtered through celite bed. The filtrate was diluted with ethyl acetate (120 mL), washed with water (2×100 mL) and brine (100 mL). The ethyl acetate layer was dried over Na₂SO₄, filtered, and the solvent was evaporated under vacuum. The residue was subjected to flash column chromatography using mixture of hexanes and EtOAc (2:1), to afford the example intermediate (3.2 g, 52%).

### Intermediate 11

### 8-[(7-Nitro-2,3-dihydro-1,4-benzodioxin-6-yl)thio]-9H-purin-6-amine

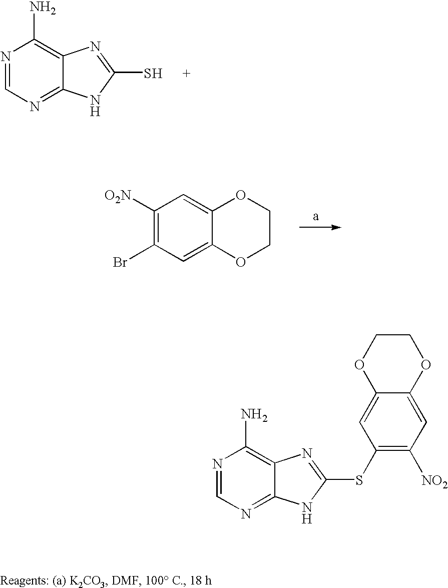

A mixture of 6-amino-9H-purine-8-thiol (0.250 g, 1.50 mmol), 6-bromo-7-nitro-2,3-dihydro-benzo[1,4]dioxine (0.390 g, 1.50 mmol) and K₂CO₃ (0.207 g, 1.50 mmol) in DMF (5 mL) heated 100° C. for 18 h. The reaction mixture was cooled and evaporated under reduced pressure, the crude obtained was chromatographed over SiO₂ using gradient of 0-20% methanol in dichloromethane to give product 8-(7-nitro-2,3-dihydro-benzo[1,4]dioxin-6-ylsulfanyl)-9H-purin-6-ylamine as yellow solid (0.120 g, 23%); LC-MS [M+H]⁺ 347.0.

### Intermediate 12

### 8-(6-Nitro-benzo[1,3]dioxol-5-ylsulfanyl)-9H-purin-6-ylamine

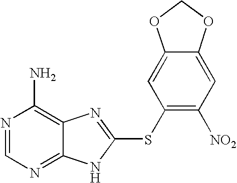

8-(6-Nitro-benzo[1,3]dioxol-5-ylsulfanyl)-9H-purin-6-ylamine was prepared according to the procedure described for intermediate 11; LC-MS [M+H]⁺ 333.0.

### Intermediate 13

### 8-[(7-Chloro-1,3-benzothiazol-2-yl)thio]-9H-purin-6-amine

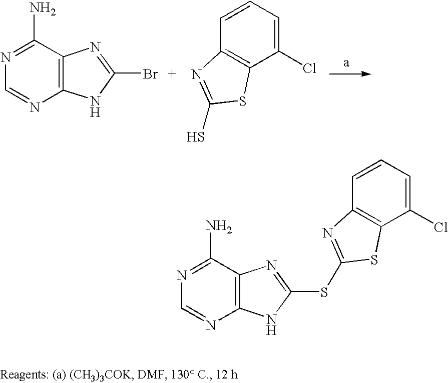

To a suspension of 8-bromoadenine (0.15 g, 0.701 mmol) and 7-chloro-benzothiazole-2-thiol (0.17 g, 0.841 mmol) in DMF was added potassium t-butoxide (0.094 μl, 0.841 mmol) at room temperature. The reaction mixture was heated at 130° C. for 12 h, then the reaction was cooled to room temperature and the crude was purified by silica gel flash column to give 8-(7-chloro-benzothiazol-2-ylsulfanyl)-9H-purin-6-ylamine (0.058 g, 25%); LC-MS [M+H]⁺ 335.0.

### Intermediate 14

### 6-[(6-amino-9H-purin-8-yl)thio]-3-oxoindane-5-carbonitrile

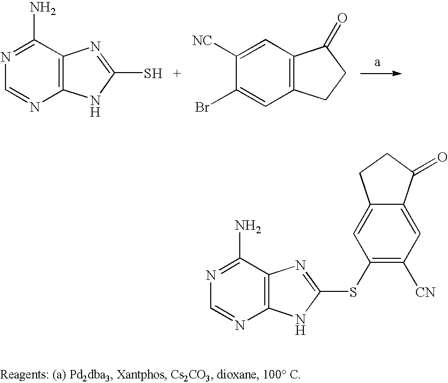

To 250 mL flask was charged with 8-mercaptoadenine (0.816 g, 4.89 mmol), 6-bromo-3-oxoindane-5-carbonitrile (1.50 g, 6.360 mmol), Pd₂ dba₃ (0.224 g, 0.245 mmol), Xantphos (0.283 g, 0.489 mmol), Cs₂CO₃ (3.19 g, 9.78 mmol), and anhydrous dioxane (14 mL). The resulting mixture was heated at 100° C. for 16 h under nitrogen. After concentration, the reaction mixture was diluted with EtOAc and water. The aqueous layer was separated and treated with AcOH (500 μL). The precipitate was filtered and dried to provide the example intermediate (0.446 g, 28%). Alternative work-up procedure: After the reaction was completed, the reaction was filtered and washed with 10% MeOH in THF. The combined filtrates were concentrated in vacuo and the residue was purified by chromatography on SiO₂ (CH₂Cl₂/EtOAc/MeOH, 2/2/0.5) to afford 6-[(6-Amino-9H-purin-8-yl)thio]-3-oxoindane-5-carbonitrile. ¹H NMR (DMSO-d₆) δ 8.19 (s, 1H), 8.16 (s, 1H), 7.56 (s, 1H), 3.11-3.08 (m, 2H), 2.68-2.65 (m, 2H); TOF LC-MS[M+H]⁺ 323.1.

### Intermediates 15-29

Intermediates 15-29 were synthesized in the same manner as described for intermediate 14, above, using appropriate starting materials, and are summarized in table 1, below.

TABLE 1
Preparation of Intermediates 15-29

| Inter- | | |
|---|---|---|
| mediate | Structure | Name and analytical data |
| 15 | 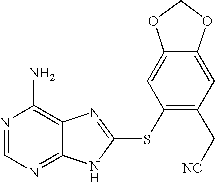 | {6-[(6-Amino-9H-purin-8-yl)thio]-1,3- benzodioxol-5-yl}acetonitrile; ¹H NMR (DMSO-d₆) δ 8.05 (s, 1H), 7.21- 7.27 (m, 2H), 6.13 (s, 2H), 4.11 (s, 2H); TOF LC-MS [M + H]⁺327.01 |
| 16 | 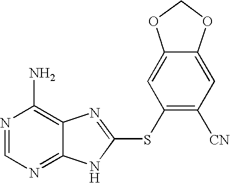 | 6-[(6-Amino-9H-purin-8-yl)thio]-1,3- benzodioxole-5-carbonitrile; 1H NMR (DMSO-d6) δ 8.08 (s, 1H), 7.59 (s, 1H), 7.29 (s, 1H), 7.26-7.16 (br s, 2H), 6.23 (s, 2H); TOF LC-MS [M + H]⁺315.0 |
| 17 | 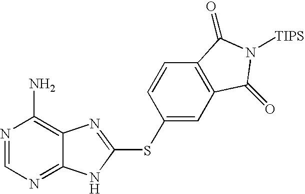 | 5-(6-Amino-9H-purin-8-ylsulfanyl)-2- triisopropylsilanyl-isoindole-1,3-dione; TOF LC-MS [M + H]⁺469.18 |
| 18 | 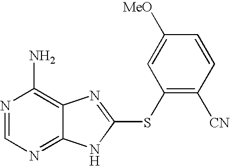 | 2-[(6-Amino-9H-purin-8-yl)thio]-4- methoxybenzonitrile; TOF LC-MS [M + H]⁺299.07 |
| 19 | 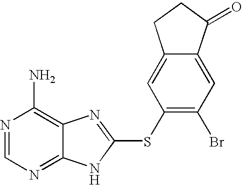 | 5-[(6-Amino-9H-purin-8-yl)thio]-6- bromoindan-1-one; ¹H NMR (DMSO-d₆) δ 8.17 (s, 1H), 7.87 (s, 1H), 7.58-7.44 (brs, 2H), 7.14 (s, 1H), 2.95-2.91 (m, 2H), 2.63-2.60 (m, 2H); TOF LC-MS [M + H]⁺375.99 |
| 20 | 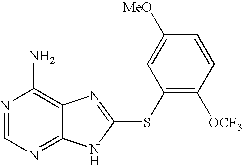 | 8-{[5-Methoxy-2- (trifluoromethoxy)phenyl]thio}-9H-purin-6- amine; TOF LC-MS [M + H]⁺358.06 |
| 21 | 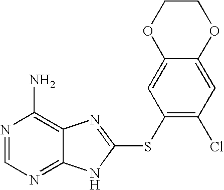 | 8-[(7-Chloro-2,3-dihydro-1,4-benzodioxin- 6-yl)thio]-9H-purin-6-amine; LC-MS [M − H]⁺333.9 |
| 22 | 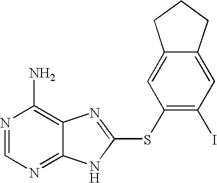 | 8-[(6-Iodo-2,3-dihydro-1H-inden-5-yl)thio]- 9H-purin-6-amine; LC-MS [M + H]⁺409.9 |
| 23 | 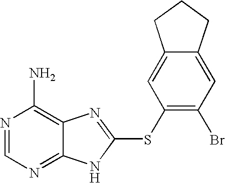 | 8-[(6-Bromo-2,3-dihydro-1H-inden-5- yl)thio]-9H-purin-6-amine; LC-MS [M + H]⁺361.8 |
| 24 | 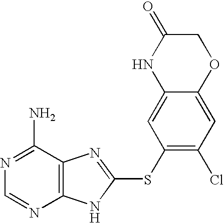 | 6-(6-Amino-9H-purin-8-ylsulfanyl)-7- bromo-4H-benzo[1,4]oxazin-3-one; ¹H NMR (DMSO-d₆) δ 10.8 (s, 1H), 8.19 (s, 1H), 7.66 (br s, 2H), 7.38 (s, 1H), 6.86 (s, 1H), 4.63 (s, 2H); TOF LC-MS [M + H]⁺392.98 |
| 25 | 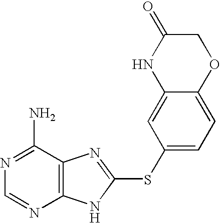 | 6-(6-Amino-9H-purin-8-ylsulfanyl)-4H- benzo[1,4]oxazin-3-one; LC-MS [M + H]⁺315.9 |
| 26 | 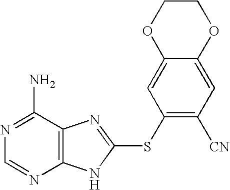 | 7-(6-Amino-9H-purin-8-ylsulfanyl)-2,3- dihydro-benzo[1,4]dioxane-6-carbonitrile; LC-MS [M + H]⁺327.0 |
| 27 | 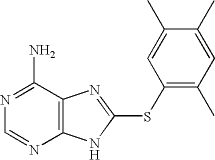 | 8-[2,4,5-trimethylphenyl)thio]-9H-purin-6- amine LC-MS [M + H]⁺286.1. |
| 28 | 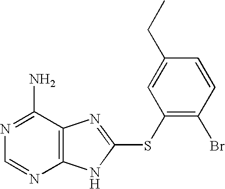 | 8-[(2-bromo-5-ethylphenyl)thio]-9H-purin- 6-amine LC-MS [M + H]⁺350.2 |
| 29 | 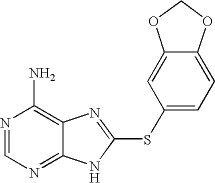 | 8-(1,3-benzodioxol-5-ylthio)-9H-purin-6- amine ¹H NMR (DMSO-d₆) δ 8.25 (s, 1H), 7.15 (s, 1H), 7.02 (d, 5.2 Hz, 2H), 6.80 (d, J = 5.2 Hz, 2H), 6.09 (s, 2H); LC-MS [M + H]⁺288.15. |

### Intermediate 30

### 2-(1-Formylpiperidine-4-yl)ethyl-4-methylbenzenesulfonate

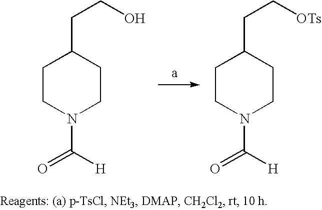

To a mixture of 4-(2-hydroxyethyl)piperidine-1-carbaldehyde (3.00 g, 19.1 mmol), NEt₃ (8.00 mL, 57.3 mmol), and DMAP (23.0 mg, 0.19 mmol) in CH₂Cl₂ (50 mL) was treated with a solution of p-toluene sulfonyl chloride (3.64 g, 19.1 mmol) in CH₂Cl₂ (10 mL) at 0° C. After stirring for 10 h at rt, the mixture was diluted with CH₂Cl₂, washed with brine, dried (Na₂SO₄), filtered, and concentrated in vacuo. The residue was purified by column chromatography on SiO₂ using of gradient of 50-100% ethyl acetate in hexanes to provide 2-(1-formylpiperidine-4-yl)ethyl-4-methylbenzenesulfonate (2.2 g, 37%); LC-MS [M+H]⁺ 311.9.

### Intermediate 31

### 2-(2-Chloro-ethyl)-1-propyl-piperidine

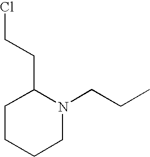

To a solution of 2-(1-propyl-piperidin-2-yl)-ethanol (0.150 g, 0.875 mmol) in dichloromethane (5 mL) was added methane sulfonylchloride (1.0 mL, 13.7 mmol) at rt and the contents refluxed for 3 h. The reaction mixture was diluted with dichloromethane and washed with NaHCO₃ (10%, W/V), followed by water. The dichloromethane layer was dried over Na₂SO₄, filtered and the solvent was evaporated to dryness to provide example intermediate in quantitative yield. The product was sufficiently pure for the next step and was used without any further purification; GC-MS m/z 189

### Intermediates 32-52

Intermediates 32-52 were synthesized in the same manner as described for intermediate 30 or intermediate 31, above, using appropriate starting materials, and are summarized in table 2, below.

TABLE 2
Preparation of Intermediates 32-52

| Intermediate | Structure | Name and analytical data |
|---|---|---|
| 32 | 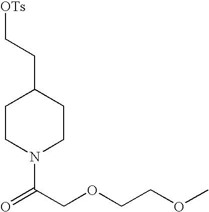 | Toluene-4-sulfonic acid 2-{1-[2-(2- methoxy-ethoxy)-acetyl]-piperidin-4-yl)- ethyl ester; LC-MS [M + H]⁺400.2 |
| 33 | 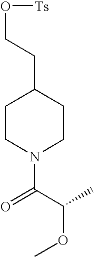 | Toluene-4-sulfonic acid 2-[1-((S)-2- methoxy-propionyl)-piperidin-4-yl]ethyl ester; LC-MS [M + H]⁺370.2 |
| 34 | 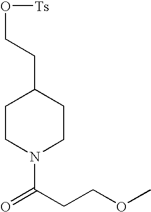 | Toluene-4-sulfonic acid 2-[1-(3- methoxy-propionyl)-piperidin-4-yl]ethyl ester; LC-MS [M + H]⁺370.2 |
| 35 | 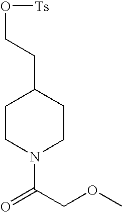 | Toluene-4-sulfonic acid 2-[1-(2- methoxy-acetyl)-piperidin-4-yl]ethyl ester; LC-MS [M + H]⁺356.1 |
| 36 | 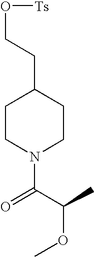 | Toluene-4-sulfonic acid 2-[1-((R)-2- methoxy-propionyl)-piperidin-4-yl]ethyl ester; LC-MS [M + H]⁺370.2 |
| 37 | 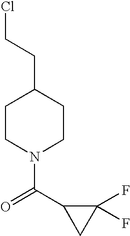 | Toluene-4-sulfonic acid 2-[1-(2,2- difluoro-cyclopropanecarbonyl)- piperidin-4-yl]ethyl ester; LC-MS [M + H]⁺388.1 |
| 38 | 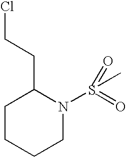 | 2-(2-Chloro-ethyl)-1- methanesulfonyl-piperidine; GC-MS m/z 224 |
| 39 | 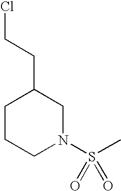 | 3-(2-Chloro-ethyl)-1- methanesulfonyl-1-piperidine; GC-MS m/z 224 |
| 40 | 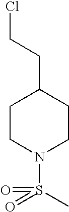 | 4-(2-Chloro-ethyl)-1- methanesulfonyl-1-piperidine; GC-MS m/z 224 |
| 41 | 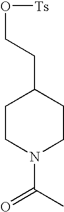 | Toluene-4-sulfonic acid 2-(1-acetyl- piperidin-4-yl)-ethyl ester; LC-MS [M + H]⁺326.0 |
| 42 | 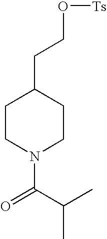 | Toluene-4-sulfonic acid 2-(1- isobutyryl-piperidin-4-yl)-ethyl ester; LC-MS [M + H]⁺354.1 |
| 43 | 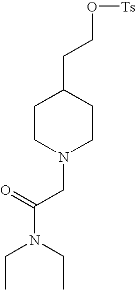 | Toluene-4-sulfonic acid 2-(1- diethylcarbamoylmethyl-piperidin-4-yl)- ethyl ester; LC-MS [M + H]⁺397.2 |
| 44 | 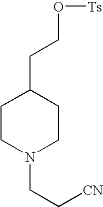 | Toluene-4-sulfonic acid 2-[1-(2- cyano-ethyl)-piperidin-4-yl]ethyl ester; LC-MS [M + H]⁺337.1 |
| 45 | 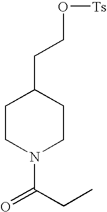 | Toluene-4-sulfonic acid 2-(1- propionyl-piperidin-4-yl)-ethyl ester; LC-MS [M + H]⁺340.1 |
| 46 | 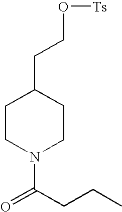 | Toluene-4-sulfonic acid 2-(1-butyryl- piperidin-4-yl)-ethyl ester; LC-MS [M + H]⁺354.1 |
| 47 | 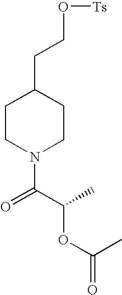 | Acetic acid (S)-1-methyl-2-oxo-2-{4- [2-(toluene-4-sulfonyloxy)-ethyl]- piperidin-l-yl}-ethyl ester; LC-MS [M + H]⁺398.3 |
| 48 | 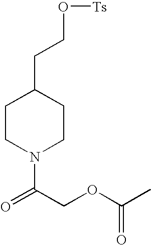 | Acetic acid 2-oxo-2- {4-[2-(toluene- 4-sulfonyloxy)-ethyl]-piperidin-l-yl}- ethyl ester; LC-MS [M + H]⁺384.2 |
| 49 | 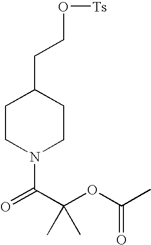 | Acetic acid 1,1-dimethyl-2-oxo-2-{4- [2-(toluene-4-sulfonyloxy)-ethyl]- piperidin-l-yl}-ethyl ester; LC-MS [M + H]⁺412.2 |
| 50 | 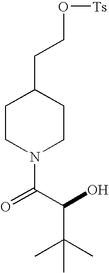 | Toluene-4-sulfonic acid 2-[1((S)-2- hydroxy-3,3-dimethyl-butyryl)-piperidin- 4-yl]-ethyl ester; TOF LC-MS [M + H]⁺392.2 |
| 51 | 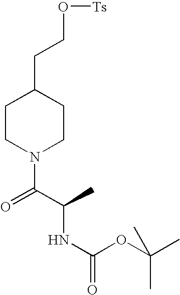 | Toluene-4-sulfonic acid 1-((R)-2- tert-butoxycarbonylamino-propionyl)- piperidin-4-ylmethyl ester; TOF LC-MS [M + Na]⁺463.1 |
| 52 | 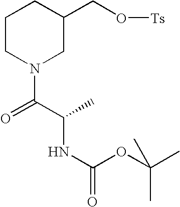 | Toluene-4-sulfonic acid 1-((S)-2-tert- butoxycarbonylamino-propionyl)- piperidin-3-ylmethyl ester TOF LC-MS [M + Na]⁺463.1 |

### Intermediate 53

### 2-{1-(1-Methyl-1H-tetrazol-5-yl)piperidin-4-yl)ethyl-4-methylbenzenesulfonate

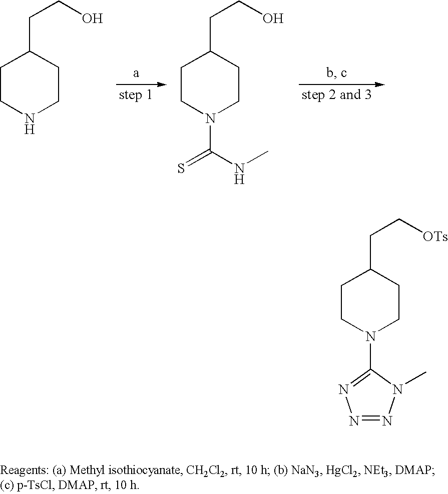

Step 1. N-Methyl-4-(2-hydroxyethyl)piperidine-1-carbothioamide: To a solution of 2-(piperidin-4-yl)ethanol (864 mg, 6.70 mmol) in CH₂Cl₂ (22 mL) was added isothiocyanatomethane (490 μL, 6.70 mmol). After stirring for 10 h at rt, the mixture was concentrated in vacuo and the residue was purified by column chromatography on SiO₂ (EtOAc) to provide the example intermediate (1.04 g, 77%).

Step 2. 2-{1-(1-Methyl-1H-tetrazol-5-yl)piperidin-4-yl}ethanol: To a mixture of N-methyl-4-(2-hydroxyethyl)piperidine-1-carbothioamide (1.63 g, 8.07 mmol), HgCl₂ (2.41 g, 8.88 mmol), and NaN₃ (1.57 g, 24.2 mmol) in DMF (20 mL) was added NEt₃ (3.37 mL, 24.2 mmol) at rt. After stirring for 10 h, the reaction mixture was filtered, and the filter cake was washed with CH₂Cl₂. The combined filtrates and washings were washed with brine, dried (Na₂SO₄), filtered and concentrated in vacuo, and the residue was purified by SiO₂ chromatograph (60% EtOAc in hexane) to afford the example intermediate (0.720 g, 42%); LC-MS [M+Na]⁺ 234.0.

Step 3. 2-{1-(1-Methyl-1H-tetrazol-5-yl)piperidin-4-yl)ethyl-4-methylbenzenesulfonate: The example intermediate (0.417 g, 80%) was obtained from 2-{1-(1-methyl-1H-tetrazol-5-yl)piperidin-4-yl}ethanol (0.399 g, 1.42 mmol) according to the procedure described for intermediate 30; LC-MS [M+Na]⁺ 365.5.

### Intermediate 54

### 2-(2-bromoethyl)-5,8-dihydro-1H-[1,2,4]triazole[1,2-a]pyridazine-1,3(2H)-dione

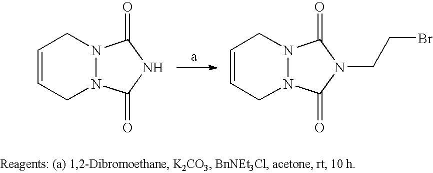

To a mixture of 5,8-dihydro-1H-[1,2,4]triazole[1,2-a]pyridazine-1,3(2H)-dione (250 mg, 1.63 mmol), K₂CO₃ (678 mg, 4.90 mmol), and BnNEt₃Cl (45 mg, 0.2 mmol) in acetone (4 mL) was added 1,2-dibromoethane (422 μL, 4.90 mmol). After stirring for 10 h at rt, acetone was removed under reduced pressure and the residue was diluted with CH₂Cl₂, washed with brine, dried (Na₂SO₄), filtered, and concentrated in vacuo. The residue was purified by column chromatography on SiO₂ (30% hexanes in EtOAc to 10% hexanes in EtOAc) to afford 2-(2-bromoethyl)-5,8-dihydro-1H-[1,2,4]triazole[1,2-a]pyridazine-1,3(2H)-dione (0.400 g, 94%); LC-MS [M+H]⁺ 259.8.

### Intermediate 55

### 2-(3-Bromopropyl)-5,8-dihydro-1H-[1,2,4]triazolo[1,2-a]pyridazine-1,3(2H)-dione

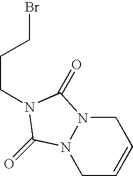

The example intermediate was prepared according to the procedure described for intermediate 54 using 5,8-dihydro-1H-[1,2,4]triazole[1,2-a]pyridazine-1,3(2H)-dione and 1,3-dibromo propane. LC-MS [M+H]⁺ 273.9.

### Intermediate 56

### 2-(4,4-Difluorocyclohexyl)ethyl 4-methylbenzenesulfonate

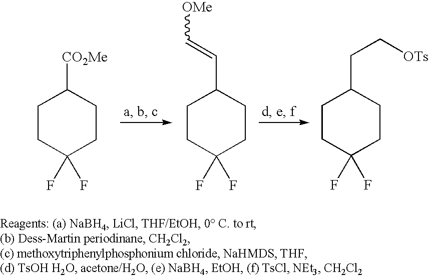

2-(4,4-Difluorocyclohexyl)ethyl 4-methylbenzenesulfonate was prepared by following a six-step sequence of straightforward transformation well known in the art: 1) reduction using NaBH₄/LiCl, 2) oxidation using Dess-Martin periodinane, 3) Wittg reaction using methoxytriphenylphosphonium chloride, 4) hydrolysis of the resulting enolether with TsOH, 5) reduction using NaBH₄, and 6) tosylation of the resulting alcohol; ¹H NMR (CDCl₃) δ 7.79 (d, J=8.4 Hz, 2H), 7.36 (d, J=8.2 Hz, 2H), 4.07 (t, J=6.0 Hz, 2H), 2.40 (s, 3H), 2.08-1.98 (m, 2H), 1.82-1.42 (m, 7H), 1.27-1.16 (m, 2H); LC-MS [M+Na]⁺ 341.1

### Example Compounds 1 and 2

### 6-({6-Amino-9-[2-(1-formylpiperidin-4-yl)ethyl]-9H-purin-8-yl}thio)-3-oxoindane-5-carbonitrile and 6-({6-amino-3-[2-(1-formylpiperidin-4-yl)ethyl]-3H-purin-8-yl}thio)-3-oxoindane-5-carbonitrile

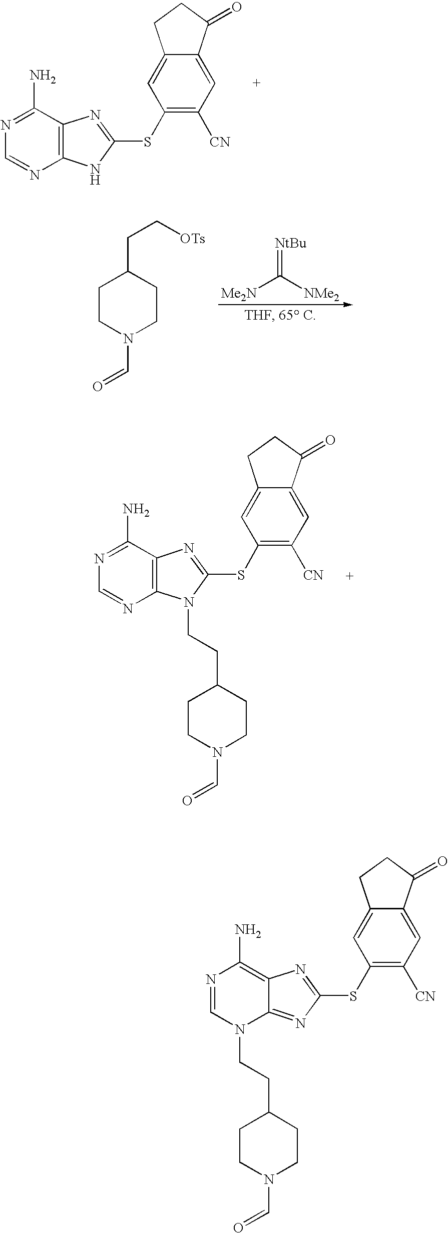

A mixture of 6-[(6-amino-9H-purin-8-yl)thio]-3-oxoindane-5-carbonitrile (100 mg, 0.310 mmol), 2-(1-formylpiperidin-4-yl)ethyl 4-methylbenzenesulfonate (126 mg, 0.400 mmol), and Barton's base (96 μL, 0.47 mmol) in THF (1.6 mL) was heated at 65-70° C. for 6-15 h. The reaction mixture was then allowed to reach ambient temperature. After removal of solvent under reduced pressure, the residue was purified by preparative HPLC and isolated via lyophilization to give the N-9 isomer (13 mg) and the N-3 isomer (10 mg). 6-({6-Amino-9-[2-(1-formylpiperidin-4-yl)ethyl]-9H-purin-8-yl}thio)-3-oxoindane-5-carbonitrile. ¹H NMR (CD₃OD) δ 8.32 (s, 1H), 8.16 (s, 1H), 7.98 (s, 1H), 7.90 (s, 1H), 4.42 (t, J=7.2 Hz, 2H), 4.29 (br d, J=13.2 Hz, 1H), 3.71 (brd, J=13.6 Hz, 1H), 3.25-3.21 (m, 2H), 3.09 (td, J=13.8, 3.2 Hz, 1H), 2.78-2.72 (m, 2H), 2.65 (td, J=12.8, 3.2 Hz, 1H), 1.96-1.84 (m, 4H), 1.64 (m, 1H), 1.21 (qd, J=12.8, 4.4 Hz, 1H), 1.13 (qd, J=12.8, 4.8 Hz, 1H); TOF LC-MS [M+H]⁺ 462.17 and 6-({6-amino-3-[2-(1-formylpiperidin-4-yl)ethyl]-3H-purin-8-yl}thio)-3-oxoindane-5-carbonitrile. TOF LC-MS [M+H]⁺ 462.17.

### Example Compounds 3-87

Example compounds 3-87 were synthesized in the same manner as described for example compounds 1 and 2, above, using appropriate starting intermediates described above, and are summarized in table 3, below.

TABLE 3
Preparation of Example Compounds 3-87

| Example | | |
|---|---|---|
| Compound | | |
| No. | Structure | Name and Analytical Data |
| 3 | 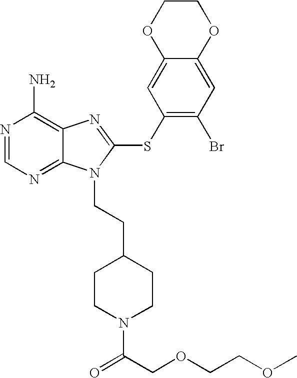 | 8-[(7-Bromo-2,3-dihydro-1,4- benzodioxin-6-yl)thio]-9-(2-{1-[(2- methoxyethoxy)acetyl]piperidin-4- yl}ethyl)-9H-purin-6-amine; ¹H NMR (DMSO-d₆) δ 6 8.10 (s, 1H), 7.48-7.40 (br s, 2H), 7.27 (s, 1H), 6.68 (s, 1H), 4.03-4.00 (m, 9H), 3.72 (d, J = 13.2 Hz, 1H), 3.55-3.50 (m, 2H), 3.46- 3.42 (m, 2H), 3.23 (s, 3H), 2.82 (t, J = 12.1 Hz, 1H), 2.42 (t, J = 10.1 Hz, 1H), 1.68 (d, J = 12.5 Hz, 2H), 1.57 (q, J = 7.0 Hz, 2H), 1.44-1.30 (m, 1H), 1.12- 0.88 (m, 2H); TOF LC-MS [M + H]⁺607.13 |
| 4 | 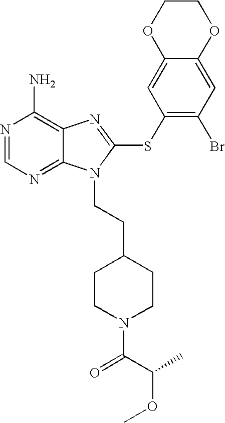 | 8-[(7-Bromo-2,3-dihydro-1,4- benzodioxin-6-yl)thio]-9-(2-{1-[(2S)-2- methoxypropanoyl]piperidin-4- yl}ethyl)-9H-purin-6 -amine; ¹H NMR (DMSO-d₆) δ 8.16 (s, 1H), 7.50-7.40 (br s, 2H), 7.27 (s, 1H), 6.68 (s, 1H), 4.30 (d, J = 13.2 Hz, 1H), 4.05-4.03 (m, 7H), 4.00-3.58 (m, 1H), 3.16 (s, 3H), 2.85 (t, J = 13.2 Hz, 1H), 2.50-2.39 (m, 1H), 1.70 (t, J = 14.0 Hz, 2H), 1.58 (q, J = 7.0 Hz, 2H), 1.46- 1.35 (m, 1H), 1.17 (t, J = 6.6 Hz, 3H), 1.09-0.88 (m, 2H); TOF LC-MS [M + H]⁺579.13 |
| 5 | 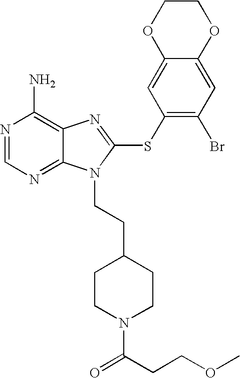 | 8-[(7-Bromo-2,3-dihydro-1,4- benzodioxin-6-yl)thio]-9-{2-[1-(3- methoxypropanoyl)piperidin-4- yl]ethyl}-9H-purin-6-amine; ¹H NMR (CD₃OD) δ 8.20 (s, 1H), 7.25 (s, 1H), 7.10 (s, 1H), 4.51 (d, J = 14.4 Hz, 1H), 4.36-4.24 (m, 6H), 3.98 (d, J = 12.5 Hz, 1H), 3.64 (t, J = 6.2 Hz, 2H), 3.08-2.98 (m, 1H), 2.69-2.55 (m, 3H), 1.90-1.73 (m, 4H), 1.52-1.64 (m, 1H),1.25-1.08 (m, 2H); TOF LC-MS [M + H]⁺579.12 |
| 6 | 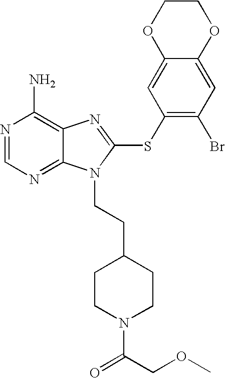 | 8-[(7-Bromo-2,3-dihydro-1,4- benzodioxin-6-yl)thio]-9-{2-[1- (methoxyacetyl)piperidin-4-yl]ethyl}- 9H-purin-6-amine; ¹H NMR (DMSO-d₆) δ 8.16 (s, 1H), 7.50-7.40 (broad s, 2H), 7.28 (s, 1H), 6.68 (s, 1H), 4.30-4.15 (m, 7H), 4.03 (q, J = 13.6 Hz, 2H), 3.68 (d, J = 12.8 Hz, 1H), 3.25 (s, 1H), 2.81 (t, J = 14.0 Hz, 1H), 2.48-2.38 (m, 1H), 1.68 (d, J = 10.9 Hz, 2H), 1.57 (q, J = 6.6 Hz, 2H), 1.43-1.30 (m, 1H), 1.10-0.88 (m, 2H); TOF LC-MS [M + H]⁺563.10 |
| 7 | 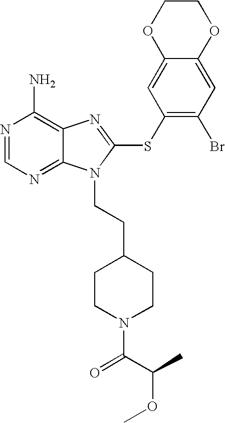 | 8-[(7-Bromo-2,3-dihydro-1,4- benzodioxin-6-yl)thio]-9-(2-{1-[(2R)- 2-methoxypropanoyl]piperidin-4- yl}ethyl)-9H-purin-6-amine; ¹H NMR (DMSO-d₆) δ 8.16 (s, 1H), 7.50-7.40 (broad s, 2H), 7.27 (s, 1H), 6.67 (s, 1H), 4.3 (d, J = 13.6 Hz, 1H), 4.26-4.14 (m, 7H), 4.03-3.9 (m, 1H), 3.16 (s, 3H), 2.85 (t, J = 12.1 Hz, 1H), 2.49-2.39 (m, 1H), 1.7 (t, J = 15.2 Hz, 2H), 1.57 (q, J = 7.0 Hz, 2H), 1.45- 1.35 (m, 1H), 1.17 (t, J = 6.2 Hz, 3H), 1.08-0.88 (m, 2H); TOF LC-MS [M + H]⁺577.12 |
| 8 | 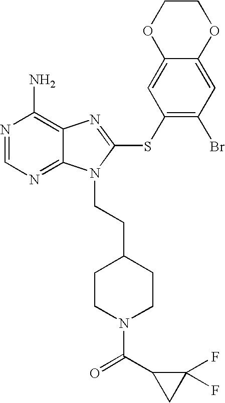 | 8-[(7-Bromo-2,3-dihydro-1,4- benzodioxin-6-yl)thio]-9-(2-{1-[(2,2- difluorocyclopropyl)carbonyl]piperidin- 4-yl}ethyl)-9H-purin-6-amine; ¹H NMR (DMSO-d₆) δ 8.22 (s, 1H), 7.85-7.65 (bs, 2H), 7.29 (d, J = 6.2 Hz, 1H), 6.73 (d, J = 5.4 Hz, 1H), 4.35-4.13 (m, 7H), 3.99 (t, J = 13.6 Hz, 1H), 3.20-2.90 (m, 2H), 2.60-2.44 (m, 1H), 1.92-1.53 (m, 6H), 1.50-1.37 (m, 1H), 1.20-0.80 (m, 2H); TOF LC-MS [M + H]⁺595.08 |
| 9 | 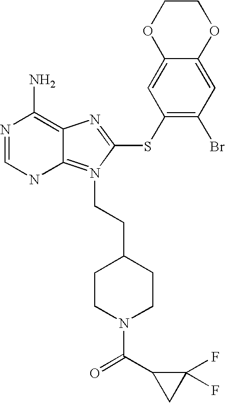 | 8-[(7-Bromo-2,3-dihydro-1,4- benzodioxin-6-yl)thio]-3-(2-{1-[(2,2- difluorocyclopropyl)carbonyl]piperidin- 4-yl}ethyl)-3H-purin-6-amine; TOF LC-MS [M + H]⁺595.08 |
| 10 | 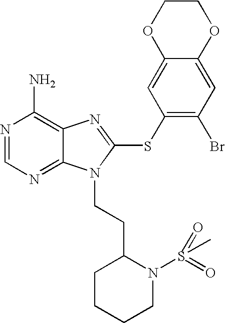 | 8-[(7-Bromo-2,3-dihydro-1,4- benzodioxin-6-yl)thio]-9-{2-[1- (methylsulfonyl)piperidin-2-yl]ethyl}- 9H-purin-6-amine; ¹H NMR (Acetone-d₆) δ 8.22 (s, 1H), 7.20 (s, 1H), 6.80 (s, 1H), 4.32- 4.30 (m, 4H), 4.20-4.10 (m, 2H), 3.20- 3.10 (m, 2H), 3.00 (s, 3H) 2.30-2.10 (m, 2H), 2.00-1.90 (m, 2H), 1.80-1.70 (m, 2H), 1.60-1.50 (m, 3H); LC-MS [M + H]⁺569.05 |
| 11 | 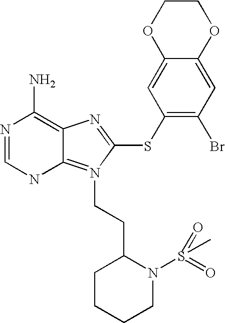 | 8-[(7-Bromo-2,3-dihydro-1,4- benzodioxin-6-yl)thio]-3-{2-[1- (methylsulfonyl)piperidin-2-yl]ethyl}- 3H-purin-6-amine; ¹H NMR (Acetone-d₆) δ 8.60 (s, 1H), 7.40 (s, 1H), 7.30 (s, 1H), 4.40- 4.30 (m, 4H), 4.30-4.10 (m, 2H), 3.00 (s, 3H) 2.70-2.60 (m, 2H), 2.00-1.90 (m, 2H), 1.83-1.80 (m, 2H), 1.60-1.50 (m, 5H); LC-MS [M + H]⁺569.07 |
| 12 | 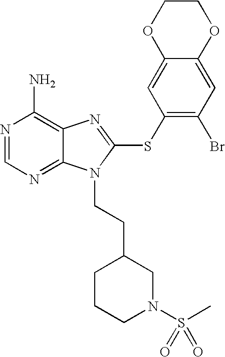 | 8-[(7-Bromo-2,3-dihydro-1,4- benzodioxin-6-yl)thio]-9-{2-[1- (methylsulfonyl)piperidin-3-yl]ethyl}- 9H-purin-6-amine; ¹H NMR (Acetone-d₆) δ 8.20 (s, 1H), 7.20 (s, 1H), 6.80 (s, 1H), 4.40- 4.30 (m, 4H), 4.30-4.20 (m, 2H), 3.50- 3.40 (m, 2H), 3.00 (s, 3H) 2.60 -2.50 (m, 2H), 2.00-1.90 (m, 2H), 1.80-1.70 (m, 2H), 1.60-1.50 (m, 3H); LC-MS [M + H]⁺569.07 |
| 13 | 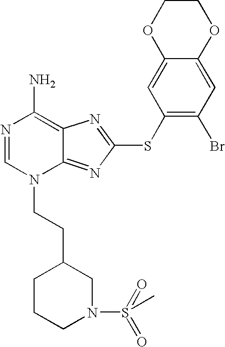 | 8-[(7-Bromo-2,3-dihydro-1,4- benzodioxin-6-yl)thio]-3-{2-[1- (methylsulfonyl)piperidin-3-yl]ethyl}- 3H-purin-6-amine; ¹H NMR (Acetone-d₆) δ 8.60 (s, 1H), 7.40 (s, 1H), 7.3 0(s, 1H), 4.50 (t, J = 7.6 Hz, 2H), 4.40-4.30 (m, 4H), 3.60-3.50 (m, 2H), 2.80 (s, 3H) 2.10- 1.90 (m, 4H), 1.80-1.70 (m, 3H), 1.60- 1.50 (m, 2H); LC-MS [M + H]⁺569.04 |
| 14 | 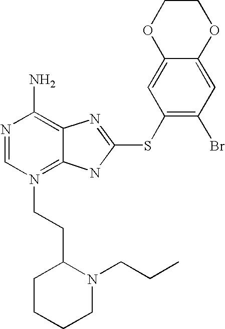 | 8-[(7-Bromo-2,3-dihydro-1,4- benzodioxin-6-yl)thio]-9-[2-(1- propylpiperidin-2-yl)ethyl]-9H-purin- 6-amine; LC-MS [M + H]⁺533.14 |
| 15 | 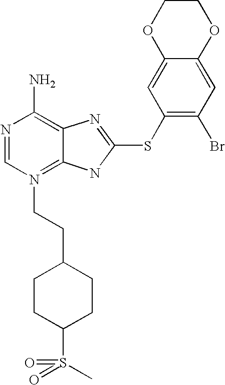 | 8-[(7-Bromo-2,3-dihydro-1,4- benzodioxin-6-yl)thio]-9-{2-[1- (methylsulfonyl)piperidin-4-yl]ethyl}- 9H-purin-6-amine; ¹H NMR (CD₃OD) δ 8.20 (s, 1H), 7.30 (s, 1H), 7.20 (s, 1H), 4.40 (t, J = 7.2 Hz, 2H), 4.30-4.20 (m, 4H), 3.71-3.70 (m, 2H), 2.80 (s, 3H) 2.70- 2.60 (m, 2H), 1.93-1.90 (m, 2H), 1.83- 1.80 (m, 2H), 1.40-1.30 (m, 3H); LC-MS [M + H]⁺569.05 |
| 16 | 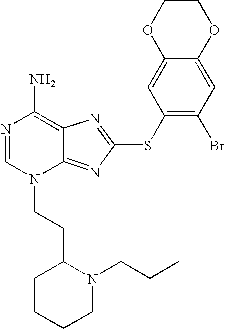 | 8-[(7-Bromo-2,3-dihydro-1,4- benzodioxin-6-yl)thio]-3-[2-(1- propylpiperidin-2-yl)ethyl]-3H-purin- 6-amine; LC-MS [M + H]⁺533.14 |
| 17 | 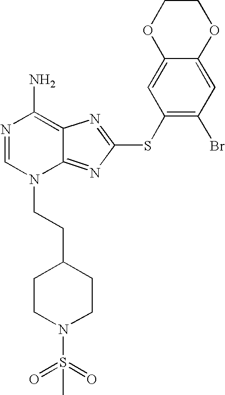 | 8-[(7-Bromo-2,3-dihydro-1,4- benzodioxin-6-yl)thio]-3-{2-[1- (methylsulfonyl)piperidin-4-yl]ethyl}- 3H-purin-6-amine; ¹H NMR (CD₃OD) δ 8.50 (s, 1H), 7.40 (s, 1H), 7.30 (s, 1H), 4.40 (t, J = 6.4 Hz, 2H), 4.35-4.30 (m, 4H), 3.71-3.70 (m, 2H), 2.80 (s, 3H) 2.72- 2.70 (m, 2H), 1.93-1.90 (m, 4H), 1.33- 1.30 (m, 3H); LC-MS [M + H]⁺569.14 |
| 18 | 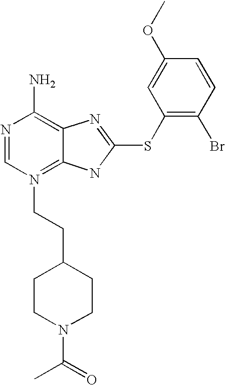 | 9-[2-(1-Acetylpiperidin-4- yl)ethyl]-8-[(2-bromo-5- methoxyphenyl)thio]-9H-purin-6- amine; ¹H NMR (DMSO-d₆) δ 8.34 (s, 1H), 7.65 (d, J = 9.2 Hz, 1H), 6.9 (dd, J = 9.2, 3.2 Hz, 1H), 6.58 (d, J = 3.2 Hz, 1H), 4.3-4.21 (m, 3H), 3.74-3.70 (m, 2H), 3.65 (s, 3H), 1.94 (s, 3H), 1.68- 1.55 (m, 4H), 1.1-0.85 (m, 4H); LC-MS [M + H]⁺505.1 |
| 19 | 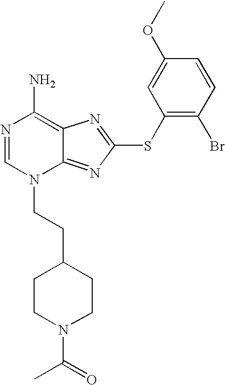 | 3-[2-(1-Acetylpiperidin-4- yl)ethyl]-8-[(2-bromo-5- methoxyphenyl)thio]-3H-purin-6- amine; LC-MS [M + H]⁺505.1 |
| 20 | 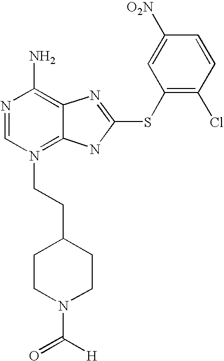 | 4-(2-{6-Amino-8-[(2-chloro-5- nitrophenyl)thio]-9H-purin-9- yl}ethyl)piperidine-1-carbaldehyde; ¹H NMR (DMSO-d₆) δ 8.24 (s, 1H), 8.15 (dd, J = 9.2, 2.8 Hz, 1H), 7.93 (d, J = 9.2 Hz, 1H), 7.92 (s, 1H), 7.76 (d, J = 2.8 Hz, 1H), 4.23 (t, J = 7.2 Hz, 2H), 4.09-4.05 (m, 1H), 3.75-3.65 (m, 2H), 1.75-1.6 (m, 4H), 1.1-0.86 (m, 4H); LC-MS [M + H]⁺462.1 |
| 21 | 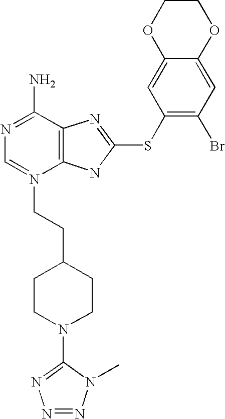 | 8-[(7-Bromo-2,3-dihydro-1,4- benzodioxin-6-yl)thio]-9-{2-[1-(1- methyl-1H-tetrazol-5-yl)piperidin-4- yl]ethyl}-9H-purin-6-amine; ¹H NMR (CD₃OD) δ 8.29 (s, 1H), 7.27 (s, 1H), 7.23 (s, 1H), 4.39 (t, J = 7.2 Hz, 2H), 4.32-4.26 (m, 4H), 3.90 (s, 3H), 3.63 (d, J = 12.8 Hz, 2H), 2.98 (td, J = 12.4, 2.0 Hz, 2H), 1.93 (d, J = 10.4 Hz, 2H), 1.86 (q, J = 7.6 Hz, 2H), 1.55 (m, 1H), 1.47 (m, 2H); TOF LC-MS [M + H]⁺573.10 |
| 22 | 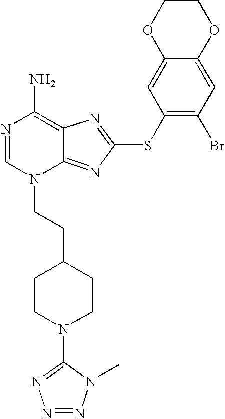 | 8-[(7-Bromo-2,3-dihydro-1,4- benzodioxin-6-yl)thio]-3-{2-[1-(1- methyl-1H-tetrazol-5-yl)piperidin-4- yl]ethyl}-3H-purin-6-amine; TOF LC-MS [M + H]⁺573.10 |
| 23 | 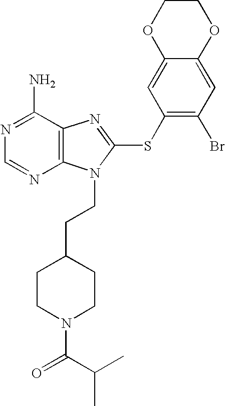 | 8-[(7-Bromo-2,3-dihydro-1,4- benzodioxin-6-yl)thio]-9-[2-(1- isobutyrylpiperidin-4-yl)ethyl]-9H- purin-6-amine; ¹H NMR (CD₃OD) δ 8.31 (s, 1H), 7.27 (s, 1H), 7.25 (s, 1H), 4.53 (brd, J = 13.2 Hz, 1H), 4.37 (t, J = 6.0 Hz, 2H), 4.30-4.27 (m, 4H), 4.05 (brd, J = 14.8 Hz, 1H), 3.06 (t, J = 13.2 Hz, 1 2.95 (m, 1H), 2.57 (t, J = 12.8 Hz, 1H), 1.95-1.79 (m, 4H), 1.60 (m, 1H), 1.24-1.10 (m, 2H), 1.10-1.06 (m, 6H); TOF LC-MS [M + H]⁺561.13 |
| 24 | 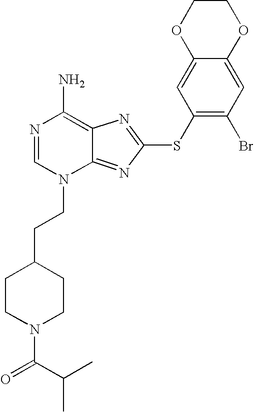 | 8-[(7-Bromo-2,3-dihydro-1,4- benzodioxin-6-yl)thio]-342-(1- isobutyrylpiperidin-4-ypethyl]-3H- purin-6-amine; TOF LC-MS [M + H]⁺561.13 |
| 25 | 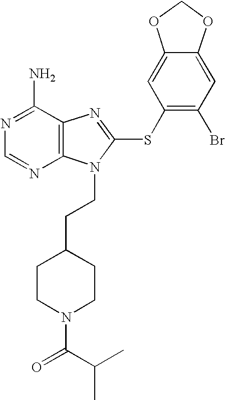 | 8-[(6-Bromo-1,3-benzodioxol-5- yl)thio]-9-[2-(1-isobutyrylpiperidin-4- yl)ethyl]-9H-purin-6-amine; ¹H NMR (CD₃OD) δ 8.23 (s, 1H), 7.27 (s, 1H), 7.13 (s, 1H), 6.08 (s, 2H), 4.52 (brd, J = 12.4 Hz, 1H), 4.34 (t, J = 6.8 Hz, 2H), 4.04 (brd, J = 14.4 Hz, 1H) 3.05 (t, J = 13.2 Hz, 1H), 2.94 (m, 1H), 2.57 (t, J = 12.4 Hz, 1H),1.99- 1.79 (m, 4H), 1.62 (m, 1H), 1.25-1.10 (m, 2H), 1.12-1.05 (m, 6H); TOF LC-MS [M + H]⁺547.11 |
| 26 | 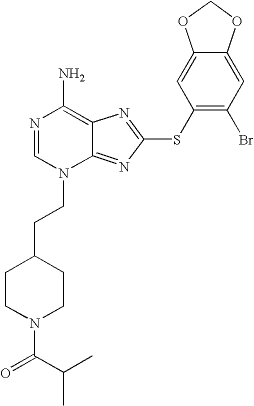 | 8-[(6-Bromo-1,3-benzodioxol-5- yl)thio]-3-[2-(1-isobutyrylpiperidin-4- yl)ethyl]-3H-purin-6-amine; TOF LC-MS [M + H]⁺547.11 |
| 27 | 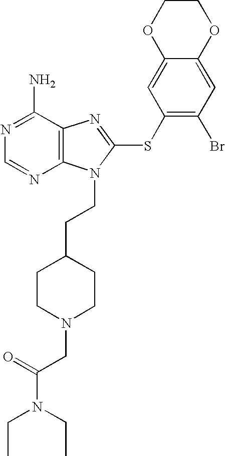 | 2-[4-(2-{6-Amino-8-[(7-bromo- 2,3-dihydro-1,4-benzodioxin-6- yl)thio]-9H-purin-9-yl}ethyl)piperidin- 1-yl]-N,N-diethylacetamide; ¹H NMR (CD₃OD) δ 8.25 (s, 1H), 7.27 (s, 1H), 7.21 (s, 1H), 4.37 (t, J = 6.8 Hz, 2H), 4.32-4.24 (m, 4H), 4.12 (s, 2H), 3.63 (brd, J = 11.2 Hz, 2H), 3.48 (q, J = 6.8 Hz, 2H), 3.32 (q, J = 6.8 Hz, 2H; overlapped with CD₃OD), 3.22-2.61 (m, 2H), 2.15-2.12 (m, 2H), 1.88-1.86 (m, 2H), 1.60-1.57 (m, 3H), 1.22 (t, J = 6.8 Hz, 3 H), 1.51 (t, J = 6.8 Hz, 3H); TOF LC-MS [M + H]⁺604.17 |
| 28 | 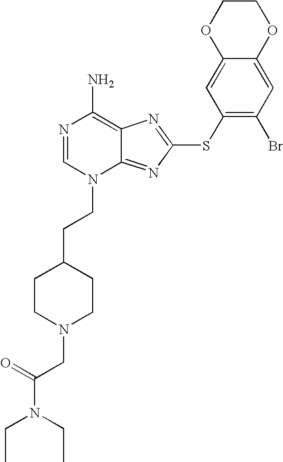 | 2-[4-(2-{6-Amino-8-[(7-bromo- 2,3-dihydro-1,4-benzodioxin-6- yl)thio]-3H-purin-3-yl}ethyl)piperidin- 1-yl]-N,N-diethylacetamide; TOF LC-MS [M + H]⁺604.17 |
| 29 | 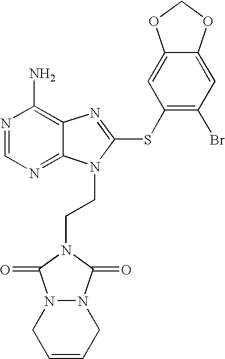 | 2-(2-{6-Amino-8-[(6-bromo-1,3- benzodioxol-5-yl)thio]-9H-purin-9- yl}ethyl)-5,8-dihydro-1H- [1,2,4]triazolo[1,2-a]pyridazine- 1,3(2H)-dione; ¹H NMR (DMSO-d₆) δ 8.09 (s, 1H), 7.34 (s, 1H), 6.87 (s, 1H), 6.08 (s, 2H), 5.89 (s, 2H), 4.44-4.38 (m, 2H), 3.86 (s, 4H), 3.84-3.78 (m, 2H); TOF LC-MS [M + H]⁺545.03 |
| 30 | 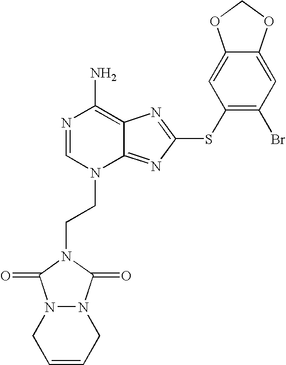 | 2-(2-{6-Amino-8-[(6-bromo-1,3- benzodioxol-5-yl)thio]-3H-purin-3- yl}ethyl)-5,8-dihydro-1H- [1,2,4]triazolo[1,2-a]pyridazine- 1,3(2H)-dione; TOF LC-MS [M + H]⁺545.03 |
| 31 | 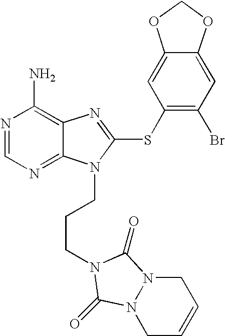 | {6-Amino-8-[(6-bromo-1,3- benzodioxol-5-yl)thio]-9H-purin-9- yl}propyl)-5,8-dihydro-1H- [1,2,4]triazolo[1,2-a]pyridazine- 1,3(2H)-dione; ¹H NMR (CDCl₃) δ 8.27 (s, 1H), 7.07 (s, 1H), 6.87 (s, 1H), 6.02 (s, 2H), 5.94 (s, 2H), 4.31 (t, J = 7.2 Hz, 2H), 4.10 (s, 4H), 3.67 (t, J = 6.8 Hz, 2H), 2.24-2.16 (m, 2H); TOF LC-MS [M + H]⁺559.05 |
| 32 | 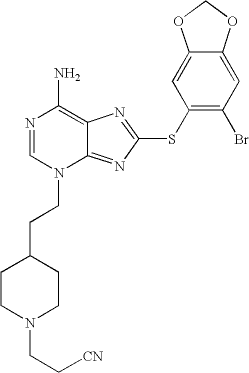 | 3-[4-(2-{6-Amino-8-[(6-bromo- 1,3-benzodioxol-5-yl)thio]-3H-purin-3- l}ethylpiperidin-1-yl]propanenitrile TOF LC-MS [M + H]⁺529.99 |
| 33 | 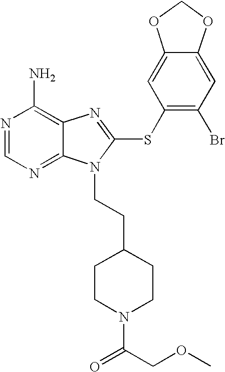 | 6-[(6-Amino-9-{2-[1- (methoxyacetyl)piperidin-4-yl]ethyl}- 9H-purin-8-yl)thio]-1,3-benzodioxole- 5-carbonitrile; ¹H NMR (DMSO-d₆) δ 8.26 (s, 1H), 7.61 (s, 1H), 7.22 (s, 1H), 6.23 (s, 2H), 4.33-4.22 (m, 3H), 4.04 (q, J = 14.0 Hz, 2H), 3.72 (d, J = 13.6 Hz, 1H), 3.26 (s, 3H), 2.87 (t, J = 12.1 Hz, 1H), 2.5-2.43 (m, 1H), 1.74 (d, J = 11.7 Hz, 2H), 1.67 (q, J = 7.0 Hz, 2H), 1.55- 1.42 (m, 1H), 1.15-0.95 (m, 2H); TOF LC-MS [M + H]⁺496.16 |
| 34 | 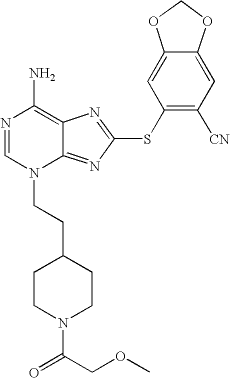 | 6-[(6-Amino-3-{2-[1- (methoxyacetyl)piperidin-4-yl]ethyl}- 3H-purin-8-yl)thio]-1,3-benzodioxole- 5-carbonitrile; TOF LC-MS [M + H]⁺496.16 |
| 35 | 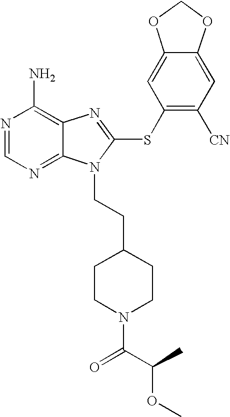 | 6-{[6-Amino-9-(2-{1-[(2R)-2- methoxypropanoyl]piperidin-4- yl}ethyl)-9H-purin-8-yl]thio}-1,3- benzodioxole-5-carbonitrile; ¹H NMR (DMSO-d₆) δ 8.3 (s, 1H), 8.28-8.05 (br s, 2H), 7.62 (s, 1H), 7.23 (s, 1H), 6.23 (s, 2H), 4.34 (d, J = 13.6 Hz, 1H), 4.25 (t, J = 7.4 Hz, 2H), 4.23- 4.13 (m, 1H), 3.99 (t, J = 12.5 Hz, 1H), 3.17 (s, 3H), 2.91 (t, J = 12.8 Hz, 1H), 2.54-2.44 (m, 1H), 1.76 (t, J = 10.9 Hz, 2H), 1.68 (q, J = 7.0 Hz, 2H), 1.56- 1.46 (m, 1H), 1.18 (t, J = 6.6 Hz, 3H), 1.15-0.94 (m, 2H); TOF LC-MS [M + H]⁺510.17 |
| 36 | 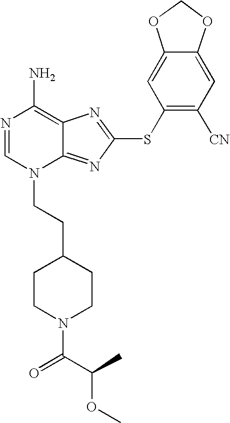 | 6-{[6-Amino-3-(2-{1-[(2R)-2- methoxypropanoyl]piperidin-4- yl}ethyl)-3H-purin-8-yl]thio}-1,3- benzodioxole-5-carbonitrile; TOF LC-MS [M + H]⁺510.18 |
| 37 | 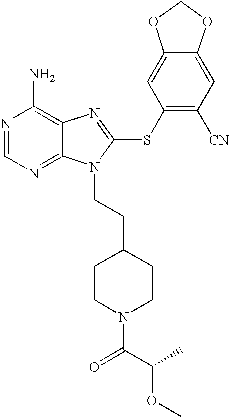 | 6-{[6-Amino-9-(2-{1-[(2S)-2- methoxypropanoyl]piperidin-4- yl}ethyl)-9H-purin-8-yl]thio}-1,3- benzodioxole-5-carbonitrile; ¹H NMR (DMSO-d₆) δ 8.30 (s, 1H), 8.28-8.05 (br s, 2H), 7.62 (s, 1H), 7.23 (s, 1H), 6.23 (s, 2H), 4.34 (d, J = 13.6 Hz, 1H), 4.25 (t, J = 7.4 Hz, 2H), 4.23-4.13 (m, 1H), 3.99 (t, J = 12.5 Hz, 1H), 3.17 (s, 3H), 2.91 (t, J = 12.8 Hz, 1H), 2.54-2.44 (m, 1H), 1.76 (t, J = 10.9 Hz, 2H), 1.68 (q, J = 7.0 Hz, 2H), 1.56-1.46 (m, 1H), 1.18 (t, J = 6.6 Hz, 3H), 1.15-0.94 (m, 2H); TOF LC-MS [M + H]⁺510.18 |
| 38 | 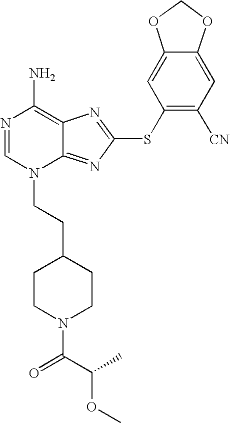 | 6-{[6-Amino-3-(2-{1-[(2S)-2- methoxypropanoyl]piperidin-4- yl}ethyl)-3H-purin-8-yl]thio}-1,3- benzodioxole-5-carbonitrile; TOF LC-MS [M + H]⁺510.17 |
| 39 | 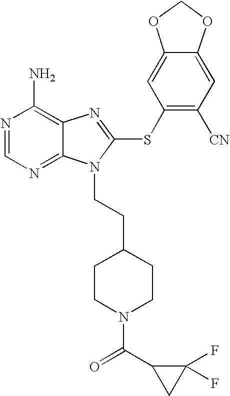 | 6-{[6-Amino-9-(2-{1-[(2,2- difluorocyclopropyl)carbonyl]piperidin- 4-yl}ethyl)-9H-purin-8-yl]thio}-1,3- benzodioxole-5-carbonitrile; ¹H NMR (DMSO-d₆) δ 8.24 (s, 1H), 7.90-7.65 (br s, 2H), 7.60 (d, J = 6.2 Hz, 1H), 7.21 (d, J = 3.1 Hz, 1H), 6.23 (s, 2H), 4.38-4.23 (m, 3H), 4.06- 3.97 (m, 1H), 3.20-2.96 (m, 2H), 2.63- 2.50 (m, 1H), 1.92-1.62 (m, 6H), 1.60- 1.45 (m, 1H), 1.09-0.9 (m, 2H); TOF LC-MS [M + H]⁺528.14 |
| 40 | 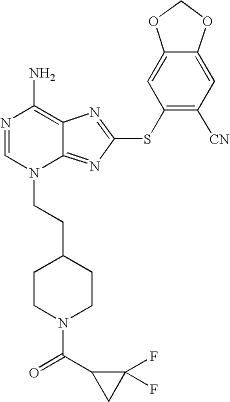 | 6-{[6-Amino-3-(2-{1-[(2,2- difluorocyclopropyl)carbonyl]piperidin- 4-yl}ethyl)-3H-purin-8-yl]thio}-1,3- benzodioxole-5-carbonitrile; TOF LC-MS [M + H]⁺528.15 |
| 41 | 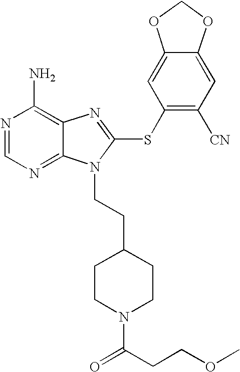 | 6-[(6-Amino-9-{2-[1-(3- methoxypropanoyl)piperidin-4- yl]ethyl}-9H-purin-8-yl)thio]-1,3- benzodioxole-5-carbonitrile; ¹H NMR (CDCl₃) δ 8.19 (s, 1H), 7.24 (s, 1H), 7.17 (s, 1H), 6.19 (s, 2H), 4.65 (d, J = 14.0 Hz, 1H), 4.34 (t, J = 7.4 Hz, 2H), 3.92 (d, J = 13.2 Hz, 1H), 3.7 (t, J = 6.6 Hz, 1H), 3.36 (s, 3H), 3.0 (t, J = 12.1 Hz, 1H), 2.61 (t, J = 6.2 Hz, 2H), 2.54 (t, J = 12.1 Hz, 1H), 1.94-1.8 (m, 4H), 1.64-1.5 (m, 1H), 1.32-1.18 (m, 2H); TOF LC-MS [M + H]⁺510.18 |
| 42 | 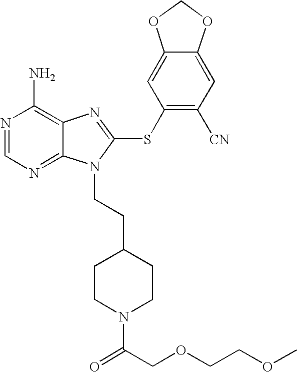 | 6-{[6-Amino-9-(2-{1-[(2- methoxyethoxy)acetyl]piperidin-4- yl}ethyl)-9H-purin-8-yl]thio}-1,3- benzodioxole-5-carbonitrile; ¹H NMR (CDCl₃) δ 8.19 (s, 1H), 7.24 (s, 1H), 7.17 (s, 1H), 6.2 (s, 2H), 4.58 (d, J = 13.6 Hz, 1H), 4.34 (t, J = 7.8 Hz, 2H), 4.22 (q, J = 13.6 Hz, 2H), 3.93 (d, J = 13.2 Hz, 1H), 3.74-3.64 (m, 2H), 3.38 (s, 3H), 2.99 (t, J = 12.1 Hz, 1H), 2.58 (t, J = 12.5 Hz, 1H), 1.95-1.8 (m, 4H), 1.64-1.5 (m, 1H), 1.3-1.0 (m, 2H); TOF LC-MS [M + H]⁺540.19 |
| 43 | 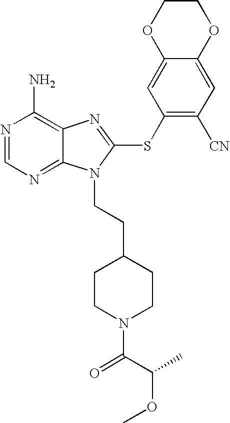 | 7-{[6-Amino-9-(2-{1-[(2S)-2- methoxypropanoyl]piperidin-4- yl}ethyl)-9H-purin-8-yl]thio}-2,3- dihydro-1,4-benzodioxine-6- carbonitrile; ¹H NMR (DMSO-d₆) δ 8.16 (s, 1H), 7.59 (s, 1H), 7.42-7.47 (br s, 2H), 7.08 (s, 1H), 4.28-4.37 (m, 4H), 4.25- 4.15 (m, 3H), 4.04-3.92 (m, 1H), 3.17 (s, 3H), 2.95-2.84 (m, 2H), 2.52-2.43 1H), 1.75 (t, J = 13.2 Hz, 2H), 1.64 (q, J = 7.4 Hz, 2H), 1.52-1.4 (m, 1H), 1.18 (t, J = 6.6 Hz, 3H), 1.12-0.9 (m, 2H); TOF LC-MS [M + H]⁺524.20 |
| 44 | 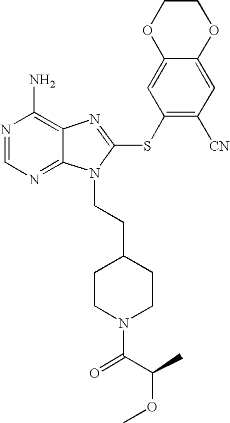 | 7-{[6-Amino-9-(2-{1-[(2R)-2- methoxypropanoyl]piperidin-4- yl}ethyl)-9H-purin-8-yl]thio}-2,3- dihydro-1,4-benzodioxine-6- carbonitrile; ¹H NMR (DMSO-d₆) δ 8.16 (s, 1H), 7.59 (s, 1H), 7.42-7.47 (bs, 2H), 7.08 (s, 1H), 4.28-4.37 (m, 4H), 4.25- 4.15 (m, 3H), 4.04-3.92 (m, 1H), 3.17 (s, 3H), 2.95-2.84 (m, 2H), 2.52-2.43 (m, 1H), 1.75 (t, J = 13.2 Hz, 2H), 1.64 (q, J = 7.4 Hz, 2H), 1.52-1.4 (m, 1H), 1.18 (t, J = 6.6 Hz, 3H), 1.12-0.9 (m, 2H); TOF LC-MS [M + H]⁺524.21 |
| 45 | 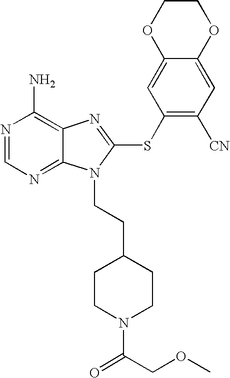 | 7-[(6-Amino-9-{2-[1- (methoxyacetyl)piperidin-4-yl]ethyl}- 9H-purin-8-yl)thio]-2,3-dihydro-1,4- benzodioxine-6-carbonitrile; ¹H NMR (CD₃OD) δ 8.15 (s, 1H), 7.39 (s, 1H), 7.3 (s, 1H), 4.45 (d, J = 12.1 Hz, 1H), 4.4-4.29 (m, 6H), 4.13 (q, J = 13.2 Hz, 2H), 3.82 (d, J = 12.8 Hz, 1H), 3.38 (s, 3H), 3.07-2.95 (m, 1H), 2.63 (t, J = 11.3 Hz, 1H), 1.94- 1.76 (m, 4H), 1.69-1.56 (m, 1H), 1.32- 1.1 (m, 2H); TOF LC-MS [M + H]⁺ 510.20 |
| 46 | 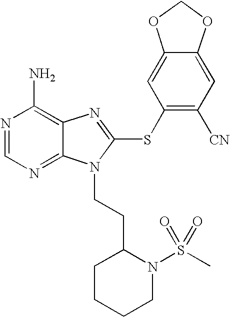 | 6-[(6-Amino-9-{2-[1- (methylsulfonyl)piperidin-2-yl]ethyl}- 9H-purin-8-yl)thio]-1,3-benzodioxole- 5-carbonitrile; ¹H NMR (CD₃OD) δ 8.3 (s, 1H), 7.4 (s, 1H), 7.3 (s, 1H), 6.2 (S, 2H), 4.4 (t, J = 8.0 Hz, 2H), 3.0 (s, 3H), 2.5-2.4 (m, 2H), 2.1-2.0 (m, 2H), 1.8 -1.7 (m, 2H), 1.7-1.6 (m, 5H), TOF LC-MS [M + H]⁺502.14 |
| 47 | 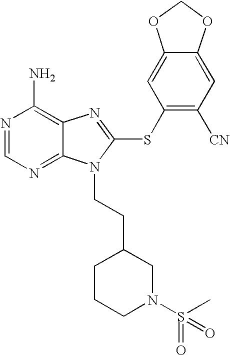 | 6-[(6-Amino-9 {2-[1- (methylsulfonyl)piperidin-3-yl]ethyl}- 9H-purin-8-yl)thio]-1,3-benzodioxole- 5-carbonitrile; ¹H NMR (CD₃OD) δ 8.20 (s, 1H), 7.40 (s, 1H), 7.30 (s, 1H), 6.20 (s, 2H), 4.40 (t, J = 8.0 Hz, 2H), 2.90 (s, 3H), 2.70-2.50 (m, 2H), 2.00-1.90 (m, 2H), 1.80 -1.70 (m, 4H), 1.60-1.50 (m, 3H); TOF LC-MS [M + H]⁺502.14 |
| 48 | 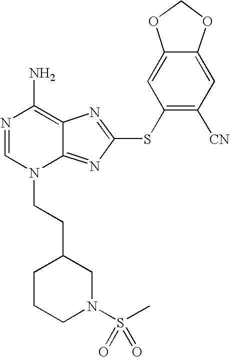 | 6-[(6-Amino-3- {2-[1- (methylsulfonyl)piperidin-3-yl]ethyl}- 3H-purin-8-yl)thio]-1,3-benzodioxole- 5-carbonitrile; ¹H NMR (CD₃OD) δ 8.50 (s, 1H), 7.40 (s, 1H), 7.30 (s, 1H), 6.20 (s, 2H), 4.40-4.30 (m, 2H), 2.90 (s, 3H), 2.70- 2.60 (m, 2H), 2.00-1.80 (m, 6H), 1.70 - 1.60 (m, 3H); TOF LC-MS [M + H]⁺ 502.14 |
| 49 | 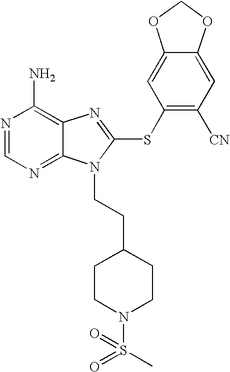 | 6-[(6-Amino-9-{2-[1- (methylsulfonyl)piperidin-4-yl]ethyl}- 9H-purin-8-yl)thio]-1,3-benzodioxole- 5-carbonitrile; ¹H NMR (DMSO-d₆) δ 8.20 (s, 1H), 7.60 (s, 1H), 7.20 (s, 1H), 6.20 (s, 2H), 4.20 (t, J = 7.2 Hz, 2H), 2.80 (s, 3H), 2.60-2.50 (m, 3H), 1.85-1.82 (m, 2H), 1.70 -1.60 (m, 2H), 1.30-1.10 (m, 4H); TOF LC-MS [M + H]⁺502.14 |
| 50 | 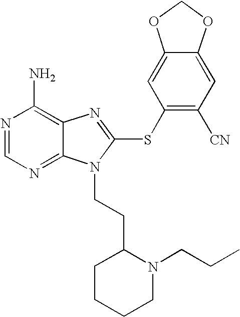 | 6-({6-Amino-9-[2-(1- propylpiperidin-2-yl)ethyl]-9H-purin- 8-yl}thio)-1,3-benzodioxole-5- carbonitrile; ¹H NMR (CD₃OD) δ 8.30 (s, 1H), 7.40 (s, 1H), 7.30 (s, 1H), 6.20 (s, 2H), 4.40 -4.30 (m, 2H), 3.00 (s, 3H), 2.60- 2.50 (m, 2H), 2.40-2.30 (m, 2H), 2.20- 2.10 (m, 2H), 2.10-1.90 (m, 2H), 1.80- 1.70 (m, 4H), 1.00-0.90 (m, 3H); TOF LC-MS [M + H]⁺466.21 |
| 51 | 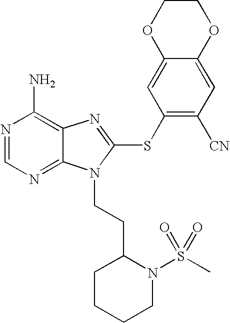 | 7-[(6-Amino-9-{2-[1- (methylsulfonyl)piperidin-2-yl]ethyl}- 9H-purin-8-yl)thio]-2,3-dihydro-1,4- benzodioxine-6-carbonitrile; ¹H NMR (CD₃OD) δ 8.20 (s, 1H), 7.40 (s, 1H), 7.30 (s, 1H), 4.40-4.30 (m, 4H), 4.30 (t, J = 7.6Hz, 2H). 3.20 (s, 3H), 2.40-2.30 (m, 2H), 2.00-1.90 (m, 1.80-1.70 (m, 2H), 1.60-1.50 (m, 5H); TOF LC-MS [M + H]⁺516.15 |
| 52 | 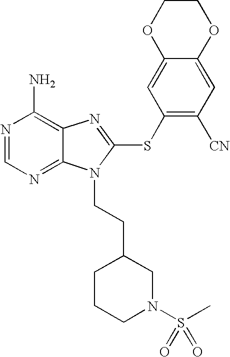 | 7-[(6-Amino-9-{2-[1- (methylsulfonyl)piperidin-3-yl]ethyl}- 9H-purin-8-yl)thio]-2,3-dihydro-1,4- benzodioxine-6-carbonitrile; ¹H NMR (CD₃OD) δ 8.20 (s, 1H), 7.40 (s, 1H), 7.30 (s, 1H), 4.40 -4.30 (m, 4H), 4.30 (t, J = 7.6Hz, 2H). 3.20 (s, 3H), 2.60-2.50 (m, 2H), 2.00-1.90 (m, 2H), 1.80-1.70 (m, 5H), 1.60-1.50 (m, 2H); TOF LC-MS [M + H]⁺516.15 |
| 53 | 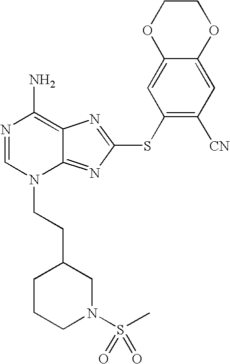 | 7-[(6-Amino-3-{2-[1- (methylsulfonyl)piperidin-3-yl]ethyl}- 3H-purin-8-yl)thio]-2,3-dihydro-1,4- benzodioxine-6-carbonitrile; TOF LC-MS [M + H]⁺516.15 |
| 54 | 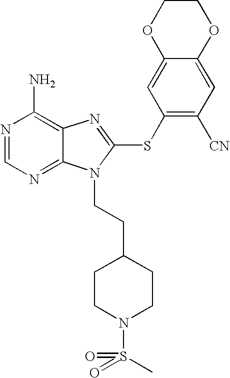 | 7-[(6-Amino-9-{2-[1- (methylsulfonyl)piperidin-4-yl]ethyl}- 9H-purin-8-yl)thio]-2,3-dihydro-1,4- benzodioxine-6-carbonitrile; ¹H NMR (CD₃OD) δ 8.30 (s, 1H), 7.40 (s, 1H), 7.30 (s, 1H), 4.40 -4.30 (m, 4H), 4.30-4.20 (m, 2H). 2.80 (s, 3H), 2.70-2.60 (m, 2H), 2.00-1.90 (m, 2H), 1.80-1.70 (m, 2H), 1.60-1.50 (m, 5H); TOF LC-MS [M + H]⁺516.15 |
| 55 | 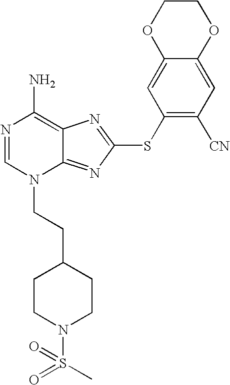 | 7-[(6-Amino-3-{2-1- (methylsulfonyl)piperidin-4-yl]ethyl}- 3H-purin-8-yl)thio]-2,3-dihydro-1,4- benzodioxine-6-carbonitrile; ¹H NMR (CD₃OD) δ 8.30 (s, 1H), 7.40 (s, 1H), 7.30 (s, 1H), 4.40 -4.30 (m, 4H), 4.30-4.20 (m, 2H). 3.10 (s, 3H), 2.70-2.60 (m, 2H), 1.90-1.80 (m, 5H), 1.40-1.20 (m, 4H); TOF LC-MS [M + H]⁺516.15 |
| 56 | 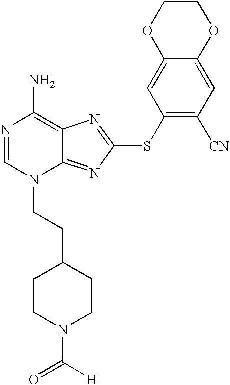 | 7-({6-Amino-3-[2-(1- formylpiperidin-4-yl)ethyl]-3H-purin- 8-yl]thio)-2,3-dihydro-1,4- benzodioxine-6-carbonitrile; LC-MS [M + H]⁺466.17 |
| 57 | 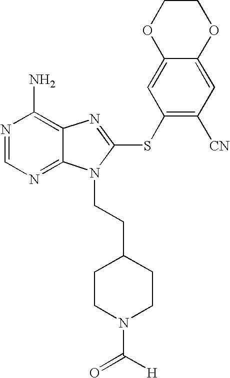 | 7-({6-Amino-9-[2-(1- formylpiperidin-4-ypethyl]-9H-purin- 8-yl]thio)-2,3-dihydro-1,4- benzodioxine-6-carbonitrile; ¹H NMR (DMSO-d₆) δ 8.26 (s, 1H), 7.95 (s, 1H), 7.60 (s, 1H), 7.15 (s, 1H), 4.40-4.30 (m, 4H), 4.15-4.10 (t, J = 7.6 Hz, 2H), 3.69-3.59 (m, 2H), 3.00-2.80 (m, 1H), 1.80-1.60 (m, 4H), 1.10-0.86 (m, 4H); TOF LC-MS [M + H]⁺466.17 |
| 58 | 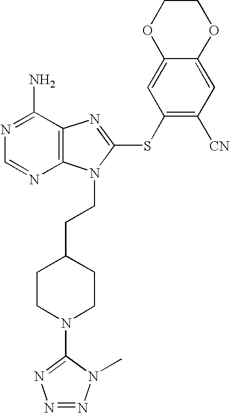 | 7-[(6-Amino-9-{2-[1-(1-methyl- 1H-tetrazol-5-yl)piperidin-4-yl]ethyl}- 9H-purin-8-yl)thio]-2,3-dihydro-1,4- benzodioxine-6-carbonitrile; ¹H NMR (CD₃OD) δ 8.30 (s, 1H), 7.44 (s, 1H), 7.41 (s, 1H), 4.42 (t, J = 8.4 Hz, 2H), 4.40-4.34 (m, 4H), 3.90 (s, 3H), 3.64 (brd, J = 12.8 Hz, 2H), 2.99 (t, J = 12.0 Hz, 2H), 1.98-1.88 (m, 4H), 1.58 (m, 1H), 1.54-1.42 (m, 2H); TOF LC-MS [M + H]⁺520.20 |
| 59 | 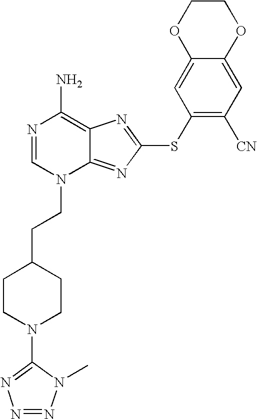 | 7-[(6-Amino-3-{2-[1-(1-methyl- 1H-tetrazol-5-yl)piperidin-4-yl]ethyl}- 3H-purin-8-yl)thio]-2,3-dihydro-1,4- benzodioxine-6-carbonitrile; TOF LC-MS [M + H]⁺520.20 |
| 60 | 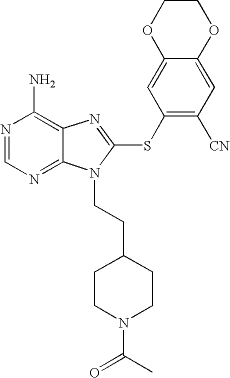 | 7-({9-[2-(1-Acetylpiperidin-4- yl)ethyl]-6-amino-9H-purin-8-yl}thio)- 2,3-dihydro-1,4-benzodioxine-6- carbonitrile; ¹H NMR (DMSO-d₆) δ 8.15 (s, 1H), 7.59 (s, 1H), 7.07 (s, 1H), 4.34- 4.3 (m, 4H), 4.22 (t, J = 7.6 Hz, 2H), 3.78-3.74 (m, 1H), 2.94-2.87 (m, 2H), 1.96 (s, 3H), 1.76-1.61 (m, 4H), 1.1- 0.86 (m, 4H); TOF LC-MS [M + H]⁺480.19 |
| 61 | 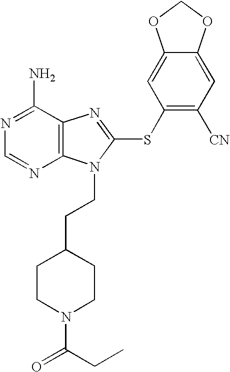 | 6-({6-Amino-9-[2-(1- propionylpiperidin-4-ypethyl]-9H- purin-8-yl]thio)-1,3-benzodioxole-5- carbonitrile; ¹H NMR (CD₃OD) δ 8.15 (s, 1H), 7.33 (s, 1H), 7.26 (s, 1H), 6.17 (s, 2H), 4.49 (brd, J = 13.2 Hz, 1H), 4.32 (t, J = 7.6 Hz, 2H), 3.92 (brd, J = 13.6 Hz, 1H) 3.04 (td, J = 13.2, 2.4 Hz, 1H), 2.59 (td, J = 13.2, 2.8 Hz, 1H), 2.39 (q, J = 7.2 Hz, 2H),1.92-1.83 (m, 2H), 1.79 (q, J = 7.6 Hz, 2H), 1.61 (m, 1H), 1.28- 1.14 (m, 2H), 1.12 (t, J = 7.2 Hz, 3H); TOF LC-MS [M + H]⁺480.20 |
| 62 | 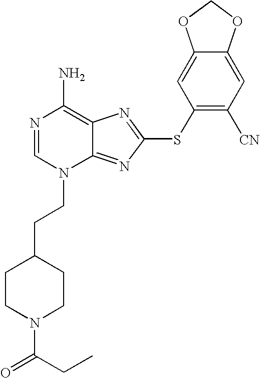 | 6-({6-Amino-3-[2-(1- propionylpiperidin-4-yl)ethyl]-3H- purin-8-yl]thio)-1,3-benzodioxole-5- carbonitrile; TOF LC-MS [M + H]⁺480.18 |
| 63 | 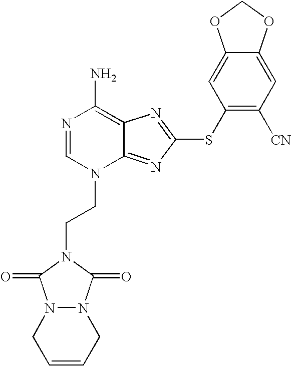 | 6-({6-Amino-3-[2-(1,3-dioxo-5,8- dihydro-1H-[1,2,4]triazolo[1,2- c]pyridazin-2(3H)-ypethyl]-3H-purin- 8-yl]thio)-1,3-benzodioxole-5- carbonitrile; TOF LC-MS [M + H]⁺492.12 |
| 64 | 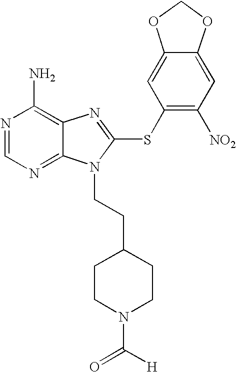 | 4-(2-{6-Amino-8-[(6-nitro-1,3- benzodioxol-5-yl)thio]-9H-purin-9- yl}ethyl)piperidine-1-carbaldehyde; 1H NMR (Acetone-d₆) δ 8.3 (s, 1H), 8.1 (s, 1H), 8.0 (s, 1H), 7.8 (s, 1H), 6.1(s, 2H), 4.3-4.2 (m, 2H), 1.9- 1.8 (m, 3H), 1.7-1.6 (m, 3H), 1.5-1.3 (m, 2H), 1.4-1.3 (m, 3H); TOF LC-MS [M + H]⁺472.15 |
| 65 | 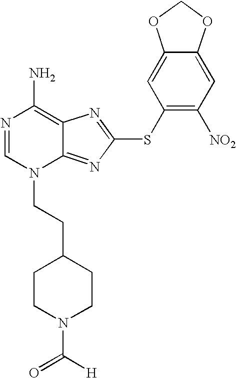 | 4-(2-{6-Amino-8-[(6-nitro-1,3- benzodioxol-5-yl)thio]-3H-purin-3- yl}ethyl)piperidine-1-carbaldehyde; TOF LC-MS [M + H]⁺472.12 |
| 66 | 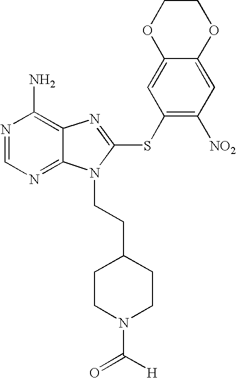 | 4-(2-{6-Amino-8-[(7-nitro-2,3- dihydro-1,4-benzodioxin-6-yl)thio]- 9H-purin-9-yl}ethyl)piperidine-1- carbaldehyde; ¹H NMR (CD₃OD) δ 8.4 (s, 1H), 8.0 (s, 1H), 7.9 (s, 1H), 6.6 (s, 1H), 4.4-4.2 (m, 6H), 3.1-3.0 (m, 2H), 2.7- 2.6 (m, 2H) 1.8 -1.7 (m, 5H), 1.6-1.5 (m, 2H); TOF LC-MS [M + H]⁺486.15 |
| 67 | 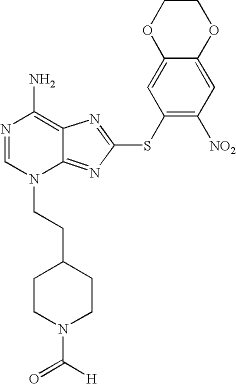 | 4-(2-{6-Amino-8-[(7-nitro-2,3- dihydro-1,4-benzodioxin-6-yl)thio]- 3H-purin-3-yl}ethyl)piperidine-l- carbaldehyde; TOF LC-MS [M + H]⁺486.15 |
| 68 | 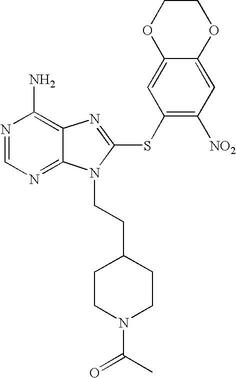 | 9-[2-(1-Acetylpiperidin-4- yl)ethyl]-8-[(7-nitro-2,3-dihydro-1,4- benzodioxin-6-yl)thio]-9H-purin-6- amine; ¹H NMR (CD₃OD) δ 8.3 (s, 1H), 7.9 (s, 1H), 6.4 (s, 1H), 4.4-4.3 (m, 6H), 3.1-3.0 (m, 2H), 2.6-2.5 (m, 2H), 2.0 (s, 3H), 1.9 -1.8 (m, 4H), 1.6 (s, 1H), 1.3-1.1 (m, 2H); TOF LC-MS [M + H]⁺500.15 |
| 69 | 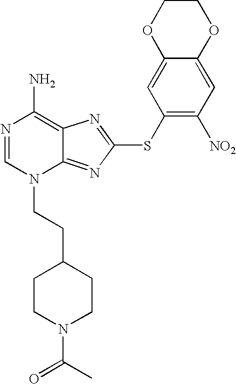 | 3-[2-(1-Acetylpiperidin-4- yl)ethyl]-8-[(7-nitro-2,3-dihydro-1,4- benzodioxin-6-yl)thio]-3H-purin-6- amine; ¹H NMR (CD₃OD) δ 8.5 (s, 1H), 7.8 (s, 1H), 7.0 (s, 1H), 4.5-4.4 (m, 2H), 4.3-4.2 (m, 4H), 3.1-3.0 (m, 2H), 2.62-2.60 (m, 2H), 2.1 (s, 3H), 1.9 -1.8 (m, 4H), 1.6 (s, 1H), 1.3-1.1 (m, 2H); TOF LC-MS [M + H]⁺500.15 |
| 70 | 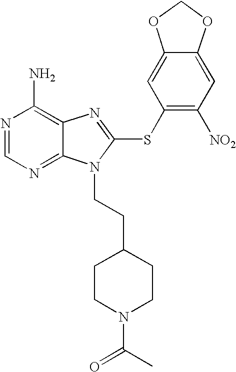 | 9-[2-(1-Acetylpiperidin-4- ypethyl]-8-[(6-nitro-1,3-benzodioxol- 5-yl)thio]-9H-purin-6-amine; TOF LC-MS [M + H]⁺486.15 |
| 71 | 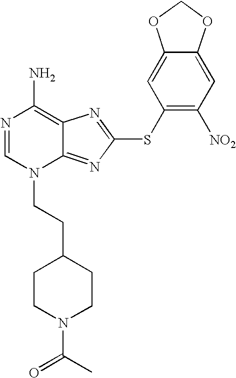 | 3-[2-(1-Acetylpiperidin-4- yl)ethyl]-8-[(6-nitro-1,3-benzodioxol- 5-yl)thio]-3H-purin-6-amine; TOF LC-MS [M + H]⁺486.15 |
| 72 | 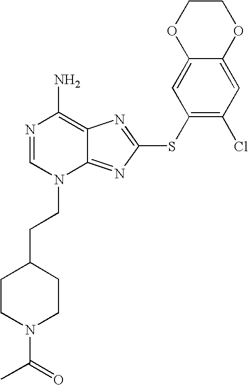 | 3-[2-(1-Acetylpiperidin-4- yl)ethyl]-8-[(7-chloro-2,3-dihydro-1,4- benzodioxin-6-yl)thio]-3H-purin-6- amine; ¹H NMR (CD₃OD) δ 8.35 (s, 1H), 7.21 (s, 1H), 7.10 (s, 1H), 4.41-4.27 (m, 6H), 3.3-3.2 (m, 3H), 2.09 (s, 3H), 1.9-1.80 (m, 4H), 1.29-1.11 (m, 4H); TOF LC-MS [M + H]⁺489.15 |
| 73 | 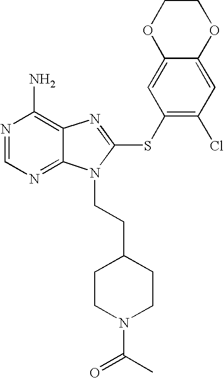 | 9-[2-(1-Acetylpiperidin-4- yl)ethyl]-8-[(7-chloro-2,3-dihydro-1,4- benzodioxin-6-yl)thio]-9H-purin-6- amine; ¹H NMR (CD₃OD) δ 8.28 (s, 1H), 7.22 (s, 1H), 7.11 (s, 1H), 4.38-4.27 (m, 6H), 3.3-3.2 (m, 3H), 2.08 (s, 3H), 1.9-1.80 (m, 4H), 1.29-1.11 (m, 4H); TOF LC-MS [M + H]⁺489.15 |
| 74 | 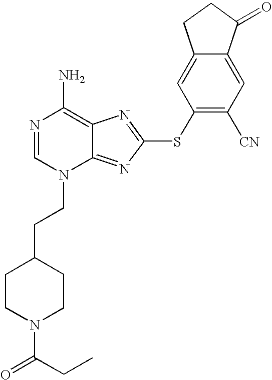 | 6-({6-Amino-3-[2-(1- propionylpiperidin-4-yl)ethyl]-3H- purin-8-yl}thio)-3-oxoindane-5- carbonitrile. TOF LC-MS [M + H]⁺490.19 |
| 75 | 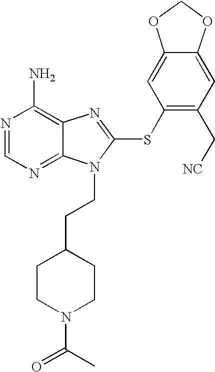 | [6-({9-[2-(1-Acetylpiperidin-4- yl)ethyl]-6-amino-9H-purin-8-yl}thio)- 1,3-benzodioxol-5-yl]acetonitrile; ¹H NMR (CD₃OD) δ 8.27 (s, 1H), 7.25 (s, 1H), 7.17 (s, 1H), 6.12 (s, 2H), 4.51 (brd, J = 13.6 Hz, 1H), 4.41-4.34 (m, 2H), 4.05 (s, 2H), 3.91 (brd, J = 14.0 Hz, 1H), 3.09 (brt, J = 11.2 Hz, 1 H), 2.61 (brt, J = 13.2 Hz, 1H), 2.10 (s, 3H), 1.98-1.78 (m, 4H), 1.62 (m, 1H), 1.27 (m, 1H), 1.16 (m, 1H); TOF LC-MS [M + H]⁺480.19 |
| 76 | 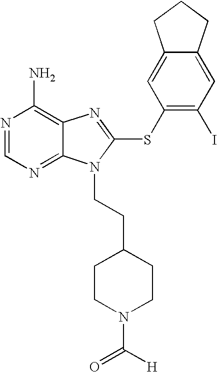 | 4-(2-{6-Amino-8-[(6-iodo-2,3- dihydro-1H-inden-5-yl)thio]-9H-purin- 9-yl}ethyl)piperidine-l-carbaldehyde; ¹H NMR (CD₃CN) δ 8.28 (s, 1H), 7.92 (s, 1H), 7.83 (s, 1H), 7.11 (s, 1H), 4.3-4.2 (m, 4H), 4.1-4.0 (m, 6H), 2.9- 2.65 (m, 4H), 1.7-1.0 (m, 5H); TOF LC-MS [M + H]⁺549.09 |
| 77 | 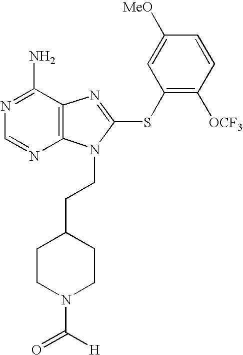 | 4-[2-(6-Amino-8-{[5-methoxy-2- (trifluoromethoxy)phenyl]thio}-9H- purin-9-yl)ethyl]piperidine-1- carbaldehyde; ¹H NMR (CD₃OD) δ 8.31 (s, 1H), 7.98 (s, 1H), 7.42 (dq, J = 8.8, 1.6 Hz, 1H), 7.11 (s, 1H), 7.10 (dd, J = 8.8, 1.6 Hz, 1H), 4.37 (t, J = 7.6 Hz, 2H), 4.29 (brd, J = 13.2 Hz, 1H), 3.80 (s, 3H), 3.70 (brd, J = 13.6 Hz, 1H), 3.07 (dt, J = 12.4, 2.8 Hz, 1H), 2.62 (dt, J = 12.8, 3.2 Hz, 1H), 1.90-1.83 (m, 2H), 1.77 (q, J = 7.6 Hz, 2H), 1.58 (m, 1H), 1.17 (qd, J = 12.4, 4.4 Hz, 1H), 1.08 (qd, J = 12.0, 4.0 Hz, 1H); TOF LC-MS [M + H]⁺497.16 |
| 78 | 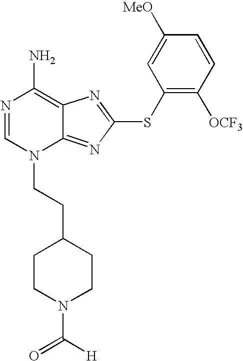 | 4-[2-(6-Amino-8-{[5-methoxy-2- (trifluoromethoxy)phenyl]thioI-3H- purin-3-ypethyl]piperidine-1- carbaldehyde TOF LC-MS [M + H]⁺497.16 |
| 79 | 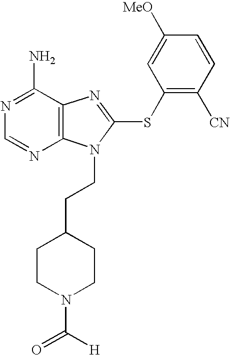 | 2-({6-Amino-9-[2-(1- formylpiperidin-4-ypethyl]-9H-purin- 8-yl]thio)-4-methoxybenzonitrile; ¹H NMR (CD₃OD) δ 8.32 (s, 1H), 7.98 (s, 1H), 7.83 (d, J = 10.4 Hz, 1H), 7.39 (d, J = 2.8 Hz, 1H), 7.19 (dd, J = 10.4, 2.8 Hz, 1H), 4.42 (t, J = 7.2 Hz, 2H), 4.30 (br d, J = 12.4 Hz, 1H), 3.90 (s, 3H), 3.71 (br d, J = 13.2 Hz, 1H), 2.99 (td, J = 12.4, 2.4 Hz, 1H), 2.65 (td, J = 12.8, 2.8 Hz, 1H), 1.97-1.84 (m, 4H), 1.64 (m, 1H), 1.26-1.08 (m, 2H); TOF LC-MS [M + H]⁺438.17 |
| 80 | 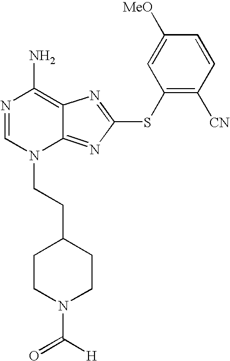 | 2-({6-Amino-3-[2-(1- formylpiperidin-4-ypethyl]-3H-purin- 8-yl]thio)-4-methoxybenzonitrile; ¹H NMR (CD₃OD) δ 8.51 (s, 1H), 7.99 (s, 1H), 7.87 (d, J = 8.8 Hz, 1H), 7.47 (d, J = 2.8 Hz, 1H), 7.24 (dd, J = 8.8, 2.4 Hz, 1H), 4.39 (t, J = 8.0 Hz, 2H), 4.26 (brd, J = 12.8 Hz, 1H), 3.92 (s, 3H), 3.68 (d, J = 13.6 Hz, 1H), 3.09 (td, J = 13.6, 3.2 Hz, 1H), 2.65 (td, J = 12.8, 3.2 Hz, 1H), 1.90-1.77 (m, 4H), 1.60 (m, 1H), 1.15 (qd, J = 12.4, 3.6 Hz, 1H), 1.03 (qd, J = 12.8, 4.8 Hz, 1H); TOF LC-MS [M + H]⁺438.17 |
| 81 | 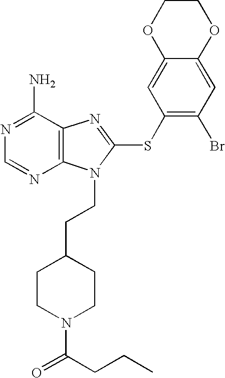 | 8-[(7-Bromo-2,3-dihydro-1,4- benzodioxin-6-yl)thio]-9-[2-(1- butyrylpiperidin-4-yl)ethyl]-9H-purin- 6-amine; ¹H NMR (CD₃OD) δ 8.17 (s, 1H), 7.49 (brs, 2H), 7.28 (s, 1H), 6.68 (s, 1H), 4.32 (brd J = 13.2 Hz, 1H), 4.24- 4.16 (m, 6H), 3.80 (brd, J = 13.2 Hz, 1H), 2.84 (brt, J = 12.8 Hz, 1H), 2.37 (brt, J = 12.8 Hz, 1H), 2.24 (t, J = 8.0 Hz, 2H), 1.72-1.62 (m, 2H), 1.57 (q, J = 7.6 Hz, 2H), 1.47 (hex, J = 7.2 Hz, 2H), 1.36 (m, 1H), 1.00 (m, 1H), 0.89 (m, 1H), 0.87 (t, J = 7.2 Hz, 3H); TOF LC-MS [M + H]⁺561.13 |
| 82 | 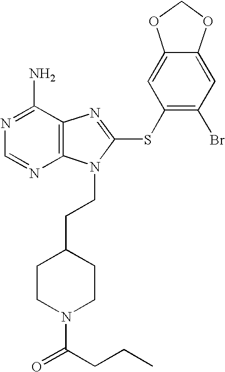 | 8-[(6-Bromo-1,3-benzodioxol-5- yl)thio]-9-[2-(1-butyrylpiperidin-4- yl)ethyl]-9H-purin-6-amine; ¹H NMR (CD₃OD) δ 8.16 (s, 1H), 7.44 (brs, 2H), 7.39 (s, 1H), 6.81 (s, 1H), 6.01 (s, 2H), 4.32 (brd, J = 12.4 Hz, 1H), 4.18 (t, J = 6.8 Hz, 2H), 3.81 (brd, J = 12.4 Hz, 1H), 2.86 (brt, J = 10.8 Hz, 1H), 2.39 (brt, J = 10.8 Hz, 1H), 2.24 (t, J = 6.8 Hz, 2H), 1.76-1.65 (m, 2H), 1.69 (q, J = 7.2 Hz, 2H), 1.48 (hex, J = 7.2 Hz, 2H), 1.40 (m, 1H), 1.01 (m, 1H), 0.92 (m, 1H), 0.87 (t, J = 7.6 Hz, 3H); TOF LC-MS [M + H]⁺547.11 |
| 83 | 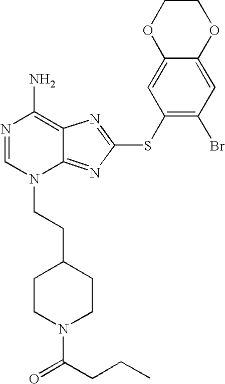 | 8-[(7-Bromo-2,3-dihydro-1,4- benzodioxin-6-yl)thio]-3-[2-(1- butyrylpiperidin-4-yl)ethyl]-3H-purin- 6-amine; TOF LC-MS [M + H]⁺561.13 |
| 84 | 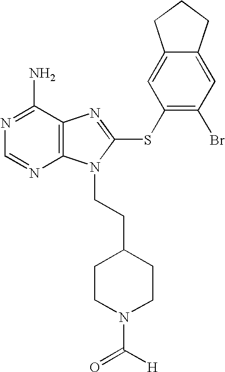 | 4-{2-[6-Amino-8-(6-bromo-indan- 5-ylsulfanyl)-Aurin-9-yl]- piperidine-l-carbaldehyde; ¹H NMR (DMSO-d₆) δ 8.17 (s, 1H), 7.92 (s, 1H), 7.59 (s, 1H), 6.91 (s, 1H), 4.16 (t, J = 6.8Hz, 2H), 4.1-4.0 (m, 1H), 3.65-3.55 (m, 1H), 2.83 (t, J = 7.6 Hz, 2H), 2.69 (t, J = 7.6 Hz, 2H), 2.57- 2.45 (m, 4H), 2.0-1.91 (m, 2H), 1.75- 1.55 (m, 4H), 1.1-0.85 (m, 1H); TOF LC-MS [M + H]⁺501.1 |
| 85 | 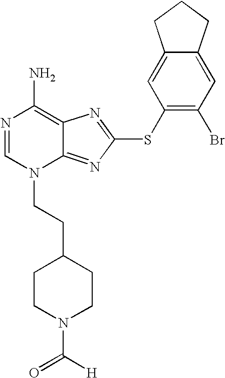 | 4-{2-[6-Amino-8-(6-bromo-indan- 5-ylsulfanyl)-purin-3-yl]- piperidine-1-carbaldehyde; ¹H NMR (DMSO-d₆) δ 8.38 (s, 1H), 7.95 (s, 1H), 7.50 (s, 1H), 7.30 (s, 1H), 4.29 (t, J = 7.2Hz, 2H), 4.1-4.0 (m, 1H), 3.65-3.55 (m, 1H), 2.83 (t, J = 7.6 Hz, 2H), 2.69 (t, J = 7.6 Hz, 2H), 2.57- 2.45 (m, 4H), 2.0-1.91 (m, 2H), 1.75- 1.55 (m, 4H), 1.1-0.85 (m, 1H); TOF LC-MS [M + H]⁺501.1 |
| 86 | 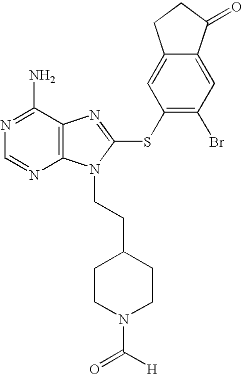 | 4-(2{6-Amino-8-[(6-bromo-l-oxo- 2,3-dihydro-1H-inden-5-yl)thio]-9H- purin-9-yl}ethyl)piperidine-1- carbaldehyde; ¹H NMR (CD₃OD) δ 8.32 (s, 1H), 7.97 (s, 1H), 7.95 (s, 1H), 7.34 (s, 1H), 4.36 (t, J = 6.8 Hz, 2H), 4.26 (brd, J = 12.8 Hz, 1H), 3.68 (brd, J = 13.2 Hz, 1H), 3.07-2.99 (m, 3H), 2.70-2.67 (m, 2H), 2.59 (td, J = 12.8, 2.4 Hz, 1H), 1.88-1.74 (m, 4H), 1.57 (m, 1H), 1.15 (m, 1H), 1.06 (m, 1H); TOF LC-MS [M + H]⁺515.09 |
| 87 | 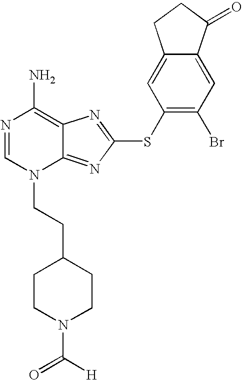 | 4-(2-{6-Amino-8-[(6-bromo-1-oxo- 2,3-dihydro-1H-inden-5-yl)thio]-3H- purin-3-yl}ethyl)piperidine-l- carbaldehyde; TOF LC-MS [M + H]⁺515.09 |

### Example Compounds 88 and 89

### (2S)-1-[4-(2-{6-Amino-8-[(7-bromo-2,3-dihydro-1,4-benzodioxin-6-yl)thio]-9H-purin-9-yl}ethyl)piperidin-1-yl]-1-oxopropan-2-ol and (2S)-1-[4-(2-{6-amino-8-[(7-bromo-2,3-dihydro-1,4-benzodioxin-6-yl)thio]-3H-purin-3-yl}ethyl)piperidin-1-yl]-1-oxopropan-2-ol

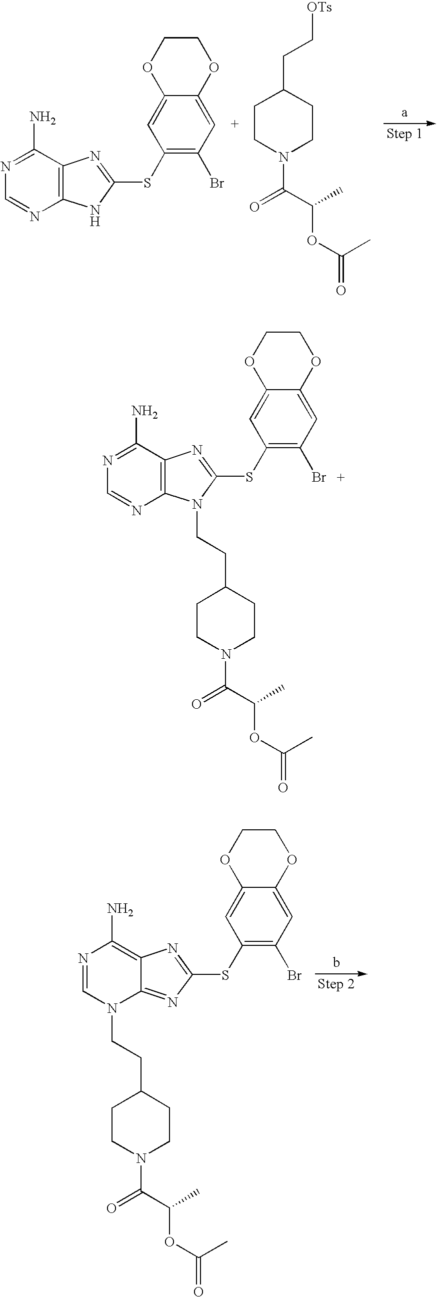

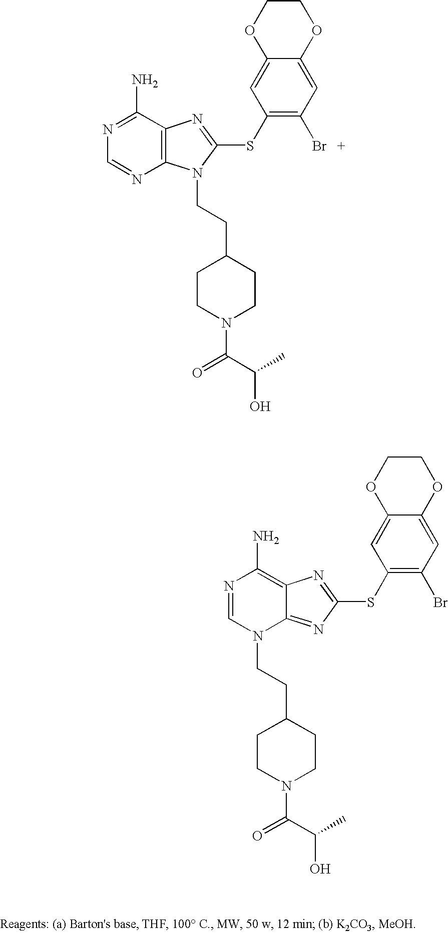

Step 1. A mixture of 8-(7-bromo-2,3-dihydro-benzo[1,4]dioxin-6-ylsulfanyl)-9H-purin-6-ylamine (0.1 g, 0.26 mmol), (1S)-1-methyl-2-[4-(2-{[(4-methylphenyl)sulfonyl]oxy}ethyl)piperidin-1-yl]-2-oxoethyl acetate (0.125 g, 0.31 mmol) and Barton's base (64 μL, 0.31 mmol) in THF (3 mL) was heated at 100° C. for 12 min under microwave irradiation with 50 w power. After cooling, the reaction mixture was concentrated under reduced pressure to afford mixture of (1S)-2-[4-(2-{6-amino-8-[(7-bromo-2,3-dihydro-1,4-benzodioxin-6-yl)thio]-9H-purin-9-yl}ethyl)piperidin-1-yl]-1-methyl-2-oxoethyl acetate and (1S)-2-[4-(2-{6-amino-8-[(7-bromo-2,3-dihydro-1,4-benzodioxin-6-yl)thio]-3H-purin-3-yl}ethyl)piperidin-1-yl]-1-methyl-2-oxoethyl acetate. The crude mixture was used for the next reaction without further purification.

Step 2. To a solution of above crude product in MeOH (5 mL) was added K₂CO₃ (0.054 g, 0.39 mmol) and the resulting mixture was stirred for overnight at room temperature. After the completion of reaction, solvent was evaporated and water (10 mL) was added, the solids were collected and washed with water (20 mL). The crude product was purified by preparative HPLC [X-Terra prep-RP18 10 um, 19×250 mm (waters), Mobile phase: solvent A: Water HPLC grade containing 0.01% TFA, and solvent B: acetonitrile containing 0.01% TFA, general eluting gradient—solvent B 15% to 80 over 15 to 25 minutes run time]. After lyophilization of HPLC fractions the example compounds were isolated as trifluoro acetate salt. (2S)-1-[4-(2-{6-Amino-8-[(7-bromo-2,3-dihydro-1,4-benzodioxin-6-yl)thio]-9H-purin-9-yl}ethyl)piperidin-1-yl]-1-oxopropan-2-ol. ¹H NMR (CD₃OD) δ 8.32 (s, 1H), 7.28-7.26 (m, 2H), 4.6-4.45 (m, 2H), 4.38 (t, J=7.0 Hz, 2H), 4.33-4.26 (m, 4H), 4.02 (d, J=14.0 Hz, 1H), 3.08-2.98 (m, 1H), 2.69-2.58 (m, 1H), 1.95-1.8 (m, 4H), 1.68-1.55 (m, 1H), 1.30 (dd, J=10.1, 6.6 Hz, 3H), 1.28-1.1 (m, 2H); TOF-MS [M+H]⁺ 563.11 and (2S)-1-[4-(2-{6-amino-8-[(7-bromo-2,3-dihydro-1,4-benzodioxin-6-yl)thio]-3H-purin-3-yl}ethyl)piperidin-1-yl]-1-oxopropan-2-ol. LC-MS [M+H]⁺ 563.2.

### Example Compounds 90-112

Example compounds 90-112 were synthesized in the same manner as described for either example compound 1 and 2 or example compounds 88 and 89, above, using appropriate starting materials and are summarized in table 4, below.

TABLE 4
Preparation of Example Compounds 90-112

| Example | | |
|---|---|---|
| Compound | Structure | Name and analytical data |
| 90 | 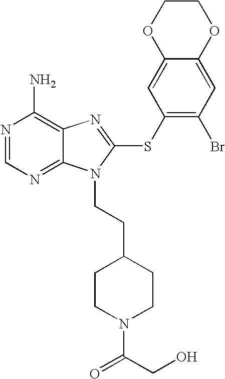 | 2-[4-(2-{6-Amino-8-[(7- bromo-2,3-dihydro-1,4- benzodioxin-6-yl)thio]-9H-purin-9- yl}ethyl)piperidin-1-yl]-2- oxoethanol; ¹H NMR (CD₃OD) δ 8.32 (s, 1H), 7.27 (s, 1H), 7.26 (s, 1H), 4.48 (d, J = 13.2 Hz, 1H), 4.37 (t, J = 7.4 Hz, 2H), 4.52-4.26 (m, 4H), 4.2 (d, J = 5.8 Hz, 2H), 3.72 (d, J = 14.4 Hz, 1H), 3.03-2.94 (m, 1H), 2.7-2.6 (m, 1H), 1.92-1.8 (m, 4H), 1.66-1.54 (m, 1H), 1.12-1.3 (m, 2H); TOF LC-MS [M + H]⁺ 549.04 |
| 91 | 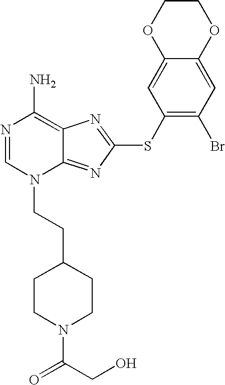 | 2-[4-(2-{6-Amino-8-[(7- bromo-2,3-dihydro-1,4- benzodioxin-6-yl)thio]-3H-purin-3- yl}ethyl)piperidin-1-yl]-2- oxoethanol; TOF LC-MS [M + H]⁺ 549.04 |
| 92 | 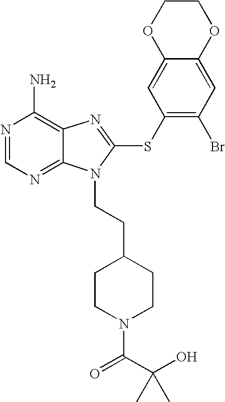 | 1-[4-(2-{6-Amino-8-[(7- bromo-2,3-dihydro-1,4- benzodioxin-6-yl)thio]-9H-purin-9- yl}ethyl)piperidin-1-yl]-2-methyl- 1-oxopropan-2-ol; TOF LC-MS [M + H]⁺ 577.11 |
| 93 | 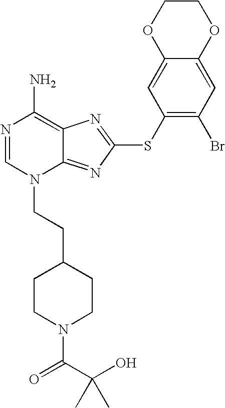 | l-[4-(2-{6-Amino-8-[(7- bromo-2,3-dihydro-1,4- benzodioxin-6-yl)thio]-3H-purin-3- yl}ethyl)piperidin-1-yl]-2-methyl- 1-oxopropan-2-ol; TOF LC-MS [M + H]⁺ 577.11 |
| 94 | 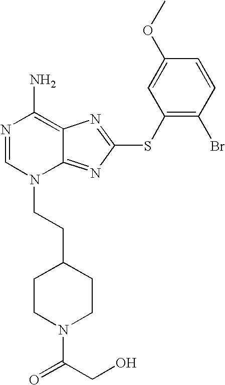 | 2-[4-(2-{6-Amino-8-[(2- bromo-5-methoxyphenyl)thio]-3H- purin-3-yl}ethyl)piperidin-1-yl]-2- oxoethanol; ¹H NMR (MeOH-d₄) δ 8.56 (s, 1H), 7.74 (d, J = 8.8 Hz, 1H), 7.46 (d, J = 3.2 Hz, 1H), 7.11 (dd, J = 8.8, 3.2 Hz, 1H), 4.47-4.39 (m, 3H), 4.22 (s, 2H), 3.84 (s, 3H), 3.4-3.2 (m, 3H), 1.91-1.75 (m, 4H), 1.3-1.1 (m, 3H); TOF LC-MS [M + H]⁺ 521.0 |
| 95 | 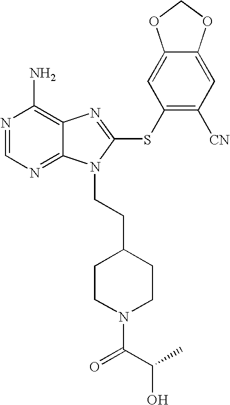 | 6-{[6-Amino-9-(2-{1-[(2S)-2- hydroxypropanoyl]piperidin-4- yl}ethyl)-9H-purin-8-yl]thio}-l,3- benzodioxole-5-carbonitrile; ¹H NMR (CD₃OD) δ 8.31 (s, 1H), 7.38 (s, 1H), 7.37 (s, 1H), 6.2 (s, 2H), 4.45-4.6 (m, 2H), 4.4 (t, J = 7.4 Hz, 2H), 4.0 (d, J = 14.0 Hz, 1H), 3.1-3.0 (m, 1H), 2.7-2.6 (m, 1H), 1.97-1.82 (m, 4H), 1.7-1.56 (m, 1H), 1.14-1.0 (m, 5H); TOF LC-MS [M]⁺ 495.93 |
| 96 | 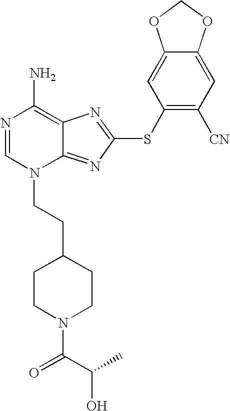 | 6-{[6-Amino-3-(2-{1-[(2S)-2- hydroxypropanoyl]piperidin-4- yl}ethyl)-3H-purin-8-yl]thio}-1,3- benzodioxole-5-carbonitrile; TOF LC-MS [M]⁺ 495.91 |
| 97 | 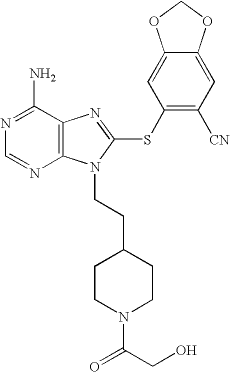 | 6-({6-Amino-9-[2-(1- glycoloylpiperidin-4-yl)ethyl]-9H- purin-8-yl}thio)-1,3-benzodioxole- 5-carbonitrile; ¹H NMR (CD₃OD) δ 8.28 (s, 1H), 7.35 (s, 1H), 7.34 (s, 1H), 6.2 (s, 2H), 4.49 (d, J = 13.6 Hz, 1H), 4.39 (t, J = 7.8 Hz, 2H), 4.2 (d, J = 5.4 Hz, 2H), 3.71 (d, J = 13.2 Hz, 1H), 3.0 (t, J = 11.7 Hz, 2H), 2.68 (t, J = 11.3 Hz, 1H), 1.96-1.84- (m, 4H), 1.69-1.55 (m, 1H), 1.35-1.15 (m, 2H); TOF LC-MS [M]⁺ 481.91 |
| 98 | 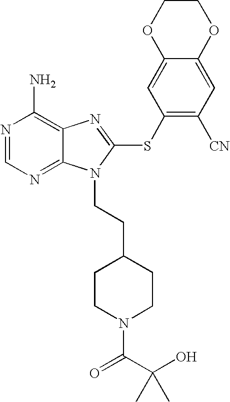 | 7-[(6-Amino-9-{2-[1-(2- hydroxy-2- methylpropanoyl)piperidin-4- yl]ethyl)-9H-purin-8-yl)thio]-2,3- dihydro-1,4-benzodioxine-6- carbonitrile; TOF LC-MS [M + H]⁺ 324.20 |
| 99 | 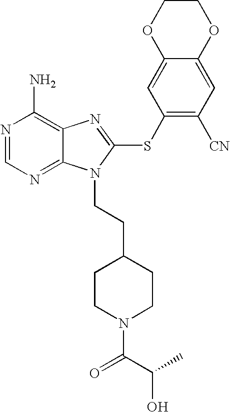 | 7-{[6-Amino-9-(2-{1-[(2S)-2- hydroxypropanoyl]piperidin-4- yl}ethyl)-9H-purin-8-yl]thio}-2,3- dihydro-1,4-benzodioxine-6- carbonitrile; ¹H NMR (DMSO-d₆) δ 8.15 (s, 1H), 7.58 (s, 1H), 7.43-7.36 (broad s, 2H), 7.07 (s, 1H), 4.8-4.76 (m, 1H), 4.56-4.44 (m, 1H), 4.44-4.28 (m, 4H), 4.24 (t, J = 6.6 Hz, 2H), 3.92 (d, J = 13.6 Hz, 1H), 2.92- 2.82 (m, 1H), 2.54-2.44 (m, 1H), 1.8-1.56 (m, 4H), 1.18-0.98 (m, 5H); TOF LC-MS [M + H]⁺ 510.19 |
| 100 | 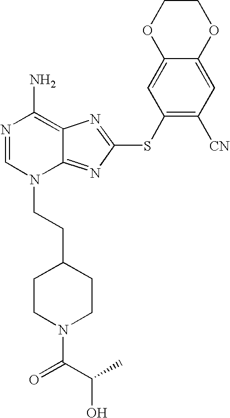 | 7-{[6-Amino-3-(2-{1-[(2S)-2- hydroxypropanoyl]piperidin-4- yl}ethyl)-3H-purin-8-yl]thio}-2,3- dihydro-1,4-benzodioxine-6- carbonitrile; TOF LC-MS [M + H]⁺ 510.18 |
| 101 | 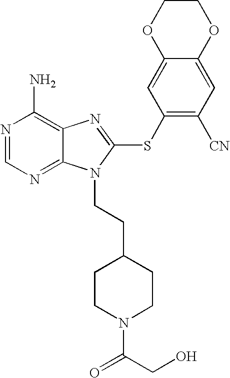 | 7-({6-Amino-9-[2-(1- glycoloylpiperidin-4-yl)ethyl]-9H- purin-8-yl}thio)-2,3-dihydro-1,4- benzodioxine-6-carbonitrile; ¹H NMR (DMSO-d₆) δ 8.26 (s, 1H), 7.61 (s, 1H), 7.14 (s, 1H), 4.37-4.22 (m, 7H), 4.04 (d, J = 6.6 Hz, 2H), 3.62 (d, J = 13.2 Hz, 1H), 2.84 (t, J = 12.5 Hz, 1H), 2.58-2.44 (m, 1H), 1.74 (d, J = 12.1 Hz, 2H), 1.66 (q, J = 6.6 Hz, 2H), 1.52-1.4 (m, 1H), 1.14-0.96 (m, 2H); TOF LC-MS [M + H]⁺ 496.17 |
| 102 | 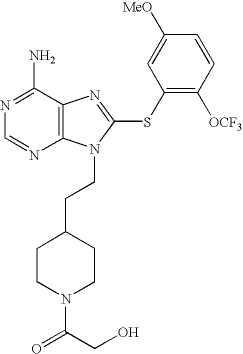 | 2-{4-[2-(6-Amino-8-{[5- methoxy-2- (trifluoromethoxy)phenyl]thio}- 9H-purin-9-yl)ethyl]piperidin-1- yl}-2-oxoethanol; ¹H NMR (CD₃OD) δ 8.30 (s, 1H). 7.41 (dq, J = 9.6, 1.6 Hz, 1H), 7.11 (s, 1H), 7.09 (dd, J = 9.6, 2.8 Hz, 1H). 4.46 (br d, J = 13.2 Hz, 1H), 4.37 (t, J = 7.6 Hz, 2H), 4.22 (d, J = 15.2 Hz, 1H), 4.17 (d, J = 15.2 Hz, 1H), 3.80 (s, 3H), 3.70 (br d, J = 14.0 Hz, 1H), 2.95 (br t, J = 12.4, 1H), 2.62 (br t, J= 11.6 Hz, 1H), 1.86-1.81 (m, 2H), 1.76 (q, J= 7.6 Hz, 2H), 1.56 (m, 1H), 1.24- 1.09 (m, 2H); TOF LC-MS [M + H]⁺ 527.17 |
| 103 | 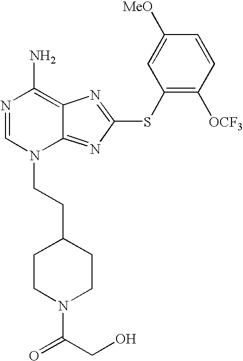 | 2-{4-[2-(6-Amino-8-{[5- methoxy-2- (trifluoromethoxy)phenyl]thio}- 3H-purin-3-yl)ethyl]piperidin-1- yl}-2-oxoethanol; TOF LC-MS [M + H]⁺ 527.17 |
| 104 | 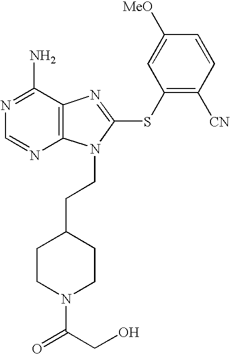 | 2-({6-Amino-9-[2-(1- glycoloylpiperidin-4-yl)ethyl]-9H- purin-8-yl}thio)-4- methoxybenzonitrile; ¹H NMR (CD₃OD) δ 8.32(s, 1H), 7.83 (d, J = 9.2 Hz, 1H), 7.39 (d, J = 2.4 Hz, 1H), 7.19 (dd, J = 9.2, 2.4 Hz, 1H), 4.47 (br d, J = 12.8.0 Hz, 1H), 4.42 (t, J = 7.2 Hz, 1H), 4.22 (d, J = 14.8 Hz, 1H), 4.18 (d, J = 14.8 Hz, 1H), 3.91 (s, 3H), 3.72 (br d, J = 8.8 Hz, 1H), 2.98 (td, J = 13.6, 2.8 Hz, 1H), 2.65 (td, J = 13.6, 2.4 Hz, 1H), 1.94-1.81 (m, 4H), 1.61 (m, 1H), 1.28-1.11 (m, 2H); TOF LC-MS [M + H]⁺ 468.18 |
| 105 | 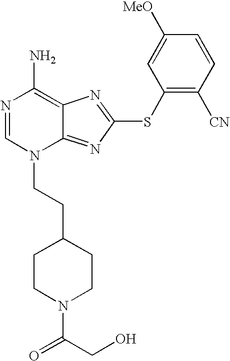 | 2-({6-Amino-3-[2-(1- glycoloylpiperidin-4-yl)ethyl]-3H- purin-8-yl}thio)-4- methoxybenzonitrile; TOF LC-MS [M + H]⁺ 468.18 |
| 106 | 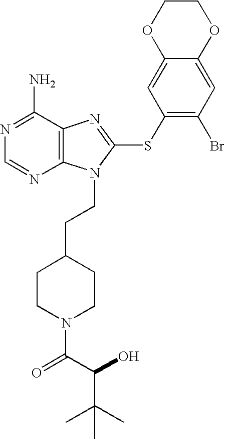 | (2S)-1-[4-(2-{6-Amino-8-[(7- bromo-2,3-dihydro-1,4- benzodioxin-6-yl)thio]-9H-purin-9- yl}ethyl)piperidin-1-yl]-3,3- dimethyl-1-oxobutan-2-ol; ¹H NMR (CDCl₃) δ 8.24 (s, 1H), 7.26 and 7.255 (s, 1H), 7.19 and 7.17 (s, 1H), 4.55 (m, 1H), 4.34 (t, J = 7.2 Hz, 2H), 4.30-4.24 (m, 5H), 4.14 (m, 1H), 3.02 (m. 1H), 2.64 (m, 1H), 1.92-1.86 (m, 2H), 1.81-1.75 (m, 2H), 1.60 (m, 1H), 1.28-1.09 (m, 2H), 0.98 and 0.95 (s, 9H); TOF LC-MS [M + H]⁺ 605.15 |
| 107 | 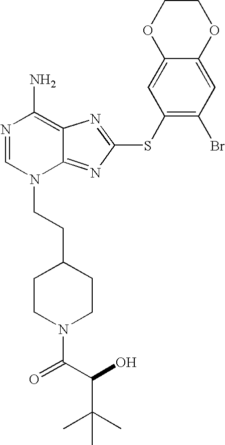 | (2S)-1-[4-(2-{6-Amino-8-[(7- bromo-2,3-dihydro-1,4- benzodioxin-6-yl)thio]-3H-purin-3- yl}ethyl)piperidin-1-yl]-3,3- dimethyl-1-oxobutan-2-ol; TOF LC-MS [M + H]⁺ 605.15 |
| 108 | 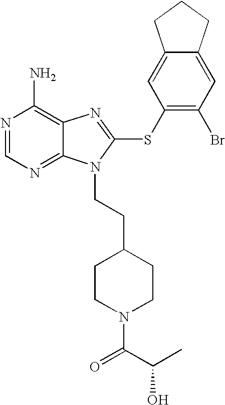 | (S)-1-(4-{2-[6-Amino-8-(6- bromo-indan-5-ylsuIfanyl)-purin-9- yl]-ethyl}-piperidin-1-yl)-2- hydroxy-propan-1-one; ¹H NMR (DMSO-d₆) δ 8.29 (s, 1H). 7.60 (s, 1H), 7.01 (s, 1H), 4.45-4.15 (m, 4H), 4.0-3.6 (m, 2H), 2.84 (t, J = 7.6 Hz, 3H), 2.72 (m, J = 7.2 Hz, 2H), 2.0-1.9 (m, 2H), 1.7-1.5 (m, 4H), 1.3-1.0 (m, 4H), 0.95-0.8 (m, 2H); TOF LC-MS [M + H]⁺ 545.13 |
| 109 | 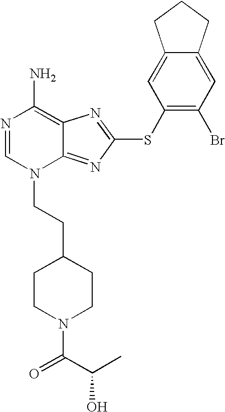 | (S)-1-(4-{2-[6-Amino-8-(6- bromo-indan-5-ylsulfanyl)-purin-3- yl]-ethyl}-piperidin-1-yl)-2- hydroxy-propan-1-one; ¹H NMR (DMSO-d₆) δ 8.64 (s, 1H), 7.70 (s, 1H), 7.60 (s, 1H ), 4.45-4.2 (m, 4H), 3.95-3.85 (m, 2H), 2.92 (t, J= 7.2 Hz, 3H), 2.82 (m, J = 7.2 Hz, 2H), 2.1-2.0 (m, 2H), 1.85-1.65 (m, 4H), 1.2-1.13 (m, 4H), 0.95-0.8 (m, 2H); TOF LC-MS [M + H]⁺ 545.13. |
| 110 | 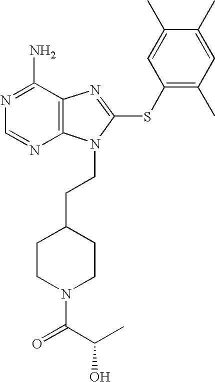 | (2S)-1-[4-(2-{6-Amino-8- [(2,4,5-trimethylphenyl)thio]-9H- purin-9-yl}ethyl)piperidin-1-yl]-1- oxopropan-2-ol ¹H NMR (DMSO-d₆) δ 8.29 (s, 1H), 7.22 (s, 1H), 7.20 (s, 1H), 4.50-4.15 (m, 4H), 4.00-3.90 (m, 1H), 3.00-2.85 (m, 2H), 2.70-2.15 (m, 5H), 2.34 (s, 3H), 2.25 (s, 3H), 2.19 (s, 3H), 1.80-1.55 (m, 2H), , 1.1-0.90 (m, 3H); LC-MS [M + H]⁺ 468.24. |
| 111 | 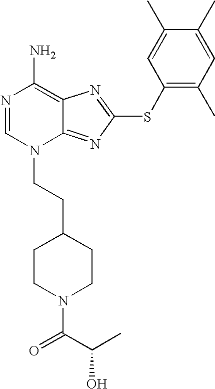 | (2S)-1-[4-(2-{6-Amino-8- [(2,4,5-trimethy)phenyl)thio]-3H- purin-3-yl}ethyl)piperidin-1-yl]-1- oxopropan-2-ol LC-MS [M + H]⁺ 468.24. |
| 112 | 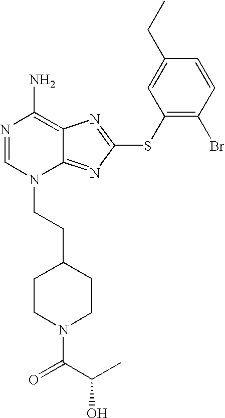 | (2S)-1-[4-(2-{6-Amino-8-[(2- bromo-5-ethylphenyl)thio]-3H- purin-3-yl}ethyl)piperidin-1-yl]-1- oxopropan-2-ol ¹H NMR (DMSO-d₆) δ 8.57 (s, 1H), 7.65 (d, J = 8.8 Hz, 1H), 7.49 (s, 1H), 7.18 (d, J = 8.8 Hz, 1H), 4.45-4.25 (m, 4H), 3.95-3.85 (m, 1H), 2.95-2.80 (m, 2H), 2.60-2.45 (m, 2H), 1.80-1.68 (m, 5H), 1.60- 1.45 (m, 2H). 1.30-1.20 (m, 2H), 1.18-1.07 (m, 4H); LC-MS [M + H]⁺ 533.13. |

### Example Compound 113

### (2R)-1-[4-(2-{6-Amino-8-[(7-bromo-2,3-dihydro-1,4-benzodioxin-6-yl)thio]-9H-purin-9-yl}ethyl)piperidin-1-yl]-1-oxopropan-2-ol

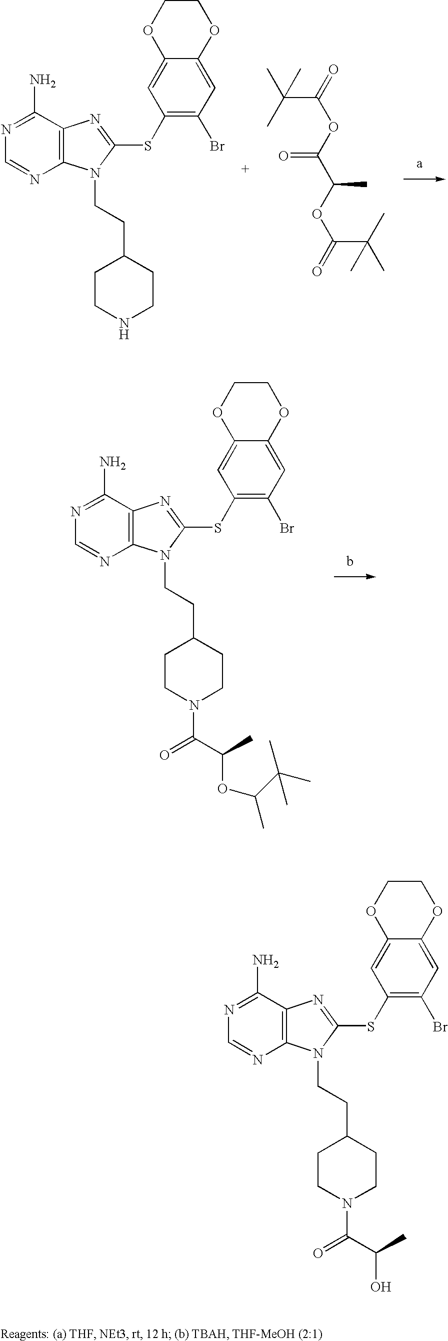

2,2-dimethylpropanoic (2R)-2-[(2,2-dimethylpropanoyl)oxy]propanoic anhydride was prepared in situ by reacting a suspension of D-lactic acid sodium salt (8.57 mmol, 0.96 g) in THF (15 mL), triethylamine (17.14 mmol, 2.38 mL) and pivaloyl chloride (17.14 mmol, 2.1 mL) at rt for overnight. To the above mixture was added 8-[(7-bromo-2,3-dihydro-1,4-benzodioxin-6-yl)thio]-9-(2-piperidin-4-ylethyl)-9H-purin-6-amine (5.71 mmol, 2.8 g) and stirred for 12 h. The reaction mixture was then diluted with saturated aq. NaHCO₃ (50 mL), and extracted with CHCl₃ (2×60 mL). The combined organic layer was dried over Na₂SO₄, filtered and the solvent was evaporated to dryness to afford (1R)-2-[4-(2-{6-amino-8-[(7-bromo-2,3-dihydro-1,4-benzodioxin-6-yl)thio]-9H-purin-9-yl}ethyl)piperidin-1-yl]-1-methyl-2-oxoethyl pivalate. LC-MS [M+H]⁺ 647.2. This product was sufficiently pure for the next reaction and used without further purification. A mixture of (1R)-2-[4-(2-{6-amino-8-[(7-bromo-2,3-dihydro-1,4-benzodioxin-6-yl)thio]-9H-purin-9-yl}ethyl)piperidin-1-yl]-1-methyl-2-oxoethyl pivalate (5.72 mmol, 3.7 g) and tetrabutylammoniumhydroxide (TBAH) 40% aq. solution (6.87 mmol, 4.2 mL) in THF-MeOH (2:1, 25 mL) was stirred for 6 h. After completion of hydrolysis, the solvent was evaporated under reduced pressure and the residue was diluted with MeOH (10 mL) and solids were collected by filtration and washed with MeOH. The product was re-crystallized form CHCl₃ and acetonitrile (1:3) to provide (2R)-1-[4-(2-{6-amino-8-[(7-bromo-2,3-dihydro-1,4-benzodioxin-6-yl)thio]-9H-purin-9-yl}ethyl)piperidin-1-yl]-1-oxopropan-2-ol (1.9 g, 59%). ¹H NMR (CD₃OD) δ 8.28 (s, 1H), 7.26 (s, 1H), 7.22 (d, J=2.3 Hz, 1H), 4.6-4.52 (m, 1H), 4.52-4.44 (m, 1H), 4.36 (t, J=7.4 Hz, 2H), 4.32-4.26 (m, 4H), 4.0 (d, J=14.4 Hz, 1H), 3.08-2.98 (m, 1H), 2.68-2.58 (m, 1H), 1.94-1.78 (m, 4H), 1.67-1.54 (m, 1H), 1.3 (dd, J=10.5, 6.6 Hz, 3H), 1.28-1.1 (m, 2H); TOF LC-MS [M+H]⁺ 565.11.

### Example Compounds 114 and 115

### tert-Butyl {(1R)-2-[4-({6-amino-8-[(7-chloro-1,3-benzothiazol-2-yl)thio]-9H-purin-9-yl}methyl)piperidin-1-yl]-1-methyl-2-oxoethyl}carbamate and tert-butyl {(1R)-2-[4-({6-amino-8-[(7-chloro-1,3-benzothiazol-2-yl)thio]-3H-purin-3-yl}methyl)piperidin-1-yl]-1-methyl-2-oxoethyl}carbamate

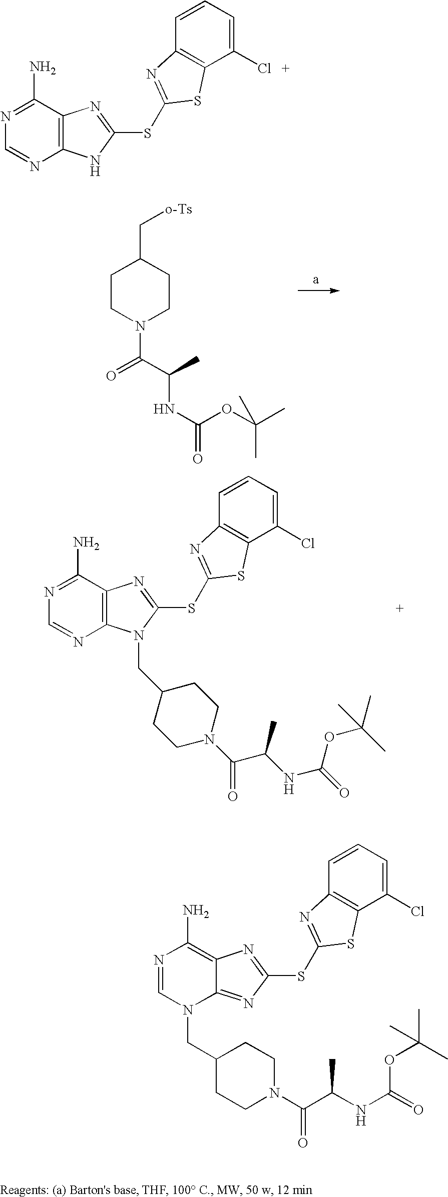

A mixture of 8-[(7-Chloro-1,3-benzothiazol-2-yl)thio]-9H-purin-6-amine (0.04 g, 0.105 mmol), toluene-4-sulfonic acid 1-((R)-2-tert-butoxycarbonylamino-propionyl)-piperidin-4-ylmethyl ester (0.05 g, 0.105 mmol) and Barton's base (43 μL, 0.21 mmol) in THF (3 mL) was heated at 100° C. for 12 min under microwave irradiation with 50 w power. After cooling, the reaction mixture was concentrated under reduced pressure and crude was purified by preparative HPLC [X-Terra prep-RP18 10 um, 19×250 mm (waters), Mobile phase: solvent A: Water HPLC grade containing 0.01% TFA, and solvent B: acetonitrile containing 0.01% TFA, general eluting gradient—solvent B 15% to 80 over 15 to 25 minutes run time]. After lyophilization of HPLC fractions to afford the example compounds: tert-Butyl {(1R)-2-[4-({6-amino-8-[(7-chloro-1,3-benzothiazol-2-yl)thio]-9H-purin-9-yl}methyl)piperidin-1-yl]-1-methyl-2-oxoethyl}carbamate (0.01 g, 16%) LC-MS [M+H]⁺ 602.94 and tert-butyl {(1R)-2-[4-({6-amino-8-[(7-chloro-1,3-benzothiazol-2-yl)thio]-3H purin-3-yl}methyl)piperidin-1-yl]-1-methyl-2-oxoethyl}carbamate. LC-MS [M+H]⁺ 603.1.

### Example Compounds 116-121

Example compounds 116-121 were synthesized in the same manner as described for example compounds 114 and 115, above, using appropriate starting materials and are summarized in table 5, below.

TABLE 5
Preparation of Example Compounds 116-121

| Example | | |
|---|---|---|
| Compound | Structure | Name and analytical data |
| 116 | 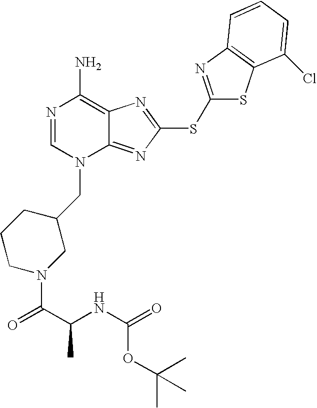 | tert-Butyl {(1S)-2-[3-({6- amino-8-[(7-chloro-1,3- benzothiazol-2-yl)thio]-3H- purin-3-yl}methyl)piperidin-1- yl]-1-methyl-2- oxoethyl}carbamate TOF LC-MS [M + H]⁺ 603.17 |
| 117 | 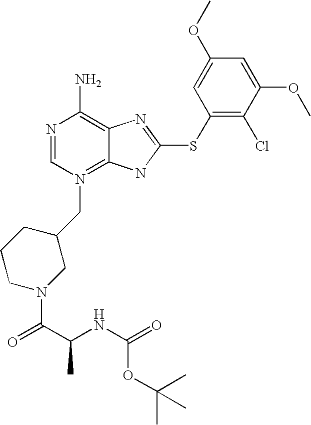 | tert-Butyl {(1S)-2-[3-({6- amino-8-[(2-chloro-3,5- dimethoxyphenyl)thio]-9H- purin-9-yl}methyl)piperidin-1- yl]-1-methyl-2- oxoethyl}carbamate TOF LC-MS [M + H]⁺ 606.23 |
| 118 | 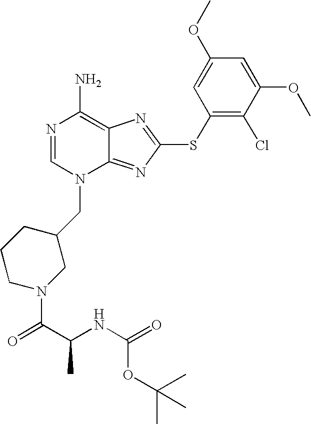 | tert-Butyl {(1S)-2[3-({6- amino-8-[(2-chloro-3,5- dimethoxyphenyl)thio]-3H- purin-3-yl}methyl)piperidin-1- yl]-1-methyl-2- oxoethyl}carbamate TOF LC-MS [M + H]⁺ 606.23 |
| 119 | 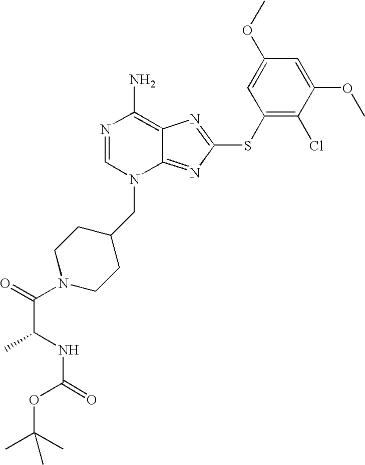 | tert-Butyl {(1R)-2-[4-({6- amino-8-[(2-chloro-3,5- dimethoxyphenyl)thio]-3H- purin-3-yl}methyl)piperidin-1- yl]-1-methyl-2- oxoethyl}carbamate; TOF LC-MS [M + H]⁺ 606.23 |
| 120 | 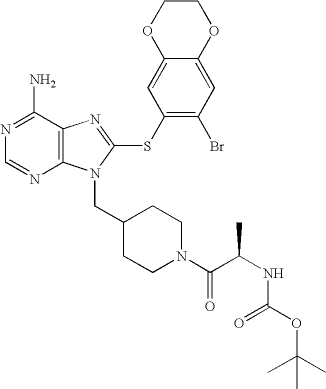 | tert-Butyl {(1R)-2-[4-({6- amino-8-[(7-bromo-2,3- dihydro-1,4-benzodioxin-6- yl)thio]-9H-purin-9- yl}methyl)piperidin-1-yl]-1- methyl-2-oxoethyl}carbamate; LC-MS [M + H]⁺ 648.1 |
| 121 | 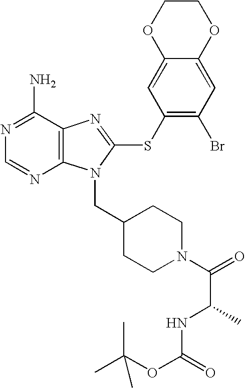 | tert-Butyl {(1S)-2-[4-({6- amino-8-[(7-bromo-2,3- dihydro-1,4-benzodioxin-6- yl)thio]-9H-purin-9- yl}methyl)piperidin-1-yl]-1- methyl-2-oxoethyl}carbamate; LC-MS [M + H]⁺ 648.1 |

### Example Compound 122

### 9-({1-[(2R)-2-Aminopropanoyl]piperidin-4-yl} methyl)-8-[(7-chloro-1,3-benzothiazol-2-yl)thio]-9H-purin-6-amine

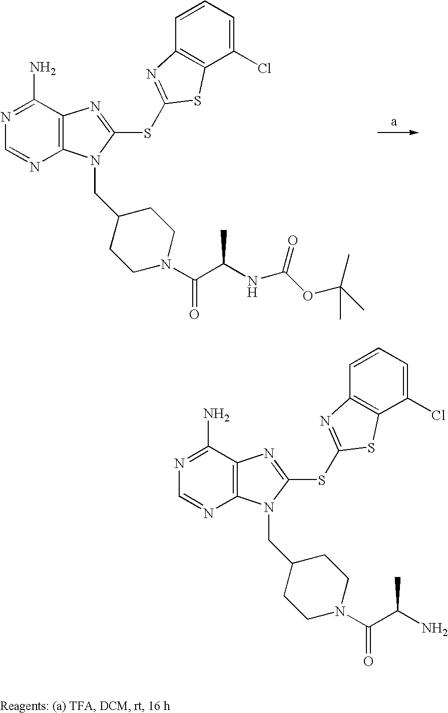

To a solution of tert-butyl {(1R)-2-[4-({6-amino-8-[(7-chloro-1,3-benzothiazol-2-yl)thio]-9H-purin-9-yl}methyl)piperidin-1-yl]-1-methyl-2-oxoethyl}carbamate (5.0 mg, 0.0083 mmol), in DCM (3 mL) was added drop wise TFA (3.2 μL) and the resulting mixture was stirred for 16 h at room temperature. After concentration under reduced pressure, the residual TFA was removed to afford the example product (7.0 mg, 78%) as a TFA salt. LC-MS [M+H]⁺ 503.1

### Example Compounds 123-125

Example compounds 123-125 were synthesized in the same manner as described for example compound 122, above, using appropriate starting materials and are summarized in table 6, below.

TABLE 6
Preparation of Example Compounds 123-125

| Example | | |
|---|---|---|
| Compound | Structure | Name and analytical data |
| 123 | 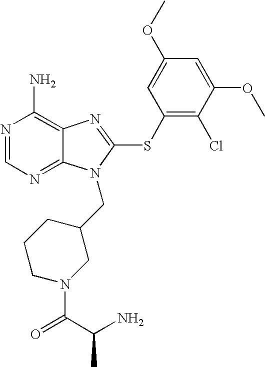 | 9-({1-[(2S)-2-Aminopropanoyl]piperidin- 3-yl}methyl)-8-[(2-chloro-3,5- dimethoxyphenyl)thio]-9H-purin-6-amine; TOF LC-MS [M + H]⁺ 506.2 |
| 124 | 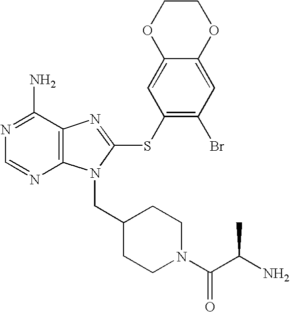 | 9-([1-[(2R)-2- Aminopropanoyl]piperidin-4-yl}methyl)-8- [(7-bromo-2,3-dihydro-1,4-benzodioxin-6- yl)thio]-9H-purin-6-amine; LC-MS [M + H]⁺ 548.1 |
| 125 | 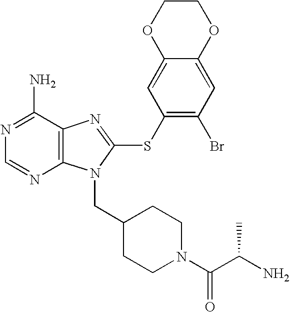 | 9-({1-[(2S)-2-Aminopropanoyl]piperidin- 4-yl}methyl)-8-[(7-bromo-2,3-dihydro-1,4- benzodioxin-6-yl)thio]-9H-purin-6-amine; LC-MS [M + H]⁺ 548.1 |

### Example Compound 126

### (2S)-N-{(1R)-2-[4-({6-Amino-8-[(7-bromo-2,3-dihydro-1,4-benzodioxin-6-yl)thio]-9H-purin-9-yl}methyl)piperidin-1-yl]-1-methyl-2-oxoethyl}-2-hydroxypropanamide

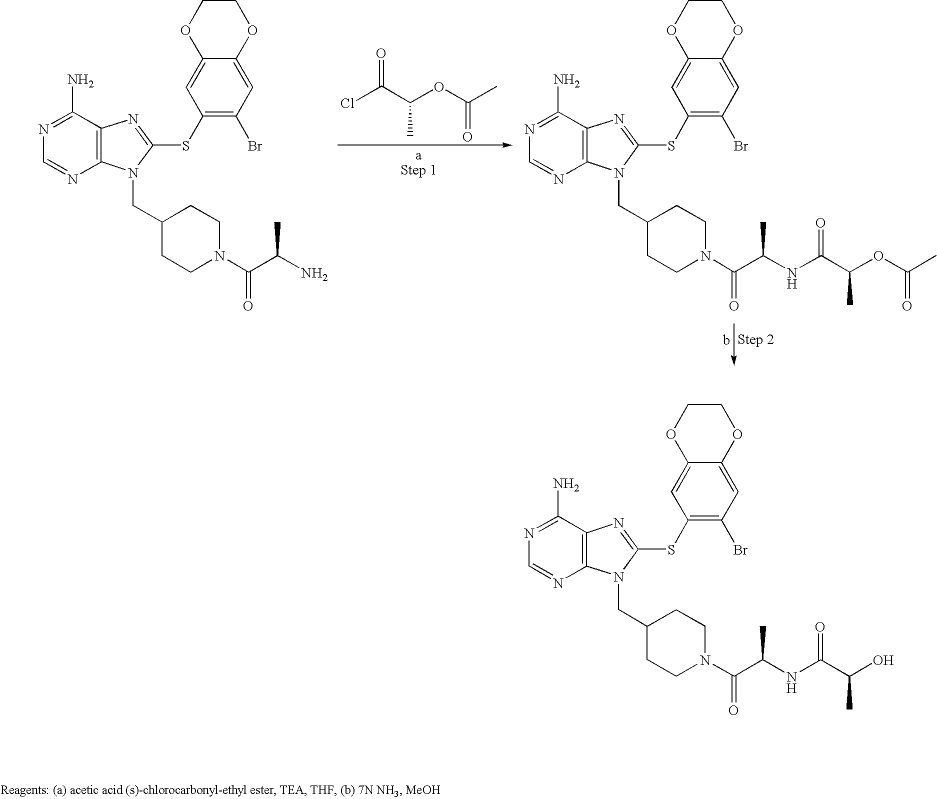

Step 1: 9-({1-[(2R)-2-Aminopropanoyl]piperidin-4-yl} methyl)-8-[(7-bromo-2,3-dihydro-1,4-benzodioxin-6-yl)thio]-9H-purin-6-amine (0.010 g, 0.018 mmol) was dissolved in THF (5 mL), followed by the addition of TEA (0.005 mL, 0.018 mmol) and acetic acid (S)-1-chlorocarbonyl-ethyl ester (0.002 mL, 0.036 mmol). After stirring for 3 h at rt, the reaction mixture was extracted with ethyl acetate and washed with water. The organic layer was dried over Na₂SO₄, filtered and concentrated in vacuum to afford (1S)-2-({(1R)-2-[4-({6-amino-8-[(7-bromo-2,3-dihydro-1,4-benzodioxin-6-yl)thio]-9H-purin-9-yl} methyl)piperidin-1-yl]-1-methyl-2-oxoethyl}amino)-1-methyl-2-oxoethyl acetate (0.010 g) as a light yellow oil; LC-MS [M+H]⁺ 662.1

Step 2: The compound obtained from step 1 was dissolved in MeOH (2 mL), followed by the addition of 7 N ammonia (1 mL). After stirring at rt for 18 h, the reaction mixture was concentrated in vacuum to afford the example compound (2S)—N-{(1R)-2-[4-({6-amino-8-[(7-bromo-2,3-dihydro-1,4-benzodioxin-6-yl)thio]-9H-purin-9-yl}methyl)piperidin-1-yl]-1-methyl-2-oxoethyl}-2-hydroxypropanamide (7.0 mg) as a light brown solid; LC-MS [M+H]⁺ 620.1

### Example Compounds 127 and 128

### (1R)-2-({(1R)-2-[4-({6-amino-8-[(7-bromo-2,3-dihydro-1,4-benzodioxin-6-yl)thio]-9H-purin-9-yl}methyl)piperidin-1-yl]-1-methyl-2-oxoethyl}amino)-1-methyl-2-oxoethyl pivalate and N-{(1R)-2-[4-({6-amino-8-[(7-bromo-2,3-dihydro-1,4-benzodioxin-6-yl)thio]-9H-purin-9-yl} methyl)piperidin-1-yl]-1-methyl-2-oxoethyl}-2,2-dimethylpropanamide

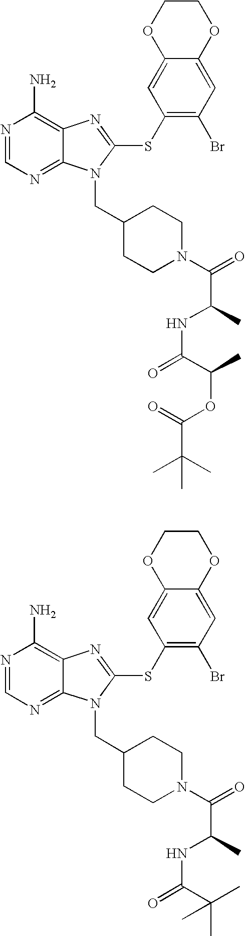

Example compound 127 was synthesized according to the procedure described for example compound 113 using 9-({1-[(2R)-2-aminopropanoyl]piperidin-4-yl}methyl)-8-[(7-bromo-2,3-dihydro-1,4-benzodioxin-6-yl)thio]-9H-purin-6-amine and D-lactic acid sodium salt, LC-MS [M+H]⁺ 704.18. The example compound 128 was isolated as a by-product LC-MS [M+H]⁺ 631.16

### Example Compound 129

### N-{(1R)-2-[4-({6-Amino-8-[(7-bromo-2,3-dihydro-1,4-benzodioxin-6-yl)thio]-9H-purin-9-yl} methyl)piperidin-1-yl]-1-methyl-2-oxoethyl}-3,3-dimethylbutanamide

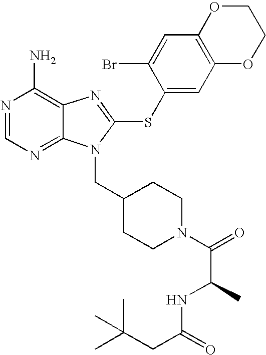

To a solution of 9-({1-[(2R)-2-aminopropanoyl]piperidin-4-yl}methyl)-8-[(7-bromo-2,3-dihydro-1,4-benzodioxin-6-yl)thio]-9H-purin-6-amine (0.011 g, 0.020 mmol) in THF (3 mL) was added 3,3-dimethyl-butyryl chloride (0.003 mL, 0.020 mmol) and Et₃N (0.005 mL) at rt and stirring continued at rt for 3 h. At the end of this period water was added and extracted with ethyl acetate. The organic layer was washed with water, dried (Na₂SO₄), filtered and the solvent was evaporated under reduced pressure to afford the example compound (7 mg), LC-MS [M+H]⁺ 646.19

### Example Compound 130

### (2S)—N-{(1S)-2-[4-({6-Amino-8-[(7-bromo-2,3-dihydro-1,4-benzodioxin-6-yl)thio]-9H-purin-9-yl} methyl)piperidin-1-yl]-1-methyl-2-oxoethyl}-2-hydroxypropanamide

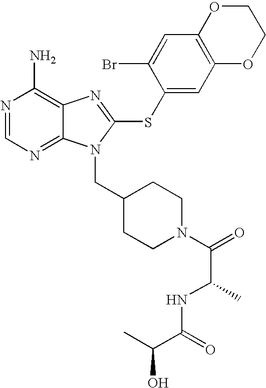

Example compound 130 was synthesized according to the procedure described for example compound 126 using 9-({1-[(2S)-2-aminopropanoyl]piperidin-4-yl}methyl)-8-[(7-bromo-2,3-dihydro-1,4-b enzodioxin-6-yl)thio]-9H-purin-6-amine and acetic acid (S)-1-chlorocarbonyl-ethyl ester. LC-MS [M+H]⁺ 620.06

### Example Compound 131

### N-{(1S)-2-[4-({6-Amino-8-[(7-bromo-2,3-dihydro-1,4-benzodioxin-6-yl)thio]-9H-purin-9-yl}methyl)piperidin-1-yl]-1-methyl-2-oxoethyl}-2-hydroxyacetamide

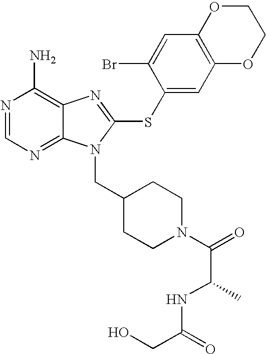

Example compound 131 was synthesized according to the procedure described for example compound 126 using 9-({1-[(2S)-2-aminopropanoyl]piperidin-4-yl}methyl)-8-[(7-bromo-2,3-dihydro-1,4-b enzodioxin-6-yl)thio]-9H-purin-6-amine and acetic acid chlorocarbonylmethyl ester. LC-MS [M+H]⁺ 606.9

### Example Compound 132

### (2R)—N-{(1R)-2-[4-({6-Amino-8-[(7-bromo-2,3-dihydro-1,4-benzodioxin-6-yl)thio]-9H-purin-9-yl} methyl)piperidin-1-yl]-1-methyl-2-oxoethyl}-2-hydroxypropanamide

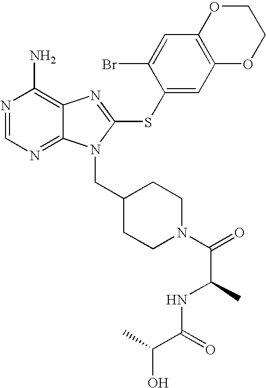

Example compound 132 was synthesized according to the procedure described for example compound 113 using 9-({1-[(2R)-2-aminopropanoyl]piperidin-4-yl}methyl)-8-[(7-bromo-2,3-dihydro-1,4-b enzodioxin-6-yl)thio]-9H-purin-6-amine and D-lactic acid sodium salt. LC-MS [M+H]⁺ 619.95

### Example Compounds 133 and 134

### 6-[(6-Amino-9-{2-[1-(3,3,3-trifluoroalanyl)piperidin-4-yl]ethyl}-9H-purin-8-yl)thio]-1,3-benzodioxole-5-carbonitrile and 6-[(6-amino-3-{2-[1-(3,3,3-trifluoroalanyl)piperidin-4-yl]ethyl}-3H-purin-8-yl)thio]-1,3-benzodioxole-5-carbonitrile.

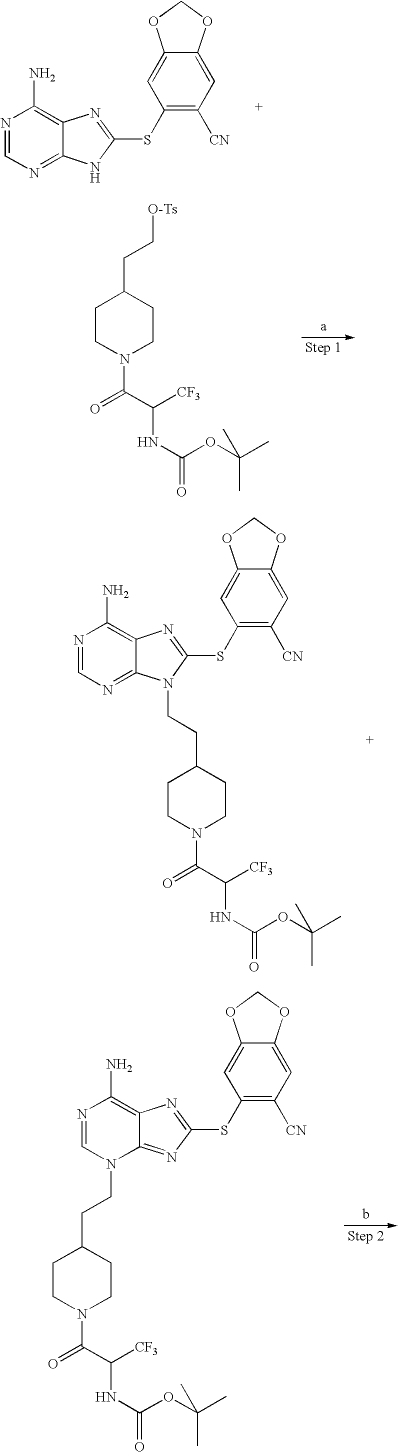

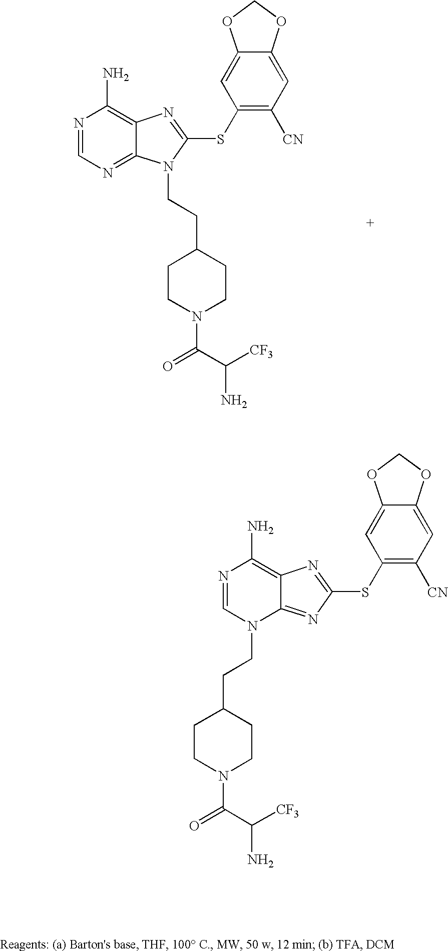

Step 1. A mixture of 6-(6-amino-9H-purin-8-ylsulfanyl)-benzo[1,3]dioxole-5-carbonitrile (0.1 g, 0.32 mmol), toluene-4-sulfonic acid 2-[1-(2-tert-butoxycarbonylamino-3,3,3-trifluoro-propionyl)-piperidin-4-yl]-ethyl ester (0.211 g, 0.41 mmol) and Barton's base (98 μL, 0.48 mmol) in THF (3 mL) was heated at 100° C. for 12 min under microwave irradiation with 50 w power. After cooling, the reaction mixture was concentrated under reduced pressure to afford mixture of tert-butyl (1-{[4-(2-{6-amino-8-[(6-cyano-1,3-benzodioxol-5-yl)thio]-9H-purin-9-yl}ethyl)piperidin-1-yl]carbonyl}-2,2,2-trifluoroethyl) carbamate; LC-MS [M+H]⁺ 649.2 and tert-butyl (1-{[4-(2-{6-amino-8-[(6-cyano-1,3-benzodioxol-5-yl)thio]-3H-purin-3-yl}ethyl)piperidin-1-yl]carbonyl}-2,2,2-trifluoroethyl) carbamate; LC-MS [M+H]⁺ 649.2. The crude mixture was used for the next reaction without further purification.

Step 2. To a solution of (0.207 g, 0.31 mmol), in DCM (5 mL) was added drop wise TFA (166 μL, 3.19 mmol) and the resulting mixture were stirred for overnight at room temperature. After concentration under reduced pressure, the residual TFA was removed, the residue was subjected to purification by preparative HPLC [X-Terra prep-RP18 10 um, 19×250 mm (waters), Mobile phase: solvent A: Water HPLC grade containing 0.01% TFA, and solvent B: acetonitrile containing 0.01% TFA, general eluting gradient—solvent B 15% to 80 over 15 to 25 minutes run time]. After lyophilization of HPLC fractions the example compounds were isolated as trifluoro acetate salt. 6-[(6-Amino-9-{2-[1-(3,3,3-trifluoroalanyl)piperidin-4-yl]ethyl}-9H-purin-8-yl)thio]-1,3-benzodioxole-5-carbonitrile ¹H NMR (CD₃OD) δ 8.28 (s, 1H), 7.38 (s, 1H), 7.36 (s, 1H), 6.2 (s, 2H), 5.47 (qd, J=33.9, 6.6 Hz, 1H), 4.55 (d, J=13.6 Hz, 1H), 4.39 (t, J=6.6 Hz, 2H), 4.03 (t, J=12.1 Hz, 1H), 3.26-3.16 (s, 1H), 2.79 (t, J=12.8 Hz, 1H), 2.06-1.83 (m, 4H), 1.75-1.6 (m, 1H), 1.25-1.02 (m, 2H); TOF LC-MS [M]⁺ 548.87 and 6-[(6-amino-3-{2-[1-(3,3,3-trifluoroalanyl)piperidin-4-yl]ethyl}-3H-purin-8-yl)thio]-1,3-benzodioxole-5-carbonitrile. TOF LC-MS [M]⁺ 548.87.

### Example Compound 135

### 6-[(6-Amino-9-{2-[1-(4,4-difluoro-L-prolyl)piperidin-4-yl]ethyl}-9H-purin-8-yl)thio]-1,3-benzodioxole-5-carbonitrile

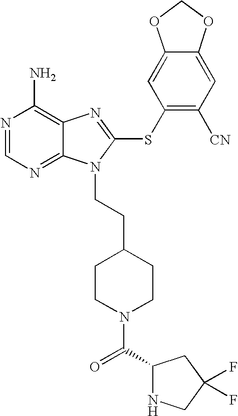

The example compound was prepared by a similar procedure described for example compounds 133 and 134 using 6-(6-amino-9H-purin-8-ylsulfanyl)-benzo[1,3]dioxole-5-carbonitrile and 4,4 difluoro-2-{4-[2-(toluene-4-sulfonyloxy)-ethyl]-piperidine-1-carbonyl}-L-pyrrolidine-1-carboxylic acid tert-butyl ester. ¹H NMR (DMSO-d₆) δ 8.3 (s, 1H), 8.29-8.14 (broad s, 2H), 7.61 (s, 1H), 7.25 (s, 1H), 6.24 (s, 1H), 4.98 (td, J=27.3, 8.5 Hz, 1H), 4.4-4.2 (m, 3H), 3.82-3.64 (m, 3H), 3.08-2.97 (m, 1H), 2.72-2.6 (m, 1H), 1.88-1.76 (m, 2H), 1.76-1.66 (m, 2H), 1.62-1.45 (m, 1H), 1.32-1.0 (m, 4H); TOF LC-MS [M+H]⁺ 557.18.

### Example Compound 136

### 9-(3-{1-[(2R)-2-Aminopropanoyl]piperidin-3-yl}propyl)-8-[(6-bromo-1,3-benzodioxol-5-yl)thio]-9H-purin-6-amine

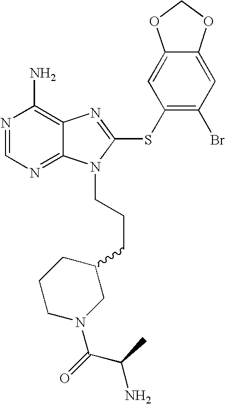

The example compound was prepared by a similar procedure to that described for example compounds 133 and 134 using 6-(6-amino-9H-purin-8-ylsulfanyl)-benzo[1,3]dioxole-5-carbonitrile and toluene-4-sulfonic acid 3-[1-((R)-2-tert-butoxycarbonylamino-propionyl)-piperidin-4-yl]-propyl ester. ¹H NMR (DMSO-d₆) δ 8.29 (s, 1H), 7.41-7.39 (m, 1H), 6.92-6.9 (m, 1H), 6.11 (s, 1H), 4.45-4.06 (m, 4H), 3.67 (q, J=12.5 Hz, 1H), 3.09-2.94 and 2.72-2.62 (two m, 1H), 2.62-2.29 (m, 1H), 1.84-1.58 (m, 4H), 1.46-1.0 (m, 8H); LC-MS [M+H]⁺ 562.1.

### Example Compounds 137 and 138

### N-{3-[6-Amino-8-(6-bromo-benzo[1,3]dioxol-5-ylsulfanyl)-purin-9-yl]-1-cyclopropyl-propyl}-acetamide and N-{3-[6-acetylamino-8-(6-bromo-benzo[1,3]dioxol-5-ylsulfanyl)-purin-9-yl]-1-cyclopropyl-propyl}-acetamide

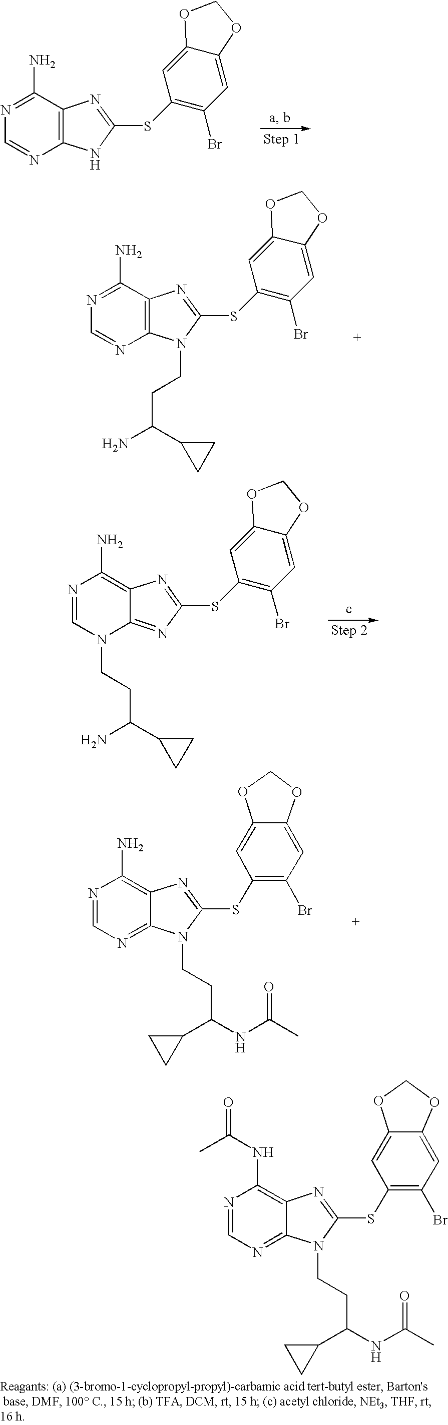

Step 1. A mixture of 8-(6-bromo-benzo[1,3]dioxol-5-ylsulfanyl)-9H-purin-6-ylamine (0.145 g, 0.396 mmol), (3-bromo-1-cyclopropyl-propyl)-carbamic acid tert-butyl ester (0.228 g, 0.82 mmol), and Barton's base (0.140 g, 0.82 mmol) in DMF (4 mL) was heated at 80-100° C. for 15 h. After cooling, the reaction mixture was concentrated under reduced pressure. The LC-MS analysis indicated presence of 2:1 mixture of tert-butyl (3-{6-amino-8-[(6-bromo-1,3-benzodioxol-5-yl)thio]-9H-purin-9-yl}-1-cyclopropylpropyl)carbamate and tert-butyl (3-{6-amino-8-[(6-bromo-1,3-benzodioxol-5-yl)thio]-3H-purin-3-yl}-1-cyclopropylpropyl)carbamate. The above mixture was dissolved in DCM and TFA (1.0 mL) was added at rt and stirring continued overnight to provide a 2:1 mixture of 9-(3-amino-3-cyclopropyl-propyl)-8-(6-bromo-benzo[1,3]dioxol-5-ylsulfanyl)-9H-purin-6-ylamine and 3-(3-amino-3-cyclopropyl-propyl)-8-(6-bromo-benzo[1,3]dioxol-5-ylsulfanyl)-3H-purin-6-ylamine. The product is used for the next step without further purification.

Step 2. A 2:1 mixture of 9-(3-amino-3-cyclopropyl-propyl)-8-(6-bromo-benzo[1,3]dioxol-5-ylsulfanyl)-9H-purin-6-ylamine and 3-(3-amino-3-cyclopropyl-propyl)-8-(6-bromo-benzo[1,3]dioxol-5-ylsulfanyl)-3H-purin-6-ylamine (0.38 g, 0.82 mmol) in THF (9.0 mL) was added acetyl chloride (0.12 mL, 1.60 mmol), Et₃N (0.34 mL, 2.46 mmol) at room temperature and stirring continued overnight. At the end of this period the solvent was removed in vacuo and mixture was purified by preparative HPLC to afford N-{3-[6-Amino-8-(6-bromo-benzo[1,3]dioxol-5-ylsulfanyl)-purin-9-yl]-1-cyclopropyl-propyl}-acetamide. ¹H NMR (CD₃OD) δ 8.17 (s, 1H), 7.25 (s, 1H), 7.07 (s, 1H), 6.06 (s, 2H), 4.44-4.39 (m, 2H), 3.2-3.1 (m, 1H), 2.4-2.35 (m, 1H), 2.1-2.08 (m, 1H), 1.96 (s, 3H), 0.92-0.87 (m, 1H), 0.54-0.42 (m, 2H), 0.29-0.1 (m, 2H); TOF LC-MS [M+H]⁺ 505.06 and N-{3-[6-acetylamino-8-(6-bromo-benzo[1,3]dioxol-5-ylsulfanyl)-purin-9-yl]-1-cyclopropyl-propyl}-acetamide. ¹H NMR (CD₃OD) δ 8.74 (s, 1H), 7.35 (s, 1H), 7.28 (s, 1H), 6.14 (s, 2H), 4.6-4.4 (m, 2H), 3.2-3.1 (m, 1H), 2.4-2.35 (m, 1H), 2.28 (s, 3H), 2.10-2.08 (m, 1H), 1.89 (s, 3H), 0.92-0.87 (m, 1H), 0.54-0.42 (m, 2H), 0.29-0.1 (m, 2H); TOF LC-MS [M+H]⁺ 505.06.

### Example Compound 139

### N-(3-{6-Amino-8-[(7-bromo-2,3-dihydro-1,4-benzodioxin-6-yl)thio]-9H-purin-9-yl}-1-cyclopropylpropyl)acetamide

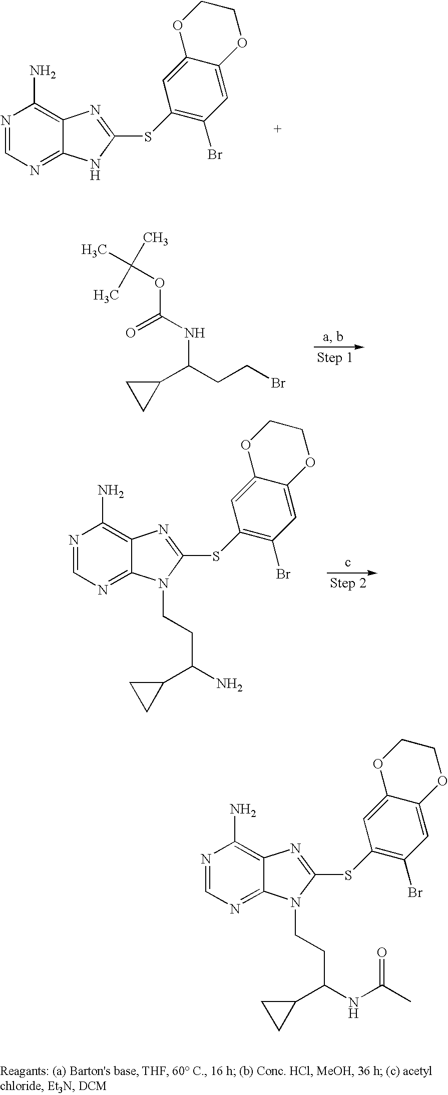

Step 1. To a mixture of (3-bromo-1-cyclopropyl-propyl)-carbamic acid tert-butyl ester (1.8 g, 6.5 mmol) and 8-[(7-bromo-2,3-dihydro-benzo[1,4]-dioxin-6-sulfanyl)-9H-purin-6-ylamine]-9H-purin-6-ylamine (1.23 g, 3.25 mmol) in THF (20 mL) was added Barton's base (1.33 mL, 6.5 mmol) at room temperature and the mixture was heated at 60° C. overnight. At the end of this period solvent was evaporated to dryness and the residue was chromatographed over SiO₂ using a mixture 2:2:0.5 DCM:EA:MeOH to afford a 2:1 mixture of tert-butyl (3-{6-amino-8-[(7-bromo-2,3-dihydro-1,4-benzodioxin-6-yl)thio]-9H-purin-9-yl}-1-cyclopropylpropyl)carbamate and 8-[(7-bromo-2,3-dihydro-1,4-benzodioxin-6-yl)thio]-3-{3-[(1-tert-butoxyvinyl)amino]-3-cyclopropylpropyl}-3H-purin-6-amine. LC-MS [M+H] 577.9. The above 2:1 mixture was taken up in MeOH (20 mL) conc. HCl (2.0 mL) was added and the mixture was stirred over night at room temperature. The solvent and the excess HCl was evaporated under reduced pressure. The residue was co evaporated with toluene to afford a 2:1 mixture of 9-(3-amino-3-cyclopropylpropyl)-8-[(7-bromo-2,3-dihydro-1,4-benzodioxin-6-yl)thio]-9H-purin-6-amine; LC-MS [M+H] 478.0 and 3-(3-amino-3-cyclopropylpropyl)-8-[(7-bromo-2,3-dihydro-1,4-benzodioxin-6-yl)thio]-3H-purin-6-amine (1.139 g, 63%); TOF LC-MS [M+H]⁺ 477.0.

Step 2. The example compound was prepared by a procedure similar to that described for example compounds 137 and 138 using a 2:1 mixture of 9-(3-amino-3-cyclopropylpropyl)-8-[(7-bromo-2,3-dihydro-1,4-benzodioxin-6-yl)thio]-9H-purin-6-amine and 3-(3-amino-3-cyclopropylpropyl)-8-[(7-bromo-2,3-dihydro-1,4-benzodioxin-6-yl)thio]-3H-purin-6-amine and acetyl chloride. ¹H NMR (DMSO-d₆) δ 8.19 (s, 1H), 7.27 (s, 1H), 6.68 (s, 1H), 4.30-4.20 (m, 6H), 3.33 (s, 3H), 2.60-2.40 (m, 4H), 1.70-1.00 (m, 5H); TOF LC-MS [M+H]⁺ 519.08.

### Example Compound 140

### 9-[2-(1-Acetylpiperidin-4-yl)ethyl]-8-[(7-bromo-2,3-dihydro-1,4-benzodioxin-6-yl)thio]-9H-purin-6-amine

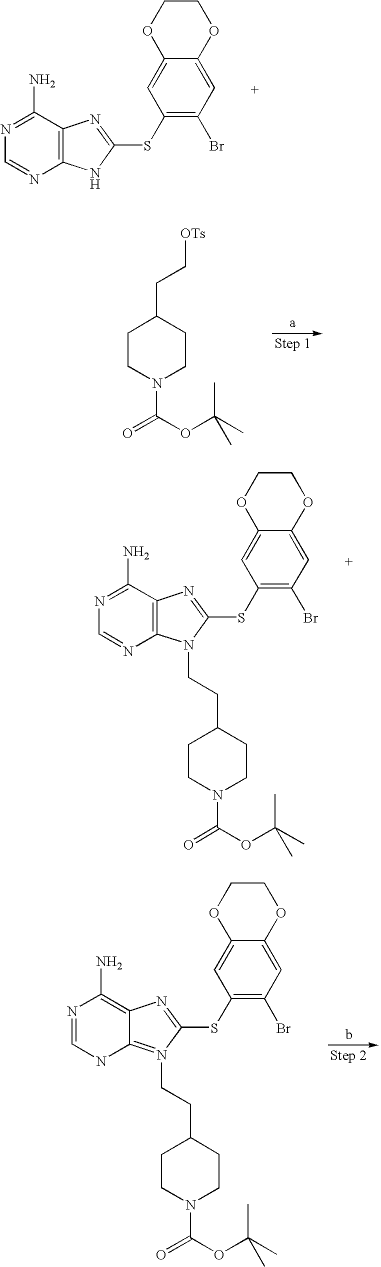

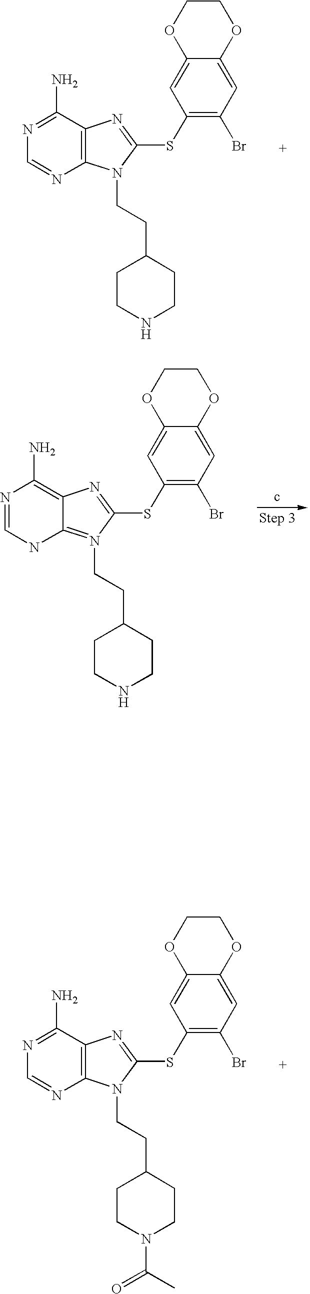

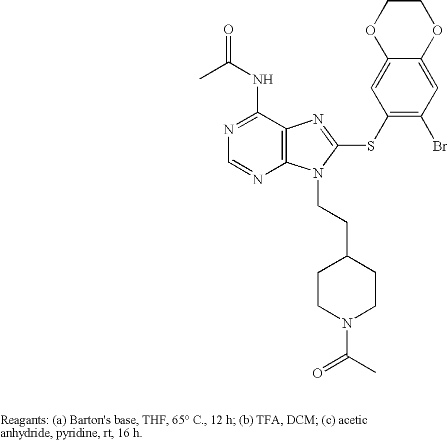

Step 1. tert-Butyl 4-(2-{6-amino-8-[(7-bromo-2,3-dihydro-1,4-benzodioxin-6-yl)thio]-9H-purin-9-yl}ethyl)piperidine-1-carboxylate and tert-butyl 4-(2-{6-amino-8-[(7-bromo-2,3-dihydro-1,4-benzodioxin-6-yl)thio]-3H-purin-3-yl}ethyl)piperidine-1-carboxylate:

To a mixture of 8-(7-bromo-2,3-dihydro-benzo[1,4]dioxin-6-ylsulfanyl)-9H-purin-6-ylamine (2.0 g, 5.26 mmol) and 4-[2-(toluene-4-sulfonyloxy)-ethyl]-piperidine-1-carboxylic acid tert-butyl ester (2.4 g, 6.31 mmol) in THF (15 mL) at room temperature was added Barton's base (1.29 mL, 6.31 mmol) and the reaction mixture was heated at 65° C. for 12 h. After completion of the reaction, the mixture was cooled down and diluted with 5 mL of methanol and evaporated under vacuum to provide the mixture of tert-butyl 4-(2-{6-amino-8-[(7-bromo-2,3-dihydro-1,4-benzodioxin-6-yl)thio]-9H-purin-9-yl}ethyl)piperidine-1-carboxylate, LC-MS [M+H]⁺ 591.2 and tert-butyl 4-(2-{6-amino-8-[(7-bromo-2,3-dihydro-1,4-benzodioxin-6-yl)thio]-3H-purin-3-yl}ethyl)piperidine-1-carboxylate, TOF LC-MS [M+H]⁺ 591.1. This crude mixture was used for the next step without further purification.

Step 2. 8-[(7-bromo-2,3-dihydro-1,4-benzodioxin-6-yl)thio]-9-(2-piperidin-4-ylethyl)-9H-purin-6-amine and 8-[(7-bromo-2,3-dihydro-1,4-benzodioxin-6-yl)thio]-3-(2-piperidin-4-ylethyl)-3H-purin-6-amine:

The mixture of products obtained from the step 1 was treated with 1:1 TFA and DCM (20 mL) and the mixture was stirred for 6 h. After completion of reaction the, mixture was evaporated under reduced pressure to remove TFA and DCM, the resulting crude was diluted with 10 mL methanol and neutralized with aq. NH₄OH and evaporated all the solvent under vacuum. The residue was triturated with MeOH and solids were collected by filtration and washed with MeOH and dried to provide 1.2 g of 8-[(7-bromo-2,3-dihydro-1,4-benzodioxin-6-yl)thio]-9-(2-piperidin-4-ylethyl)-9H-purin-6-amine. ¹H NMR (DMSO-d₆) δ 8.16 (s, 1H), 7.52-7.4 (broad s, 2H), 7.28 (s, 1H), 6.69 (s, 1H), 4.28-4.15 (m, 6H), 3.23 (d, J=12.5 Hz, 2H), 2.82-2.7 (m, 2H), 1.85 (d, J=12.1 Hz, 2H), 1.62 (q, J=6.6 Hz, 2H), 1.48-1.36 (m, 1H), 1.33-1.2 (m, 2H); TOF LC-MS [M+H]⁺ 491.2. The LC-MS analysis indicated the purity of the product is 95% along with trace quantity of 8-[(7-bromo-2,3-dihydro-1,4-benzodioxin-6-yl)thio]-3-(2-piperidin-4-ylethyl)-3H-purin-6-amine: TOF LC-MS [M+H]⁺ 491.2.

Step 3. 9-[2-(1-Acetylpiperidin-4-yl)ethyl]-8-[(7-bromo-2,3-dihydro-1,4-benzodioxin-6-yl)thio]-9H-purin-6-amine and N-{9-[2-(1-acetylpiperidin-4-yl)ethyl]-8-[(7-bromo-2,3-dihydro-1,4-benzodioxin-6-yl)thio]-9H-purin-6-yl}acetamide:

To a suspension of 8-[(7-bromo-2,3-dihydro-1,4-benzodioxin-6-yl)thio]-9-(2-piperidin-4-ylethyl)-9H-purin-6-amine (0.102 g, 0.20 mmol) in pyridine (5.0 mL) was added acetic anhydride (51 μL, 0.6 mmol) at rt and stirring for 16 h at rt. The reaction mixture was evaporated dryness under reduced pressure. The product was purified by preparative HPLC to afford the example compound: 9-[2-(1-Acetylpiperidin-4-yl)ethyl]-8-[(7-bromo-2,3-dihydro-1,4-benzodioxin-6-yl)thio]-9H-purin-6-amine. ¹H NMR (CD₃OD) δ 8.15 (s, 1H), 7.24 (s, 1H), 7.10 (s, 1H), 4.33-4.25 (m, 6H), 3.30-3.20 (m, 3H), 2.07 (s, 3H), 1.9-1.72 (m, 4H), 1.3-1.1 (m, 4H); LC-MS [M+H]⁺ 534.1 and N-{9-[2-(1-acetylpiperidin-4-yl)ethyl]-8-[(7-bromo-2,3-dihydro-1,4-benzodioxin-6-yl)thio]-9H-purin-6-yl}acetamide. ¹H NMR (DMSO-d₆) δ 10.72 (s, 1H), 8.63 (s, 1H), 7.32 (s, 1H), 6.59 (s, 1H), 4.33-4.20 (m, 4H) 3.41-3.25 (m, 8H), 2.60-2.40 (m, 6H), 1.75-1.55 (m, 4H), 1.1-0.85 (m, 1H); TOF LC-MS [M+H]⁺ 575.10.

### Example Compound 141

### N-(3-{6-Amino-8-[(6-bromo-1,3-benzodioxol-5-yl)thio]-9H-purin-9-yl}-1-cyclopropylpropyl)-2-hydroxyacetamide

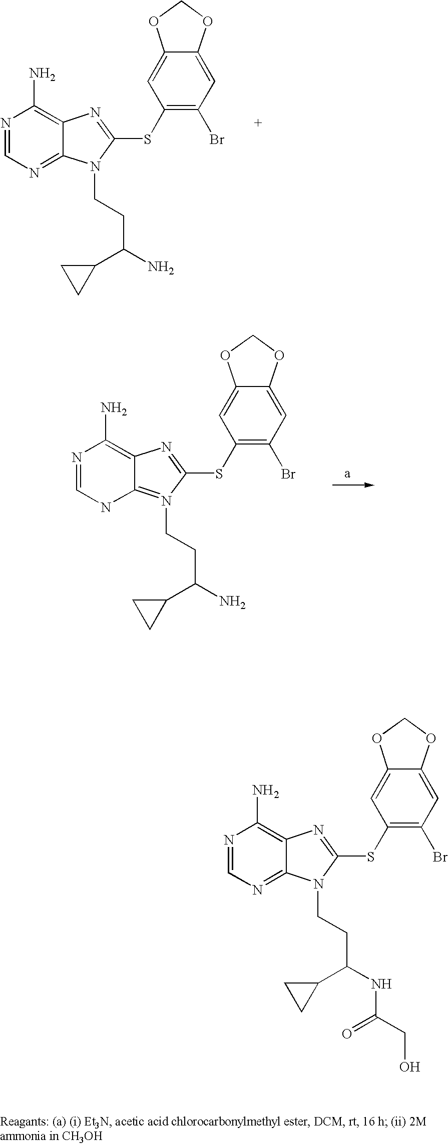

To a 2:1 mixture of 9-(3-amino-3-cyclopropyl-propyl)-8-(6-bromo-benzo[1,3]dioxol-5-ylsulfanyl)-9H-purin-6-ylamine and 3-(3-amino-3-cyclopropyl-propyl)-8-(6-bromo-benzo[1,3]dioxol-5-ylsulfanyl)-3H-purin-6-ylamine (0.301 g, 0.64 mmol) in DCM was added acetic acid chlorocarbonylmethyl ester (0.139 mL, 1.29 mmol) and triethylamine (0.269 mL, 1.93 mmol) at room temperature and stirring continued 16 h. The solvent was evaporated to dryness and the residue was suspended in methanol. To the mixture 2 M ammonia in methanol (6 mL) was added and stirred at room temperature for 4 h at which time a white particulates separated were collected and washed with cold methanol to afford N-(3-{6-amino-8-[(6-bromo-benzo[1,3]dioxol-5-yl)thio]-9H-purin-9-yl}-1-cyclopropylpropyl)-2-hydroxyacetamide. ¹H NMR (DMSO-d₆) δ 8.14 (s, 1H), 7.37 (s, 1H), 6.79 (s, 1H), 6.09 (s, 2H), 4.2-4.1 (m, 5H), 3.82 (s, 2H), 2.1-1.95 (m, 3H), 1.1-0.96 (m, 2H); TOF LC-MS [M+H]⁺ 521.0.

### Example Compounds 142, 143 and 144

### 8-[(7-Bromo-2,3-dihydro-1,4-benzodioxin-6-yl)thio]-9-[3-cyclopropyl-3-(dimethylamino)propyl]-9H-purin-6-amine, 8-[(7-bromo-2,3-dihydro-1,4-benzodioxin-6-yl)thio]-3-[3-cyclopropyl-3-(dimethylamino)propyl]-3H-purin-6-amine, and 8-(7-bromo-2,3-dihydro-benzo[1,4]dioxin-6-ylsulfanyl)-9-(3-cyclopropyl-3-methylamino-propyl)-9H-purin-6-ylamine

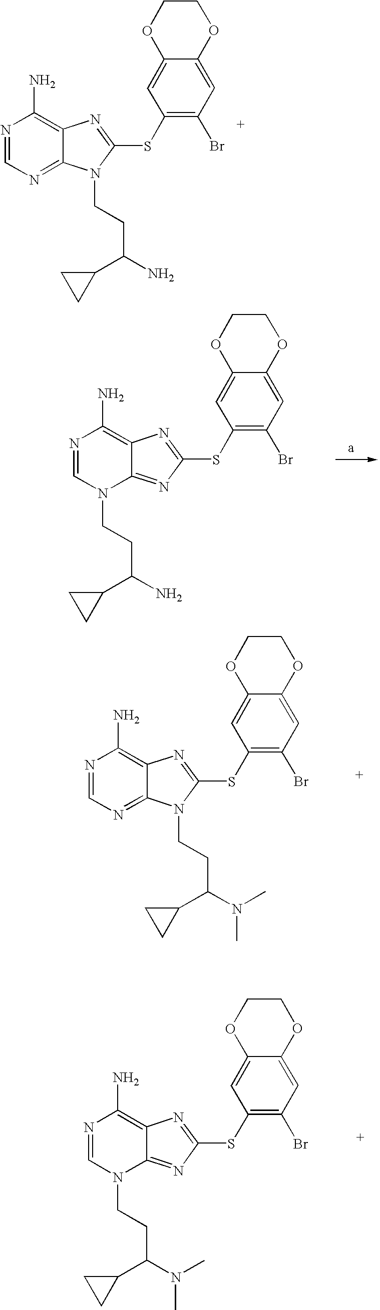

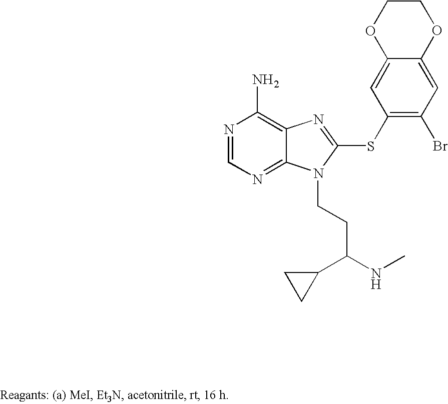

To a 2:1 mixture of 9-(3-amino-3-cyclopropylpropyl)-8-[(7-bromo-2,3-dihydro-1,4-benzodioxin-6-yl)thio]-9H-purin-6-amine and 3-(3-amino-3-cyclopropylpropyl)-8-[(7-bromo-2,3-dihydro-1,4-benzodioxin-6-yl)thio]-3H-purin-6-amine (0.199 g, 0.418 mmol) in anhydrous acetonitrile (5 mL) was added iodomethane (0.053 mL, 0.849 mmol) followed by triethyl amine (0.175 mL, 1.25 mmol) at room temperature and stirring continued for 16 h. The solvent was removed in vacuo and the reaction mixture was purified by preparative HPLC to yield the example compounds. 8-[(7-bromo-2,3-dihydro-1,4-benzodioxin-6-yl)thio]-9-[3-cyclopropyl-3-(dimethylamino)propyl]-9H-purin-6-amine. ¹H NMR (DMSO-d₆) δ 8.16 (s, 1H), 7.27 (s, 1H), 6.69 (s, 1H), 4.3-4.2 (m, 6H), 2.6-2.4 (m, 6H), 2.14-2.08 (m, 2H), 1.88-1.82 (m, 2H), 0.77-0.71 (m, 1H), 0.43-0.1 (m, 3H); TOF LC-MS [M+H]⁺ 505.10. 8-[(7-Bromo-2,3-dihydro-1,4-benzodioxin-6-yl)thio]-3-[3-cyclopropyl-3-(dimethylamino)propyl]-3H-purin-6-amine. ¹H NMR (DMSO-d₆) δ 8.34 (s, 1H), 7.18 (s, 1H), 6.97 (s, 1H), 4.4-4.2 (m, 6H), 2.6-2.4 (m, 6H), 2.3-1.95 (m, 3H), 0.9-0.7 (m, 1H), 0.5-0.1 (m, 3H); TOF LC-MS [M+H]⁺ 505.10 and 8-(7-bromo-2,3-dihydro-1,4-benzodioxin-6-ylsulfanyl)-9-(3-cyclopropyl-3-methylamino-propyl)-9H-purin-6-ylamine. TOF LC-MS [M+H]⁺ 491.08.

### Example Compounds 145 and 146

### 4-(2-{6-Amino-8-[(2-bromo-4,5-dihydroxyphenyl)thio]-9H-purin-9-yl}ethyl)piperidine-1-carbaldehyde and 4-(2-{6-amino-8-[(2-bromo-4,5-dihydroxyphenyl)thio]-3H-purin-3-yl}ethyl)piperidine-1-carbaldehyde

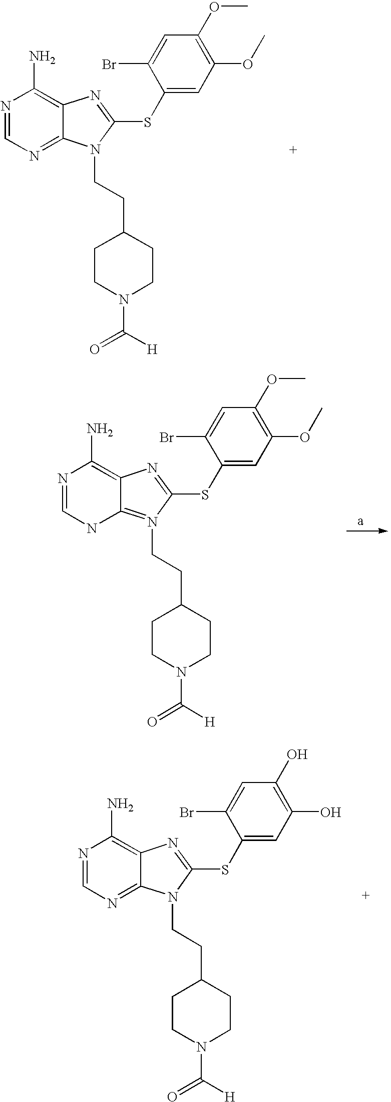

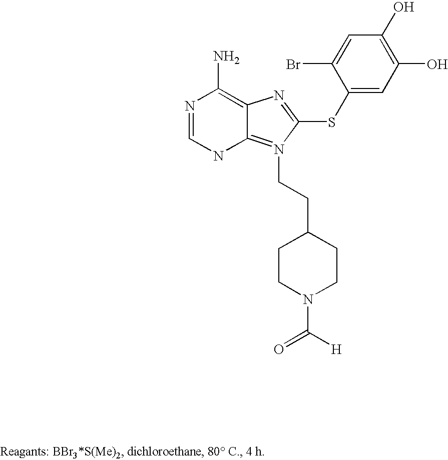

To a solution of 2:1 mixture of 4-(2-{6-amino-8-[(2-bromo-4,5-dimethoxyphenyl)thio]-9H-purin-9-yl}ethyl)piperidine-1-carbaldehyde and 4-(2-{6-amino-8-[(2-bromo-4,5-dimethoxyphenyl)thio]-3H-purin-3-yl}ethyl)piperidine-1-carbaldehyde (0.356 g, 0.68 mmol) in dichloroethane (10 mL) was added boron tribromide-dimethyl sulfide complex (1M solution in dichloromethane, 16.4 mL, 16.4 mmol) at room temperature and the reaction mixture was refluxed for 4 h. At the end of this time the reaction mixture was cooled to room temperature, water (20 mL) and 2M aqueous hydrochloric acid (100 mL) was added. The aqueous layer was collected and neutralized to pH 7 with aqueous sodium hydroxide (15%; w/v) and extracted into ethyl acetate (2×200 mL). The organic layer was washed with brine (100 mL), dried over sodium sulfate, filtered and the solvent was removed in vacuo. The reaction mixture was purified by preparative HPLC to yield the example compounds. 4-(2-{6-amino-8-[(2-bromo-4,5-dihydroxyphenyl)thio]-9H-purin-9-yl}ethyl)piperidine-1-carbaldehyde; ¹H NMR (DMSO-d₆) δ 8.18 (s, 1H), 7.93 (s, 1H), 7.02 (s, 1H), 6.55 (s, 1H), 4.2-4.06 (m, 3H), 3.66-3.58 (m, 1H), 2.92-2.85 (m, 1H), 1.7-0.5 (m, 7H); TOF LC-MS [M+H]⁺ 493.06 and 4-(2-{6-amino-8-[(2-bromo-4,5-dihydroxyphenyl)thio]-3H-purin-3-yl}ethyl)piperidine-1-carbaldehyde; ¹H NMR (DMSO-d₆) δ 8.64 (s, 1H), 7.96 (s, 1H), 7.2-7.1 (m, 2H), 4.35-4.30 (m, 2H), 4.14-4.10 (m, 1H), 3.66-3.60 (m, 1H), 3.0-2.9 (m, 2H), 1.9-0.5 (m, 7H); TOF LC-MS [M+H]⁺ 493.06.

### Example Compound 147

### 8-[(6-Bromo-1,3-benzodioxol-5-yl)thio]-9-(2-piperidin-4-ylethyl)-9H-purin-6-amine

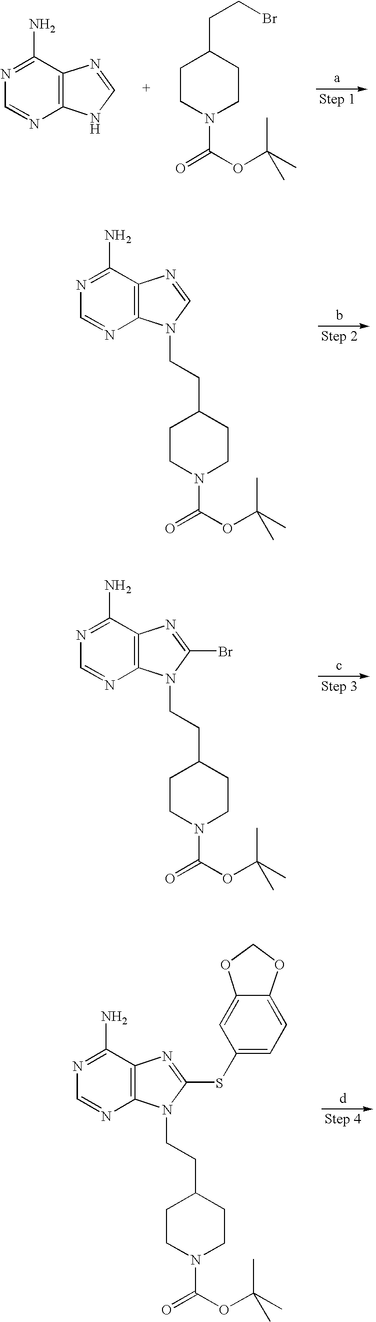

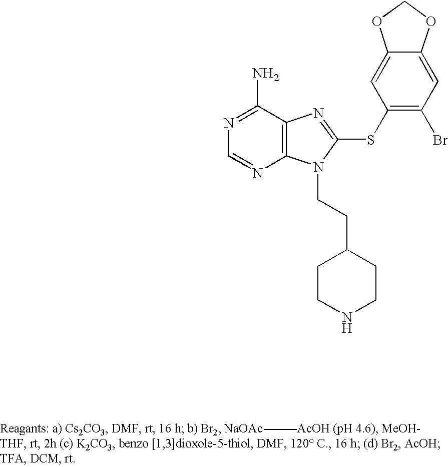

Step 1: tert-Butyl 4-[2-(6-amino-9H-purin-9-yl)ethyl]piperidine-1-carboxylate and tert-butyl 4-[2-(6-amino-3H-purin-3-yl)ethyl]piperidine-1-carboxylate

### Method A

A mixture of adenine (82.1 mmol, 11.1 g), 4-(2-bromo-ethyl)-piperidine-1-carboxylic acid tert-butyl ester (68.4 mmol, 20 g) and Cs₂CO₃ (136.9 mmol, 44.6 g) in DMF (100 mL) was stirred at room temperature for 12 h. The reaction mixture was filtered to remove Cs₂CO₃ and the filtrate was diluted with CHCl₃ (250 mL) and washed with water (2×200 mL) and brine (200 mL), organic layer was dried over Na₂SO₄, filtered and evaporated under vacuum to afford a 9:1 mixture of N-9 and N-3 isomers (19.2 g, 82%) LC-MS [M+H]⁺ 347.1. This product was used for the next step without further purifications.

### Method B

To a solution of N-boc-4-piperidineethanol (15.0 g, 65.4 mmol) in DCM (100 mL) was added Et₃N (23 mL, 164.0 mmol, 2.5 eq) and MsCl (11.0 mL, 131.0 mmol, 2.0 eq) at ˜5° C. and stirring continued at room temperature ˜16 h. At the end of this period reaction was diluted with DCM (100 mL) and washed with water (2×100 mL), dried over Na₂SO₄, filtered and the solvent was evaporated to dryness under reduced pressure to provide tert-butyl 4-{2-[(methylsulfonyl)oxy]ethyl}piperidine-1-carboxylate (20.0 g) in quantitative yield. This product was sufficiently pure for the next step and used without purifications. The example compounds tert-butyl 4-[2-(6-amino-9H-purin-9-yl)ethyl]piperidine-1-carboxylate and tert-butyl 4-[2-(6-amino-3H-purin-3-yl)ethyl]piperidine-1-carboxylate were prepared in 9:1 mixture by a similar procedure described for Step 1 of Method A using tert-butyl 4-{2-[(methylsulfonyl)oxy]ethyl}-piperidine-1-carboxylate, adenine and K₂CO₃. The product was used for the next step without further purifications.

Step 2: tert-Butyl 4-[2-(6-amino-8-bromo-9H-purin-9-yl)ethyl]piperidine-1-carboxylate and tert-butyl 4-[2-(6-amino-8-bromo-3H-purin-3-yl)ethyl]piperidine-1-carboxylate:

A 9:1 mixture of tert-butyl 4-[2-(6-amino-9H-purin-9-yl)ethyl]piperidine-1-carboxylate and tert-butyl 4-[2-(6-amino-3H-purin-3-yl)ethyl]piperidine-1-carboxylate (55.49 mmol, 19.2 g) was dissolved in NaOAc-AcOH buffer (pH=4.6) and 1:1 MeOH-THF (60 mL) by stirring at room temperature. To the above clear solution was added bromine (99.8 mmol, 5.13 mL) drop wise while maintaining the temperature at ˜25° C. and stirring continued at rt for further 2 h. The reaction mixture was diluted with CHCl₃ (200 mL) and neutralize by adding NH₄OH and followed by 3 mL of hydrazine monohydrate to quench the excess bromine. The contents were taken in separatory funnel and organic layer was collected. The aqueous layer was extracted with CHCl₃ (150 mL). The combined organic layers were dried over Na₂SO₄ and evaporated to give crude product. The crude is dissolved in DCM and diluted with equal amounts of hexanes and left aside till solids separated out in the mixture. The solids were collected by filtration and washed with hexane. Filtrate was evaporated and the process was repeated until maximum product obtained (16 g, 67%). ¹H NMR (DMSO-d₆): δ 8.13 (s, 1H), 7.5-7.38 (broad s, 2H), 4.15 (t, J=7.4 Hz, 2H), 3.95-3.84 (m, 2H), 2.74-2.33 (m, 2H), 1.73 (d, J=12.8 Hz, 2H), 1.67 (q, J=6.6 Hz, 2H), 1.44-1.34 (m, 10H), 1.02 (dq, J=8.2, 3.5 Hz, 2H); LC-MS [M+H]⁺ 447.0.

Step 3. tert-Butyl 4-{2-[6-amino-8-(1,3-benzodioxol-5-ylthio)-9H-purin-9-yl]ethyl}piperidine-1-carboxylate: To a solution of tert-butyl 4-[2-(6-amino-8-bromo-9H-purin-9-yl)ethyl]piperidine-1-carboxylate (31.05 mmol, 13.2 g) in DMF (60 mL) at rt benzo[1,3]dioxole-5-thiol (46.58 mmol, 7.17 g) and K₂CO₃ (93.17 mmol, 12.87 g) were added. The reaction mixture was stirred at 100° C. for 3-6 h. At the end of this period reaction mixture was cooled to room temperature, filtered and the filtrate was diluted with EtOAc (120 mL). The EtOAc was washed with water (2×100 mL), brine (100 mL). The organic layer was dried over Na₂SO₄, filtered and the solvent volume was reduced to 30 mL by evaporation and diluted with 50 mL hexane, a solid was separated in the mixture that was collected by filtration and washed with 1:2 EtOAc-hexanes to afford the example product (14.5 g, 94%). ¹H NMR (CDCl₃): δ 8.3 (s, 1H), 7.02 (dd, J=7.8, 1.9 Hz, 1H), 6.96 (d, J=1.9 Hz, 1H), 6.8 (d, J=7.8 Hz, 1H), 5.99 (s, 2H), 5.59 (s, 2H), 4.23 (t, J=7.4 Hz, 2H), 4.38-3.98 (m, 2H), 2.3-2.15 (m, 2H), 1.82-1.66 (m, 4H), 1.49-1.35 (m, 10H), 1.22-1.1 (m, 2H); LC-MS [M+H]⁺ 499.1.

Step 4. 8-[(6-Bromo-1,3-benzodioxol-5-yl)thio]-9-(2-piperidin-4-ylethyl)-9H-purin-6-amine: To the clear solution of tert-butyl 4-{2-[6-amino-8-(1,3-benzodioxol-5-ylthio)-9H-purin-9-yl]ethyl}piperidine-1-carboxylate (29.11 mmol, 14.5 g) in AcOH (100 mL), bromine (64.05 mmol, 3.29 mL) was added slowly and the reaction was allowed to stir at room temperature. The complete bromination occurred in 1 h followed by a partial boc-group deprotection. To the reaction mixture 5 mL of water was added to quench the excess bromine and contents were evaporated under vacuum at 70° C. The crude was treated with DCM-TFA (9:1) and stirred for 1 hr. After completion of the reaction, the contents were evaporated under vacuum the crude was dissolved in water and neutralized with NH₄OH solution. The solid separated was collected by filtration, washed with water and dried under vacuum (13 g, 87%). ¹H NMR (DMSO-d₆): δ 8.16 (s, 1H), 7.52-7.43 (broad s, 2H), 7.39 (s, 1H), 6.81 (s, 1H), 6.09 (s, 2H), 4.18 (t, J=7.0 Hz, 2H), 3.23 (d, J=11.2 Hz, 2H), 3.16 (s, 1H), 2.83-2.7 (m, 2H), 1.86 (d, J=12.5 Hz, 2H), 1.64 (q, J=7.0 Hz, 2H), 1.5-1.39 (m, 1H), 1.32-1.2 (m, 2H); LC-MS [M+H]⁺ 476.9.

### Example Compound 148

### 4-(2-{6-Amino-8-[(6-bromo-1,3-benzodioxol-5-yl)thio]-9H-purin-9-yl}ethyl)piperidine-1-sulfonamide

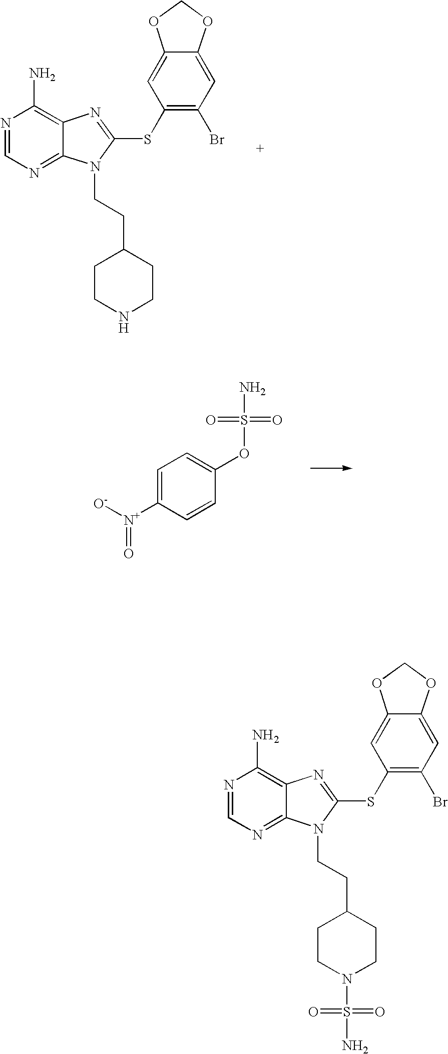

A mixture of 4-nitrophenyl sulfamate (0.206 g, 0.43 mmol), 8-[(6-bromo-1,3-benzodioxol-5-yl)thio]-9-(2-piperidin-4-ylethyl)-9H-purin-6-amine (0.180 g, 0.82 mmol) and triethylamine (0.16 mL, 1.15 mmol) in acetonitrile (4.0 mL) was heated to 37° C. for 65 hours. At the end of this period solvent was evaporated and the crude mixture was purified by preparative HPLC to yield the target compound; ¹H NMR (DMSO-d₆) δ 8.17 (s, 1H), 7.39 (s, 1H), 6.80 (s, 1H), 6.09 (s, 2H), 4.22-4.16 (m, 2H), 2.60-2.30 (m, 2H), 1.88-0.80 (m, 9H); LC-MS/TOF [M+H] 556.04.

### Example Compound 149

### 2-[4-(2-{6-Amino-8-[(6-bromo-1,3-benzodioxol-5-yl)thio]-9H-purin-9-yl}ethyl)piperidin-1-yl]ethanol

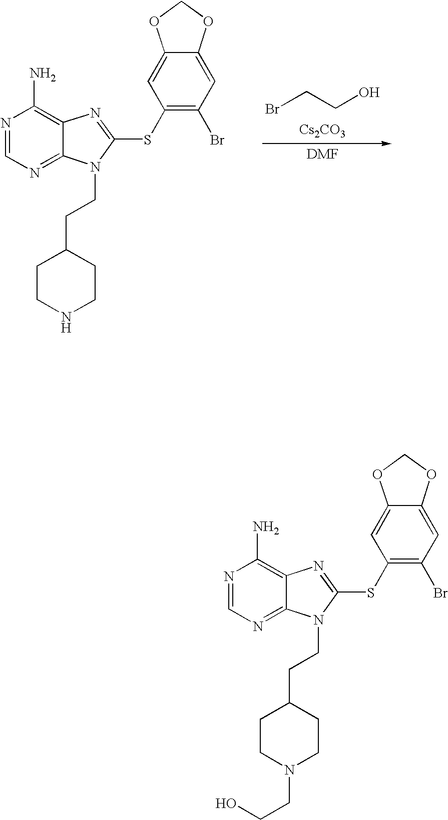

To a mixture of 8-[(6-bromo-1,3-benzodioxol-5-yl)thio]-9-(2-piperidin-4-ylethyl)-9H-purin-6-amine (70 mg, 0.15 mmol) and Cs₂CO₃ (41 mg, 0.29 mmol) in a mixture of THF (0.5 mL) and DMF (0.5 mL) was added 2-bromo-1-ethanol (21 μL, 0.29 mmol). After stirring for 10 h at rt, the mixture was diluted with CH₂Cl₂, filtered, and concentrated in vacuum. The residue was purified by prep HPLC to provide the target compound; ¹H NMR (DMSO-d₆) δ 8.28 (s, 1H), 7.29 (s, 1H), 7.18 (s, 1H), 6.09 (s, 2H), 4.37 (t, J=7.2 Hz, 2H), 3.87-3.85 (m, 2H), 3.64 (br d, J=12.8 Hz, 2H), 3.21-3.19 (m, 2H), 2.96 (br t, J=11.2 Hz, 2H), 2.14 (m, 2H), 1.87 (m, 2H), 1.65-1.53 (m, 3H); LC-MS [M+H]⁺ 521.10

### Example Compound 150

### 3-[4-(2-{6-Amino-8-[(6-bromo-1,3-benzodioxol-5-yl)thio]-9H-purin-9-yl}ethyl)piperidin-1-yl]propan-1-ol

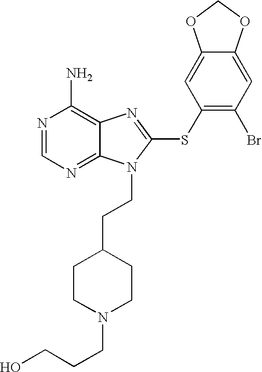

The example compound was prepared by a similar procedure described for example compound 149, using of 8-[(6-bromo-1,3-benzodioxol-5-yl)thio]-9-(2-piperidin-4-ylethyl)-9H-purin-6-amine and 3-bromo-propan-1-ol. ¹H NMR (DMSO-d₆) δ 8.27 (s, 1H), 7.28 (s, 1H), 7.18 (s, 1H), 6.09 (s, 2H), 4.37 (t, J=7.2 Hz, 2H), 3.69-3.66 (m, 2H), 3.62 (br d, J=10.8 Hz, 2H), 3.25-3.17 (m, 2H), 2.91 (br t, J=13.2 Hz, 2H), 2.16 (br d, J=13.2 Hz, 2H), 1.96-1.90 (m, 2H), 1.89-1.83 (m, 2H), 1.63 (m, 1H), 1.56-1.45 (m, 2H); LC-MS [M+H]⁺ 535.11.

### Example Compound 151

### (3S)-3-Amino-4-[4-(2-{6-amino-8-[(7-bromo-2,3-dihydro-1,4-benzodioxin-6-yl)thio]-9H-purin-9-yl}ethyl)piperidin-1-yl]-4-oxobutanamide

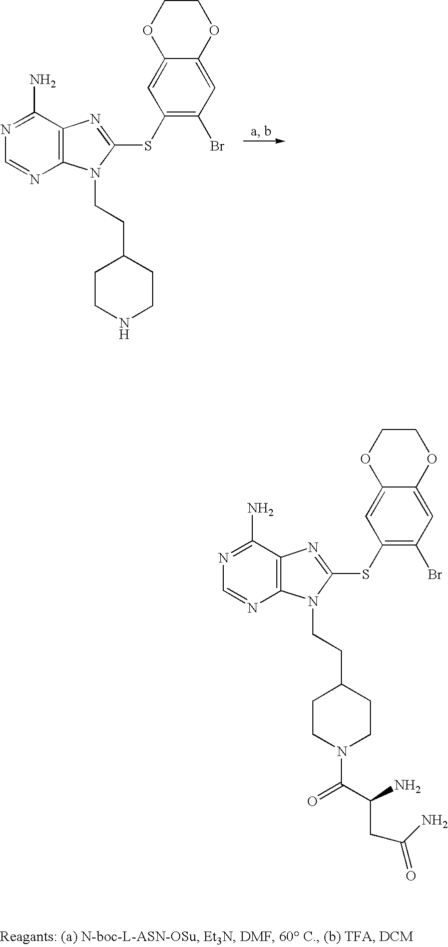

To a solution of 8-[(7-bromo-2,3-dihydro-1,4-benzodioxin-6-yl)thio]-9-(2-piperidin-4-ylethyl)-9H-purin-6-amine (0.3 g, 0.61 mmol) and Et₃N (187 μL, 1.34 mmol) in DMF (3 mL) was added (S)-2-tert-butoxycarbonylamino-succinamic acid 2,5-dioxo-pyrrolidin-1-yl ester (0.221 g, 0.67 mmol) at rt and the mixture was stirred at 60° C. for 10 h. The reaction mixture was evaporated under reduced pressure to afford Boc-protected product; LC-MS [M+H]⁺ 705.2. The product was used for the next step without further purification. The crude was dissolved in DCM (5 mL) and added TFA (1 mL) at room temperature and stirred the reaction mixture for 12 h. The reaction mixture was concentrated under vacuum and purified by preparative HPLC to afford example product (0.195 g); TOF LC-MS [M+H]⁺ 605.1.

### Example Compound 152

### (3S)-3-Amino-4-[4-(2-{6-amino-8-[(6-bromo-1,3-benzodioxol-5-yl)thio]-9H-purin-9-yl}ethyl)piperidin-1-yl]-4-oxobutanamide

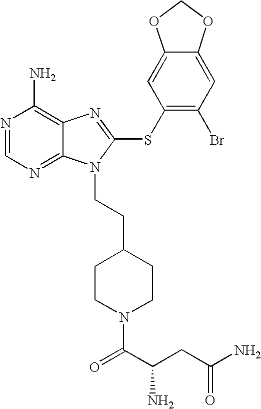

The example compound was prepared by a procedure similar to that described for example compound 151 using 8-[(6-bromo-1,3-benzodioxol-5-yl)thio]-9-(2-piperidin-4-ylethyl)-9H-purin-6-amine and (S)-2-tert-butoxycarbonylamino-succinamic acid 2,5-dioxo-pyrrolidin-1-yl ester. TOF LC-MS [M+H]⁺ 591.11

### Example Compounds 153 and 154

### 4-[4-(2-{6-Amino-8-[(7-bromo-2,3-dihydro-1,4-benzodioxin-6-yl)thio]-9H-purin-9-yl}ethyl)piperidin-1-yl]-4-oxobutanamide and 5-[4-(2-{6-amino-8-[(7-bromo-2,3-dihydro-1,4-benzodioxin-6-yl)thio]-9H-purin-9-yl}ethyl)piperidin-1-yl]-3,4-dihydro-2H-pyrrol-2-one

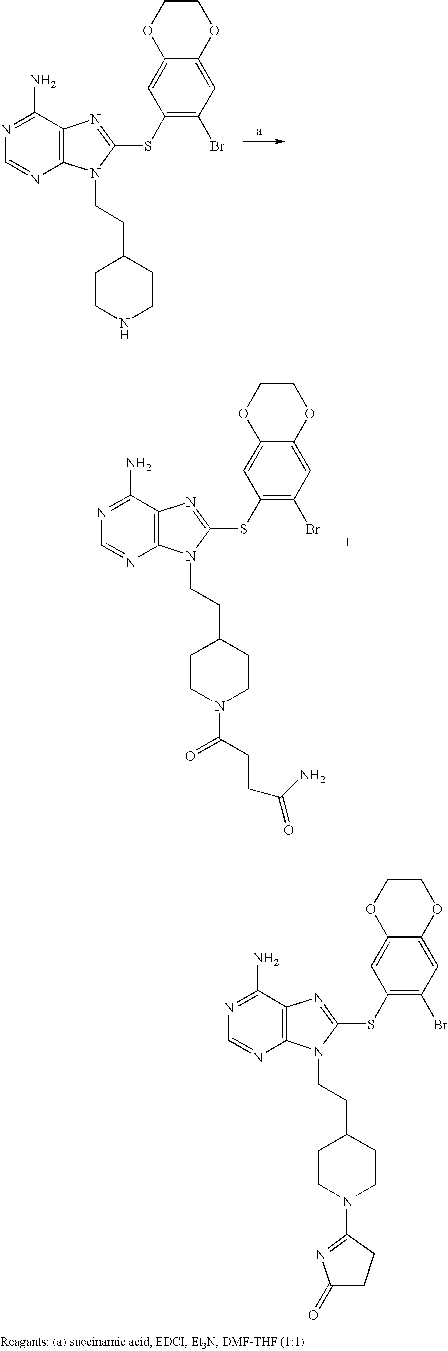

To a mixture of triethylamine (420 μL, 3.06 mmol), and EDCI (0.293 g, 1.53 mmol) in DMF-THF (7 mL) was added succinamic acid (0.179 g, 1.53 mmol), then followed by the addition of 8-[(7-bromo-2,3-dihydro-1,4-benzodioxin-6-yl)thio]-9-(2-piperidin-4-ylethyl)-9H-purin-6-amine (0.5 g, 1.02 mmol). The reaction mixture was allowed to stir at room temperature for 12 h. The reaction mixture was evaporated under reduced pressure, and the crude was purified by Isco silica gel flash column using DCM-MeOH (9:1) to obtain 4-[4-(2-{6-amino-8-[(7-bromo-2,3-dihydro-1,4-benzodioxin-6-yl)thio]-9H-purin-9-yl}ethyl)piperidin-1-yl]-4-oxobutanamide (0.11 g); TOF-MS [M+H]⁺ 590.1 and 5-[4-(2-{6-amino-8-[(7-bromo-2,3-dihydro-1,4-benzodioxin-6-yl)thio]-9H-purin-9-yl}ethyl)piperidin-1-yl]-3,4-dihydro-2H-pyrrol-2-one (0.078 g); TOF LC-MS [M+H]⁺ 572.1

### Example Compounds 155 and 156

### 4-[4-(2-{6-Amino-8-[(6-bromo-1,3-benzodioxol-5-yl)thio]-9H-purin-9-yl}ethyl)piperidin-1-yl]-4-oxobutanamide and 5-[4-(2-{6-amino-8-[(6-bromo-1,3-benzodioxol-5-yl)thio]-9H-purin-9-yl}ethyl)piperidin-1-yl]-3,4-dihydro-2H-pyrrol-2-one

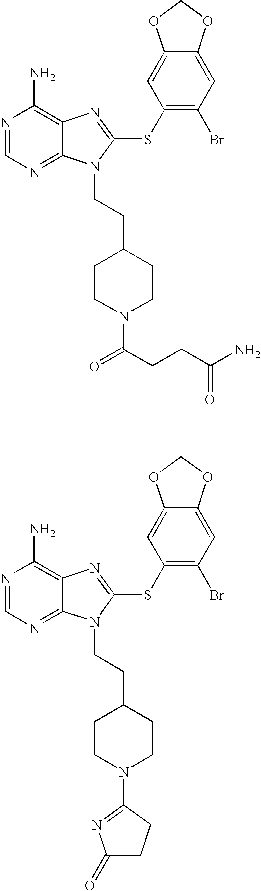

The example compounds were prepared by a procedure similar to that described for example compounds 153 and 154, using 8-[(6-bromo-1,3-benzodioxol-5-yl)thio]-9-(2-piperidin-4-ylethyl)-9H-purin-6-amine and succinamic acid. 4-[4-(2-{6-Amino-8-[(6-bromo-1,3-benzodioxol-5-yl)thio]-9H-purin-9-yl}ethyl)piperidin-1-yl]-4-oxobutanamide; ¹H NMR (DMSO-d₆) δ 8.16 (s, 1H), 7.48-7.4 (broad s, 2H), 7.39 (s, 1H), 7.3-7.25 (broad s, 1H), 6.81 (s, 1H), 6.74-6.69 (broad s, 1H), 6.09 (s, 2H), 4.3 (d, J=12.5 Hz, 1H), 4.1 (t, J=7.4 Hz, 2H), 3.82 (t, J=12.1 Hz, 1H), 2.86 (t, J=12.1 Hz, 1H), 2.5-2.35 (m, 3H), 2.26 (t, J=7.0 Hz, 2H), 1.69 (t, J=17.1 Hz, 2H), 1.59 (q, J=6.6 Hz, 2H), 1.46-1.4 (m, 1H), 1.12-0.85 (m, 2H); TOF-MS [M+H]⁺ 576.1. 5-[4-(2-{6-Amino-8-[(6-bromo-1,3-benzodioxol-5-yl)thio]-9H-purin-9-yl}ethyl)piperidin-1-yl]-3,4-dihydro-2H-pyrrol-2-one; ¹H NMR (DMSO-d₆) δ 8.16 (s, 1H), 7.48-7.4 (broad s, 2H), 7.38 (d, J=0.7 Hz, 1H), 6.81 (d, J=0.7 Hz, 1H), 6.08 (s, 2H), 4.31 (d, J=12.5 Hz, 1H), 4.18 (t, J=7.4 Hz, 2H), 3.75 (d, J=11.7 Hz, 1H), 2.86 (t, J=11.7 Hz, 1H), 2.7-2.65 (m, 2H), 2.6-2.6 (m, 2H), 2.46 (t, J=10.9 Hz, 1H), 1.75-1.66 (m, 2H), 1.6 (q, J=7.4 Hz, 2H), 1.46-1.35 (m, 1H), 1.14-0.9 (m, 2H); TOF-MS [M+H]⁺ 558.09.

### Example Compound 157

### N-{2-[4-(2-{6-Amino-8-[(7-bromo-2,3-dihydro-1,4-benzodioxin-6-yl)thio]-9H-purin-9-yl}ethyl)piperidin-1-yl]-2-oxoethyl}methanesulfonamide

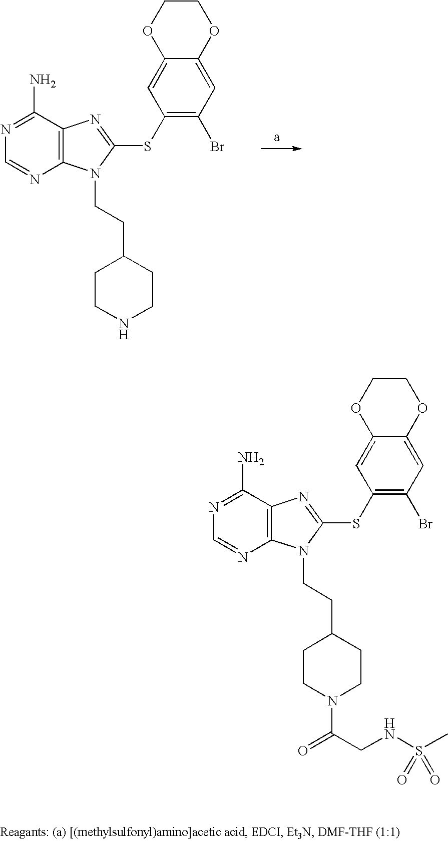

To a mixture of triethylamine (420 μL, 3.06 mmol), and EDCI (0.293 g, 1.53 mmol) in DMF-THF (7 mL) was added [(methylsulfonyl)amino]acetic acid (0.179 g, 1.53 mmol), followed by the addition of 8-[(7-bromo-2,3-dihydro-1,4-benzodioxin-6-yl)thio]-9-(2-piperidin-4-ylethyl)-9H-purin-6-amine (0.5 g, 1.02 mmol). The reaction mixture was allowed to stir at room temperature for 12 h. The reaction mixture was diluted with chloroform (60 mL) washed with saturated aq. NaHCO₃ (50 mL), brine (50 mL), organic layer was dried over Na₂SO₄, filtered and the solvent was evaporated to dryness. The crude was purified by Isco silica gel flash column using DCM-MeOH (9:1) to obtain example product (0.17 g). ¹H NMR (CDCl₃) δ 8.31 (d, J=2.7 Hz, 1H), 7.15 (d, J=3.1 Hz, 1H), 6.86 (d, J=3.5 Hz, 1H), 6.04-5.92 (brs, 2H), 5.74-5.66 (brs, 1H), 4.53 (d, J=12.8 Hz, 1H), 4.3-4.18 (m, 6H), 4.0-3.96 (brs, 2H), 3.63 (d, J=14.0 Hz, 1H), 3.04-2.93 (m, 4H), 2.59 (t, J=12.8 Hz, 1H), 1.95-1.81 (m, 2H), 1.79-1.7 (m, 2H), 1.58-1.46 (m, 1H), 1.23-1.1 (m, 2H); LC-MS [M+H]⁺ 626.1.

### Example Compound 158

### N-{2-[4-(2-{6-Amino-8-[(6-bromo-1,3-benzodioxol-5-yl)thio]-9H-purin-9-yl}ethyl)piperidin-1-yl]-2-oxoethyl}methanesulfonamide

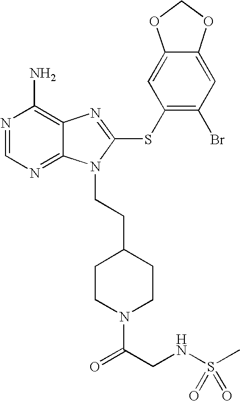

The example compound was prepared by a procedure similar to that described for example compound 157, using 8-[(6-bromo-1,3-benzodioxol-5-yl)thio]-9-(2-piperidin-4-ylethyl)-9H-purin-6-amine and [(methylsulfonyl)amino]acetic acid. ¹H NMR (CDCl₃) δ 8.32 (s, 1H), 7.08 (d, J=1.5 Hz, 1H), 6.8 (d, J=1.1 Hz, 1H), 6.12-6.06 (brs, 2H), 5.99 (s, 2H), 5.89-5.82 (brs, 1H), 4.53 (d, J=12.8 Hz, 1H), 4.25 (t, J=7.4 Hz, 2H), 4.0-3.95 (brs, 2H), 3.64 (d, J=12.5 Hz, 1H), 3.02-2.9 (m, 4H), 2.59 (t, J=12.8 Hz, 1H), 1.87 (t, J=16.4 Hz, 2H), 1.78-1.7 (m, 2H), 1.58-1.45 (m, 1H), 1.24-1.1 (m, 2H); LC-MS [M+H]⁺ 612.1.

### Example Compound 159

### N-{2-[4-(2-{6-Amino-8-[(7-bromo-2,3-dihydro-1,4-benzodioxin-6-yl)thio]-9H-purin-9-yl}ethyl)piperidin-1-yl]-2-oxoethyl}acetamide

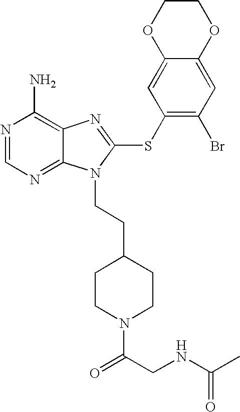

The example compound was prepared by a procedure similar to that described for example compound 157, using 8-[(7-bromo-2,3-dihydro-1,4-benzodioxin-6-yl)thio]-9-(2-piperidin-4-ylethyl)-9H-purin-6-amine and acetylamino-acetic acid. ¹H NMR (CDCl₃) δ 8.33 (s, 1H), 7.16 (s, 1H), 6.86 (s, 1H), 6.7-6.64 (broad s, 1H), 5.83-5.75 (broad s, 2H), 4.55 (d, J=13.2 Hz, 1H), 4.28-4.2 (m, 6H), 4.03 (dq, J=3.9, 17.1 Hz, 2H), 3.71 (d, J=13.2 Hz, 1H), 2.94 (t, J=12.8 Hz, 1H), 2.58 (t, J=12.5 Hz, 1H), 2.05 (s, 3H), 1.92-1.8 (m, 2H), 1.73 (q, J=7.0 Hz, 2H), 1.58-1.46 (m, 1H), 1.15 (dq, J=4.2, 12.8 Hz, 2H); LC-MS [M+H]⁺ 590.1.

### Example Compound 160

### N-{(1S)-2-[4-(2-{6-Amino-8-[(7-bromo-2,3-dihydro-1,4-benzodioxin-6-yl)thio]-9H-purin-9-yl}ethyl)piperidin-1-yl]-1-methyl-2-oxoethyl}acetamide

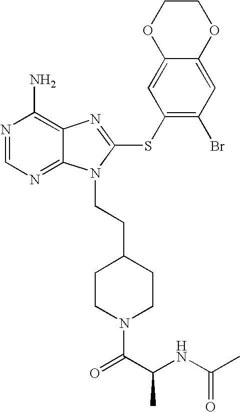

The example compound was prepared by a procedure similar to that described for example 157 using 8-[(7-bromo-2,3-dihydro-1,4-benzodioxin-6-yl)thio]-9-(2-piperidin-4-ylethyl)-9H-purin-6-amine and (S)-2-acetylamino-propionic acid. ¹H NMR (CDCl₃): δ 8.31 (s, 1H), 7.15 (d, J=1.9 Hz, 1H), 6.85 (d, J=1.1 Hz, 1H), 6.78-6.72 (m, 1H), 5.96-5.86 (brs, 2H), 4.87 (sextet, J=7.0 Hz, 1H), 4.55 (t, J=12.5 Hz, 1H), 4.3-4.16 (m, 6H), 3.92-3.83 (m, 1H), 3.04-2.93 (m, 1H), 2.55 (t, J=12.8 Hz, 1H), 2.0 (d, J=4.6 Hz, 3H), 1.94-1.8 (m, 2H), 1.78-1.68 (m, 2H), 1.58-1.46 (m, 1H), 1.3 (dd, J=6.6, 12.1 Hz, 3H), 1.22-1.09 (m, 2H); LC-MS [M+H]⁺ 604.1.

### Example Compound 161

### N-{(1R)-2-[4-(2-{6-Amino-8-[(7-bromo-2,3-dihydro-1,4-benzodioxin-6-yl)thio]-9H-purin-9-yl}ethyl)piperidin-1-yl]-1-methyl-2-oxoethyl}acetamide

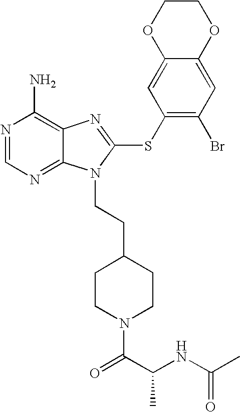

The example compound was prepared by a procedure similar to that described for example compound 157 using 8-[(7-bromo-2,3-dihydro-1,4-benzodioxin-6-yl)thio]-9-(2-piperidin-4-ylethyl)-9H-purin-6-amine and (R)-2-acetylamino-propionic acid. ¹H NMR (CDCl₃): δ 8.31 (s, 1H), 7.15 (d, J=1.9 Hz, 1H), 6.85 (d, J=1.1 Hz, 1H), 6.78-6.72 (m, 1H), 5.96-5.86 (brs, 2H), 4.87 (sextet, J=7.0 Hz, 1H), 4.55 (t, J=12.5 Hz, 1H), 4.3-4.16 (m, 6H), 3.92-3.83 (m, 1H), 3.04-2.93 (m, 1H), 2.55 (t, J=12.8 Hz, 1H), 2.0 (d, J=4.6 Hz, 3H), 1.94-1.8 (m, 2H), 1.78-1.68 (m, 2H), 1.58-1.46 (m, 1H), 1.3 (dd, J=6.6, 12.1 Hz, 3H), 1.22-1.09 (m, 2H); LC-MS [M+H]⁺ 604.1.

### Example Compound 162

### 2-[4-(2-{6-Amino-8-[(7-bromo-2,3-dihydro-1,4-benzodioxin-6-yl)thio]-9H-purin-9-yl}ethyl)piperidin-1-yl]-2-oxoethanethiol

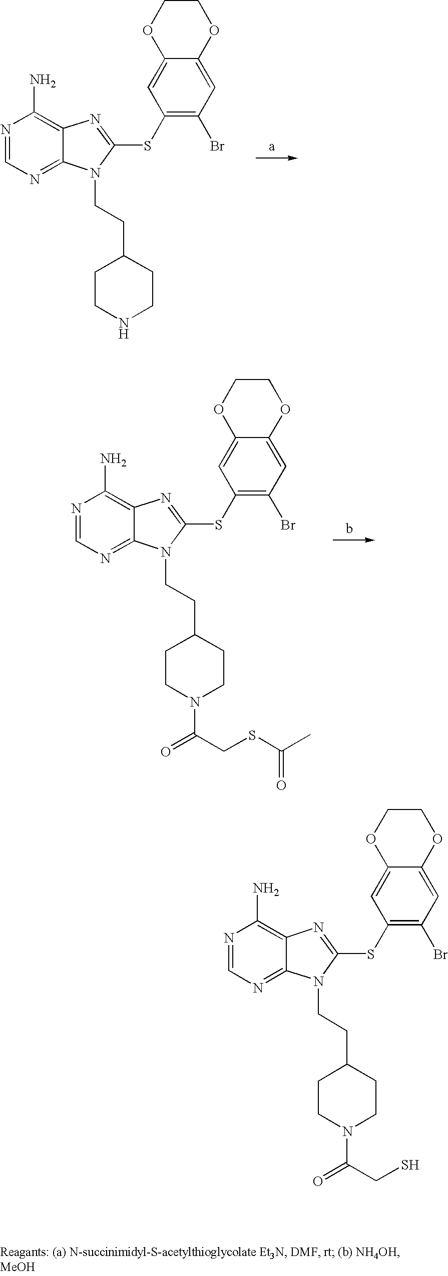

A mixture of 8-[(7-bromo-2,3-dihydro-1,4-benzodioxin-6-yl)thio]-9-(2-piperidin-4-ylethyl)-9H-purin-6-amine (0.40 mmol, 0.2 g), N-succinimidyl-S-acetylthioglycolate (0.48 mmol, 0.113 g), and triethylamine (0.81 mmol, 113 μL) in DMF (2 mL) was stirred at room temperature for 12 h. The reaction mixture was evaporated under reduced pressure and the crude was dissolved in MeOH (5 mL) and added NH₄OH solution (3 mL) and stirred at room temperature for 3 h. At the end of this period the reaction mixture was evaporated under reduced pressure and the crude was purified by preparative HPLC [X-Terra prep-RP18 10 um, 19×250 mm (waters), Mobile phase: solvent A: Water HPLC grade containing 0.01% TFA, and solvent B: acetonitrile containing 0.01% TFA, general eluting gradient—solvent B 15% to 80 over 15 to 25 minutes run time]. After lyophilization of HPLC fractions the example compound was isolated as trifluoro acetate salt. ¹H NMR (CD₃OD) δ 8.24 (d, J=1.1 Hz, 1H), 7.22 (d, J=3.5 Hz, 1H), 7.16 (d, J=3.1 Hz, 1H), 4.47 (d, J=12.5 Hz, 1H), 4.36-4.24 (m, 6H), 3.97 (d, J=11.3 Hz, 1H), 3.82 (dd, J=5.4, 13.6 Hz, 1H), 3.72 (dd, J=5.0, 13.6 Hz, 1H), 3.15-3.06 (m, 1H), 2.69-2.6 (m, 1H), 1.94-1.76 (m, 4H), 1.69-1.55 (m, 1H), 1.38-1.12 (m, 2H); TOF-MS [M+H]⁺ 565.06.

### Example Compounds 163 and 164

### (2S)-1-[4-(2-{6-amino-8-[(3-bromo-5,6,7,8-tetrahydronaphthalen-2-yl)thio]-9H-purin-9-yl}ethyl)piperidin-1-yl]-1-oxopropan-2-ol and (2S)-1-[4-(2-{6-amino-8-[(3-bromo-5,6,7,8-tetrahydronaphthalen-2-yl)thio]-3H-purin-3-yl}ethyl)piperidin-1-yl]-1-oxopropan-2-ol

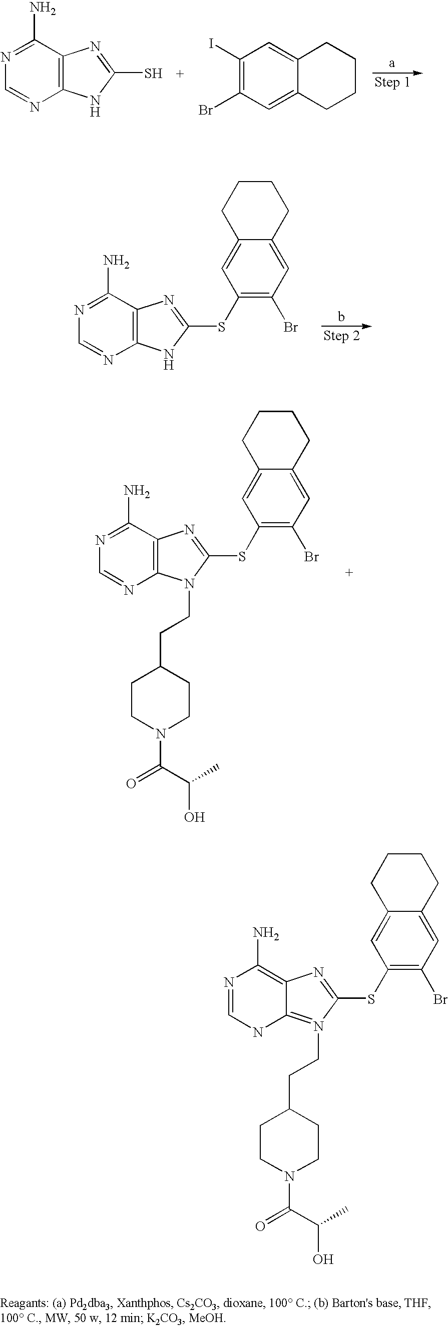

Step 1. 8-(3-Bromo-5,6,7,8-tetrahydro-naphthalen-2-ylsulfanyl)-9H-purin-6-ylamine: The example compounds were prepared using palladium-catalyzed coupling reaction of 8-mercaptoadenine with 6-bromo-7-iodo-1,2,3,4-tetrahydronaphthalene (J. Am. Chem. Soc. 1977, 99, 4058) as described for intermediate 14; LC-MS [M+H]⁺ 376.2.

Step 2. (2S)-1-[4-(2-{6-amino-8-[(3-bromo-5,6,7,8-tetrahydronaphthalen-2-yl)thio]-9H-purin-9-yl}ethyl)piperidin-1-yl]-1-oxopropan-2-ol and (2S)-1-[4-(2-{6-amino-8-[(3-bromo-5,6,7,8-tetrahydronaphthalen-2-yl)thio]-3H-purin-3-yl}ethyl)piperidin-1-yl]-1-oxopropan-2-ol: Alkylation of the 8-(3-bromo-5,6,7,8-tetrahydro-naphthalen-2-ylsulfanyl)-9H-purin-6-ylamine from step 1 with acetic acid (S)-1-methyl-2-oxo-2-{4-[2-(toluene-4-sulfonyloxy)-ethyl]-piperidin-1-yl}-ethyl ester and subsequent deprotection of the acetate group according to the procedure described for example compounds 88 and 89 to provide the target compounds. (25)-1-[4-(2-{6-amino-8-[(3-bromo-5,6,7,8-tetrahydronaphthalen-2-yl)thio]-9H-purin-9-yl}ethyl)piperidin-1-yl]-1-oxopropan-2-ol; ¹H NMR (CD₃OD) δ 8.27 (s, 1H), 7.48 (s, 1H), 7.32 (s, 1H), 4.5-4.3 (m, 4H), 4.0-3.9 (m, 1H), 3.2-2.6 (m, 5H), 1.9-1.7 (m, 8H), 1.3-1.1 (m, 5H); LC-MS [M+H]⁺ 559.15. (2S)-1-[4-(2-{6-amino-8-[(3-bromo-5,6,7,8-tetrahydronaphthalen-2-yl)thio]-3H-purin-3-yl}ethyl)piperidin-1-yl]-1-oxopropan-2-ol; ¹H NMR (CD₃OD) δ 8.49 (s, 1H), 7.57 (s, 1H), 7.54 (s, 1H), 4.6-4.35 (m, 4H), 4.0-3.9 (m, 1H), 2.9-2.6 (m, 5H), 1.9-1.7 (m, 8H), 1.3-1.1 (m, 5H); LC-MS [M+H]⁺ 559.15.

### Example Compounds 165 and 166

### (2S)-1-[4-(2-{6-amino-8-[(2-bromo-4,5-dimethylphenyl)thio]-9H-purin-9-yl}ethyl)piperidin-1-yl]-1-oxopropan-2-ol and (2S)-1-[4-(2-{6-amino-8-[(2-bromo-4,5-dimethylphenyl)thio]-3H-purin-3-yl}ethyl)piperidin-1-yl]1-oxopropan-2-ol

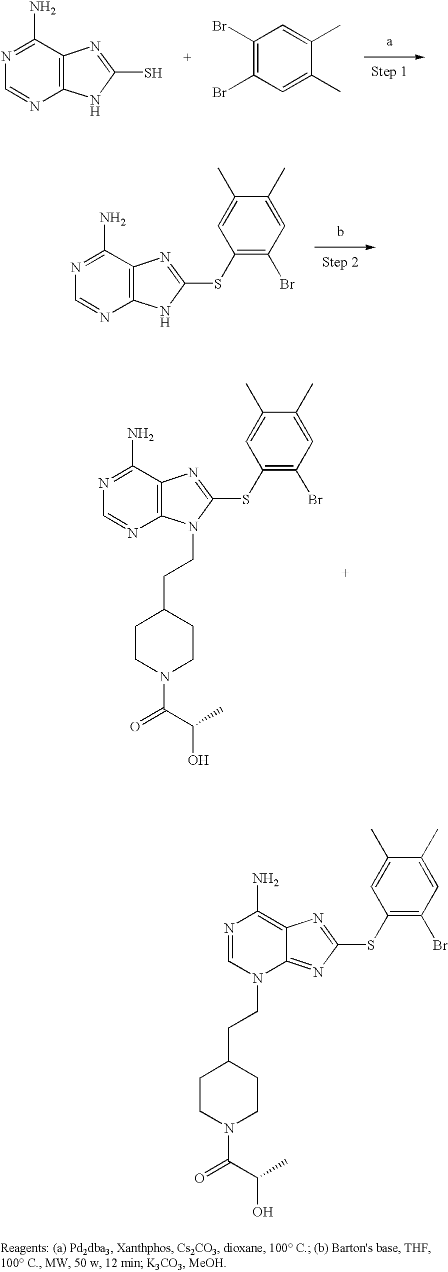

Step 1. Synthesis of 8-(2-bromo-4,5-dimethyl-phenylsulfanyl)-9H-purin-6-ylamine: The example compounds were prepared using palladium-catalyzed coupling reaction of 8-mercaptoadenine with 4,5-dibromo-o-xylene as described for intermediate 14; LC-MS [M+H]⁺ 350.

Step 2. (2S)-1-[4-(2-{6-amino-8-[(2-bromo-4,5-dimethylphenyl)thio]-9H-purin-9-yl}ethyl)piperidin-1-yl]-1-oxopropan-2-ol and (2S)-1-[4-(2-{6-amino-8-[(2-bromo-4,5-dimethylphenyl)thio]-3H-purin-3-yl}ethyl)piperidin-1-yl]-1-oxopropan-2-ol: Alkylation of the 8-(2-bromo-4,5-dimethyl-phenylsulfanyl)-9H-purin-6-ylamine from step 1 with acetic acid (S)-1-methyl-2-oxo-2-{4-[2-(toluene-4-sulfonyloxy)-ethyl]-piperidin-1-yl}-ethyl ester and subsequent deprotection of acetate group according to the procedure described for example compounds 88 and 89 to provide the target compounds. (2S)-1-[4-(2-{6-amino-8-[(2-bromo-4,5-dimethylphenyl)thio]-9H-purin-9-yl}ethyl)piperidin-1-yl]-1-oxopropan-2-ol; ¹H NMR (DMSO-d₆) δ 8.21 (s, 1H), 7.54 (s, 1H), 6.95 (s, 1H), 4.4-4.1 (m, 4H), 3.95-3.85 (m, 1H), 2.9-2.8 (m, 1H), 2.55-2.4 (m, 2H), 2.19 (s, 3H), 2.08 (s, 3H), 1.7-1.5 (m, 4H), 1.45-1.3 (m, 1H), 1.15-0.8 (m, 4H); LC-MS [M+H]⁺ 533.13. (2S)-1-[4-(2-{6-amino-8-[(2-bromo-4,5-dimethylphenyl)thio]-3H-purin-3-yl}ethyl)piperidin-1-yl]-1-oxopropan-2-ol; ¹H NMR (DMSO-d₆) δ 8.65 (s, 1H), 7.66 (s, 1H), 7.59 (s, 1H), 4.5-4.2 (m, 4H), 3.95-3.85 (m, 1H), 2.95-2.85 (m, 1H), 2.55-2.4 (m, 2H), 2.19 (s, 3H), 2.08 (s, 3H), 1.7-1.5 (m, 4H), 1.45-1.3 (m, 1H), 1.15-0.8 (m, 4H); LC-MS [M+H]⁺ 533.13.

### Intermediate 57

### 2-Ethylhexyl 3-[(5-bromo-2,3-dihydro-1-benzofuran-6-yl)thio]propanoate

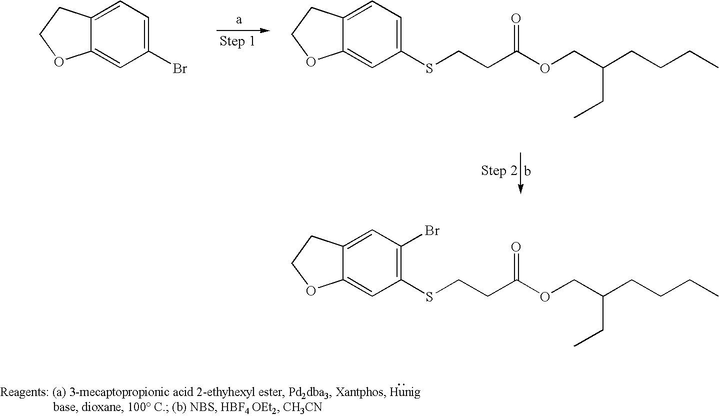

2-Ethylhexyl 3-[(5-bromo-2,3-dihydro-1-benzofuran-6-yl)thio]propanoate was prepared in two-step reaction sequence starting from 6-bromodihydrobenzofuran.

Step 1: Synthesis of 2-ethylhexyl 3-(2,3-dihydro-1-benzofuran-6-ylthio)propanoate: 6-Bromodihydrobenzofuran (2.39 g, 12.02 mmol), which was prepared according to the literature procedure (Org. Lett. 2001, 3, 3351), 3-mercaptopropionic acid 2-ethyhexyl ester (2.76 g, 12.6 mmol), Pd2 dba3 (165 mg, 0.180 mmol) and Xantphos (279 mg, 0.481 mmol) were placed in a flask. A degassed dioxane (40 mL) and Hünig base (4.2 mL, 0.18 mol) were then added under nitrogen and the mixture was heated at 100° C. After 10 h, the reaction mixture was cooled to rt, diluted with CH₂Cl₂, filtered, and concentrated in vacuo. The residue was purified by SiO₂ chromatograph (EtOAc/hexane, 0 to 30%) to afford 1.3 g (33%) of 2-ethylhexyl 3-(2,3-dihydro-1-benzofuran-6-ylthio)propanoate LC-MS [M+Na]+ 359.3.

Step 2: Synthesis of 2-ethylhexyl 3-[(5-bromo-2,3-dihydro-1-benzofuran-6-yl)thio]propanoate: To a solution of 2-ethylhexyl 3-(2,3-dihydro-1-benzofuran-6-ylthio)propanoate (500 mg, 1.49 mmol) in CH3CN (3 mL) was treated with HBF4 OEt2 (204 μL, 1.50 mmol) at −20° C., followed by NBS (267 mg, 1.50 mmol) in portionwise. The reaction mixture was slowly warmed up to 5-10° C. and quenched with 10% aq. NaHSO3 (7 mL) and extracted with Et2O. The combined extracts were washed with H2O, brine, and the organic layer was dried over Na2SO4, filtered, and concentrated in vacuo. The residue was purified by SiO2 chromatograph (EtOAc/hexane, 0 to 30%) to afford 0.455 g (71%) of 2-ethylhexyl 3-[(5-bromo-2,3-dihydro-1-benzofuran-6-yl)thio]propanoate. ¹H NMR (CDCl₃) δ 7.35 (t, J=0.8 Hz, 1H), 6.76 (s, 1H), 4.59 (t, J=8.8 Hz, 2H), 4.04 (m, 2H), 3.19 (td, J=8.8, 0.8 Hz, 2H), 3.16 (t, J=8.0 Hz, 2H), 2.67 (t, J=7.2 Hz, 2H), 1.56 (m, 1H), 1.39-1.25 (m, 8H), 0.89 (two t, J=6.8 Hz, 6H).

### Intermediates 58-64

Intermediates 58-64 were synthesized in the similar manner as described for intermediate 57, above, using appropriate starting materials, and are summarized in table 7, below.

TABLE 7
Preparation of Intermediates 58-64

| Inter- | | |
|---|---|---|
| mediate | Structure | Name and analytical data |
| 58 | 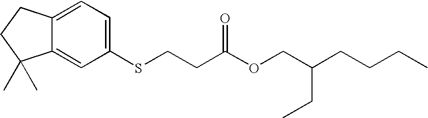 | 2-Ethylhexyl 3-[(3,3-dimethyl- 2,3-dihydro-1H-inden-5- yl)thio]propanoate GC-MS m/z = 362 |
| 59 | 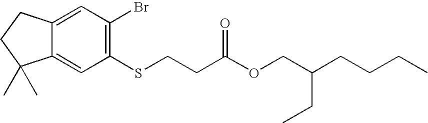 | 2-Ethylhexyl 3-[(6-bromo-3,3- dimethyl-2,3-dihydro-1H-inden-5- yl)thio]propanoate GC-MS m/z = 440 |
| 60 | 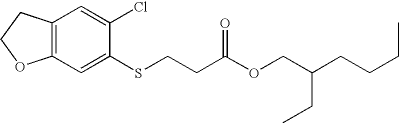 | 2-Ethylhexyl 3-[(5-chloro-2,3- dihydro-1-benzofuran-6- yl)thio]propanoate GC-MS m/z = 370 |
| 61 and 62 | 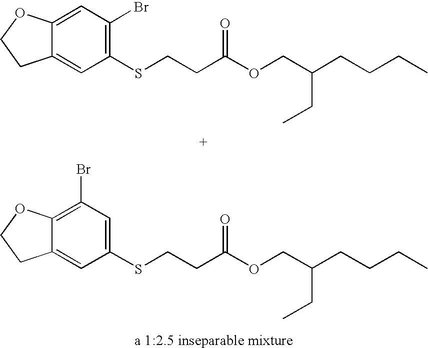 | 2-Ethylhexyl 3-[(6-bromo-2,3- dihydro-1-benzofuran-5- yl)thio]propanoate and 2- ethylhexyl 3-[(7-bromo-2,3- dihydro-1-benzofuran-5- yl)thio]propanoate GC/MS m/z = 416 |
| 63 | 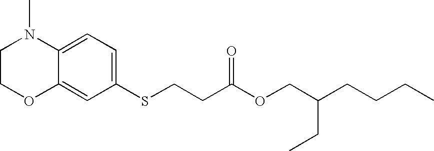 | 2-Ethylhexyl 3-[(4-methyl-3,4- dihydro-2H-1,4-benzoxazin-7- yl)thio]propanoate GC-MS m/z = 365 |
| 64 | 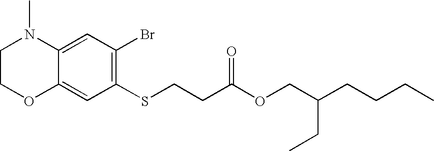 | 2-Ethylhexyl 3-[(6-bromo-4- methyl-3,4-dihydro-2H-1,4- benzoxazin-7-yl)thio]propanoate GC-MS m/z = 445 |

### Intermediate 65

### 2-Ethylhexyl 3-[(5-bromo-1-benzofuran-6-yl)thio]propanoate

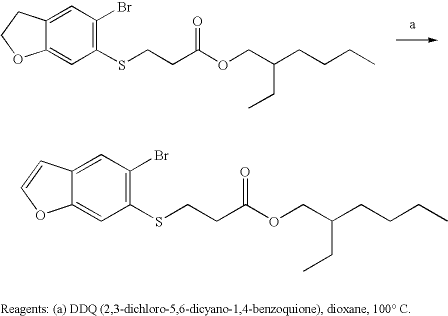

To a solution of 2-ethylhexyl 3-[(5-bromo-2,3-dihydro-1-benzofuran-6-yl)thio]propanoate (114 mg, 0.274 mmol) in dioxane (1.7 mL) was added DDQ (81 mg, 0.36 mmol), and the mixture was heated at 100° C. After 10 h, the mixture was cooled to rt, diluted with Et2O, filtered, and concentrated in vacuo. The residue was purified by SiO2 chromatograph (EtOAc/hexane, 0 to 10%) to afford the example compound (92 mg, 99%): ¹H NMR (CDCl₃) δ 7.83 (s, 1H), 7.60 (d, J=2.0 Hz, 1H), 7.36 (d, J=0.8 Hz, 1H), 6.70 (dd, J=2.0 Hz, 0.8 Hz, 1H), 4.02 (m, 2H), 3.23 (t, J=7.6 Hz, 2H), 2.69 (t, J=7.6 Hz, 2H), 1.59 (m, 1H), 1.42-1.25 (m, 8H), 0.89 (two t, J=7.6 Hz, 6H); GC-MS [M]+ 412.

### Intermediate 66

### 2-Ethylhexyl 3-[(5-chloro-1-benzofuran-6-yl)thio]propanoate

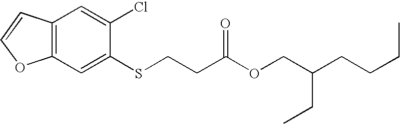

The example intermediate was prepared in the similar manner as described for intermediate 65 using 2-ethylhexyl 3-[(5-chloro-2,3-dihydro-1-benzofuran-6-yl)thio]propanoate. GC-MS m/z=370

### Intermediates 67 and 68

### 2-Ethylhexyl 3-[(6-bromo-1-benzofuran-5-yl)thio]propanoate and 2-ethylhexyl 3-[(7-bromo-1-benzofuran-5-yl)thio]propanoate

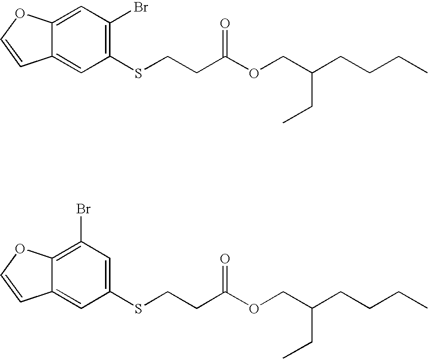

The example intermediates were prepared as an inseparable mixture by a similar manner as described for intermediate 65 using a mixture of 2-ethylhexyl 3-[(6-bromo-2,3-dihydro-1-benzofuran-5-yl)thio]propanoate and 2-ethylhexyl 3-[(7-bromo-2,3-dihydro-1-benzofuran-5-yl)thio]propanoate. ¹H NMR (CDCl₃) (the major isomer) δ 7.70 (d, J=2.4 Hz, 1H), 7.62 (d, J=2.0 Hz, 1H), 7.55 (d, J=2.0 Hz, 1H), 6.80 (d, J=2.4 Hz, 1H), 4.03-3.96 (m, 2H), 3.15 (t, J=7.6 Hz, 2H), 2.60 (t, J=7.2 Hz, 2H), 1.55 (m, 1H), 1.39-1.27 (m, 8H), 0.89 (two t, J=7.6 and 7.2 Hz, 6H).

### Intermediate 69

### Synthesis of 2-ethylhexyl 3-[(6-formyl-1,3-benzodioxol-5-yl)thio]propanoate

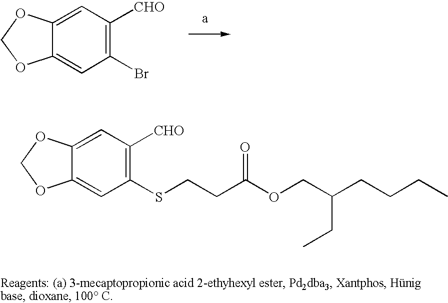

2-Ethylhexyl 3-[(6-formyl-1,3-benzodioxol-5-yl)thio]propanoate was prepared by a procedure similar described for step 1 of intermediate 57, using 6-bromo-benzo[1,3]dioxole-5-carbaldehyde and 3-mercaptopropionic acid 2-ethyhexyl ester. ¹H NMR (CDCl₃) δ10.42. s, 1H), 7.36 (s, 1H), 7.00 (s, 1H), 6.08 (s, 2H), 4.45-3.97 (m, 2H), 3.13 (t, J=7.2 Hz, 2H), 2.61 (t, J=7.6 Hz, 2H), 1.56 (m, 1H), 1.39-1.25 (m, 8H), 0.89 (m, 6H).

### Intermediates 70-77

Intermediates 70-77 were synthesized in the same manner as described for step 1 of intermediate 57, above, using appropriate starting materials, and are summarized in table 8, below.

TABLE 8
Preparation of Intermediates 70-77.

| Inter- | | |
|---|---|---|
| mediate | Structure | Name and analytical data |
| 70 | 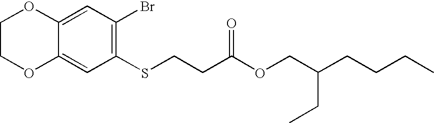 | 2-Ethylhexyl 3-[(7-bromo-2,3- dihydro-1,4-benzodioxin-6- yl)thio]propanoate ¹H NMR (CDCl₃) δ 7.12 (s, 1H), 6.97 (s, 1H), 4.25 (s, 4H), 4.06-3.98 (m, 2H), 3.10 (t, J = 7.6 Hz, 2H), 2.62 (t, J = 7.6 Hz, 2H), 1.56 (m, 1H), 1.39-1.26 (m, 8H), 0.89 (t, J = 7.6 Hz, 6H) |
| 71 | 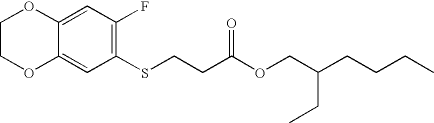 | [2-Ethylhexyl 3-[(7-fluoro-2,3- dihydro-1,4-benzodioxin-6- yl)thio]propanoate GC-MS m/z = 370 |
| 72 | 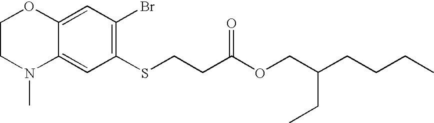 | 2-Ethylhexyl 3-[(7-bromo-4- methyl-3,4-dihydro-2H-1,4- benzoxazin-6-yl)thio]propanoate GC-MS m/z = 443 |
| 73 | 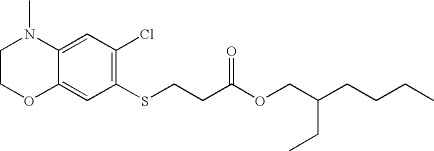 | 2-Ethylhexyl 3-[(6-chloro-4- methyl-3,4-dihydro-2H-1,4- benzoxazin-7-yl)thio]propanoate GC-MS m/z = 399 |
| 74 | 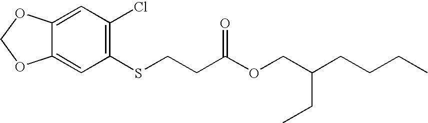 | 2-Ethylhexyl 3-[(6-chloro-1,3- benzodioxol-5-yl)thio]propanoate |
| 75 | 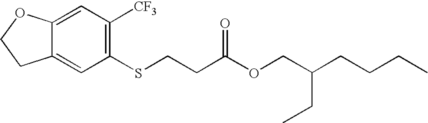 | 2-Ethylhexyl 3-{[6- (trifluoromethyl)-2,3-dihydro-1- benzofuran-5-yl]thio}propanoate GC-MS m/z = 404 |
| 76 | 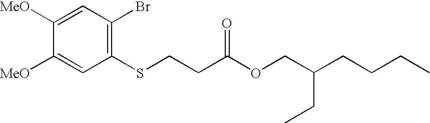 | 2-Ethylhexyl 3-[(2-bromo-4,5- dimethoxyphenyl)thio]propanoate GC-MS m/z 432 |
| 77 | 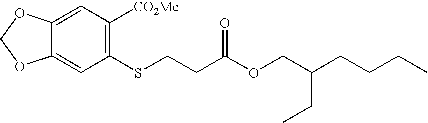 | Methyl 6-({3-[(2-ethylhexyl)oxy]- 3-oxopropyl}thio)-1,3-benzodioxole- 5-carboxylate. GC-MS [M]⁺ 396. |

### Intermediate 78

### 2-Ethylhexyl 3-({6-[(dimethylamino)carbonyl]-1,3-benzodioxol-5-yl}thio)propanoate

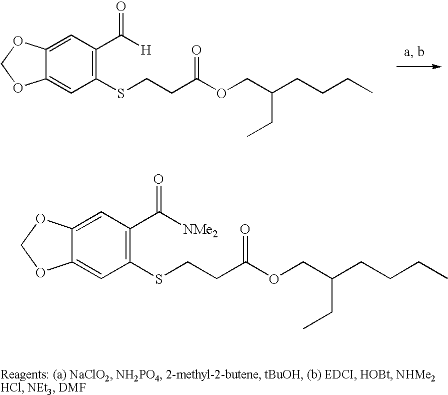

2-Ethylhexyl 3-({6-[(dimethylamino)carbonyl]-1,3-benzodioxol-5-yl}thio)propanoate was prepared from 2-ethylhexyl 3-[(6-formyl-1,3-benzodioxol-5-yl)thio]propanoate by standard two-step sequence of Lindgren-Pinnick oxidation and EDCI mediated amide coupling reaction; LC-MS [M+Na]⁺ 432.3

### Example Compound 167

### 4-(2-{6-Amino-8-[(5-bromo-2,3-dihydro-1-benzofuran-6-yl)thio]-9H-purin-9-yl} ethyl)piperidine-1-carbaldehyde

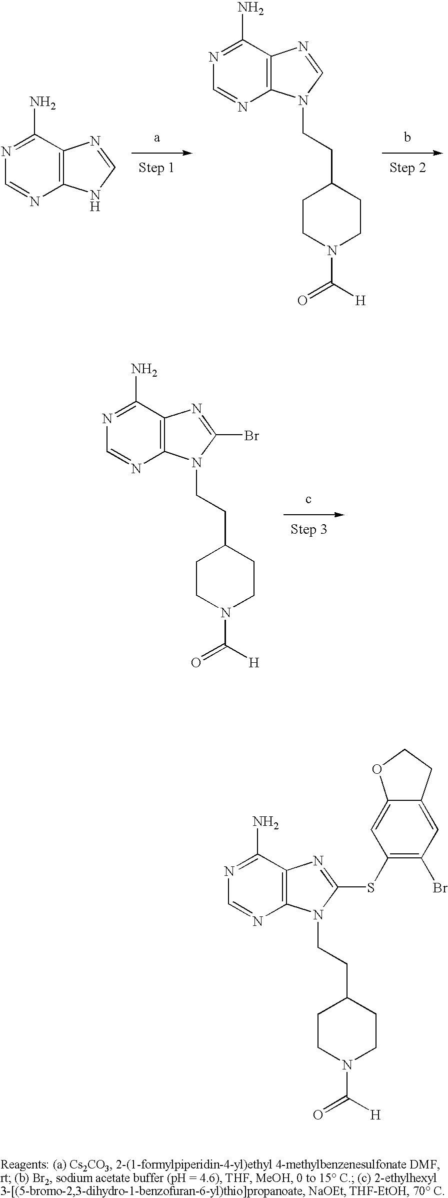

Step 1. 4-[2-(6-Amino-9H-purin-9-yl)ethyl]piperidine-1-carbaldehyde:

Title compound (2.6 g, 26%) was prepared by a procedure similar to that described for step 1 of example compound 147, using adenine (5.0 g, 37 mmol), 2-(1-formylpiperidin-4-yl)ethyl 4-methylbenzenesulfonate (9.59 g, 30.8 mmol) and Cs2CO3 (24.1 g, 74.0 mmol). ¹H NMR (DMSO-d6) δ 8.18 (s, 1H), 8.14 (s, 1H), 7.95 (s, 1H), 7.21 (s, 2H), 4.18 (t, J=7.2 Hz, 2H), 4.12 (br d, J=13.2 Hz, 1H), 3.63 (br d, J=13.2 Hz, 1H), 2.93 (td, J=12.4, 2.8 Hz, 1H), 2.56-2.48 (m, 3H), 1.80-1.72 (m, 4H), 1.43 (m, 1H), 1.04 (qd, J=12.0, 4.0 Hz, 1H), 0.96 (qd, J=12.4, 4.8 Hz, 1H); TOF LC-MS [M+H]+ 275.16.

Step 2. 4-[2-(6-Amino-8-bromo-9H-purin-9-yl)ethyl]piperidine-1-carbaldehyde:

To a solution of 4-[2-(6-amino-9H-purin-9-yl)ethyl]piperidine-1-carbaldehyde (2.08 g, 7.58 mmol) in a mixture of NaOAc buffer (pH=4.6), MeOH (15 mL), and THF (15 mL) was added bromine (1.17 ml, 22.8 mmol) dropwise at 0° C., and the reaction mixture was slowly warmed up to 10-15° C. over 1 h. The mixture was quenched with sodium metabisulfite and the pH of the reaction mixture was adjusted to ˜8 by addition of satd. Na2CO3 solution. The product portion was extracted with CH2Cl2, washed with H2O/brine, dried (Na2SO4), filtered, and concentrated in vacuo to afford the example compound (2.41 g, 99%): TOF LC-MS [M+Na]+ 296.13.

Step 3. 4-(2-{6-Amino-8-[(5-bromo-2,3-dihydro-1-benzofuran-6-yl)thio]-9H-purin-9-yl}ethyl)piperidine-1-carbaldehyde:

To a mixture of 4-[2-(6-amino-8-bromo-9H-purin-9-yl)ethyl]piperidine-1-carbaldehyde (100 mg, 0.283 mmol), 2-ethylhexyl 3-[(5-bromo-2,3-dihydro-1-benzofuran-6-yl)thio]propanoate (235 mg, 0.566 mmol) in THF (2.8 mL)/EtOH (0.28 mL) was added NaOEt (41 mg, 0.57 mmol), and the reaction mixture was heated at 70° C. After 10 h, the mixture was quenched with H₂O and the product portion was extracted with EtOAc. The combined organic layers were washed with H₂O and brine, dried (Na2SO4), filtered, and concentrated in vacuo. The residue was triturated with EtOAc to provide the example compound (87 mg, 61%). 4-(2-{6-Amino-8-[(5-bromo-2,3-dihydro-1-benzofuran-6-yl)thio]-9H-purin-9-yl}ethyl)piperidine-1-carbaldehyde. 1H NMR (DMSO-d6) δ 8.19 (s, 1H), 7.93 (s, 1H), 7.57 (s, 1H), 7.56-7.46 (br s, 2H), 6.33 (s, 1H), 4.52 (t, J=8.8 Hz, 2H), 4.17 (t, J=7.2 Hz, 2H), 4.07 (br d, J=13.2 Hz, 1H), 3.60 (br d, J=12.8 Hz, 1H), 3.17 (t, J=8.0 Hz, 2H), 2.86 (td, J=12.8, 2.8 Hz, 1H), 2.44 (td, J=12.4, 3.2 Hz, 1H), 1.76-1.64 (m, 2H), 1.59 (q, J=6.8 Hz, 2H), 1.39 (m, 1H), 0.97 (qd, J=12.8, 4.4 Hz, 1H), 0.87 (qd, J=12.4, 4.4 Hz, 1H); TOF LC-MS [M+H]+ 503.09.

### Example Compound 168

### 4-(2-{6-Amino-8-[(5-bromo-1-benzofuran-6-yl)thio]-9H-purin-9-yl}ethyl)piperidine-1-carbaldehyde

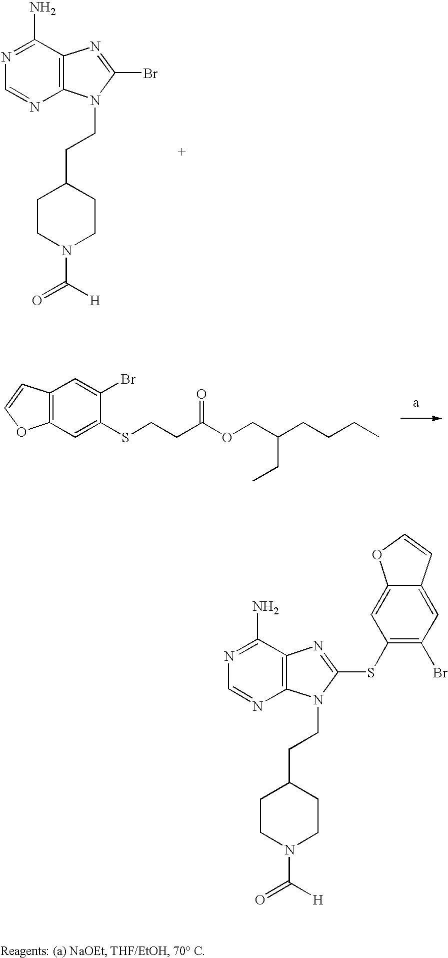

The example compound was prepared by a procedure similar to that described for step 3 of example compound 167, using 2-ethylhexyl 3-[(5-bromo-1-benzofuran-6-yl)thio]propanoate and 4-[2-(6-amino-8-bromo-9H-purin-9-yl)ethyl]piperidine-1-carbaldehyde. ¹H NMR (DMSO-d6) δ 8.17 (s, 1H), 8.07 (s, 1H), 8.05 (d, J=2.0 Hz, 1H), 7.89 (s, 1H), 7.48-7.43 (br s, 2H), 6.95 (dd, J=2.0, 0.8 Hz, 1H), 4.19 (t, J=6.8 Hz, 2H), 4.03 (br d, J=12.4, 1H), 3.55 (br d, J=13.5 1H), 2.81 (td, J=12.4, 2.8 Hz, 1H), 2.39 (td, J=12.4, 2.8 Hz, 1H), 1.67-1.55 (m, 4H), 1.37 (m, 1H), 0.92 (qd, J=12.4, 4.0 Hz, 1H), 0.83 (qd, J=12.4, 3.6 Hz, 1H); TOF LC-MS [M+H]+ 501.07.

### Example Compound 169

### tert-Butyl 4-(2-{6-amino-8-[(6-formyl-1,3-benzodioxol-5-yl)thio]-9H-purin-9-yl}ethyl)piperidine-1-carboxylate

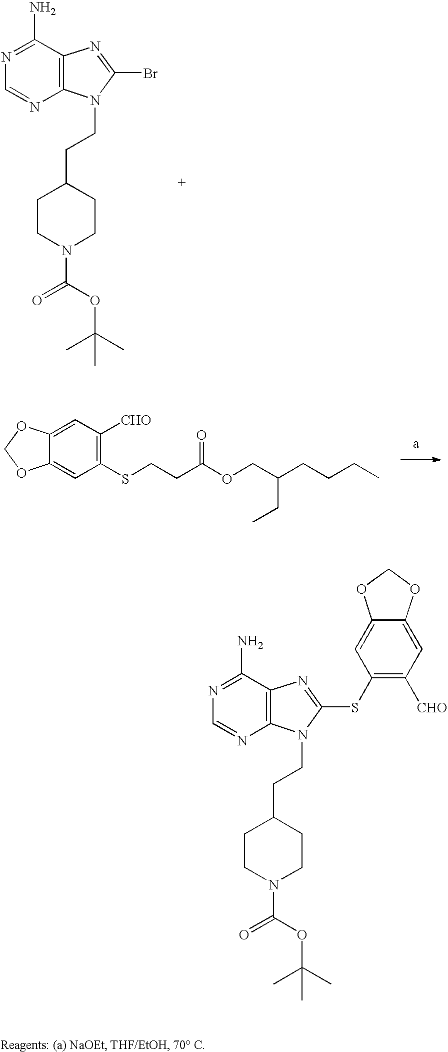

The example compound was prepared by a procedure similar to that described for step 3 of example compound 167, using 2-ethylhexyl 3-[(6-formyl-1,3-benzodioxol-5-yl)thio]propanoate and 4-[2-(6-amino-8-bromo-9H-purin-9-yl)ethyl]piperidine-1-carbaldehyde. ¹H NMR (CDCl₃) δ 10.29 (s, 1H), 8.32 (s, 1H), 7.42 (s, 1H), 6.76 (s, 1H), 6.09 (s, 2H), 5.74-5.67 (br s, 2H), 4.27 (t, J=6.4 Hz, 2H), 4.16-3.98 (m, 2H), 2.71-2.58 (m, 2H), 1.78-1.70 (m, 5H), 1.46 (s, 9H), 1.22-1.11 (m, 2H); TOF LC-MS [M+H]+ 527.21.

### Example Compounds 170 and 171

### Methyl 6-({6-amino-9-[2-(1-formylpiperidin-4-yl)ethyl]-9H-purin-8-yl}thio)-1,3-benzodioxole-5-carboxylate and ethyl 6-({6-amino-9-[2-(1-formylpiperidin-4-yl)ethyl]-9H-purin-8-yl}thio)-1,3-benzodioxole-5-carboxylate

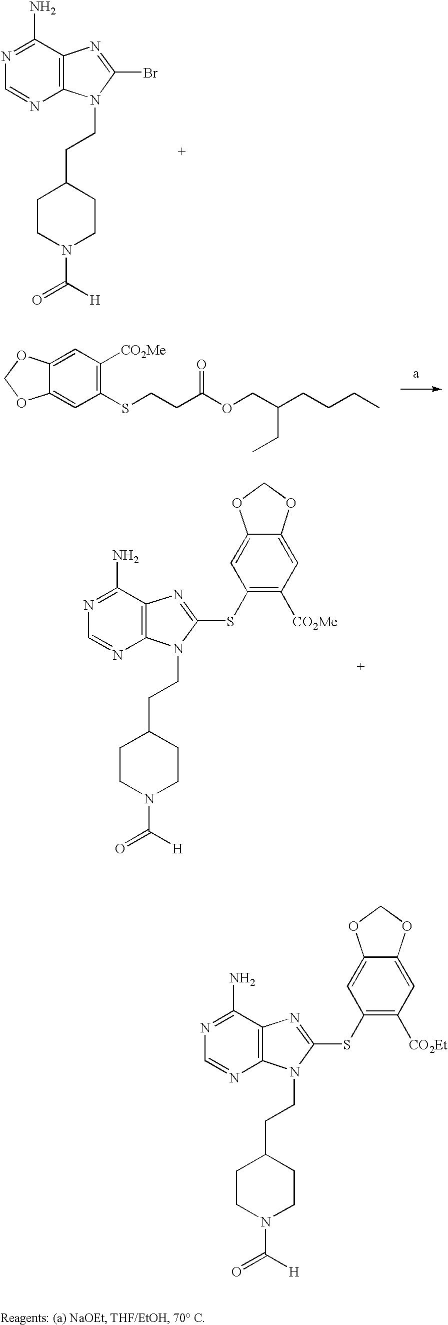

The reaction of 4-[2-(6-amino-8-bromo-9H-purin-9-yl)ethyl]piperidine-1-carbaldehyde with methyl 6-({3-[(2-ethylhexyl)oxy]-3-oxopropyl}thio)-1,3-benzodioxole-5-carboxylate, under identical condition depicted for step 3 of example compound 167, provides a mixture of two products, which were separated by preparative HPLC. After lyophilization, HPLC fractions products were isolated as a trifluoroacetic acid salt. Methyl 6-({6-amino-9-[2-(1-formylpiperidin-4-yl)ethyl]-9H-purin-8-yl}thio)-1,3-benzodioxole-5-carboxylate; 1H NMR (DMSO-d6) δ 8.34 (s, 1H), 7.97 (s, 1H), 7.51 (s, 1H), 6.34 (s, 1H), 6.07 (s, 2H), 4.32 (t, J=6.8 Hz, 2H), 4.26 (br d, J=13.6 Hz, 1H) 3.89 (s, 3H), 3.68 (br d, J=13.2 Hz, 1H), 3.05 (td, J=12.4, 2.0 Hz, 1H), 2.61 (td, J=12.8, 3.2 Hz, 1H), 1.82 (br t, J=10.8 Hz, 2H), 1.73 (q, J=6.8 Hz, 2H), 1.54 (m, 1H), 1.13 (qd, J=12.4, 4.0 Hz, 1H), 1.04 (qd, J=12.0, 3.6 Hz, 1H); TOF LC-MS [M+H]+ 485.16. Ethyl 6-({6-amino-9-[2-(1-formylpiperidin-4-yl)ethyl]-9H-purin-8-yl}thio)-1,3-benzodioxole-5-carboxylate; 1H NMR (DMSO-d6) δ 8.32 (s, 1H), 7.96 (s, 1H), 7.50 (s, 1H), 6.60 (s, 1H), 6.01 (s, 2H), 4.34 (q, J=7.2 Hz, 2H), 4.31 (t, J=7.6 Hz, 2H), 4.26 (br d, J=13.2 Hz, 1H), 3.67 (br d, J=13.6 Hz, 1H), 3.04 (qd, J=13.2, 2.8 Hz, 1H), 2.60 (qd, J=12.8, 3.2 Hz, 1H), 1.86-1.77 (m, 2H), 1.72 (q, J=7.2 Hz, 2H), 1.53 (m, 1H), 1.37 (t, J=7.2 Hz, 3H), 1.12 (qd, J=12.8, 4.4 Hz, 1H), 1.04 (qd, J=12.0, 4.4 Hz, 1H); TOF LC-MS [M+H]+ 485.16.

### Example Compound 172

### tert-Butyl 4-(2-{6-amino-8-[(5-bromo-2,3-dihydro-1-benzofuran-6-yl)thio]-9H-purin-9-yl}ethyl)piperidine-1-carboxylate

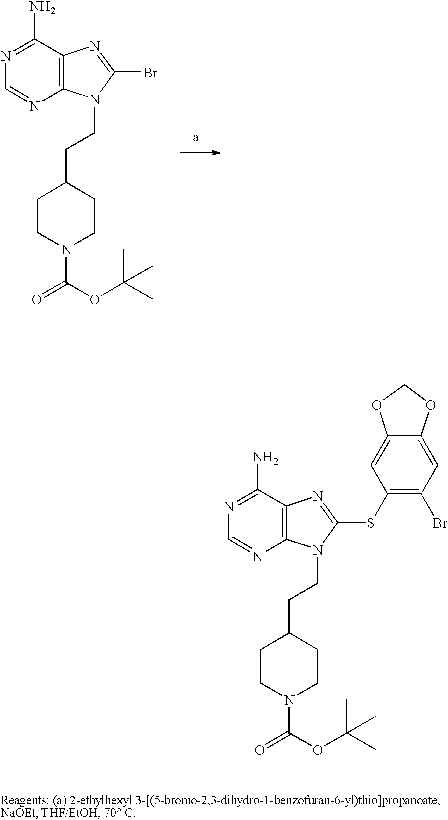

The example compound was synthesized by a similar procedure described for step 3 of example compound 167, using tert-butyl 4-[2-(6-amino-8-bromo-9H-purin-9-yl)ethyl]piperidine-1-carboxylate and 2-ethylhexyl 3-[(5-bromo-2,3-dihydro-1-benzofuran-6-yl)thio]propanoate. ¹H NMR (CDCl₃) δ 8.36 (s, 1H) 7.40 (t, J=0.8 Hz, 1H), 6.49 (s, 1H), 5.64-5.57 (br s, 2H), 4.56 (t, J=8.8 Hz, 2H), 4.24 (t, J=7.2 Hz, 2H), 4.11-3.96 (m, 2H), 3.19 (td, J=8.8, 0.8 Hz, 2H), 2.68-2.54 (m, 2H), 1.74-1.66 (m, 5H), 1.45 (s, 9H), 1.17-1.04 (m, 2H); TOF LC-MS [M+H]⁺ 575.14.

### Example Compound 173

### (2S)-1-[4-(2-{6-Amino-8-[(5-bromo-2,3-dihydro-1-benzofuran-6-yl)thio]-9H-purin-9-yl}ethyl)piperidin-1-yl]-1-oxopropan-2-ol

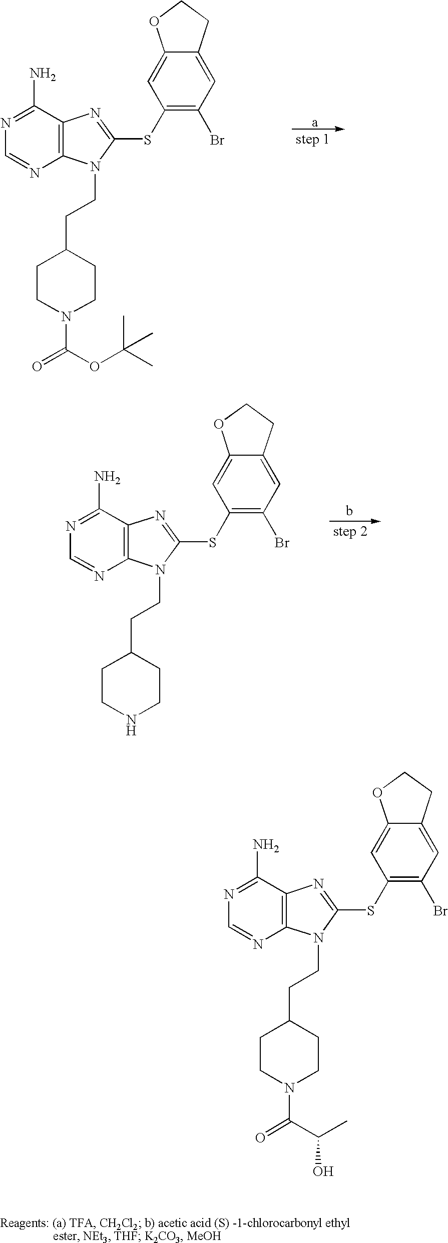

The example compound was prepared in two-step reaction sequence.

Step 1. 8-[(5-bromo-2,3-dihydro-1-benzofuran-6-yl)thio]-9-(2-piperidin-4-ylethyl)-9H-purin-6-amine:

The example compound (74 mg) was prepared by a procedure similar to that described in step 2 of example compound 140, using tert-butyl 4-(2-{6-amino-8-[(5-bromo-2,3-dihydro-1-benzofuran-6-yl)thio]-9H-purin-9-yl}ethyl)piperidine-1-carboxylate. TOF LC-MS [M+H]⁺ 475.09.

Step 2. (2S)-1-[4-(2-{6-amino-8-[(5-bromo-2,3-dihydro-1-benzofuran-6-yl)thio]-9H-purin-9-yl}ethyl)piperidin-1-yl]-1-oxopropan-2-ol

(2S)-1-[4-(2-{6-amino-8-[(5-bromo-2,3-dihydro-1-benzofuran-6-yl)thio]-9H-purin-9-yl}ethyl)piperidin-1-yl]-1-oxopropan-2-ol (10 mg, 12%) was prepared by reacting 8-[(5-bromo-2,3-dihydro-1-benzofuran-6-yl)thio]-9-(2-piperidin-4-ylethyl)-9H-purin-6-amine and acetic acid (5)-1-chlorocarbonyl ethyl ester in THF using Et₃N as a base followed by deprotection as described for example compounds 88 and 89. ¹H NMR (CD₃OD) δ 8.18 (s, 1H), 7.54 (s, 1H), 6.68 (s, 1H), 4.57 (t, J=8.4 Hz, 2H), 4.53 (m, 1H), 4.44 (br d, J=11.6 Hz, 1H), 4.29 (t, J=7.6 Hz, 2H), 3.97 (br t, J=14.0 Hz, 1H), 3.23 (t, J=8.0 Hz, 2H), 2.97 (m, 1H), 2.58 (m, 1H), 1.88-1.76 (m, 2H), 1.75-1.67 (m, 2H), 1.55 (m, 1H), 1.30 and 1.27 (two d, J=6.4 Hz, 3H), 1.24-1.05 (m, 2H); TOF LC-MS [M+H]⁺ 547.11.

### Example Compounds 174 and 175

### Methyl 6-{[6-amino-9-(2-piperidin-4-ylethyl)-9H-purin-8-yl]thio}-1,3-benzodioxole-5-carboxylate and ethyl 6-{[6-amino-9-(2-piperidin-4-ylethyl)-9H-purin-8-yl]thio}-1,3-benzodioxole-5-carboxylate

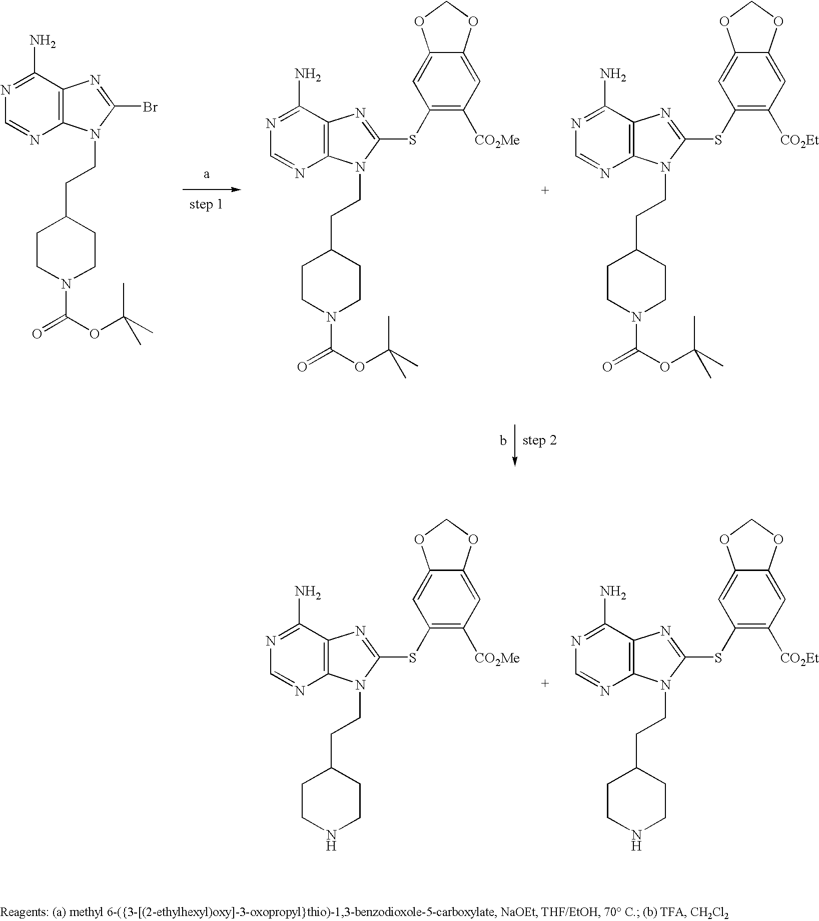

The example compounds were obtained in two-step reaction sequence in a similar manner as described for step 3 of example compound 167, using tert-butyl 4-[2-(6-amino-8-bromo-9H-purin-9-yl)ethyl]piperidine-1-carboxylate and methyl 6-({3-[(2-ethylhexyl)oxy]-3-oxopropyl}thio)-1,3-benzodioxole-5-carboxylate, followed by Boc-deprotection to afford a mixture of methyl 6-{[6-amino-9-(2-piperidin-4-ylethyl)-9H-purin-8-yl]thio}-1,3-benzodioxole-5-carboxylate and ethyl 6-{[6-amino-9-(2-piperidin-4-ylethyl)-9H-purin-8-yl]thio}-1,3-benzodioxole-5-carboxylate which were purified by preparative HPLC and isolated as a trifluoro acetate salt after lyophilization of HPLC fractions. Methyl 6-{[6-amino-9-(2-piperidin-4-ylethyl)-9H-purin-8-yl]thio}-1,3-benzodioxole-5-carboxylate, ¹H NMR (CD₃OD) δ 8.31 (s, 1H), 7.51 (s, 1H), 6.60 (s, 1H), 6.07 (s, 2H), 4.32 (br t, J=6.8 Hz, 2H), 3.89 (s, 3H), 3.37-3.33 (m, 2H), 2.91 (br t, J=12.8 Hz, 2H), 2.25-1.98 (m, 2H), 1.82-1.75 (m, 2H), 1.57 (m, 1H), 1.44-1.35 (m, 2H); TOF LC-MS [M+H]⁺ 457.16. Ethyl 6-{[6-amino-9-(2-piperidin-4-ylethyl)-9H-purin-8-yl]thio}-1,3-benzodioxole-5-carboxylate, ¹NMR (CD₃OD) δ 8.30 (s, 1H), 7.51 (s, 1H), 6.59 (s, 1H), 6.06 (s, 2H), 4.33 (q, J=6.8 Hz, 2H), 4.32 (br t, J=7.6 Hz, 2H), 3.39-3.30 (m, 2H), 2.91 (td, J=12.8, 2.8 Hz, 2H), 2.00 (br d, J=14.4 Hz, 2H), 1.78 (q, J=7.6 Hz, 2H), 1.58 (m, 1H), 1.43-1.32 (m, 2H), 1.37 (t, J=6.8 Hz, 3H); TOF LC-MS [M+H]⁺ 471.18.

### Example Compounds 176-208

Example compounds 176-208 were synthesized in the same manner as described for step 3 of example compounds 167, above, using appropriate starting intermediates described above, and are summarized in Table 9, below.

TABLE 9
Preparation of Example Compounds 176-208.

| Example | | |
|---|---|---|
| Compound | Structure | Name and analytical data |
| 176 | 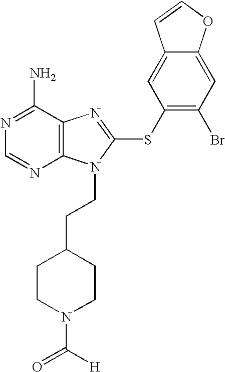 | 4-(2-{6-Amino-8-[(6-bromo-1-benzofuran- 5-yl)thio]-9H-purin-9-yl}ethyl)piperidine-1- carbaldehyde ¹H NMR (DMSO-d₆) δ 8.31 (s, 1H), 8.09 (s, 1H), 8.05 (d, J = 1.2 Hz, 1H), 7.98 (s, 1H), 7.90 (d, J = 2.4 Hz, 1H), 6.94 (dd, J = 2.4, 1.2 Hz, 1H), 4.41 (t, J = 7.6 Hz, 2H), 4.30 (br d, J = 13.6, 1H), 3.71 (br d, J = 13.6 1H), 3.09 (td, J = 12.8, 2.8 Hz, 1H), 2.64 (td, J = 12.4, 2.8 Hz, 1H), 1.95-1.84 (m, 4H), 1.63 (m, 1H) 1.21 (qd, J = 12.0, 4.0 Hz, 1H), 1.13 (qd, J = 12.4, 4.4 Hz, 1H); LC-TOF MS [M + H]⁺501.07 |
| 177 | 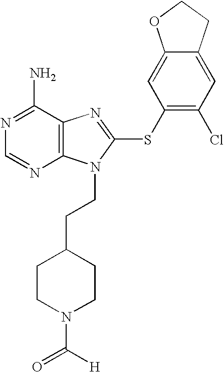 | 4-(2-{6-Amino-8-[(5-chloro-2,3-dihydro- 1-benzofuran-6-yl)thio]-9H-purin-9- yl}ethyl)piperidine-1-carbaldehyde ¹H NMR (CD₃OD) δ 8.29 (s, 1H), 7.97 (s, 1H), 7.42 (t, J = 1.0 Hz, 1H), 6.87 (s, 1H), 4.60 (t, J = 8.4 Hz, 2H), 4.36 (t, J = 7.2 Hz, 2H), 4.28 (brd, J = 13.2 Hz, 1H), 3.70 (brd, J = 13.2, 1H), 3.25 (tt, J = 7.2, 1.0 Hz, 2H), 3.06 (td, J = 12.8, 3.2 Hz, 1H), 2.31 (td, J = 12.8, 2.8 Hz, 1H), 1.87 (brt, J = 15.6 Hz, 2H), 1.78 (q, J = 7.2 Hz, 2H), 1.58 (m, 1H), 1.68 (qd, J = 11.6, 4.0 Hz, 1H), 1.09 (qd, J = 12.4, 4.4 Hz, 1H); TOF LC-MS [M + H]⁺458.14 |
| 178 | 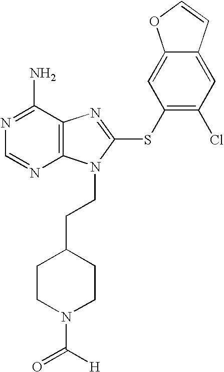 | 4-(2-{6-Amino-8-[(5-chloro-1-benzofuran- 6-yl)thio]-9H-purin-9-yl}ethyl)piperidine-1- carbaldehyde ¹H NMR (CD₃OD) δ 8.23 (s, 1H), 7.97 (s, 1H), 7.92 (d, J = 2.4 Hz, 1H), 7.89 (s, 1H), 7.84 (d, J = 0.8 Hz, 1H), 6.93 (d, J = 2.4, 0.8 Hz, 1H), 4.38 (t, J = 7.6 Hz, 2H), 4.27 (brd, J = 13.2 Hz, 1H), 3.69 (brd, J = 13.2 Hz, 1H), 3.05 (td, J = 12.8, 2.4, 1H), 2.62 (td, J = 12.8, 3.2 Hz, 1H), 1.86 (brt, J = 16 Hz, 2H), 1.79 (q, J = 7.2, Hz, 2H), 1.59 (m, 1H), 1.17 (qd, J = 12.8, 4.4 Hz, 1H), 1.09 (qd, J = 12.8, 4.8 Hz, 1H); TOF LC-MS [M + H]⁺457.12 |
| 179 | 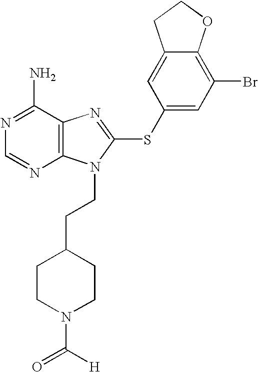 | 4-(2-{6-Amino-8-[(7-bromo-2,3-dihydro- 1-benzofuran-5-yl)thio]-9H-purin-9- yl}ethyl)piperidine-1-carbaldehyde TOF LC-MS [M + H]⁺503.08 |
| 180 | 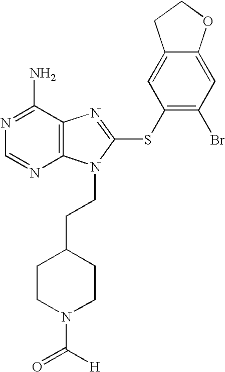 | 4-(2-{6-Amino-8-[(6-bromo-2,3-dihydro- 1-benzofuran-5-yl)thio]-9H-purin-9- yl}ethyl)piperidine-1-carbaldehyde ¹H NMR (CD₃OD) δ 8.28 (s, 1H), 7.99 (s, 1H), 7.60 (d, J = 2.4 Hz, 1H), 7.19 (s, 1H), 4.67 (t, J = 9.7 Hz, 2H), 4.37 (t, J = 7.2 Hz, 2H), 4.31 (brd, J = 13.2 Hz, 1H), 3.72 (brd, J = 13.6 Hz, 1H), 3.22 (t, J = 9.7 Hz, 2H), 3.10 (td, J = 12.8, 2.8 Hz, 1H), 2.66 (td, J = 12.8, 3.2 Hz, 1H), 1.92 (brt, J = 16 Hz, 2H), 1.79 (q, J = 7.2, Hz, 2H), 1.64 (m, 1H), 1.22 (qd, J = 11.6, 4.8 Hz, 1H), 1.13 (qd, J = 12.0, 4.4 Hz, 1H); TOF LC-MS [M + H]⁺503.09 |
| 181 | 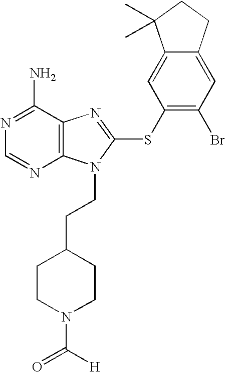 | 4-(2-{6-Amino-8-[(6-bromo-3,3-dimethyl- 2,3-dihydro-1H-inden-5-yl)thio]-9H-purin-9- yl}ethyl)piperidine-1-carbaldehyde ¹H NMR (DMSO-d₆) δ 8.17 (s, 1H), 7.92 (s, 1H), 7.57 (s, 1H), 7.46 (brs, 2H), 6.90 (s, 1H), 4.16 (d, J = 6.8 Hz, 2H), 4.06 (brd, J = 14.0 Hz, 1H), 3.58 (brd, J = 12.8 Hz, 1H), 2.87 (td, J = 12.4, 2.8 Hz, 1H), 2.84 (t, J = 7.2 Hz, 2H), 2.45 (td, J = 12.4, 2.4 Hz, 1H), 1.84 (t, J = 7.2 Hz, 2H), 1.72-1.59 (m, 2H), 1.54 (q, J = 8.0 Hz, 2H), 1.39 (m, 1H), 1.06 (two s, 6H), 0.92 (qd, J = 11.6, 4.4 Hz, 1H), 0.82 (qd, J = 12.0, 4.0 Hz, 1H); TOF LC-MS [M + H]⁺529.14 |
| 182 | 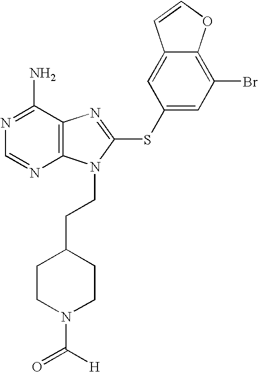 | 4-(2-{6-Amino-8-[(7-bromo-1-benzofuran- 5-yl)thio]-9H-purin-9-yl}ethyl)piperidine-1- carbaldehyde TOF LC-MS [M + H]⁺501.07 |
| 183 | 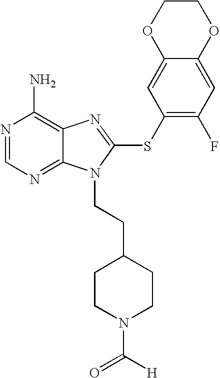 | 4-(2-{6-Amino-8-[(7-fluoro-2,3-dihydro- 1,4-benzodioxin-6-yl)thio]-9H-purin-9- yl}ethyl)piperidine-1-carbaldehyde ¹H NMR (DMSO-d₆) δ 8.31 (s, 1H), 7.95 (s, 1H), 7.11 (d, J = 7.6 Hz, 1H), 7.01 (d, J = 9.6 Hz, 1H), 4.32-4.23 (m, 7H), 4.15-4.10 (m, 2H), 3.66-3.60 (m, 1H), 2.99-2.90 (m, 1H), 1.95-0.95 (m, 6H); LC-MS [M + H]⁺459.16. |
| 184 | 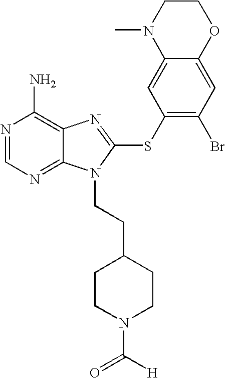 | 4-(2-{6-Amino-8-[(7-bromo-4-methyl-3,4- dihydro-2H-1,4-benzoxazin-6-yl)thio]-9H- purin-9-yl}ethyl)piperidine-1-carbaldehyde LC-MS [M + H]⁺532.11. |
| 185 | 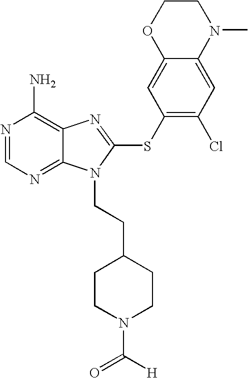 | 4-(2-{6-Amino-8-[(6-chloro-4-methyl-3,4- dihydro-2H-1,4-benzoxazin-7-yl)thio]-9H- purin-9 -yl}ethyl)piperidine-1-carbaldehyde ¹H NMR (DMSO-d₆) δ 8.16 (s, 1H), 7.93 (s, 1H), 6.98 (s, 1H), 6.83 (s, 1H), 4.22-4.17 (m, 4H), 4.15-4.09 (m, 2H), 3.64-3.57 (m, 1H), 3.05-3.02 (m, 2H), 2.92-2.84 (m, 1H), 2.75 (s, 3H), 1.74-1.30 (m, 5H), 1.00-0.80 (m, 2H); LC-MS [M + H]⁺488.16. |
| 186 | 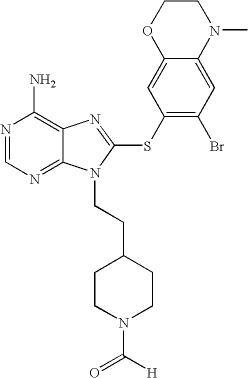 | 4-(2-{6-Amino-8-[(6-bromo-4-methyl-3,4- dihydro-2H-1,4-benzoxazin-7-yl)thio]-9H- purin-9-yl}ethyl)piperidine-1-carbaldehyde ¹H NMR (DMSO-d₆) δ 8.24 (s, 1H), 7.94 (s, 1H), 6.95 (s, 1H), 6.68 (s, 1H), 4.22-4.15 (m, 4H), 4.12-4.08 (m, 2H), 3.64-3.57 (m, 1H), 3.29-3.26 (m, 2H), 2.95-2.87 (m, 1H), 2.86 (s, 3H), 1.86-1.40 (m, 5H), 1.34-0.80 (m, 2H); LC-MS [M + H]⁺532.11. |
| 187 | 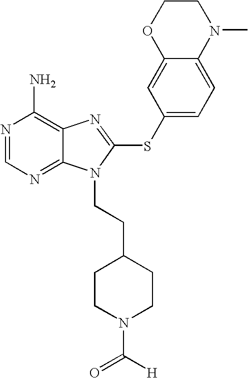 | 4-(2-{6-Amino-8-[(4-methyl-3,4-dihydro- 2H-1,4-benzoxazin-7-yl)thio]-9H-purin-9- yl}ethyl)piperidine-1-carbaldehyde LC-MS [M + H]⁺454.20. |
| 188 | 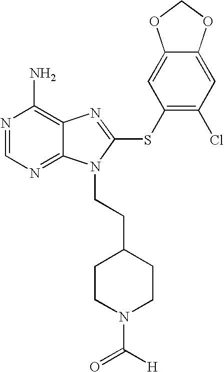 | 4-(2-{6-Amino-8-[(6-chloro-1,3- benzodioxol-5-yl)thio]-9H-purin-9- yl}ethyl)piperidine-1-carbaldehyde ¹H NMR (DMSO-d₆) δ 8.21 (s, 1H), 7.94 (s, 1H), 7.29 (s, 1H), 6.91 (s, 1H), 6.11 (s, 2H), 4.25-4.19 (m, 2H), 4.12-4.05 (m, 1H), 3.66-3.60 (m, 1H), 2.99-2.85 (m, 1H), 1.90-0.95 (m, 8H); LC-MS [M + H]⁺461.12. |
| 189 | 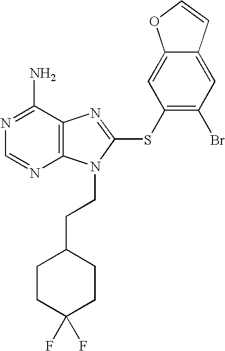 | 8-[(5-Bromo-1-benzofuran-6-yl)thio]-9-[2- (4,4-difluorocyclohexyl)ethyl]-9H-purin-6- amine ¹H NMR (DMSO-d₆) δ 8.18 (s, 1H), 8.09 (s, 1H), 8.07 (d, J = 2.0 Hz, 1H), 7.47 (brs, 2H), 7.44 (d, J = 0.8 Hz, 1H), 6.97 (dd, J = 2.0, 0.8 Hz, 1H), 4.20 (t, J = 6.8 Hz, 2H), 1.94-1.84 (m, 2H), 1.73-1.64 (m, 4H), 1.62-1.57 (m, 2H), 1.26 (m, 1H), 1.14-1.03 (m, 2H); TOF LC-MS [M + H]⁺508.06 |
| 190 | 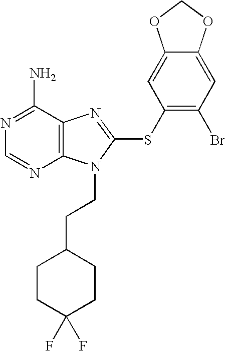 | 8-[(6-Bromo-1,3-benzodioxol-5-yl)thio]-9- [2-(4,4-difluorocyclohexyl)ethyl]-9H-purin-6- amine ¹H NMR (DMSO-d₆) δ 8.16 (s, 1H), 7.43 (brs, 2H), 7.39 (s, 1H), 6.81 (s, 1H), 6.09 (s, 2H), 4.17 (t, J = 7.2 Hz, 2H), 2.00-1.90 (m, 2H), 1.83-1.68 (m, 4H), 1.65-1.57 (m, 2H), 1.32 (m, 1H), 1.19-1.10 (m, 2H); TOF LC-MS [M + H]⁺512.06 |
| 191 | 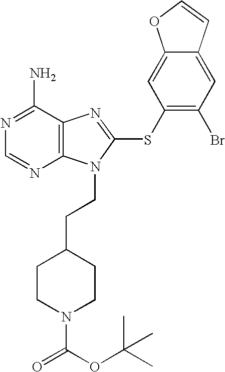 | tert-Butyl 4-(2-{6-amino-8-[(5-bromo-1- benzofuran-6-yl)thio]-9H-purin-9- yl}ethyl)piperidine-1-carboxylate ¹H NMR (CDCl₃) δ 8.36 (s, 1H), 7.88 (s, 1H), 7.62 (d, J = 2.4 Hz, 1H), 7.43 (d, J = 0.8 Hz, 1H), 6.73 (dd, J = 2.4, 0.8 Hz, 1H), 4.27 (t, J = 7.2 Hz, 2H), 4.13-3.94 (m, 2H), 2.68-2.52 (m, 2H), 1.72-1.67 (m, 4H), 1.45 (s, 9H), 1.37 (m, 1H), 1.10 (qd, J = 12.4, 4.4 Hz, 2H); TOF LC-MS [M + H]⁺573.13 |
| 192 | 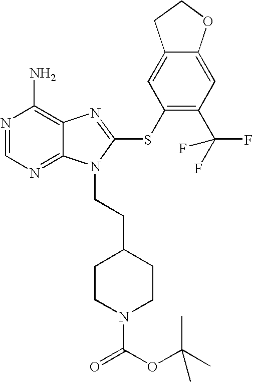 | tert-Butyl 4-[2-(6-amino-8-{[6- (trifluoromethyl)-2,3-dihydro-1- benzofuran-5- yl]thio}-9H-purin-9-yl)ethyl]piperidine-1- carboxylate ¹H NMR (CD₃OD) δ 8.25 (s, 1H), 7.65 (s, 1H), 7.24 (s, 1H), 4.72 (t, J = 8.8 Hz, 2H), 4.33 (t, J = 8.8 Hz, 2H), 4.08-4.02 (m, 2H), 1.83-1.78 (m, 4H), 1.45 (s, 9H), 1.36-1.08 (m, 7H); LC-MS [M + H]⁺565.22. |
| 193 | 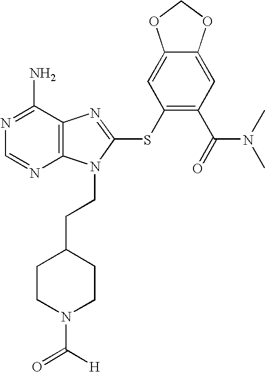 | 6-({6-amino-9-[2-(1-formylpiperidin-4- yl)ethyl]-9H-purin-8-yl}thio)-N,N-dimethyl- 1,3-benzodioxole-5-carboxamide ¹H NMR (CD₃OD) δ 8.81 (s, 1H), 7.99 (s, 1H), 7.24 (s, 1H), 6.95 (s, 1H), 6.12 (s, 2H), 4.32 (t, J = 7.2 Hz, 2H), 4.30 (m, 1H), 3.72 (brd, J = 14.0 Hz, 1H), 3.11 (td, J = 12.0, 2.4 Hz, 1H), 3.20 (s, 3H), 2.89 (s, 3H), 2.66 (td, J = 13.2, 3.2 Hz, 2H), 1.91 (brt, J = 13.2 Hz, 2H), 1.77 (q, J = 8.0 Hz, 2H), 1.61 (m, 1H), 1.21 (m, 1H), 1.12 (m, 1H); TOF LC-MS [M + H]⁺498.19 |
| 194 | 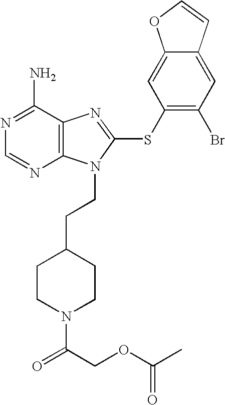 | 2-[4-(2-{6-Amino-8-[(5-bromo-1- benzofuran-6-yl)thio]-9H-purin-9- yl}ethyl)piperidin-1-yl]-2-oxoethyl acetate TOF LC-MS [M + H]⁺575.14 |
| 195 | 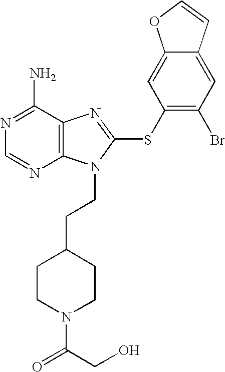 | 2-[4-(2-{6-Amino-8-[(5-bromo-1- benzofuran-6-yl)thio]-9H-purin-9- yl}ethyl)piperidin-1-yl]-2-oxoethanol ¹H NMR (DMSO-d₆) δ 8.18 (s, 1H), 8.09 (s, 1H), 8.07 (d, J = 2.4 Hz, 1H), 7.48(br s, 2H), 7.45 (d, J = 0.4 Hz, 1H), 6.97 (dd, J = 2.0, 0.4 Hz, 1H), 4.43 (t, J = 5.6 Hz, 1H, —OH), 4.25-4.18 (m, 3H), 4.02 (m, 2H), 3.55 (br d, J = 13.2 Hz, 1H), 2.75 (br t, J = 12.0 Hz, 1H), 2.42 (br t, J = 12.4 Hz, 1H), 1.68-1.54 (m, 4H), 1.36 (s, 1H), 1.98 (m, 1H), 1.89 (m, 1H); TOF LC-MS [M + H]⁺531.08 |
| 196 | 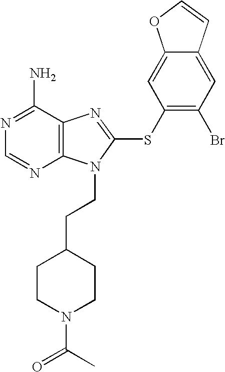 | 9- [2-(1-Acetylpiperidin-4-yl)ethyl]-8-[(5- bromo-1-benzofuran-6-yl)thio]-9H-purin-6- amine ¹H NMR (DMSO-d₆) δ 8.18 (s, 1H), 8.09 (s, 1H), 8.07 (d, J = 2.0 Hz, 1H), 7.47 (brs, 2H), 7.45 (s, 1H), 6.97 (d, J = 2.0 Hz, 1H), 4.26-4.17 (m, 3H), 3.68 (br d, J =13.6, 1H), 2.81 (t, J =12.4 1H), 2.31 (t, J = 10.8 Hz, 1H), 1.93 (s, 3H), 1.65-1.56 (m, 4H), 1.34 (m, 1H), 0.98 (qd, J = 11.6, 3.6 Hz, 1H), 0.85 (qd, J = 12.0, 4.4 Hz, 1H); TOF LC-MS [M + H]⁺501.07 |
| 197 | 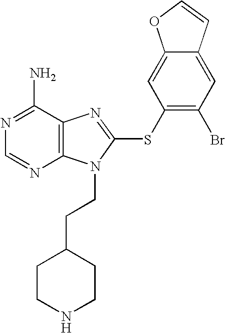 | 8-[(5-Bromo-1-benzofuran-6-yl)thio]-9-(2- piperidin-4-ylethyl)-9H-purin-6-amine TOF LC-MS [M + H]⁺473.07 |
| 198 | 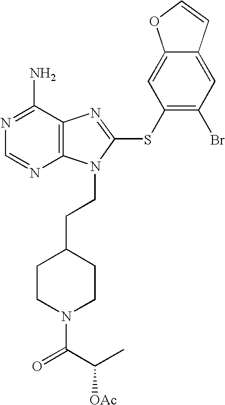 | (1S)-2-[4-(2-{6-Amino-8-[(5-bromo-1- benzofuran-6-yl)thio]-9H-purin-9- yl}ethyl)piperidin-1-yl]-1-methyl-2-oxoethyl acetate ¹H NMR (CDCl₃) δ 8.18 (s, 1H), 8.056 and 8.053 (two s, 1H), 7.88 (d, J = 2.0 Hz, 1H), 7.747 and 7.739 (two s, 1H), 6.91 (dd, J = 2.0, 0.8 Hz, 1H), 5.39 (m, 1H), 4.09 (m, 1H), 4.34 (t, J = 7.6 Hz, 2H), 3.92 (m, 1H), 3.05 (m, 1H), 2.58 (m, 1H), 2.08 and 2.07 (two s, 3H), 1.92-1.71 (m, 5H), 1.39 and 1.36 (two d, J = 6.4 and 7.2 Hz, 3H), 1.34 (m, 1H), 1.14 (m, 1H); TOF LC-MS [M + H]⁺587.11 |
| 199 | 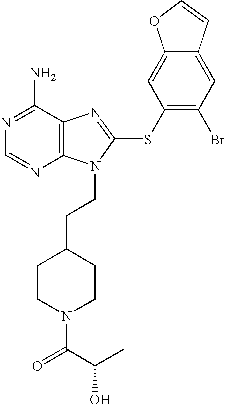 | (2S)-1-[4-(2-{6-Amino-8-[(5-bromo-1- benzofuran-6-yl)thio]-9H-purin-9- yl}ethyl)piperidin-1-yl]-1-oxopropan-2-ol ¹H NMR (DMSO-d₆) δ 8.18 (s, 1H), 8.09 (s, 1H), 8.07 (d, J = 2.4 Hz, 1H), 7.47 (brs, 2H), 7.45 (s, 1H), 6.97 (dd, J = 2.4, 0.8 Hz, 1H), 4.75(d, J = 6.8 Hz, 1H), 4.36 (m, 1H), 4.29-4.18 (m, 3H), 3.86 (brd, J = 13.2 Hz, 1H), 2.79 (m, 1H), 2.39 (m, 1H), 1.70-1.56 (m, 4H), 1.37 (m, 1H), 1.14-1.11 (m, 3H), 1.06-0.84 (m, 2H); TOF LC-MS [M + H]⁺545.10 |
| 200 | 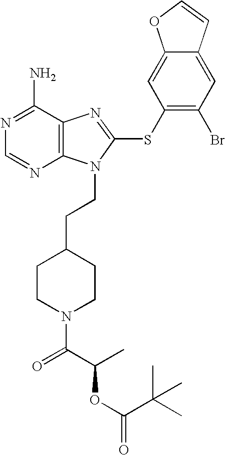 | (1R)-2-[4-(2-{6-Amino-8-[(5-bromo-1- benzofuran-6-yl)thio]-9H-purin-9- yl}ethyl)piperidin-1-yl]-1- methyl-2-oxoethyl pivalate ¹H NMR (DMSO-d₆) δ 8.18 (s, 1H), 8.09 (s, 1H), 8.08 (d, J = 2.0 Hz, 1H), 7.49 (brs, 2H), 7.455 and 7.444 (two s, 1H), 6.97 (dd, J = 2.0, 0.8 Hz, 1H), 5.30 (m, 1H), 4.23-4.18 (m, 3H), 3.75 (m, 1H), 2.84 (m, 1H), 2.39 (m, 1H), 1.74-1.56 (m, 5H), 1.30-1.23 (m, 3H), 1.15-1.13 (m, 9H), 1.06-0.81 (m, 2H); TOF LC-MS [M + H]⁺629.15 |
| 201 | 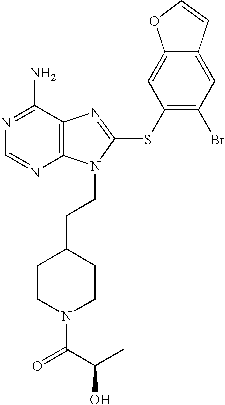 | (2R)-1-[4-(2-{6-Amino-8-[(5-bromo-1- benzofuran-6-yl)thio]-9H-purin-9- yl}ethyl)piperidin-1-yl]-1-oxopropan-2-ol. ¹H NMR (DMSO-d₆) δ 8.18 (s, 1H), 8.09 (s, 1H), 8.07 (d, J = 2.4 Hz, 1H), 7.47 (brs, 2H), 7.45 (s, 1H), 6.97 (dd, J = 2.4, 0.8 Hz, 1H), 4.75 (d, J = 6.8 Hz, 1H), 4.36 (m, 1H), 4.29-4.18 (m, 3H), 3.86 (m, 1H), 2.79 (m, 1H), 2.39 (m, 1H), 1.70-1.56 (m, 4H), 1.38 (m, 1H), 1.14-1.11 (m, 3H), 1.06-0.84 (m, 2H); TOF LC-MS [M + H]⁺545.10 |
| 202 | 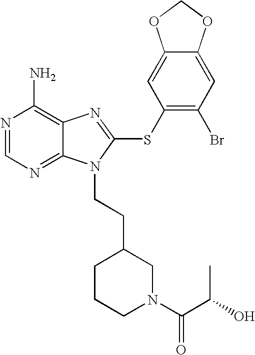 | (2S)-1-[3-(2-{6-Amino-8-[(6-bromo-1,3- benzodioxol-5-yl)thio]-9H-purin-9- yl}ethyl)piperidin-1-yl]-1-oxopropan-2-ol ¹H NMR (DMSO-d₆) δ 8.36 (s, 1H), 7.41 (s, 1H), 6.97 (s, 1H), 6.12 (s, 2H), 4.47-4.37 (m, 1H), 4.27-4.21 (m, 3H), 4.03-3.97 (m, 1H), 3.87-3.72 (m, 1H), 3.10-3.00 (m, 1H), 2.80-2.55 (m, 2H), 1.99-1.00 (m, 5H), 0.94 (d, J = 6.8 Hz, 3H); LC-MS [M + H]⁺594.09. |
| 203 | 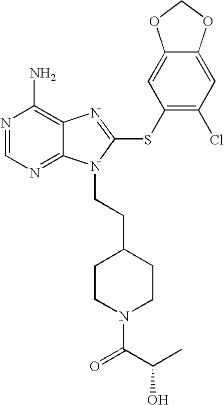 | (2S)-1-[4-(2-{6-Amino-8-[(6-chloro-1,3- benzodioxol-5-yl)thio]-9H-purin-9- yl}ethyl)piperidin-1-yl]-1-oxopropan-2-ol. ¹H NMR (DMSO-d₆) δ 8.26 (s, 1H), 7.30 (s, 1H), 6.95 (s, 1H), 6.11 (s, 2H), 4.42-4.37 (m, 1H), 4.36-4.25 (m, 1H), 4.24-4.19 (m, 2H), 3.98-3.88 (m, 1H), 2.96-2.80 (m, 1H), 2.50-2.40 (m, 1H), 1.78-1.68 (m, 2H), 1.66-1.58 (m, 2H), 1.50-1.38 (m, 1H), 1.17-1.12 (m, 3H), 1.10-0.90 (m, 2H); LC-MS/TOF [M + H]⁺505.14. |
| 204 | 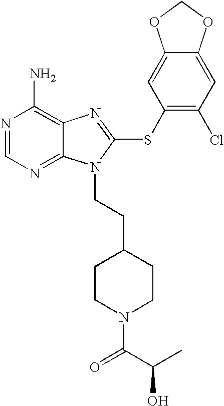 | (2R)-1-[4-(2-{6-Amino-8-[(6-chloro-1,3- benzodioxol-5-yl)thio]-9H-purin-9- yl}ethyl)piperidin-1-yl]-1-oxopropan-2-ol. ¹H NMR (DMSO-d₆) δ 8.16 (s, 1H), 7.28 (s, 1H), 6.87 (s, 1H), 6.11 (s, 2H), 4.42-4.37 (m, 1H), 4.36-4.25 (m, 1H), 4.21-4.16 (m, 2H), 3.98-3.88 (m, 1H), 2.94-2.80 (m, 1H), 2.50-2.40 (m, 1H), 1.78-1.68 (m, 2H), 1.64-1.55 (m, 2H), 1.50-1.38 (m, 1H), 1.17-1.12 (m, 3H), 1.10-0.90 (m, 2H); TOF LC-MS [M + H]⁺505.14. |
| 205 | 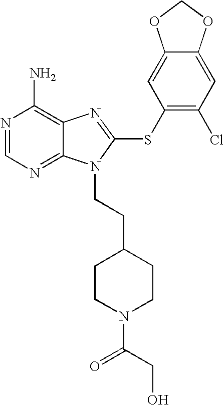 | 2-[4-(2-{6-Amino-8-[(6-chloro-1,3- benzodioxol-5-yl)thio]-9H-purin-9- yl}ethyl)piperidin-1-yl]-2-oxoethanol ¹H NMR (DMSO-d₆) δ 8.15 (s, 1H), 7.28 (s, 1H), 6.87 (s, 1H), 6.10 (s, 2H), 4.45-4.41 (m, 1H), 4.34-4.25 (m, 1H), 4.22-4.17 (m, 2H), 4.07-4.01 (m, 2H), 3.63-3.58 (m, 1H), 2.59-2.78 (m, 1H), 1.74-1.56 (m, 4H), 1.42-0.95 (m, 3H); TOF LC-MS [M + H]⁺491.13. |
| 206 | 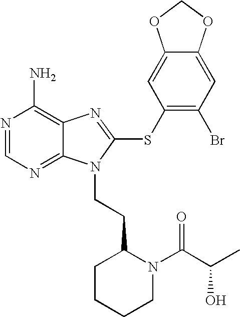 | (2S)-1-[(2R)-2-(2-{6-Amino-8-[(6-bromo- 1,3-benzodioxol-5-yl)thio]-9H-purin-9- yl}ethyl)piperidin-1-yl]-1-oxopropan-2-ol TOF LC-MS [M + H]⁺549.09. |
| 207 | 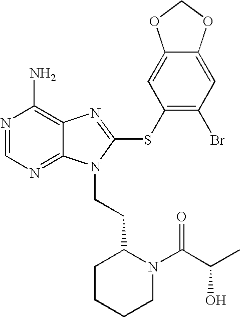 | (2S)-1-[(2S)-2-(2-{6-Amino-8-[(6-bromo- 1,3-benzodioxol-5-yl)thio]-9H-purin-9- yl}ethyl)piperidin-1-yl]-1-oxopropan-2-ol TOF LC-MS [M + H]⁺549.09. |
| 208 | 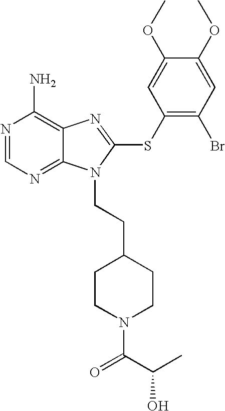 | (2S)-1-[4-(2-{6-Amino-8-[(2-bromo-4,5- dimethoxyphenyl)thio]-9H-purin-9- yl}ethyl)piperidin-1-yl]-1-oxopropan-2-ol. ¹H NMR (DMSO-d₆) δ 8.29 (s, 1H), 7.32 (s, 1H), 7.05 (s, 1H), 4.41-4.37 (m, 5H), 3.97-3.89 (m, 1H), 3.80 (s, 3H), 3.66 (s, 3H), 2.94-2.80 (m, 1H), 1.76-1.58 (m, 4H), 1.52-1.40 (m, 1H), 1.17-0.90 (m, 5H); LC-MS [M + H]⁺565.12. |

### Example Compound 209

### 4-{[6-Amino-9-(2-{1-[(2S)-2-hydroxypropanoyl]piperidin-4-yl}ethyl)-9H-purin-8-yl]thio}-5-bromobenzene-1,2-diol

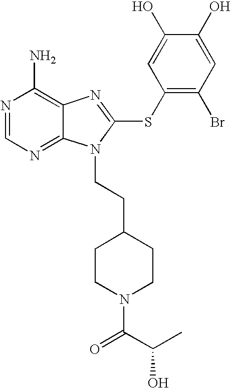

The example compound was prepared by a similar procedure to that described for example compounds 145 and 146 using (2S)-1-[4-(2-{6-amino-8-[(2-bromo-4,5-dimethoxyphenyl)thio]-9H-purin-9-yl}ethyl)piperidin-1-yl]-1-oxopropan-2-ol. ¹H NMR (DMSO-d6) δ 8.31 (s, 1H), 7.04 (s, 1H), 6.70 (s, 1H), 4.41-4.16 (m, 5H), 3.97-3.86 (m, 1H), 2.94-2.80 (m, 1H), 1.76-1.52 (m, 4H), 1.50-1.38 (m, 1H), 1.17-0.90 (m, 5H); LC-MS [M+H]+ 537.09.

### Example Compound 210

### tert-Butyl 4-(6-amino-8-bromo-9H-purin-9-yl)piperidine-1-carboxylate

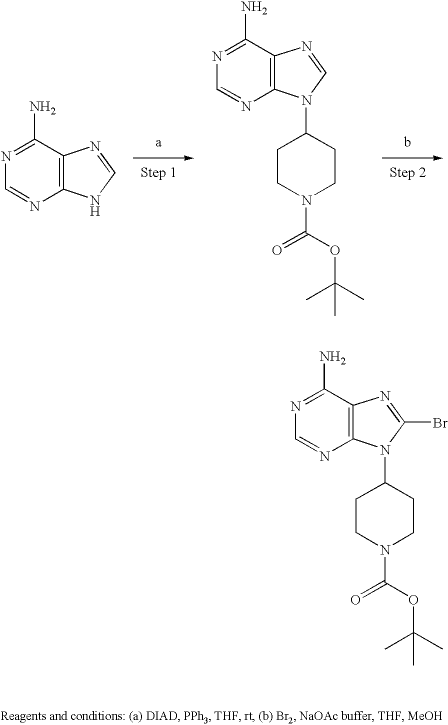

Step 1: tert-Butyl 4-(6-amino-9H-purin-9-yl)piperidine-1-carboxylate

Adenine (2.0 g, 15.0 mmol) was dissolved in THF (50 mL), followed by the addition of 1-boc-4-hydroxypiperidine (3.90 g, 19.2 mmol), triphenylphosphine (8.4 g, 33.0 mmol) and finally DIAD (14.8 mL, 75.0 mmol). After stirring for 18 h, the reaction mixture was concentrated in vacuum and the residue was purified by HPLC to give tert-butyl 4-(6-amino-9H-purin-9-yl)piperidine-1-carboxylate (800 mg, 17%) as a white solid. LC-MS [M+H]+ 319.27

Step 2: tert-butyl 4-(6-amino-8-bromo-9H-purin-9-yl)piperidine-1-carboxylate

tert-Butyl 4-(6-amino-9H-purin-9-yl)piperidine-1-carboxylate (0.800 g, 2.5 mmol) was dissolved in THF-MeOH (v/v 5 mL), followed by the addition of NaOAc buffer solution (5 mL). After 5 min, Br2 (0.272 mL, 4.5 mmol) was added dropwise and the mixture was stirred at room temperature for 18 h. The reaction was then concentrated in vacuum and the residue was purified by HPLC to yield the example compound: tert-butyl 4-(6-amino-8-bromo-9H-purin-9-yl)piperidine-1-carboxylate (0.80 g, 80%) as a light brown solid; LC-MS [M+H]+ 397.10

### Example Compound 211

### 8-[(6-Bromo-1,3-benzodioxol-5-yl)thio]-9-piperidin-4-yl-9H-purin-6-amine

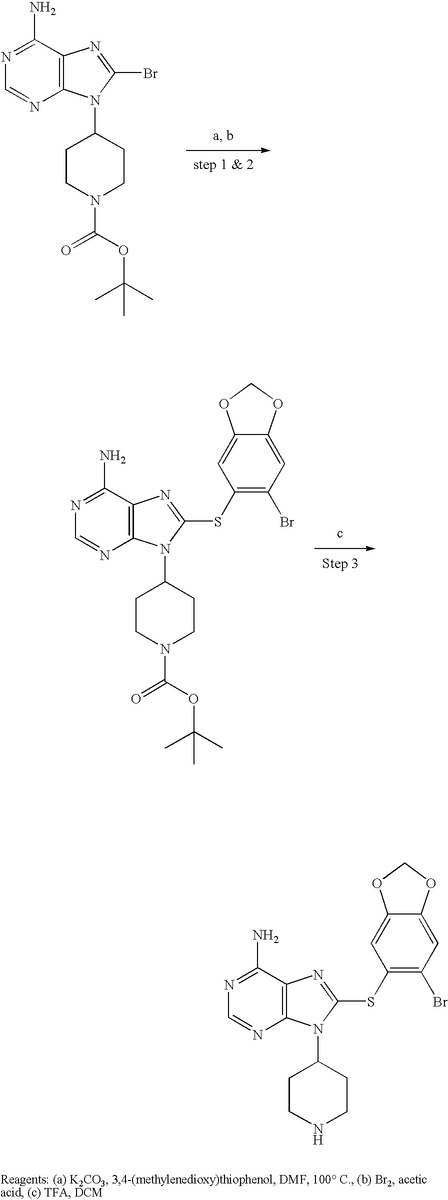

Step 1: tert-Butyl 4-{6-amino-8-[(1,3-benzodioxol-5-yl)thio]-9H-purin-9-yl}piperidine-1-carboxylate

tert-butyl 4-(6-amino-8-bromo-9H-purin-9-yl)piperidine-1-carboxylate

(0.250 g, 0.629 mmol) was dissolved in DMF (2 mL), followed by the addition of 3,4-(methylenedioxy)thiophenol (0.116 g, 0.755 mmol) and K2CO3 (0.174 g, 1.3 mmol) and the mixture was heated to 100° C. for 18 h. After cooling to r.t the reaction mixture was concentrated in vacuum and the residue was purified by HPLC to afford tert-butyl 4-{6-amino-8-[(1,3-benzodioxol-5-yl)thio]-9H-purin-9-yl}piperidine-1-carboxylate as a white solid (37 mg, 12.5%); LC-MS [M+H]+ 471.17.

Step 2. tert-Butyl 4-{6-amino-8-[(6-bromo-1,3-benzodioxol-5-yl)thio]-9H-purin-9-yl}piperidine-1-carboxylate

tert-Butyl 4-{6-amino-8-[(1,3-benzodioxol-5-yl)thio]-9H-purin-9-yl}piperidine-1-carboxylate (0.037 g, 0.079 mmol) was dissolved in acetic acid (5 mL), followed by the addition of bromine (0.009 mL, 0.173 mmol). After stirring for 4 h at rt, the reaction mixture was quenched with several drops of water, concentrated in vacuum and dried to afford tert-butyl 4-{6-amino-8-[(6-bromo-1,3-benzodioxol-5-yl)thio]-9H-purin-9-yl}piperidine-1-carboxylate as a dark oil (37 mg, 86%); LC-MS [M+H]+ 549.1

Step 3: 8-[(6-Bromo-1,3-benzodioxol-5-yl)thio]-9-piperidin-4-yl-9H-purin-6-amine

The product from step 2 (0.037 g, 0.067 mmol) was dissolved in DCM (3 mL), followed by the addition of TFA (3 mL). After stirring for 2 h, the reaction mixture was concentrated in vacuum to provide 8-[(6-bromo-1,3-benzodioxol-5-yl)thio]-9-piperidin-4-yl-9H-purin-6-amine (25 mg, 83%) as a dark oil; LC-MS [M+H]+ 449.09.

### Example Compound 212

### (2S)-1-(4-{6-amino-8-[(6-bromo-1,3-benzodioxol-5-yl)thio]-9H-purin-9-yl}piperidin-1-yl)-1-oxopropan-2-ol

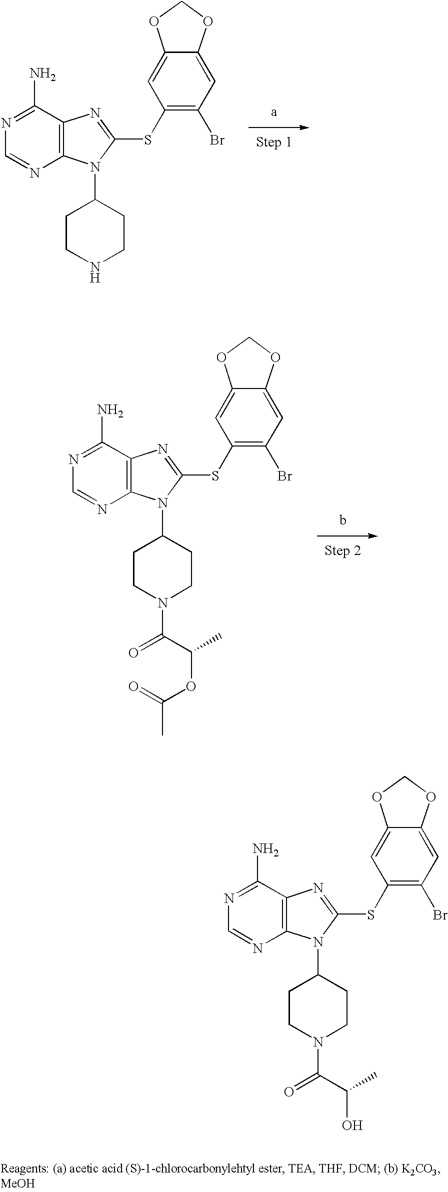

Step 1. (1S)-2-(4-{6-amino-8-[(6-bromo-1,3-benzodioxol-5-yl)thio]-9H-purin-9-yl}piperidin-1-yl)-1-methyl-2-oxoethyl acetate:

8-[(6-bromo-1,3-benzodioxol-5-yl)thio]-9-piperidin-4-yl-9H-purin-6-amine (0.010 g, 0.022 mmol) was dissolved in THF: MeOH (2 mL), followed by the addition of TEA (0.006 mL, 0.044 mmol) and acetic acid (S)-1-chlorocarbonyl-ethyl ester (0.003 mL, 0.022 mmol). After stirring for 4 h, the reaction was concentrated in vacuum and the residue was purified by HPLC to afford (1S)-2-(4-{6-amino-8-[(6-bromo-1,3-benzodioxol-5-yl)thio]-9H-purin-9-yl}piperidin-1-yl)-1-methyl-2-oxoethyl acetate (4 mg, 31%) as a white solid; LC-MS [M+H]⁺ 563.08

Step 2. (2S)-1-(4-{6-Amino-8-[(6-bromo-1,3-benzodioxol-5-yl)thio]-9H-purin-9-yl}piperidin-1-yl)-1-oxopropan-2-ol:

The product from step 1 (4 mg) was dissolved in MeOH (2 mL), followed by the addition of K₂CO₃ (2 mg). After stirring for 4 h, the reaction was filtered and concentrated in vacuum to afford the example compound (3 mg, 75%) as a white solid; ¹H NMR (CD₃OD) δ 8.12 (s, 1H), 7.25 (s, 1H), 7.06 (s, 1H), 6.07 (s, 2H), 4.84-4.70 (m, 1H), 4.66-4.61 (m, 1H). 2.82-2.60 (m, 4H), 3.28-3.25 (m, 2H), 3.25-3.16 (m, 2H), 1.43-1.39 (d, J=6.4 Hz, 3H); LC-MS [M+H]⁺ 521.10

### Example Compound 213

### 4-{6-Amino-8-[(6-bromo-1,3-benzodioxol-5-yl)thio]-9H-purin-9-yl}piperidine-1-carbaldehyde

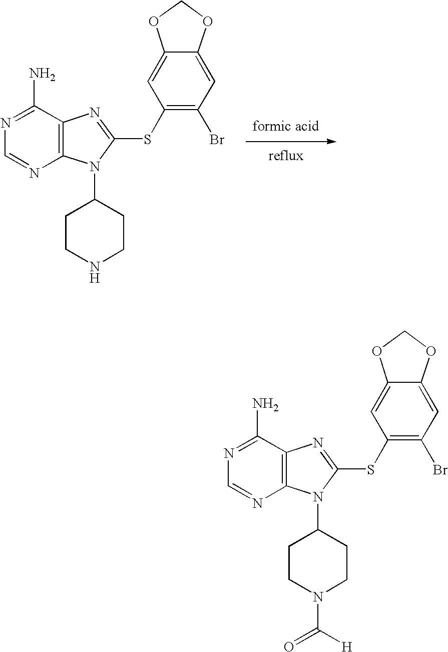

8-[(6-bromo-1,3-benzodioxol-5-yl)thio]-9-piperidin-4-yl-9H-purin-6-amine (0.015 g, 0.0334 mmol) was dissolved in formic acid (5 mL) and the mixture was refluxed for 48 h. After cooling to rt, the mixture was concentrated in vacuum and the residue was purified by HPLC to afford the example compound as white solid (5 mg, 31%); ¹H NMR (CD₃OD) δ 8.23(s, 1H), 8.09 (s, 1H), 7.28 (s, 1H), 7.18 (s, 1H), 6.08 (s, 2H). 4.57-4.52 (m, 1H), 2.87-2.80 (m, 2H), 2.71-2.66 (m, 2H), 2.64-2.55 (m, 2H), 1.97-1.80 (m, 2H); LC-MS [M+H]⁺ 477.03

### Biological and Pharmacological Assays and Examples Hsp90 Binding Assay

Binding of the Example Compounds to purified Hsp90 was assayed by measuring the displacement of BODIPY-labeled geldanamycin (BODIPY-GM) from purified human Hsp90, using a fluorescence polarization assay adapted from Kim et al. (J. Biomolec. Screening, 9(5):375-381 (2004)). Compound dilutions (in 100% DMSO) were added to black-bottom 96-well plates (Greiner Bio-One, Monroe, N.C.) in 2% DMSO final, and equal volumes of BODIPY-GM (10 nM final) and purified human Hsp90 (Stressgen, SPP-770; 30 nM final) (Assay Designs, Ann Arbor, Mich.) in assay buffer (20 mM HEPES-KOH pH 7.3, 50 mM KCl, 5 mM MgCl₂, 20 mM Na₂MoO₄, 0.01% NP-40, 0.1 mg/mL bovine gamma globulin [P2045; Invitrogen, Carlsbad, Calif.], 2 mM DTT) were added sequentially to yield a final volume of 50 microliters. Plates were incubated overnight at room temperature. Parallel and perpendicular fluorescence measurements were read (Analyst AD plate reader; LJL BioSystems, Middletown, Conn.) with excitation/emission wavelengths of 485/530 nm. Background fluorescence (buffer only) was subtracted, and fluorescence polarization (FP) values, expressed in mP units, were calculated from parallel and perpendicular fluorescence readings as follows:

FP=(parallel−perpendicular)/(parallel+perpendicular)*1000.

Percent inhibition was calculated by normalizing the FP values to those obtained in parallel reactions containing DMSO and subtracting these normalized values from 100%. Intrinsic compound fluorescence was independently monitored, and FP data points confounded by compound fluorescence were excluded from the analysis. Results from this assay are given in the third column of Table 10, below. In one embodiment, the invention provides compounds of Formulae Ia, Ib, IIa, IIb, IIIa, IIIc, IVa, IVb, Va, Vb, VIIa, VIIb, VIIa, VIIb, VIIIa, VIIIb, IXa, IXb, Xa, Xb, XIa, XIb, XIIa, XIIb, XIIIa, XIIIb, XIVa, XIVb, XVa, XVb, XVIa, XVIb, XVIII, and XIX, wherein the compounds have an IC₅₀ as measured by this assay (the IC₅₀ values are in the fourth column of Table 10, below) of 10 μM or less, 5 μM or less, 1 μM or less, 0.5 μM or less, 0.25 μM or less, or 0.1 μM or less.

TABLE 10
Binding of example compounds to purified human Hsp90 as
measured by the displacement of BODIPY-labeled
geldanamycin (GM) using fluorescence polarization (FP).

| | GM displacement | |
|---|---|---|
| | from purified | GM displacement |
| | Hsp90 measured | from purified |
| | by FP | Hsp90 measured |
| Example | Inhibition by | by FP |
| Compound | 5 μM compd. | IC 50 |
| No. | (%) | (μM) |
| 1 | | 0.230 |
| 2 | 24.0 | |
| 3 | | 0.080 |
| 4 | | 0.080 |
| 5 | | 0.063 |
| 6 | | 0.070 |
| 7 | | 0.095 |
| 8 | | 0.090 |
| 9 | 87.0 | |
| 10 | | 0.160 |
| 11 | 31.0 | |
| 12 | | 0.065 |
| 13 | 38.0 | |
| 14 | | 1.30 |
| 15 | | 0.170 |
| 16 | 73.0 | |
| 17 | 33.0 | |
| 18 | | 0.450 |
| 19 | 10.0 | |
| 20 | | 3.80 |
| 21 | | 0.099 |
| 22 | 46.0 | |
| 23 | | 0.195 |
| 24 | 28.0 | |
| 25 | | 0.110 |
| 26 | 55.0 | |
| 27 | | 0.110 |
| 28 | 40.0 | |
| 29 | | 1.80 |
| 30 | 23.0 | |
| 31 | | 0.900 |
| 32 | 9.0 | |
| 33 | | 0.300 |
| 34 | 88.0 | |
| 35 | | 0.500 |
| 36 | 35.0 | |
| 37 | | 0.550 |
| 38 | 36.0 | |
| 39 | | 0.200 |
| 40 | 33.0 | |
| 41 | | 0.480 |
| 42 | | 0.500 |
| 43 | | 0.320 |
| 44 | | 0.300 |
| 45 | | 0.220 |
| 46 | | 0.700 |
| 47 | | 0.090 |
| 48 | 77.0 | |
| 49 | | 0.480 |
| 50 | | 1.20 |
| 51 | | 0.370 |
| 52 | | 0.150 |
| 53 | 60.0 | |
| 54 | | 0.550 |
| 55 | 26.0 | |
| 56 | ND | ND |
| 57 | | 0.120 |
| 58 | | 0.180 |
| 59 | ND | ND |
| 60 | | 0.060 |
| 61 | | 0.200 |
| 62 | ND | ND |
| 63 | 34.0 | |
| 64 | | 1.5 |
| 65 | 25.0 | |
| 66 | | 0.073 |
| 67 | ND | ND |
| 68 | | 0.050 |
| 69 | 46.0 | |
| 70 | | 0.455 |
| 71 | 23.0 | |
| 72 | 23.0 | |
| 73 | | 0.260 |
| 74 | 18.0 | |
| 75 | | 2.50 |
| 76 | | 0.070 |
| 77 | | 0.400 |
| 78 | 14.0 | |
| 79 | | 0.700 |
| 80 | 34.0 | |
| 81 | | 0.100 |
| 82 | | 0.295 |
| 83 | 19.0 | |
| 84 | | 0.020 |
| 85 | 35.0 | |
| 86 | | 0.060 |
| 87 | 20.0 | |
| 88 | | 0.080 |
| 89 | ND | ND |
| 90 | | 0.090 |
| 91 | 32.0 | |
| 92 | | 0.090 |
| 93 | 53.0 | |
| 94 | 10.0 | |
| 95 | | 0.700 |
| 96 | 13.0 | |
| 97 | | 0.350 |
| 98 | | 0.450 |
| 99 | | 0.490 |
| 100 | 27.0 | |
| 101 | | 0.200 |
| 102 | | 0.280 |
| 103 | 18.0 | |
| 104 | | 0.800 |
| 105 | 17.0 | |
| 106 | | 0.070 |
| 107 | 59.0 | |
| 108 | | 0.160 |
| 109 | 58.0 | |
| 110 | | >5.0 |
| 111 | 21.0 | |
| 112 | 13.0 | |
| 113 | | 0.072 |
| 115 | ND | ND |
| 116 | 15.0 | |
| 117 | | 5.0 |
| 118 | 12.0 | |
| 119 | ND | ND |
| 120 | | 0.400 |
| 123 | | 3.80 |
| 124 | | 0.295 |
| 125 | | 0.500 |
| 126 | | >5.0 |
| 127 | | >5.0 |
| 128 | | 0.440 |
| 129 | | 3.00 |
| 130 | | 1.10 |
| 131 | | 1.90 |
| 132 | | ND |
| 133 | | 0.505 |
| 134 | 9.0 | |
| 135 | | 0.900 |
| 136 | | 0.110 |
| 137 | | 0.240 |
| 139 | | 0.240 |
| 140 | | 0.045 |
| 141 | | 0.170 |
| 142 | | 0.205 |
| 143 | 27.0 | |
| 145 | | 0.600 |
| 146 | 87.0 | |
| 147 | | 0.470 |
| 148 | | 0.400 |
| 149 | | 0.450 |
| 150 | | 0.600 |
| 151 | | 0.057 |
| 152 | | 0.114 |
| 153 | | 0.175 |
| 154 | | 0.105 |
| 155 | | 0.300 |
| 156 | | 0.160 |
| 157 | | 0.195 |
| 158 | | 0.360 |
| 159 | | 0.150 |
| 160 | | 0.200 |
| 161 | | 0.140 |
| 162 | | 0.060 |
| 163 | | 0.205 |
| 165 | | 0.240 |
| 166 | 75.0 | |
| 167 | | 0.042 |
| 168 | | 0.095 |
| 169 | | 1.90 |
| 170 | | 0.041 |
| 171 | | 0.068 |
| 172 | | 0.3 |
| 173 | | 0.035 |
| 174 | | 0.15 |
| 175 | | 0.07 |
| 176 | | 0.900 |
| 177 | | 0.047 |
| 178 | | 0.305 |
| 179 | | 4.00 |
| 180 | | 0.400 |
| 181 | | 1.50 |
| 182 | | >5.0 |
| 183 | | 2.90 |
| 184 | | 0.310 |
| 185 | | 0.400 |
| 186 | | 0.065 |
| 187 | | 5.00 |
| 188 | | 0.205 |
| 189 | | >5.0 |
| 190 | | 0.650 |
| 191 | | 2.50 |
| 192 | | >5.0 |
| 193 | | 4.300 |
| 194 | | 0.150 |
| 195 | | 0.045 |
| 196 | | 0.100 |
| 197 | | 0.150 |
| 198 | | 0.110 |
| 199 | | 0.190 |
| 200 | | 0.185 |
| 201 | | 0.200 |
| 202 | | 0.180 |
| 203 | | 0.395 |
| 204 | | 0.400 |
| 205 | | 0.497 |
| 206 | | >5.0 |
| 207 | | >5.00 |
| 208 | | >5.00 |
| 209 | | 2.10 |
| 210 | ND | ND |
| 211 | | 0.950 |
| 212 | | 0.350 |
| 213 | | 0.580 |

### Her2-Luciferase Assay:

HCT116 cells stably transfected with a Her2 (kinase domain)-Luciferase fusion expression cassette are seeded into black 96-well plates at 10,000 cells per well in 100 microliters (DMEM supplemented with 10% serum) and incubated overnight. Compound dilutions (in 100% DMSO) are added to individual wells (0.4% DMSO final), and plates are incubated for four hours. Plates are equilibrated to room temperature (5 min), and 100 microliters Steady-Glo reagent (#E2520; Promega, Madison, Wis.) is added per well, and plates are incubated at room temperature for 5 minutes. Luminescence is then measured (TopCount, Perkin-Elmer, Waltham, Mass.).

### Cytotoxicity Assay:

HCT116 cells are seeded into black 96-well plates at 5,000 cells per well in 100 microliters (DMEM supplemented with 10% serum) and are incubated overnight. Compound dilutions (in 100% DMSO) are added to individual wells (0.4% DMSO final), and plates are incubated for 72 hours. Plates are equilibrated to room temperature (5 min). Fifty microliters lysis buffer followed by 50 microliters substrate solution (ATPLite [2 step], #601941, Perkin-Elmer, Waltham, Mass.) is added to each well, and plates are incubated at room temperature 5 minutes. Luminescence is then measured (TopCount, Perkin-Elmer, Waltham, Mass.).

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

What is claimed is:

1. A compound having a structure according to Formulae Ia or Ib: 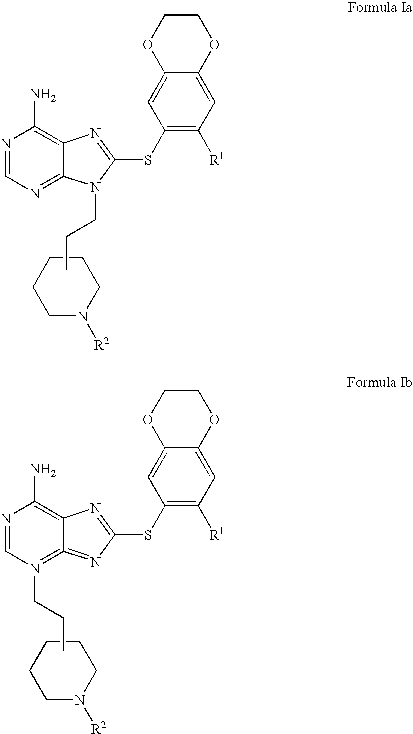
or a pharmaceutically acceptable salt thereof; wherein
R¹ is halo, nitro, cyano, or —C(═O)R¹¹ wherein R¹¹ is hydro or optionally substituted C₁-C₆ alkoxy; for example, R¹ can be —C(═O)H, —C(═O)OCH₃, or —C(═O)OC₂H₅; and
R² is
(a) hydro;
(b) C₁-C₆ alkyl, optionally substituted with 1, 2, 3, 4, or 5 substituents, each independently selected from halo, hydroxyl, amino, cyano, and —C(═O)R²¹ wherein R²¹ is amino;
(c) —C(═O)R³, wherein R³ is selected from the group consisting of:
(1) hydro;
(2) C₁-C₁₀ (e.g., C₁-C₆) alkyl, optionally substituted with 1, 2, 3, 4, or 5 substituents each independently selected from halo, hydroxyl, thiol, cyano, C₁-C₆ haloalkyl (e.g., trifluoromethyl), C₁-C₆ alkoxy (e.g., methoxy) optionally substituted with C₁-C₆ alkoxy (e.g., methoxy), C-amido, N-amido, sulfonyl, and —N(R²²)(R²³), wherein R²² and R²³ are independently selected from hydro, C₁-C₆ alkyl, sulfonyl, and C-carboxy;
(3) C₁-C₆ cycloalkyl, optionally substituted with 1, 2, 3, 4, or 5 substituents independently selected from halo, hydroxyl, amino, cyano, and C₁-C₆ haloalkyl (e.g., trifluoromethyl); and
(4) C₁-C₆ alkoxy, optionally substituted with 1, 2, 3, 4, or 5 substituents independently selected from halo, hydroxyl, amino, cyano, and C₁-C₆ haloalkyl (e.g., trifluoromethyl),
(d) heterocycle or heterocyclylalkyl, optionally substituted with 1, 2, 3, 4, or 5 substituents independently selected from halo, hydroxyl, amino, cyano, trihalomethyl, and C₁-C₄ alkyl optionally substituted with 1, 2, 3, or 4 substituents independently selected from halo, hydroxyl, amino, cyano, and C₁-C₆ haloalkyl (e.g., trifluoromethyl) (e.g., tetrazole-5-yl optionally substituted with 1, 2, 3, or 4 C₁-C₄ alkyl);
(e) sulfonyl; and
(f) optionally substituted heteroaryl;
with the proviso that the compound according to Formulae Ia, is not

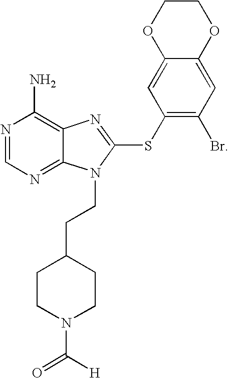

2. A compound according to claim 1, or a pharmaceutically acceptable salt thereof, wherein R¹ is halo, and R² is

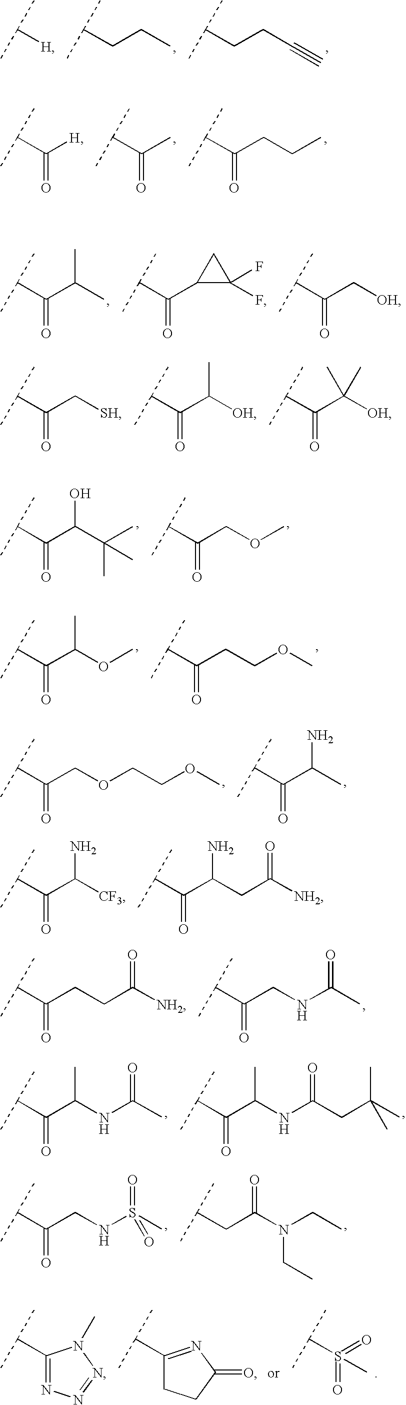

3. A compound according to claim 1, wherein the compound is 8-[(7-bromo-2,3-dihydro-1,4-benzodioxin-6-yl)thio]-9-(2-{1-[(2-methoxyethoxy)acetyl]piperidin-4-yl}ethyl)-9H-purin-6-amine; 8-[(7-bromo-2,3-dihydro-1,4-benzodioxin-6-yl)thio]-9-(2-{1-[(2S)-2-methoxypropanoyl]piperidin-4-yl}ethyl)-9H-purin-6-amine; 8-[(7-bromo-2,3-dihydro-1,4-benzodioxin-6-yl)thio]-9-{2-[1-(3-methoxypropanoyl)piperidin-4-yl]ethyl}-9H-purin-6-amine; 8-[(7-bromo-2,3-dihydro-1,4-benzodioxin-6-yl)thio]-9-{2-[1-(methoxyacetyl)piperidin-4-yl]ethyl}-9H-purin-6-amine; 8-[(7-bromo-2,3-dihydro-1,4-benzodioxin-6-yl)thio]-9-(2-{1-[(2R)-2-methoxypropanoyl]piperidin-4-yl}ethyl)-9H-purin-6-amine; 8-[(7-bromo-2,3-dihydro-1,4-benzodioxin-6-yl)thio]-9-(2-{1-[(2,2-difluorocyclopropyl)carbonyl]piperidin-4-yl}ethyl)-9H-purin-6-amine; 8-[(7-bromo-2,3-dihydro-1,4-benzodioxin-6-yl)thio]-3-(2-{1-[(2,2-difluorocyclopropyl)carbonyl]piperidin-4-yl}ethyl)-3H-purin-6-amine; 8-[(7-bromo-2,3-dihydro-1,4-benzodioxin-6-yl)thio]-9-{2-[1-(methylsulfonyl)piperidin-2-yl]ethyl}-9H-purin-6-amine; 8-[(7-bromo-2,3-dihydro-1,4-benzodioxin-6-yl)thio]-3-{2-[1-(methylsulfonyl)piperidin-2-yl]ethyl}-3H-purin-6-amine; 8-[(7-bromo-2,3-dihydro-1,4-benzodioxin-6-yl)thio]-9-{2-[1-(methylsulfonyl)piperidin-3-yl]ethyl}-9H-purin-6-amine; 8-[(7-bromo-2,3-dihydro-1,4-benzodioxin-6-yl)thio]-3-{2-[1-(methylsulfonyl)piperidin-3-yl]ethyl}-3H-purin-6-amine; 8-[(7-bromo-2,3-dihydro-1,4-benzodioxin-6-yl)thio]-9-[2-(1-propylpiperidin-2-yl)ethyl]-9H-purin-6-amine; 8-[(7-bromo-2,3-dihydro-1,4-benzodioxin-6-yl)thio]-9-{2-[1-(methylsulfonyl)piperidin-4-yl]ethyl}-9H-purin-6-amine; 8-[(7-bromo-2,3-dihydro-1,4-benzodioxin-6-yl)thio]-3-[2-(1-propylpiperidin-2-yl)ethyl]-3H-purin-6-amine; 8-[(7-bromo-2,3-dihydro-1,4-benzodioxin-6-yl)thio]-3-{2-[1-(methylsulfonyl)piperidin-4-yl]ethyl}-3H-purin-6-amine; 8-[(7-bromo-2,3-dihydro-1,4-benzodioxin-6-yl)thio]-9-{2-[1-(1-methyl-1H-tetrazol-5-yl)piperidin-4-yl]ethyl}-9H-purin-6-amine; 8-[(7-bromo-2,3-dihydro-1,4-benzodioxin-6-yl)thio]-3-{2-[1-(1-methyl-1H-tetrazol-5-yl)piperidin-4-yl]ethyl}-3H-purin-6-amine; 8-[(7-bromo-2,3-dihydro-1,4-benzodioxin-6-yl)thio]-9-[2-(1-isobutyrylpiperidin-4-yl)ethyl]-9H-purin-6-amine; 8-[(7-bromo-2,3-dihydro-1,4-benzodioxin-6-yl)thio]-3-[2-(1-isobutyrylpiperidin-4-yl)ethyl]-3H-purin-6-amine; 2-[4-(2-{6-amino-8-[(7-bromo-2,3-dihydro-1,4-benzodioxin-6-yl)thio]-9H-purin-9-yl}ethyl)piperidin-1-yl]-N,N-diethylacetamide; 2-[4-(2-{6-amino-8-[(7-bromo-2,3-dihydro-1,4-benzodioxin-6-yl)thio]-3H-purin-3-yl}ethyl)piperidin-1-yl]-N,N-diethylacetamide; 7-{[6-amino-9-(2-{1-[(2S)-2-methoxypropanoyl]piperidin-4-yl}ethyl)-9H-purin-8-yl]thio}-2,3-dihydro-1,4-benzodioxine-6-carbonitrile; 7-{[6-amino-9-(2-{1-[(2R)-2-methoxypropanoyl]piperidin-4-yl}ethyl)-9H-purin-8-yl]thio}-2,3-dihydro-1,4-benzodioxine-6-carbonitrile; 7-[(6-amino-9-{2-[1-(methoxyacetyl)piperidin-4-yl]ethyl}-9H-purin-8-yl)thio]-2,3-dihydro-1,4-benzodioxine-6-carbonitrile; 7-[(6-amino-9-{2-[1-(methylsulfonyl)piperidin-2-yl]ethyl}-9H-purin-8-yl)thio]-2,3-dihydro-1,4-benzodioxine-6-carbonitrile; 7-[(6-amino-9-{2-[1-(methylsulfonyl)piperidin-3-yl]ethyl}-9H-purin-8-yl)thio]-2,3-dihydro-1,4-benzodioxine-6-carbonitrile; 7-[(6-amino-3-{2-[1-(methylsulfonyl)piperidin-3-yl]ethyl}-3H-purin-8-yl)thio]-2,3-dihydro-1,4-benzodioxine-6-carbonitrile; 7-[(6-amino-9-{2-[1-(methylsulfonyl)piperidin-4-yl]ethyl}-9H-purin-8-yl)thio]-2,3-dihydro-1,4-benzodioxine-6-carbonitrile; 7-[(6-amino-3-{2-[1-(methylsulfonyl)piperidin-4-yl]ethyl}-3H-purin-8-yl)thio]-2,3-dihydro-1,4-benzodioxine-6-carbonitrile; 7-({6-amino-3-[2-(1-formylpiperidin-4-yl)ethyl]-3H-purin-8-yl}thio)-2,3-dihydro-1,4-benzodioxine-6-carbonitrile; 7-({6-amino-9-[2-(1-formylpiperidin-4-yl)ethyl]-9H-purin-8-yl}thio)-2,3-dihydro-1,4-benzodioxine-6-carbonitrile; 7-[(6-amino-9-{2-[1-(1-methyl-1H-tetrazol-5-yl)piperidin-4-yl]ethyl}-9H-purin-8-yl)thio]-2,3-dihydro-1,4-benzodioxine-6-carbonitrile; 7-[(6-amino-3-{2-[1-(1-methyl-1H-tetrazol-5-yl)piperidin-4-yl]ethyl}-3H-purin-8-yl)thio]-2,3-dihydro-1,4-benzodioxine-6-carbonitrile; 7-({9-[2-(1-acetylpiperidin-4-yl)ethyl]-6-amino-9H-purin-8-yl}thio)-2,3-dihydro-1,4-benzodioxine-6-carbonitrile; 4-(2-{6-amino-8-[(7-nitro-2,3-dihydro-1,4-benzodioxin-6-yl)thio]-9H-purin-9-yl}ethyl)piperidine-1-carbaldehyde; 4-(2-{6-amino-8-[(7-nitro-2,3-dihydro-1,4-benzodioxin-6-yl)thio]-3H-purin-3-yl}ethyl)piperidine-1-carbaldehyde; 9-[2-(1-acetylpiperidin-4-yl)ethyl]-8-[(7-nitro-2,3-dihydro-1,4-benzodioxin-6-yl)thio]-9H-purin-6-amine; 3-[2-(1-acetylpiperidin-4-yl)ethyl]-8-[(7-nitro-2,3-dihydro-1,4-benzodioxin-6-yl)thio]-3H-purin-6-amine; 3-[2-(1-acetylpiperidin-4-yl)ethyl]-8-[(7-chloro-2,3-dihydro-1,4-benzodioxin-6-yl)thio]-3H-purin-6-amine; 9-[2-(1-acetylpiperidin-4-yl)ethyl]-8-[(7-chloro-2,3-dihydro-1,4-benzodioxin-6-yl)thio]-9H-purin-6-amine; 8-[(7-bromo-2,3-dihydro-1,4-benzodioxin-6-yl)thio]-9-[2-(1-butyrylpiperidin-4-yl)ethyl]-9H-purin-6-amine; 8-[(7-bromo-2,3-dihydro-1,4-benzodioxin-6-yl)thio]-3-[2-(1-butyrylpiperidin-4-yl)ethyl]-3H-purin-6-amine; (2S)-1-[4-(2-{6-amino-8-[(7-bromo-2,3-dihydro-1,4-benzodioxin-6-yl)thio]-9H-purin-9-yl}ethyl)piperidin-1-yl]-1-oxopropan-2-ol; (2S)-1-[4-(2-{6-amino-8-[(7-bromo-2,3-dihydro-1,4-benzodioxin-6-yl)thio]-3H-purin-3-yl}ethyl)piperidin-1-yl]-1-oxopropan-2-ol; 2-[4-(2-{6-amino-8-[(7-bromo-2,3-dihydro-1,4-benzodioxin-6-yl)thio]-9H-purin-9-yl}ethyl)piperidin-1-yl]-2-oxoethanol; 2-[4-(2-{6-amino-8-[(7-bromo-2,3-dihydro-1,4-benzodioxin-6-yl)thio]-3H-purin-3-yl}ethyl)piperidin-1-yl]-2-oxoethanol; 1-[4-(2-{6-amino-8-[(7-bromo-2,3-dihydro-1,4-benzodioxin-6-yl)thio]-9H-purin-9-yl}ethyl)piperidin-1-yl]-2-methyl-1-oxopropan-2-ol; 1-[4-(2-{6-amino-8-[(7-bromo-2,3-dihydro-1,4-benzodioxin-6-yl)thio]-3H-purin-3-yl}ethyl)piperidin-1-yl]-2-methyl-1-oxopropan-2-ol; 7-[(6-amino-9-{2-[1-(2-hydroxy-2-methylpropanoyl)piperidin-4-yl]ethyl}-9H-purin-8-yl)thio]-2,3-dihydro-1,4-benzodioxine-6-carbonitrile; 7-{[6-amino-9-(2-{1-[(2S)-2-hydroxypropanoyl]piperidin-4-yl}ethyl)-9H-purin-8-yl]thio}-2,3-dihydro-1,4-benzodioxine-6-carbonitrile; 7-{[6-amino-3-(2-{1-[(2S)-2-hydroxypropanoyl]piperidin-4-yl}ethyl)-3H-purin-8-yl]thio}-2,3-dihydro-1,4-benzodioxine-6-carbonitrile; 7-({6-amino-9-[2-(1-glycoloylpiperidin-4-yl)ethyl]-9H-purin-8-yl}thio)-2,3-dihydro-1,4-benzodioxine-6-carbonitrile; (2S)-1-[4-(2-{6-amino-8-[(7-bromo-2,3-dihydro-1,4-benzodioxin-6-yl)thio]-9H-purin-9-yl}ethyl)piperidin-1-yl]-3,3-dimethyl-1-oxobutan-2-ol; (2S)-1-[4-(2-{6-amino-8-[(7-bromo-2,3-dihydro-1,4-benzodioxin-6-yl)thio]-3H-purin-3-yl}ethyl)piperidin-1-yl]-3,3-dimethyl-1-oxobutan-2-ol; (2R)-1-[4-(2-{6-amino-8-[(7-bromo-2,3-dihydro-1,4-benzodioxin-6-yl)thio]-9H-purin-9-yl}ethyl)piperidin-1-yl]-1-oxopropan-2-ol; 9-[2-(1-acetylpiperidin-4-yl)ethyl]-8-[(7-bromo-2,3-dihydro-1,4-benzodioxin-6-yl)thio]-9H-purin-6-amine; 8-[(7-bromo-2,3-dihydro-1,4-benzodioxin-6-yl)thio]-9-(2-piperidin-4-ylethyl)-9H-purin-6-amine; (3S)-3-amino-4-[4-(2-{6-amino-8-[(7-bromo-2,3-dihydro-1,4-benzodioxin-6-yl)thio]-9H-purin-9-yl}ethyl)piperidin-1-yl]-4-oxobutanamide; 4-[4-(2-{6-amino-8-[(7-bromo-2,3-dihydro-1,4-benzodioxin-6-yl)thio]-9H-purin-9-yl}ethyl)piperidin-1-yl]-4-oxobutanamide; 5-[4-(2-{6-amino-8-[(7-bromo-2,3-dihydro-1,4-benzodioxin-6-yl)thio]-9H-purin-9-yl}ethyl)piperidin-1-yl]-3,4-dihydro-2H-pyrrol-2-one; N-{2-[4-(2-{6-amino-8-[(7-bromo-2,3-dihydro-1,4-benzodioxin-6-yl)thio]-9H-purin-9-yl}ethyl)piperidin-1-yl]-2-oxoethyl}methanesulfonamide; N-{2-[4-(2-{6-amino-8-[(7-bromo-2,3-dihydro-1,4-benzodioxin-6-yl)thio]-9H-purin-9-yl}ethyl)piperidin-1-yl]-2-oxoethyl}acetamide; N-{(1S)-2-[4-(2-{6-amino-8-[(7-bromo-2,3-dihydro-1,4-benzodioxin-6-yl)thio]-9H-purin-9-yl}ethyl)piperidin-1-yl]-1-methyl-2-oxoethyl}acetamide; N-{(1R)-2-[4-(2-{6-amino-8-[(7-bromo-2,3-dihydro-1,4-benzodioxin-6-yl)thio]-9H-purin-9-yl}ethyl)piperidin-1-yl]-1-methyl-2-oxoethyl}acetamide; or 2-[4-(2-{6-amino-8-[(7-bromo-2,3-dihydro-1,4-benzodioxin-6-yl)thio]-9H-purin-9-yl}ethyl)piperidin-1-yl]-2-oxoethanethiol;
or a pharmaceutically acceptable salt thereof.

4. A compound according to claim 3, wherein said compound is (2S)-1-[4-(2-{6-amino-8-[(7-bromo-2,3-dihydro-1,4-benzodioxin-6-yl)thio]-9H-purin-9-yl}ethyl)piperidin-1-yl]-1-oxopropan-2-ol; 2-[4-(2-{6-amino-8-[(7-bromo-2,3-dihydro-1,4-benzodioxin-6-yl)thio]-9H-purin-9-yl}ethyl)piperidin-1-yl]-2-oxoethanol; (2R)-1-[4-(2-{6-amino-8-[(7-bromo-2,3-dihydro-1,4-benzodioxin-6-yl)thio]-9H-purin-9-yl}ethyl)piperidin-1-yl]-1-oxopropan-2-ol; 8-[(7-bromo-2,3-dihydro-1,4-benzodioxin-6-yl)thio]-9-(2-piperidin-4-ylethyl)-9H-purin-6-amine; 9-[2-(1-acetylpiperidin-4-yl)ethyl]-8-[(7-bromo-2,3-dihydro-1,4-benzodioxin-6-yl)thio]-9H-purin-6-amine; or 2-[4-(2-{6-amino-8-[(7-bromo-2,3-dihydro-1,4-benzodioxin-6-yl)thio]-9H-purin-9-yl}ethyl)piperidin-1-yl]-2-oxoethanethiol;
or a pharmaceutically acceptable salt thereof.

5. A pharmaceutical composition comprising at least one compound according to claim 1, and a pharmaceutically acceptable carrier or excipient.

6. A pharmaceutical composition comprising at least one compound according to claim 2, and a pharmaceutically acceptable carrier or excipient.

7. A pharmaceutical composition comprising at least one compound according to claim 3, and a pharmaceutically acceptable carrier or excipient.

8. A pharmaceutical composition comprising at least one compound according to claim 4, and a pharmaceutically acceptable carrier or excipient.

9. A method of treating an Hsp90 inhibitor-sensitive cancer, comprising identifying a patient in need of such treatment and administering to said patient a therapeutically effective amount of a compound of claim 1.

10. The method of claim 9, wherein said cancer is Hodgkin's disease, non-Hodgkin's lymphoma, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, or prostatic carcinoma.

11. A method of treating an Hsp90 inhibitor-sensitive cancer, comprising identifying a patient in need of such treatment and administering to said patient a therapeutically effective amount of a compound of claim 2.

12. The method of claim 11, wherein said cancer is Hodgkin's disease, non-Hodgkin's lymphoma, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, or prostatic carcinoma.

13. A method of treating an Hsp90 inhibitor-sensitive cancer, comprising identifying a patient in need of such treatment and administering to said patient a therapeutically effective amount of a compound of claim 3.

14. The method of claim 13, wherein said cancer is Hodgkin's disease, non-Hodgkin's lymphoma, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, or prostatic carcinoma.

15. A method of treating an Hsp90 inhibitor-sensitive cancer, comprising identifying a patient in need of such treatment and administering to said patient a therapeutically effective amount of a compound of claim 4, wherein said cancer is Hodgkin's disease, non-Hodgkin's lymphoma, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, or prostatic carcinoma.

16. A compound according to Formulae XIIIa: 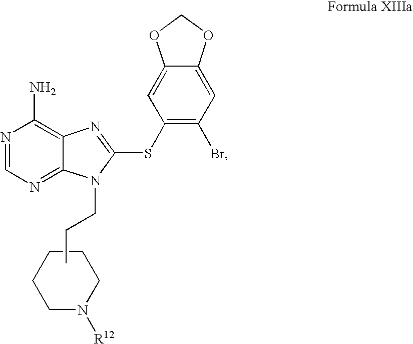
or a pharmaceutically acceptable salt thereof; wherein
R¹² is hydro,

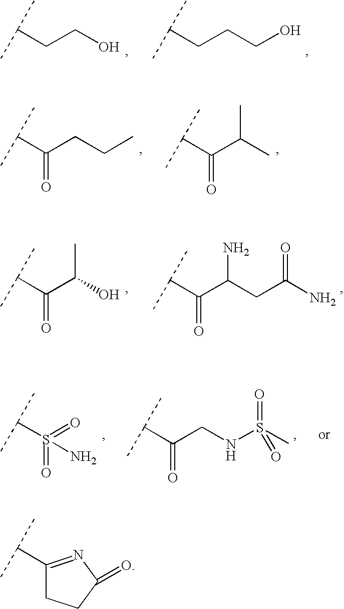

17. A compound according to claim 16, wherein R¹² is hydro, and said compound is 8-[(6-bromo-1,3-benzodioxol-5-yl)thio]-9-(2-piperidin-4-ylethyl)-9H-purin-6-amine, or a pharmaceutically acceptable salt thereof.

18. A method of making the compound of claim 17, comprising the steps of
(a) reacting adenine and 4-(2-bromo-ethyl)-piperidine-1-carboxylic acid tert-butyl ester in the presence of organic or inorganic base in organic solvent at 20-130° C., using suitable reagents under suitable conditions to yield tert-butyl 4-[2-(6-amino-9H-purin-9-yl)ethyl]piperidine-1-carboxylate;
(b) brominating tert-butyl 4-[2-(6-amino-9H-purin-9-yl)ethyl]piperidine-1-carboxylate using suitable reagents under suitable conditions to yield tert-butyl 4-[2-(6-amino-8-bromo-9H-purin-9-yl)ethyl]piperidine-1-carboxylate;
(c) reacting tert-butyl 4-[2-(6-amino-8-bromo-9H-purin-9-yl)ethyl]piperidine-1-carboxylate with benzo[1,3]dioxole-5-thiol using suitable reagents under suitable conditions to yield tert-butyl 4-{2-[6-amino-8-(1,3-benzodioxol-5-ylthio)-9H-purin-9-yl]ethyl}piperidine-1-carboxylate; and
(d) brominating tert-butyl 4-{2-[6-amino-8-(1,3-benzodioxol-5-ylthio)-9H-purin-9-yl]ethyl}piperidine-1-carboxylate using suitable reagents under suitable conditions to yield 8-[(6-bromo-1,3-benzodioxol-5-yl)thio]-9-(2-piperidin-4-ylethyl)-9H-purin-6-amine.

19. A compound that is 2-ethylhexyl 3-[(7-bromo-2,3-dihydro-1,4-benzodioxin-6-yl)thio]propanoate.

20. A method of making the compound of claim 19 comprising reacting reacting 6,7-dibromo-2,3-dihydro-1,4-benzodioxine with 2-ethylhexyl-3-mercaptopropionate using suitable reagents under suitable conditions to yield 2-ethylhexyl 3-[(7-bromo-2,3-dihydro-1,4-benzodioxin-6-yl)thio]propanoate.
